(19) United States
(12) Patent Application Publication
(10) Pub. No.: US 2021/0223248 A1
(43) Pub. Date: Jul. 22, 2021

(54) BIOMARKERS, USES THEREOF FOR SELECTING IMMUNOTHERAPY INTERVENTION, AND IMMUNOTHERAPY METHODS

(71) Applicant: FRED HUTCHINSON CANCER RESEARCH CENTER, Seattle, WA (US)

(72) Inventors: Cameron J. TURTLE, Seattle, WA (US); Kevin A. HAY, Vancouver (CA); Alexandre Vinaud HIRAYAMA, Seattle, WA (US); Jordan GAUTHIER, Seattle, WA (US)

(21) Appl. No.: 15/734,205

(22) PCT Filed: May 31, 2019
(86) PCT No.: PCT/US2019/035054

§ 371 Date: Dec. 1, 2020

Related U.S. Application Data
(60) Provisional application No. 62/679,468, filed on Jun. 1, 2018.
(60) Provisional application No. 62/751,466, filed on Oct. 26, 2018.
(60) Provisional application No. 62/754,524, filed on Nov. 1, 2018.

(51) Int. Cl. G01N 33/574

(52) U.S. Cl. CPC G01N 33/57426; G01N 2333/904; G01N 33/57484

(57) ABSTRACT

The instant disclosure provides biomarkers and methods for identifying subjects at risk of relapse or suitable for allogeneic hematopoietic stem cell transplant after adoptive immunotherapy to guide preemptive intervention, modified therapy, or the like. Exemplary biomarkers include pre-lymphodepletion levels of serum lactate dehydrogenase (LDH), pre-lymphodepletion levels of platelets, levels of MCP-1, levels of IL-17, and pre-treatment regimen disease pathology. Based on the determined risk-relapse profile, an at-risk subject may be treated with pre-emptive therapy, while a subject not at risk for relapse may not receive further treatment, or may receive an allogeneic hematopoietic stem cell transplant. Also provided are methods for treating a hematological malignancy, wherein certain embodiments of the methods comprise adoptive cell therapy in the context of BTK-inhibitor therapy and/or BTK-inhibitor therapy in the context of adoptive cell therapy. Also provided are methods for treating follicular lymphoma (FL).

### STATEMENT OF GOVERNMENT INTEREST

This invention was made with government support under CA136551, CA015704, and DK056465, awarded by the National Institutes of Health. The government has certain rights in the invention.

### BACKGROUND

Lymphodepletion chemotherapy followed by infusion of T cells that are genetically modified to express a chimeric antigen receptor (i.e., CAR-modified T cells) has produced high response rates in clinical studies, such as in refractory B-cell acute lymphoblastic leukemia (B-ALL), chronic lymphocytic leukemia (CLL), and non-Hodgkin's lymphoma (NHL) (Davila et al., Sci. Transl. Med. 6:224ra25, 2014; Kochenderfer et al., J. Clin. Oncol. 33:540, 2015; Maude et al., N. Engl. J. Med. 371:1507, 2014; Porter et al., Sci. Transl. Med. 7:303ra139, 2015; Turtle et al. I, J. Clin. Invest. 126:2123, 2016; Turtle et al. II, Sci. Transl. Med. 8:355ra116, 2016). Durable complete responses (CRs) without subsequent anti-tumor therapy have been observed in a subset of patients who received CD19 CAR-T cell therapy, demonstrating the potential of this approach (Turtle et al. I and II, 2016; Porter et al., 2015).

For example, when infused antigen-specific CAR-modified T cells encounter an antigen positive target cell, in vivo signaling through the CAR induces CAR-T cell proliferation, cytokine secretion, and target cell lysis (Turtle et al., Clin. Pharmacol. Ther. 100:252, 2016). However, knowledge of which patient and treatment factors are associated with and/or predictive of achieving complete responses and long-term disease-free or progression-free survival following CAR-T cell therapy is developing.

Hence, there remains a need in the art for biomarkers and treatment factors to identify a patient as being at risk for relapse or having a positive long-term prognosis following CAR-T cell therapy. The present disclosure meets such needs, and further provides other related advantages.

### BRIEF DESCRIPTION OF THE DRAWINGS

FIG. 1 provides a schematic diagram of a transgene construct encoding an anti-CD19 chimeric antigen receptor (CAR) according to the present disclosure.

FIGS. 2A and 2B show (A) Disease-Free Survival (DFS) and (B) Overall Survival (OS) of B-ALL patients who did or did not achieve MRD-negative CR following CAR-T cell therapy. FIG. 2C shows DFS in the MRD-negative CR patients who had a leukemic clone detected by high-throughput sequencing (HTS, n=28) prior to CAR-T cell therapy. DFS of patients in whom a leukemic clone was not detected following CAR-T cell therapy (“HTS-neg”) is represented by the upper curve. DFS of patients in whom a leukemic clone was detected following CAR-T cell therapy (“HTS-pos”) is represented by the lower curve. The top graph in each figure shows the survival probability of each patient strata. The bottom graph in each figure shows the number of patients within each strata who were considered to be at risk for relapse over time following CAR-T cell infusion. Data are based on a median follow-up of 30.9 months following administration of CAR-T cells.

FIGS. 3A and 3B show (A) the concentration CAR-T cells from B-ALL patients achieving or not achieving MRD-negative CR following CAR-T cell infusion, as determined by quantitative PCR (qPCR) measuring the concentration of the CAR lentiviral marker FlapEF1α DNA in overall DNA, and (B) the peak number of CAR-T cells observed in each patient group.

FIGS. 4A and 4B show 30-month (A) DFS and (B) OS curves of B-ALL patients who, prior to CAR-T cell infusion, did or did not (i) have normal serum levels of lactate dehydrogenase (LDH; <210 U/L) prior to lymphodepleting chemotherapy, and (ii) receive Cy/Flu lymphodepleting chemotherapy. FIGS. 4C and 4D show 30-month (C) DFS and (D) OS curves of B-ALL patients who, prior to CAR-T infusion, did or did not have normal levels of LDH, serum platelet counts above 100 U/L prior to lymphodepleting chemotherapy, and received Cy/Flu lymphodepleting chemotherapy. The top graph in each figure shows the survival probability of each patient strata. The bottom graph in each figure shows the number of patients within each strata considered to be at risk for relapse over time following CAR-T cell infusion.

FIGS. 5A and 5B show 30-month (A) DFS and (B) OS curves of B-ALL patients who, following treatment with CAR-T cells, did or did not receive allo-HSCT while in MRD-negative CR. The top graph in each figure shows the survival probability of the two patient strata. The bottom graph in each figure shows the number of patients within each strata who were considered to be at risk for relapse over time following CAR-T cell infusion.

FIGS. 6A and 6B show (A) the cumulative incidence of relapse (CIR) and (B) non-relapse mortality (NRM) observed in B-ALL patients who, following treatment with lymphodepleting chemotherapy and CAR-T cell infusion, received allo-HSCT while in MRD-negative CR.

FIGS. 7A and 7B show 30-month (A) DFS and (B) OS curves of B-ALL patients who (i) had normal serum LDH and platelet counts >100 prior to receiving Cy/Flu and (ii), following infusion with CAR-T cells, did or did not receive allo-HSCT while in MRD-negative CR. The top graph in each figure shows the survival probability of the two patient strata. The bottom graph in each figure shows the number of patients within each strata who were considered to be at risk for relapse over time following CAR-T cell infusion.

FIGS. 8A and 8B show (top) the association of “good risk” factors (pre-lymphodepletion serum LDH of less than 210 U/L; pre-lymphodepletion platelet counts at or above 100 U/L; received Cy/Flu lymphodepletion chemotherapy) and “bad risk” factors (pre-lymphodepletion serum LDH of 210 U/L or more; pre-lymphodepletion platelet counts below 100 U/L; did not receive Cy/Flu), as determined by multivariate analysis, with (A) DFS and (B) OS in patients who achieved MRD-negative CR following CAR-T cell therapy; (bottom) the number of patients within each strata considered to be at risk for relapse over time following CAR-T cell infusion.

FIGS. 9A and 9B show (top) the probability of PFS over 30-months post CAR-T infusion in NHL patients with (A) indolent or aggressive tumor histology or with (B) an International Prognostic Index (IPI) score of 0-1 or 2-4.

FIGS. 9C and 9D show (top) the probability of PFS (C) and OS (D) in NHL patients who achieved (upper curve) or did not achieve (lower curve) CR after CAR-T infusion.

FIGS. 9E and 9F show (top) the probability of PFS (E) and OS (F) in NHL patients with indolent lymphoma who achieved (upper line) or did not achieve (lower line; no lower line for OS) CR after CAR-T infusion. FIGS. 9G and 9H show (top) the probability of PFS (G) and OS (H) in NHL patients with aggressive lymphoma who achieved (upper curve) or did not achieve (lower curve) CR after CAR-T infusion. The bottom graph in each of FIGS. 9A-9H shows the number of patients within each stratum that were considered to be at risk for relapse over time following CAR-T cell infusion.

FIG. 10 shows the best response rates (Lugano classification system) of NHL patients (aggressive disease strata vs. indolent disease histology strata vs. all patients) to Cy/Flu followed by CAR-T cell infusion.

FIGS. 11A and 11B show that complete remission (CR) is associated with better (A) progression-free survival (PFS) and (B) overall survival (OS) in NHL patients who received Cy/Flu followed by CAR-T cell infusion, according to the present disclosure. CI=confidence interval. Medial follow-up in CR patients was 20.21 months after CAR-T cell infusion (95% CI: 16.5-28.03 mo.). Response was not evaluable in one patient. P-values were calculated using Logrank test.

FIGS. 12A-12F show that serum LDH, serum MCP-1, and in vivo CAR-T cell kinetics are associated with responses of NHL patients to CAR-T cell therapy. (A) CD8+ CAR-T cell counts in the indicated patient strata following infusion up to Day-28. (B) Peak CD8+ CAR-T cell counts in best-responding patients. (C) Estimated probability of achieving CR (y-axis) versus peak of CD8+ CAR-T cells (x-axis). (D) Estimated probability of achieving CR (y-axis) versus pre-lymphodepletion serum LDH (x-axis). (E), (F) Serum MCP-1 in patients who received bridging therapy (“Yes”) and in those who did not receive bridging therapy (“No”) [(E), pre-lymphodepletion (log 10 pg/mL); (F), day 0 (log 10 pg/mL)]. Each point represents data from a single patient. Box and whisker plots show the median (bar) and interquartile range (box). P-values were calculated using the Wilcoxon rank-sum test.

FIGS. 13A-13E show analysis of serum biomarkers associated with durable PFS in aggressive NHL. (A) The association of pre-lymphodepletion (pre-LD) serum LDH with the hazard of a PFS event, adjusting for day 0 MCP-1 and peak IL-7. (B) The association of serum day 0 MCP-1 with the hazard of a PFS event, adjusting for pre-lymphodepletion LDH and peak IL-7. (C) Estimated combined effect of pre-lymphodepletion serum LDH and the day 0 MCP-1 concentration on hazard of a PFS event. (D) The association of serum peak IL-7 with the hazard of a PFS event, adjusting for day 0 MCP-1 and pre-lymphodepletion LDH. (E) Estimated combined effect of pre-lymphodepletion serum LDH and the peak IL-7 concentration on hazard of a PFS event. The hazard ratios are shown in the bars to the right of each figure. Serum biomarkers were modeled as a cubic spline with three knots.

FIGS. 14A-14C show that IL-7 and IL-18 kinetics are associated with PFS in NHL patients who received CAR-T cell therapy according to the present disclosure. (A) Serum IL-7 concentration prior to and following CAR-T cell infusion in patients with ongoing CR and who experienced subsequent disease progression. (B) Serum IL-18 concentration prior to and following CAR-T cell infusion in patients with ongoing CR and who experienced subsequent disease progression. (C) (Top) Survival curves of patients with cytokine kinetics as indicated; (bottom) number of patients within each strata considered to be at risk of relapse over time following CAR-T cell infusion.

FIGS. 14D and 14E show Kaplan-Meier estimates of PFS (D) and OS (E) in aggressive NHL patients who achieved CR and had serum IL-7 peak above the median (upper curve) compared to those who had serum IL-7 peak below the median (lower curve). The number of patients at risk at each timepoint are indicated. Log-rank tests were used to compare between-group differences in survival curves.

FIGS. 14F-14J show higher serum MCP-1 and IL-7, and higher CAR-T cell counts in aggressive NHL patients who received more intensive lymphodepletion. (F-H) Biomarkers in patients who received high-intensity Cy/Flu lymphodepletion and in those who received low-intensity Cy/Flu lymphodepletion (F), serum MCP-1 delta from lymphodepletion to day 0 (log 10 pg/mL); Serum day 0 MCP-1 (G) and peak IL-7 (H) concentrations in patients who received high-intensity Cy/Flu lymphodepletion and in those who received low-intensity Cy/Flu lymphodepletion. Each point represents data from a single patient. Box and whisker plots show the median (bar) and interquartile range (box). Adjusted P values were calculated using the Wilcoxon rank-sum test. (I) Serum day 0 MCP-1 and peak IL-7 concentrations according to lymphodepletion intensity (Spearman correlation r=0.52, P=0.00014). Each point represents data from a single patient. (J) Higher CAR-T cell counts in aggressive NHL patients with high-intensity lymphodepletion or a favorable cytokine profile. (Top and middle graphs) qPCR showing CAR-T cell copies/μg DNA in patients who received high-intensity Cy/Flu lymphodepletion and in those who received low-intensity Cy/Flu lymphodepletion (P-value=0.0007 for the difference in the peak of CAR-T cells between the groups; top), and in patients with serum day 0 MCP-1 concentration above and below the median (P-value<0.0001 for the difference in the peak of CAR-T cells between the groups; middle). Smoothing curves with 95% confidence intervals were used to summarize the data. Adjusted P values were calculated using the Wilcoxon rank-sum test. (Bottom graphs) Association of day 28 CAR-T cell counts by qPCR (log 10 copies/μg DNA) and the hazard of PFS event when CAR-T cell counts is modeled as a cubic spline with three knots. Tick marks represent data from individual patients.

FIGS. 14K-14N show in vivo CAR-T cell expansion in the blood correlated with peak serum IL-7, MCP-1 delta from lymphodepletion to day 0, and day 0 serum MCP-1 in aggressive NHL. (K, L) CAR-T cell AUC0-28 (L) or AUCpeak-28 (M) according to peak serum IL-7 (pg/mL). (M, N) Peak of CD8+ CAR-T cells/μL (M) or CAR transgene copies/μg of DNA (N) according to serum MCP-1 delta from lymphodepletion to day 0 (log 10 pg/mL).

FIGS. 14O-14Q show slower CAR-T cell contraction in aggressive NHL patients with ongoing CR. (O) CAR-T cell AUCpeak-28 by qPCR (copies/μg DNA) in patients with ongoing CR and in those who relapsed. Each point represents data from a single patient. Box and whisker plots show the median (bar) and interquartile range (box). Adjusted P-values were calculated using the Wilcoxon rank-sum test. (P) Association of AUCpeak-28 and the hazard of PFS event when AUCpeak-28 is modeled as a cubic spline with three knots. (Q) Association of day 28 CAR-T cell counts by qPCR (copies/μg DNA) and the hazard of PFS event when cell counts is modeled as a cubic spline with three knots. Tick marks represent data from individual patients.

FIG. 14R shows better PFS in aggressive NHL patients who received high-intensity lymphodepletion and in those with high serum MCP-1 and IL-7. Top: Kaplan-Meier estimates of PFS according to lymphodepletion intensity (high intensity=“HI Cy/Flu,” upper curve; low intensity=“LI Cy/Flu,” lower curve) in all aggressive NHL patients. Bottom: the numbers of patients at risk at each timepoint. Log-rank tests were used to compare between-group differences in survival curves.

FIGS. 14S-14X show better PFS in aggressive NHL patients with higher MCP-1 and IL-7 and with lower LDH and impact of lymphodepletion and serum MCP-1 and IL-7 on PFS. (S-T, W-X) Kaplan-Meier estimates of PFS according to lymphodepletion intensity and serum day 0 MCP-1 and IL-7 peak concentrations (above or below median) (S), according to pre-lymphodepletion LDH concentration below (black) or above (red) the upper limit of normal (T), according to lymphodepletion intensity in aggressive NHL patients with pre-lymphodepletion LDH >upper limit of normal (ULN) (W), according to high serum MCP-1 day 0 (>median) and high serum IL-7 peak (>median) versus one or both low concentrations in aggressive NHL patients with LDH above ULN (X). The numbers of patients at risk at each timepoint are indicated. Log-rank tests were used to compare between-group differences in survival curves. (U) Association of high-intensity and low-intensity lymphodepletion and the hazard of a PFS event according to pre-lymphodepletion serum LDH. (V) Association of favorable or unfavorable cytokine profile and the hazard of a PFS event according to pre-lymphodepletion serum LDH. Cytokines were modeled as a cubic spline with three knots.

FIG. 14Y Serum concentrations of MCP-1 in CR and non-CR patients prior to lymphodepletion and at Day 0 pre-infusion with CAR-T cells.

FIGS. 15A and 15B summarize ibrutinib treatments of CLL patients who also received anti-CD19 CAR-T cell therapy according of the present disclosure. (A) Twelve patients (63%), all of whom had failed ibrutinib (progressive disease, PD, n=11; stable disease, SD, n=1), were still receiving ibrutinib at study enrollment (median time on ibrutinib before leukapheresis, 726 days; range, 78-2185). (B) Seven patients (37%) who had previously ceased ibrutinib due to PD recommenced it shortly before leukapheresis (median time on ibrutinib before leukapheresis, 24 days; range, 15-30). Each horizontal bar indicates the timing and duration of ibrutinib therapy prior to and during study for a single patient. For these patients, only the last ibrutinib treatment prior to study participation is shown. Vertical lines represent the time of CAR-T cell infusion. Double arrows=discontinuation of ibrutinib; single arrow=continuation of ibrutinib at last follow-up.

FIG. 16A shows (top) progression-free survival (PFS) probability curves for patients in the ibrutinib cohort based on IgH sequencing of bone marrow and (bottom) the number of patients negative or positive for a diseased B cell clone (IgH sequencing) who were at risk of disease progression over time, following treatment with CAR-T cells. FIG. 16B shows PFS probability curves (top) and risk/time summaries (bottom) for the patients in the no-ibrutinib cohort.

FIGS. 17A-17D show CAR-T cells in patients receiving concurrent ibrutinib and CD19 CAR-T cells. CAR-T transgene peaks measured by qPCR (FLAP-EF1α copies/μg of genomic DNA in blood). (A, B) CAR-T transgene copies in responders by iwCLL (A) and iwCLL CT (B) criteria. (C, D) CAR-T cell transgene copies in patients without detectable marrow disease by flow cytometry (C) or by IGH sequencing (D). P values per Wilcoxon Rank Sum test (one-sided). Abbreviations: CAR, chimeric antigen receptor; CRS, cytokine release syndrome; CR, complete response; PR, partial response; iwCLL, international workshop chronic lymphocytic leukemia.

FIGS. 18A-18D show overall and progression-free survival probabilities after CD19 CAR-T cell therapy with concurrent ibrutinib. In responding patients by iwCLL criteria the 1-year probability of OS (A) and PFS (B) were 80% (95% CI: 57-100) and 49% (95% CI: 23-100), respectively. In patients who achieved an MRD-negative marrow response by flow cytometry (n=13), the 1-year probabilities of OS and PFS were 92% (95% CI: 79-100) and 58% (95% CI: 29-100), respectively. The depth of marrow response was associated with PFS. Among patients who cleared disease from marrow by flow cytometry four weeks after CAR-T infusion, achieving MRD-negativity. In patients who achieved MRD-negative marrow response by IGH sequencing (n=11), the 1-year OS (C) and PFS (D) probabilities were 100% (95% CI: 12-100) and 62% (95% CI: 32-100), respectively. In this group, two late relapses were observed at 10.2 and 12 months after infusion. Abbreviations: CAR, chimeric antigen receptor; CR, complete response; PR, partial response; iwCLL, international workshop chronic lymphocytic leukemia; MRD, minimal residual disease. P values per log rank test.

FIGS. 19A and 19B show overall survival (A) and progression-free survival (B) of the entire patient cohort.

FIGS. 20A-20D show retrospective Bayesian comparisons of toxicity and efficacy between the concurrent ibrutinib cohort and the no-ibrutinib cohort. Shown are the estimated posterior probability distributions, using a non-informative prior probability distribution. Discontinued lines show the 95% credible interval. Abbreviations: Con-ibr, concurrent ibrutinib; No-ibr, ibrutinib discontinued prior to lymphodepletion; CRS, cytokine release syndrome; iwCLL, international workshop chronic lymphocytic leukemia.

FIGS. 21A-21F show numbers of CAR-T cells measured in the peripheral blood of patients in the concurrent ibrutinib cohort. All patients received 2×10⁶ CAR-T cells/kg and CyFlu lymphodepletion. Seven patients were excluded (2 early deaths, 5 patients received 2×10⁵ CAR-T cells/kg). (A, C) The bolded curves are polynomial regression lines using the LOESS (locally estimated scatterplot smoothing) method, the shaded area showing the 95% CI of the estimate. (B, D, E, F): P values per Wilcoxon Sum Rank test (one-sided). (E, F): CAR-T cells per CRS grade per modified Lee consensus criteria. Abbreviations: Con-ibr, concurrent ibrutinib; No-ibr, ibrutinib discontinued prior to lymphodepletion CAR, chimeric antigen receptor; CRS, cytokine release syndrome.

FIGS. 22A-22H show in vivo kinetics of cytokines associated with CRS and/or and neurotoxicity (NT) before and after CAR-T cell infusion (“0” on y-axes) in the two patient cohorts. Cytokines analyzed were: IL-8 (A, B); IL-15 (C, D); MCP-1 (E, F); and IL-6 (G). Soluble IL-2Rα was also measured (H). Kinetics over time are shown in FIGS. 22A, 22C, and 22E. Peak concentrations are shown in FIGS. 22B, 22D, 22F, 22G, and 22H.

FIGS. 23A-23C show in vivo expansion and persistence of CAR-T cells during CRS events of varying severity (key at right) in the two patient cohorts. (A) log 10 count of CD8⁺ CAR-T cells/μL by flow cytometry. (B) log 10 count of CD4⁺ CAR-T cells/μL by flow cytometry. (C) Persistence of CAR-T cells, as measured using qPCR.

FIGS. 24A-24D show progression-free (PFS) and overall survival (OS) in patients with follicular lymphoma after CD19 CAR-T cell immunotherapy. Kaplan-Meier estimates of PFS (A-B) and OS (C-D) in patients with follicular lymphoma (A-C) and transformed follicular lymphoma (B-D) who achieved complete remission and in all patients. The numbers of patients at risk at 6-month intervals are indicated.

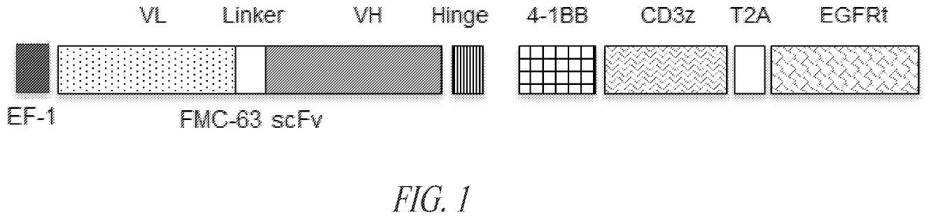

### DETAILED DESCRIPTION

The instant disclosure provides biomarkers, risk factors, and related methods for diagnosing or detecting the risk of relapse of a hematological malignancy in a subject who has achieved a complete response following a treatment regimen comprising lymphodepleting chemotherapy followed by one or more infusion of modified immune cells containing a heterologous polynucleotide encoding a binding protein that specifically binds to an antigen expressed by or associated with the hematological malignancy.

Also provided herein are methods for diagnosing or detecting the risk of relapse following a cellular immunotherapy, or of failing to achieve a complete response following a cellular immunotherapy, wherein the subject has a hematologic malignancy.

Risk factors that are associated with the likelihood of disease relapse were identified before and after lymphodepletion and CAR-T cell infusion, allowing identification of patients at high risk for relapse and who are candidates for early intervention. In particular, various biomarkers and factors examined individually and in various combinations indicate what therapies to apply, what therapeutic regimens to apply, what therapies to adjust, what therapies to avoid, or any combination thereof that will be the most beneficial to a subject at risk of having a relapse following immunotherapy.

Exemplary biomarkers of this disclosure include pre-lymphodepletion levels of serum lactate dehydrogenase (LDH), pre-lymphodepletion levels of platelets, IL-7, IL-18, and pre-treatment regimen disease pathology. Exemplary predictive characteristics include in vivo expansion of the CAR-modified T cells, and extramedullary disease. The instant disclosure also provides biomarkers, risk factors, and related methods for predicting whether a patient with a hematological malignancy who receives CAR-T cell therapy according to the present disclosure is likely to achieve a complete response (CR) and/or progression-free survival (PFS). Also provided are methods for administering a further treatment to patients unlikely to achieve CR and/or PFS.

The instant disclosure also provides methods for treating various hematological malignancies, and compositions for use in managing or treating hematological malignancies as disclosed herein.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated.

As used herein, the terms “about” and “consisting essentially of” mean ±20% of the indicated range, value, or structure, unless otherwise indicated. In particular embodiments, the term “about” means ±2.5% of the indicated range or value for each of the following terms only: “sensitivity,” “specificity,” and “temperature.”

It should be understood that the terms “a” and “an” as used herein refer to “one or more” of the enumerated components. The use of the alternative (e.g., “or”) should be understood to mean either one, both, or any combination thereof of the alternatives or enumerated components. As used herein, the terms “include,” “have” and “comprise” are used synonymously, which terms and variants thereof are intended to be construed as non-limiting.

The term “consisting essentially of” is not equivalent to “comprising,” and refers to the specified materials or steps, or to those that do not materially affect the basic characteristics of a claimed invention. For example, a protein domain, region, or module (e.g., a binding domain, hinge region, linker module) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “hyperproliferative disorder” refers to excessive growth or proliferation as compared to a normal or undiseased cell. Representative hyperproliferative disorders include tumors, cancers, neoplastic tissue, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like). In certain embodiments, a hyperproliferative disorder comprises a hematologic malignancy, such as a lymphoma, a leukemia or a myeloma. Certain diseases that involve abnormal or excessive growth that occurs more slowly than in the context of a hyperproliferative disease can be referred to as “proliferative diseases”, and include certain tumors, cancers, neoplastic tissue, carcinoma, sarcoma, malignant cells, pre-malignant cells, as well as non-neoplastic or non-malignant disorders

As used herein, “relapse” or “recurrence” of a disease (e.g., a hematological malignancy, such as a hematological cancer) refers to a return of the disease to a detectable level or higher following a period of time (e.g., days, weeks, months or years) during which the disease was not detectable. A relapse can occur in the same site (e.g., tissue or organ) in which the disease originated or was first (or last) observed (sometimes called a local relapse or recurrence), or in a nearby site (sometimes called a regional relapse or recurrence) or in a different site (sometimes called a distal relapse or recurrence).

In some embodiments, “progression” of a disease (e.g., a hematological malignancy such as CLL) refers to an increase in the size, volume (i.e., growth) and/or spread of the disease within a site or from a first site to a second site within the body (e.g., metastasis). In some embodiments, a tumor is “progressive” when there is a measured growth of the tumor of at least about 10%, about 15%, about 20%, or about 25% or more. In some embodiments, progression refers to a change in the status of a cancer from a hyperplastic state to a dysplastic state, or from a dysplastic state to a carcinoma.

As used herein, “complete response” (“CR”), also referred to as “complete remission,” means the disappearance of all signs of disease (e.g., cancer) in response to treatment. “Minimal-residual disease” (“MRD”) refers to a condition in which small amounts of disease (e.g., cancer cells) persist despite there being no evidence of disease effects. Techniques for detecting MRD include those described in Van Dongen et al., Blood 125:3996 (2015), which techniques are hereby incorporated by reference. In certain embodiments, a subject has or has achieved a “MRD-negative CR” wherein fewer than one malignant cell is found per million cells.

“Disease-free survival” (“DFS”), as used herein, refers to the length of time following primary treatment (e.g., a treatment comprising lymphodepleting chemotherapy followed by infusion with CAR-T cells) that the treated patient survives without any signs or symptoms of cancer.

“Progression-free survival (“PFS”), as used herein, is the length of time during and after treatment of a disease (e.g., cancer) that the patient survives with the disease but the disease does not get worse (e.g., does not grow or spread).

“Aggressive” when referring to a hyperproliferative disease (e.g., a cancer), means that the disease that forms, grows, or spreads quickly (i.e., within a site of origin, within a local area or tissue, or from a site of origin to a distal area or tissue).

“Indolent”, when referring to a hyperproliferative disease (e.g., a cancer), means that the disease grows, forms, or spreads slowly. In certain embodiments, hematological malignancies such as B cell cancers can be classified as aggressive or indolent using, for example, tumor histology, MRI, PET, X-rays, flow cytometry, or the like.

As used herein, “risk” is the likelihood (probability) of a subject developing, or failing to develop, a disease-related condition as disclosed herein. In certain embodiments, risk is the likelihood that a subject receiving therapy according to the present disclosure will fail to achieve a complete response following a therapy or therapeutic regimen. In other embodiments, risk refers to the probability that a subject who has achieved a complete response following therapy will experience a relapse, death, and/or disease progression. Risk is a representation of the likelihood that a subject will develop, fail to develop, or have a recurrence of the indicated condition within a period of time after treatment (such as hours, days, weeks, or months later). A “high risk” indicates a greater than 50% chance that the subject will develop, not develop, or have a relapse of the indicated condition after a treatment. In certain embodiments, a high risk indicates that there is a greater than 60%, 70%, 80%, or 90% chance that a subject will experience a relapse when in CR after a treatment, or will not achieve CR after a treatment. Conversely, a “low risk” indicates a less than 50% chance that the subject will experience a relapse when in CR after a treatment, or will not achieve CR after a treatment. In certain embodiments, a low risk indicates that there is a less than 10%, 20%, 30%, or 40% chance of developing, or failing to develop, the indicated condition after a treatment.

In some embodiments, a subject is at risk because the subject belongs to a subpopulation identified by specific characteristics, such as biomarkers of this disclosure, as well as age, gender, diet, ethnicity, or a combination thereof. A subject of a subpopulation is, for example, a human subject that is up to 6 years old, is from 6 years old to 17 years old, or is at least 17 years of age or older.

As used herein, “prognosis” is the likelihood of the clinical outcome for a subject afflicted with a specific disease or disorder. With regard to hematological malignancies such as hematological cancers, the prognosis is a representation of the likelihood (probability) that the subject will survive (such as for 1, 2, 3, 4 or 5 years) with or without relapse, disease, and/or disease progression. A “poor prognosis” indicates a greater than 50% chance that the subject will not survive (or survive without disease progression or relapse) to a specified time point (such as 1, 2, 3, 4 or 5 years), and/or a greater than 50% chance that death, relapse, and/or disease progression will occur. In several examples, a poor prognosis indicates that there is a greater than 60%, 70%, 80%, or 90% chance that the subject will not survive and/or a greater than 60%, 70%, 80% or 90% chance that death, relapse, or disease progression will occur. Conversely, a “good prognosis” indicates a greater than 50% chance that the subject will survive to a specified time point (such as 1, 2, 3, 4, or 5 years), and/or a greater than 50% chance that death, relapse, or disease progression will not occur. In several examples, a good prognosis indicates that there is a greater than 60%, 70%, 80%, or 90% chance that the subject will survive and/or a greater than 60%, 70%, 80% or 90% chance that a severe adverse event will not occur.

Certain methods disclosed herein are used to detect biomarkers that indicate the risk, diagnosis, probability, or prognosis of a subject achieving a complete response (CR), disease-free survival (DFS), and/or progression-free survival (PFS) following treatment for a hyperproliferative disorder, such as a hematologic malignancy, that comprises lymphodepleting chemotherapy followed by infusion with modified cells according to the present disclosure.

As used herein, “factor” refers to a subject-related, disease-related, or therapy-related variable according to the presently disclosed therapeutic and diagnostic methods. Examples of subject-related factors include patient age; patient gender; ECOG-score; whether the subject has one or more other disease; whether the subject has one or more biomarkers associated with the hyperproliferative disease or disorder (e.g., a chromosomal rearrangement known to be associated with some hematological malignancies). Examples of disease-related factors include disease burden, serum LDH, International Prognostic Index (IPI) score, the presence of abnormal marrow B cells, and the like. Examples of therapy-related factors include the type, dose, and/or frequency of lymphodepleting chemotherapy a subject has received; the type of modified immune cell and binding protein a subject has received; the frequency and dosing of the modified immune cell therapy; in vivo expansion of modified immune cells following infusion into a subject; biomarker levels prior to or following administration of a therapy as provided herein; whether a subject has previously received an allogeneic hematopoietic stem cell transplant (allo-HSCT), steroids, or the like; whether the subject has experienced a cytokine release syndrome (CRS) event following infusion with modified immune cells, or the like.

“Biomarker” refers to a cell, particle, molecule, compound, or other chemical entity or biologic structure that is an indicator of an abnormal biological condition (e.g., disease or disorder). Exemplary biomarkers include proteins (e.g., antigens or antibodies), carbohydrates, cells, microparticles, viruses, nucleic acids, or small organic molecules. For example, a biomarker may be a gene product that (a) is expressed at higher or lower levels, (b) has an altered ratio relative to another biomarker, (c) is present at higher or lower levels, (d) is a variant or mutant of the gene product, or (e) is simply present or absent, in a cell or tissue sample from a subject having or suspected of having a disease as compared to an undiseased tissue or cell sample from the subject having or suspected of having a disease, or as compared to a cell or tissue sample from a subject or a pool of subjects not having or suspected of having the disease. That is, one or more gene products are sufficiently specific to the test sample that one or more may be used to identify, predict, or detect the presence of disease, risk of disease, risk of a given event or change in disease status, or provide information for a proper or improved therapeutic regimen. A biomarker may refer to two or more components or a ratio thereof (e.g., proteins, nucleic acids, carbohydrates, or a combination thereof) that bind together, associate non-covalently to form a complex, disrupt the association of a complex or two or more molecules or proteins.

By “subject” is meant an organism having a hyperproliferative disease, such as a hematologic malignancy (e.g., lymphoma, leukemia, myeloma), or at risk of experiencing a relapse of such a disease following achievement of complete remission. A subject may benefit from a particular therapeutic regimen described herein, which can be based on, for example, a biomarker level selected from serum LDH, platelet counts, IL-7, IL-18, in-vivo expansion of the modified immune cell, aggressive versus indolent disease histology, or any combination thereof. “Subject” also refers to an organism to which a small molecule, chemical entity, nucleic acid molecule, peptide, polypeptide or other therapy of this disclosure can be administered to treat, ameliorate, minimize the risk, or prevent recurrence or disease progression of hyperproliferative disease, such as a hematologic malignancy (e.g., lymphoma, leukemia, myeloma) and to minimize the risk of an adverse event (e.g., relapse, neurotoxicity). In certain embodiments, a subject is an animal, such as a mammal or a primate. In other embodiments, a subject is a human or a non-human primate.

The term “biological sample” includes a blood sample, biopsy specimen, tissue explant, organ culture, biological fluid or specimen (e.g., blood, serum, plasma, ascites, mucosa, lung sputum, saliva, feces, cerebrospinal fluid (CSF)) or any other tissue or cell or other preparation from a subject or a biological source. A “biological source” may be, for example, a human or non-human animal subject, a primary cell or cell culture or culture adapted cell line including cell lines genetically engineered by human intervention to contain chromosomally integrated or episomal heterologous or recombinant nucleic acid molecules, somatic cell hybrid cell lines, immortalized or immortalizable cells or cell lines, differentiated or differentiatable cells or cell lines, transformed cells or cell lines, or the like. In a preferred embodiment, a biological sample is from a human, such as a serum sample. By “human patient” is intended a human subject who is afflicted with, at risk of developing or relapsing with, any disease or condition associated with a hyperproliferative disorder, or of having an adverse event associated with or following the treatment of such a hyperproliferative disorder.

A biological sample is referred to as a “test sample” when being tested or compared to a “control.” A “control,” as used herein, refers to an undiseased sample from the same patient and same tissue, a sample from a subject not having or suspected of having the disease of interest, a pool of samples from various subjects not having or suspected of having the disease of interest (e.g., including samples from two to about 100 subjects to about 1,000 subjects to about 10,000 subjects to about 100,000 subjects), or data from one or more subjects having or suspected of having the disease of interest and having received a different therapy than the subject of interest, possessing one or more biomarker or other factor that is different than the subject of interest, or both (e.g., including samples from two to about 100 subjects to about 1,000 subjects to about 10,000 subjects to about 100,000 subjects). In certain embodiments, a “test sample” is analyzed and the results (i.e., biomarker levels or activity) compared to a “control” comprising an average or certain identified baseline level calculated from a database having data derived from a plurality of analyzed undiseased or normal samples.

A “reference” or “standard” may optionally be included in an assay, which provides a measure of a standard or known baseline level of a target molecule, structure, or activity (e.g., “normal” level). In certain embodiments, a reference sample is a pool of samples (e.g., 2, 3, 4, 5, 6, 7, 8, 9, 10, or up to 100 or 1,000 or 10,000 samples combined) from healthy individuals (i.e., not having or suspected of having the disease of interest). In certain instances, a “test sample” and a “control sample” will be examined in an assay of the instant disclosure along with a reference sample. In these instances, the “test” and “control” samples may be collectively referred to as the “target samples” since they are being compared to a reference sample.

When referring to the level of the one or more biomarker in a test sample, “elevated” or “increased” or “reduced” compared to a control, as used herein, means a statistically significant increase (or reduction, as the context dictates) in level or activity. In certain embodiments, the level or activity of biomarker(s) in a test sample is elevated compared to a control or reference in a statistically significant manner. In further embodiments, the level or activity of biomarker(s) in a test sample is increased in a statistically significant manner. For example, the difference between test and control levels or control may be about 2-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, about 10-fold, about 15-fold, about 20-fold, about 30-fold, or more. In certain instances, a statistically significant difference includes when a biomarker or related activity is present in a test sample but is absent or undetectable in the control. In certain embodiments, a reference level is determined according to a level observed in or obtained from a group of subjects having the same disease and receiving therapy. For example, in some embodiments, observed IL-7 cytokine levels (within a given time period or at a given time point) or observed IL-7 cytokine peak levels may be increased as compared to the median level (or median peak level) observed within the treatment group and within an indicated time period (or at a given time point). In certain embodiments, observed IL-18 cytokine levels may be reduced as compared to the median level observed within the treatment group and within an indicated time period. In certain embodiments, an observed serum MCP-1 level (within a given time period or at a given time point) may be increased as compared to a reference median level within the treatment group.

As used herein, “sensitivity” refers to a measure of the proportion of subjects having a disease (e.g., humans) who test positive for one or more biomarkers before or shortly after receiving treatment for the disease and who develop one or more adverse events shortly after the treatment over the total population of subjects who develop one or more adverse events (usually expressed as a percentage). In other words, “high sensitivity” (e.g., a sensitivity of at least 85%, 86%, 87%, 88%, 89%, 90%, 91%, 92%, 93%, 94%, 95%, 96%, 97%, 98%, 99% or 100%) means there are few or a low percentage of false negatives present and “low sensitivity” (e.g., a sensitivity below about 70%) means there are many or a high percentage of false negatives present.

As used herein, “specificity” refers to a measure of the proportion of subjects having a disease (e.g., humans) who test negative for the one or more biomarkers or risk factors before or shortly after receiving treatment for the disease and who do not show statistically significant disease progression following the treatment or do not relapse after achieving a complete response over the total population of subjects who do not show statistically significant disease progression or do not relapse (usually expressed as a percentage). In other words, “high specificity” (e.g., a sensitivity of at least 85%, 86%, 87%, 88%, 89%, 90%, 91%, 92%, 93%, 94%, 95%, 96%, 97%, 98%, 99% or 100%) means there are few or a low percentage of false positives present and “low specificity” (e.g., a sensitivity below about 70%) means there are many or a high percentage of false positives present.

In certain embodiments, any of the methods described herein have a sensitivity of at least about 85%, about 86%, about 87%, about 88%, about 89%, about 90%, about 91%, about 92%, about 93%, about 94%, about 95%, about 96%, about 97%, about 98%, about 99%, about 100%, or 100%. In some embodiments, the sensitivity for pre-diagnostic, pre-treatment, or post-treatment detection of the risk for relapse following therapy (e.g., CAR-T cell therapy), is about 100% or is 100%.

In certain embodiments, any of the methods described herein have a specificity that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

As used herein, “nucleic acid” or “nucleic acid molecule” or “polynucleotide” refers to any of deoxyribonucleic acid (DNA), ribonucleic acid (RNA), oligonucleotides, fragments generated, for example, by the polymerase chain reaction (PCR) or by in vitro translation, and fragments generated by any of ligation, scission, endonuclease action, or exonuclease action. In certain embodiments, the nucleic acids of the present disclosure are produced by PCR. Nucleic acids may be composed of monomers that are naturally occurring nucleotides (such as deoxyribonucleotides and ribonucleotides), analogs of naturally occurring nucleotides (e.g., α-enantiomeric forms of naturally-occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules can be either single stranded or double stranded. In certain embodiments, a sequence of two or more linked nucleic acid molecules is referred to as a polynucleotide.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region “leader and trailer” as well as intervening sequences (introns) between individual coding segments (exons).

The “percent identity” or “sequence identity,” as used herein, refers to the percentage of nucleic acid or amino acid residues in one sequence that are identical with the nucleic acid or amino acid residues in a reference polynucleotide or polypeptide sequence, respectively, (i.e., % identity=number of identical positions/total number of positions×100) after aligning the sequences and introducing gaps, if necessary, to achieve the maximum percent sequence identity of two or more sequences. For proteins, conservative substitutions are not considered as part of the sequence identity. The comparison of sequences and determination of percent identity between two or more sequences is accomplished using a mathematical algorithm, such as BLAST and Gapped BLAST programs at their default parameters (e.g., Altschul et al., J. Mol. Biol. 215:403, 1990; Altschul et al., Nucleic Acids Res. 25:3389, 1997; see also BLASTN or BLASTP at www.ncbi.nlm.nih.gov/BLAST).

A “conservative substitution” is recognized in the art as a substitution of one amino acid for another amino acid that has similar properties. Exemplary conservative substitutions are well known in the art (see, e.g., WO 97/09433, page 10, published Mar. 13, 1997; Lehninger, Biochemistry, Second Edition; Worth Publishers, Inc. NY:N.Y. (1975), pp. 71-77; Lewin, Genes IV, Oxford University Press, NY and Cell Press, Cambridge, Mass. (1990), p. 8).

A “patient in need” or “subject in need” refers to a patient or subject at risk of, or suffering from, a disease, disorder or condition (e.g., leukemia, lymphoma, myeloma) that is amenable to treatment or amelioration with an early intervention or altered therapy regimen or therapy regimen as provided herein.

“Treatment,” “treating” or “ameliorating” refers to either a therapeutic treatment or prophylactic/preventative treatment. A treatment is therapeutic if at least one symptom of disease (e.g., leukemia, lymphoma, myeloma) in an individual receiving treatment improves or a treatment may delay worsening of a progressive disease in an individual, or prevent onset of additional associated diseases or symptoms. In general, an appropriate dose and treatment regimen provide one or more of a binding protein specific for an antigen of interest, or a host cell expressing such a binding protein, and optionally in combination with an adjunctive therapy (e.g., a cytokine such as IL-2, IL-15, IL-21, or any combination thereof; chemotherapy, radiation therapy such as localized radiation therapy), in an amount sufficient to provide therapeutic or prophylactic benefit. Therapeutic or prophylactic benefit resulting from therapeutic treatment or prophylactic or preventative methods include, for example an improved clinical outcome, wherein the object is to prevent or retard or otherwise reduce (e.g., decrease in a statistically significant manner relative to an untreated control) an undesired physiological change or disorder, or to prevent, retard or otherwise reduce the expansion or severity of such a disease or disorder. Beneficial or desired clinical results from treating a subject include abatement, lessening, or alleviation of symptoms that result from or are associated the disease or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; or overall survival. “Treatment” can also mean prolonging survival when compared to expected survival if a subject were not receiving treatment. Subjects in need of the methods and compositions described herein include those who already have the disease or disorder, as well as subjects prone to have or at risk of developing the disease or disorder. Subjects in need of prophylactic treatment include subjects in whom the disease, condition, or disorder is to be prevented (i.e., decreasing the likelihood of occurrence or recurrence of the disease or disorder, and/or providing a prophylactic regimen or action to prevent or reduce the risk or severity of a relapse or progression of disease). The clinical benefit provided by the compositions (and preparations comprising the compositions) and methods described herein can be evaluated by design and execution of in vitro assays, preclinical studies, and clinical studies in subjects to whom administration of the compositions is intended to benefit, as described in the examples.

A “therapeutically effective amount (or dose)” or “effective amount (or dose)” refers to that amount of compound sufficient to result in amelioration of one or more symptoms of the disease being treated (e.g., leukemia, lymphoma, myeloma) in a statistically significant manner, or minimizing the risk of an adverse event or other disease-related event (e.g., death, relapse, and/or disease progression). When referring to an individual active ingredient administered alone, a therapeutically effective dose refers to that ingredient alone. When referring to a combination, a therapeutically effective dose refers to combined amounts of the active ingredients that result in the therapeutic effect, whether administered serially or simultaneously (in the same formulation or in separate formulations).

The term “pharmaceutically acceptable” refers to molecular entities and compositions that do not produce allergic or other serious adverse reactions when administered using routes well known in the art.

The term “construct” refers to any polynucleotide that contains a recombinantly engineered nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Exemplary vectors are those capable of autonomous replication (episomal vector) or expression of nucleic acid molecules to which they are linked (expression vectors).

Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

The term “operably linked” refers to the association of two or more nucleic acid molecules on a single nucleic acid fragment so that the function of one is affected by the other. For example, a promoter is operably-linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself In the present specification, “plasmid,” “expression plasmid,” “virus” and “vector” are often used interchangeably.

As used herein, the term “expression level” refers to the quantity of protein or gene expression by a cell or population of cells. Techniques for detecting and measuring protein expression are known to those of skill in the art and include, for example, immunostaining, immunoprecipitation, fluorescence-labeling, BCA, and Western blot. Techniques for detecting and measuring gene expression are known to those of skill in the art and include, for example, RT-PCR, in situ hybridization, fluorescence-labeled oligonucleotide probes, radioactively labeled oligonucleotide probes, and Northern blot.

### Compositions

In certain embodiments, presently disclosed methods, kits, and uses comprise: a modified immune cell containing a heterologous polynucleotide that encodes a binding protein that specifically binds to an antigen expressed by or associated with a disease or disorder, such as a hematological malignancy; a chemotherapeutic agent; a Bruton's tyrosine kinase inhibitor; a biologic agent or therapy such as a cytokine or an antibody, or the like, or any combination thereof.

Accordingly, in certain embodiments, the present disclosure provides binding proteins that specifically bind to an antigen of interest and can be heterologously expressed by a modified immune cell. A “binding domain” (also referred to as a “binding region” or “binding moiety”), as used herein, refers to a molecule or portion thereof (e.g., peptide, oligopeptide, polypeptide, protein) that possesses the ability to specifically and non-covalently associate, unite, or combine with a target. A binding domain includes any naturally occurring, synthetic, semi-synthetic, or recombinantly produced binding partner for a biological molecule, a molecular complex (i.e., complex comprising two or more biological molecules), or other target of interest. Exemplary binding domains include single chain immunoglobulin variable regions (e.g., scFv), receptor ectodomains, ligands (e.g., cytokines, chemokines), or synthetic polypeptides selected for their specific ability to bind to a biological molecule, a molecular complex or other target of interest.

As used herein, “specifically binds” or “specific for” refers to an association or union of a binding protein or a binding domain (or fusion protein thereof) to a target molecule with an affinity or Kₐ (i.e., an equilibrium association constant of a particular binding interaction with units of 1/M) equal to or greater than 10⁵ M⁻¹ (which equals the ratio of the on-rate [kₒₙ] to the off-rate [k_{off}] for this association reaction), while not significantly associating or uniting with any other molecules or components in a sample. Binding proteins or binding domains (or fusion proteins thereof) may be classified as “high affinity” binding proteins or binding domains (or fusion proteins thereof) or as “low affinity” binding proteins or binding domains (or fusion proteins thereof). “High affinity” binding proteins or binding domains refer to those binding proteins or binding domains having a Kₐ of at least 10⁷ M⁻¹, at least 10⁸ M⁻¹, at least 10⁹ M⁻¹ at least 10¹⁰ M⁻¹, at least 10¹¹ M⁻¹, at least 10¹² M⁻¹, or at least 10¹³ M⁻¹. “Low affinity” binding proteins or binding domains refer to those binding proteins or binding domains having a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M).

A variety of assays are known for identifying binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

The term “T cell receptor,” as used herein, refers to an heterodimeric antigen binding receptor derived from a T lymphocyte, comprising a an alpha/beta polypeptide dimer or a gamma/delta polypeptide dimer, each dimer comprising a variable region, a constant region, and an antigen binding site. In some contexts, a TCR is an immunoglobulin superfamily member (having a variable binding domain, a constant domain, a transmembrane region, and a short cytoplasmic tail; see, e.g., Janeway et al., Immunobiology: The Immune System in Health and Disease, 3^{rd} Ed., Current Biology Publications, p. 4:33, 1997) capable of specifically binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). Like other immunoglobulins (e.g., antibodies), the extracellular portion of TCR chains (e.g., α-chain, β-chain) contain two immunoglobulin domains, a variable domain (e.g., α-chain variable domain or V_{α}, β-chain variable domain or V_{β}; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or C_{α}, typically amino acids 117 to 259 based on Kabat, β-chain constant domain or C_{β}, typically amino acids 117 to 295 based on Kabat) adjacent to the cell membrane. Also like other immunoglobulins, the variable domains contain complementary determining regions (CDRs) separated by framework regions (FRs) (see, e.g., Jores et al., Proc. Nat'l Acad. Sci. U.S.A. 87:9138, 1990; Chothia et al., EMBO 1 7:3745, 1988; see also Lefranc et al., Dev. Comp. Immunol. 27:55, 2003). In certain embodiments, a TCR is found on the surface of T cells (or T lymphocytes) and associates with the CD3 complex. The source of a TCR as used in the present disclosure may be from various animal species, such as a human, mouse, rat, rabbit or other mammal.

In certain embodiments, any of the aforementioned antigen specific binding proteins are each a chimeric antigen receptor or an antigen-binding fragment of a TCR, any of which can be chimeric, humanized or human. In further embodiments, an antigen-binding fragment of a TCR comprises a single chain TCR (scTCR) or is contained in a chimeric antigen receptor (CAR). Methods for producing engineered TCRs are described in, for example, Bowerman et al. (Mol. Immunol. 46:3000, 2009), the techniques of which are herein incorporated by reference. In certain embodiments, an antigen-specific binding domain comprises a CAR comprising an antigen-specific TCR binding domain (see, e.g., Walseng et al., Scientific Reports 7:10713, 2017; the TCR CAR constructs of which are hereby incorporated by reference in their entirety). Methods for making CARs are also described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; and Brentjens et al., Clin. Cancer Res. 13:5426, 2007, the techniques of which are herein incorporated by reference.

In any of the embodiments disclosed herein, the encoded binding protein can comprise a chimeric antigen receptor (CAR) and/or a scTCR. In certain embodiments, the encoded binding protein comprises a CAR comprising an extracellular component comprising a binding domain specific for the antigen and a hinge region, an intracellular component, and a transmembrane component disposed between the extracellular component and the intracellular component, wherein the hinge region is disposed between the binding domain and the transmembrane component.

As used herein, the terms “antibody” or “binding fragment,” or “antibody fragment” refer to their standard meanings within the art; that is, an intact immunoglobulin molecule or a fragment thereof that is capable of binding an antigen.

In certain embodiments, the binding protein comprises a T-ChARM. In certain embodiments, a T-ChARM comprises an extracellular component and an intracellular component connected by a hydrophobic portion, wherein the extracellular component comprises a binding domain that specifically binds a target, a tag cassette, and a connector region comprising a hinge, and wherein the intracellular component comprises an effector domain. In further embodiments, a CAR comprises an extracellular component and an intracellular component connected by a hydrophobic portion, wherein the extracellular component comprises a binding domain that specifically binds a target, and a connector region comprising a hinge, and wherein the intracellular component comprises an effector domain. In certain embodiments, a T-ChARM or CAR binding domain is a scFv, scTCR, receptor ectodomain, or ligand. T-ChARMs as disclosed in PCT Publication of WO 2015/095895 are incorporated herein by reference in their entirety. In certain embodiments, a T-ChARM or CAR binding domain is specific for CD19, CD20, CD22, CD37, or the like.

In any of the embodiments disclosed herein, the antigen is CD19. In certain embodiments, the binding domain of the encoded binding protein is derived from FMC-63 antibody (see, e.g., Zola et al. Immunol. Cell Biol. 69(Pt 6):411-22 (1991) and WO 2015/095895, which FMC-63 antibody and scFv sequences are incorporated herein by reference), MOR208 (see, e.g., Horton et al., Cancer Res. 68(19): (2008); see also Meeker et al. Hybridoma 3:305 (1984)), blinatumomab (see, e.g., Mølhøj et al., Mol. Immunol. 44(8):1935 (2007)), MEDI-551 (see, e.g., Herbst et al., J. Pharmacol. Exp. Ther. 335:213 (2010)), Merck patent anti-CD19 antibody, Xmab5871 aka obexelimab, or MDX-1342; and/or the hinge region is derived from IgG4; and/or the transmembrane component is derived from CD28; and/or the intracellular component comprises a 4-1BB signaling domain and a CD3ζ domain.

Antigen-specific binding proteins or domains, as described herein, may be functionally characterized according to methodologies used for assaying T cell activity, including determination of T cell binding, activation or induction and also including determination of T cell responses that are antigen-specific. Examples include determination of T cell proliferation, T cell cytokine release, antigen-specific T cell stimulation, MHC restricted T cell stimulation, CTL activity (e.g., by detecting ⁵¹Cr release from pre-loaded target cells), changes in T cell phenotypic marker expression, and other measures of T-cell functions. Procedures for performing these and similar assays are may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein.

As used herein, the term “modified” or “genetically engineered” refers to a cell, microorganism, nucleic acid molecule, or vector that has been recombinantly created by human intervention—that is, modified by introduction of a heterologous nucleic acid molecule (or polynucleotide), or refers to a cell or microorganism that has been altered such that expression of an endogenous nucleic acid molecule or gene is controlled, deregulated or constitutive. Human-generated genetic alterations may include, for example, modifications that introduce nucleic acid molecules (which may include an expression control element, such as a promoter) that encode one or more proteins or enzymes, or other nucleic acid molecule additions, deletions, substitutions, or other functional disruption of or addition to a cell's genetic material. Exemplary modifications include those in coding regions or functional fragments thereof of heterologous or homologous polypeptides from a reference or parent molecule. As used herein, “heterologous” or “exogenous” nucleic acid molecule, construct or sequence refers to a nucleic acid molecule or portion of a nucleic acid molecule (or polynucleotide) that is not native to a host cell, but may be homologous to a nucleic acid molecule or portion of a nucleic acid molecule from the host cell. The source of the heterologous or exogenous nucleic acid molecule (or polynucleotide), construct or sequence may be from a different genus or species. In certain embodiments, a heterologous or exogenous nucleic acid molecule is added (i.e., not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein or other activity encoded by an exogenous nucleic acid molecule introduced into the host cell, even if the host cell encodes a homologous protein or activity.

As described herein, more than one heterologous or exogenous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. For example, as disclosed herein, a host cell can be modified to express two or more heterologous or exogenous nucleic acid molecules encoding desired binding protein. When two or more exogenous nucleic acid molecules are introduced into a host cell, it is understood that the two or more exogenous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, an “immune system cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Exemplary immune system cells include a CD4+ T cell, a CD8+ T cell, a CD4− CD8− double negative T cell, a γδ T cell, a stem cell memory T cell, a regulatory T cell, a natural killer cell, a natural killer T cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

In some embodiments, the human immune cell comprises a hematopoietic stem cell, a lymphoid progenitor cell, a T cell, a NK cell, a NK-T cell, a B cell, a myeloid progenitor cell, a monocyte, a macrophage, a dendritic cell, a megakaryocyte, a granulocyte, or any combination thereof. In certain embodiments, the immune cell comprises a CD4+ T cell, a CD8+ T cell, or both.

The level of a CTL immune response may be determined by any one of numerous immunological methods described herein and routinely practiced in the art. The level of a CTL immune response may be determined prior to and following administration of any one of the herein described Merkel cell polyomavirus T antigen-specific binding proteins expressed by, for example, a T cell. Cytotoxicity assays for determining CTL activity may be performed using any one of several techniques and methods routinely practiced in the art (see, e.g., Henkart et al., “Cytotoxic T-Lymphocytes” in Fundamental Immunology, Paul (ed.) (2003 Lippincott Williams & Wilkins, Philadelphia, Pa.), pages 1127-50, and references cited therein).

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

Cells expressing a binding protein specific for an antigen as described herein may be administered to a subject in a pharmaceutically or physiologically acceptable or suitable excipient or carrier. Pharmaceutically acceptable excipients are biologically compatible vehicles, e.g., physiological saline, which are described in greater detail herein, that are suitable for administration to a human or other non-human mammalian subject.

A therapeutically effective dose is an amount of host cells (expressing a binding protein) used in adoptive transfer that is capable of producing a clinically desirable result (i.e., a sufficient amount to induce or enhance a specific T cell immune response against cells expressing an antigen (e.g., a cytotoxic T cell (CTL) response in vivo or cell lysis in vitro in the presence of the specific antigen epitope or peptide) in a statistically significant manner) in a treated human or non-human mammal. The dosage for any one patient can depend upon many factors, including the patient's size, weight, body surface area, age, the particular therapy to be administered, sex, time and route of administration, general health, and other drugs being administered concurrently. Doses will vary, but a preferred dose for administration of a host cell comprising a recombinant expression vector as described herein is about 10⁵ cells/m², about 5×10⁵ cells/m², about 10⁶ cells/m², about 5×10⁶ cells/m² about 10⁷ cells/m², about 5×10⁷ cells/m², about 10⁸ cells/m², about 5×10⁸ cells/m², about 10⁹ cells/m², about 5×10⁹ cells/m², about 10¹⁰ cells/m², about 5×10¹⁰ cells/m², or about 10¹¹ cells/m².

Unit doses are also provided herein which comprise a host cell (e.g., a modified immune cell comprising a polynucleotide of the present disclosure) or host cell composition of this disclosure. In certain embodiments, a unit dose comprises (i) a composition comprising at least about 30%, at least about 40%, at least about 50%, at least about 60%, at least about 70%, at least about 80%, at least about 85%, at least about 90%, or at least about 95% modified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified CD8⁺ T cells, in about a 1:1 ratio, wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a patient sample having a comparable number of PBMCs).

In some embodiments, a unit dose comprises (i) a composition comprising at least about 50% modified CD4⁺ T cells, combined with (ii) a composition comprising at least about 50% modified CD8⁺ T cells, in about a 1:1 ratio, wherein the unit dose contains a reduced amount or substantially no naïve T cells. In further embodiments, a unit dose comprises (i) a composition comprising at least about 60% modified CD4⁺ T cells, combined with (ii) a composition comprising at least about 60% modified CD8⁺ T cells, in about a 1:1 ratio, wherein the unit dose contains a reduced amount or substantially no naïve T cells. In still further embodiments, a unit dose comprises (i) a composition comprising at least about 70% modified CD4⁺ T cells, combined with (ii) a composition comprising at least about 70% modified CD8⁺ T cells, in about a 1:1 ratio, wherein the unit dose contains a reduced amount or substantially no naïve T cells. In some embodiments, a unit dose comprises (i) a composition comprising at least about 80% modified CD4⁺ T cells, combined with (ii) a composition comprising at least about 80% modified CD8⁺ T cells, in about a 1:1 ratio, wherein the unit dose contains a reduced amount or substantially no naïve T cells. In some embodiments, a unit dose comprises (i) a composition comprising at least about 85% modified CD4⁺ T cells, combined with (ii) a composition comprising at least about 85% modified CD8⁺ T cells, in about a 1:1 ratio, wherein the unit dose contains a reduced amount or substantially no naïve T cells. In some embodiments, a unit dose comprises (i) a composition comprising at least about 90% modified CD4⁺ T cells, combined with (ii) a composition comprising at least about 90% modified CD8⁺ T cells, in about a 1:1 ratio, wherein the unit dose contains a reduced amount or substantially no naïve T cells.

In any of the embodiments described herein, a unit dose comprises equal, or approximately equal numbers of modified CD45RA⁻ CD3⁺ CD8⁺ and modified CD45RA⁻ CD3⁺ CD4⁺ T_{M} cells.

In any of the embodiments described herein, a unit dose comprises equal, or approximately equal numbers of modified CD4+ CD25− T cells and and modified CD8+ CD62L+ T cells.

Also contemplated are pharmaceutical compositions that comprise binding proteins, or cells expressing the binding proteins, or other compositions as disclosed herein, and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's disease, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

The pharmaceutical compositions described herein may be presented in unit-dose or multi-dose containers, such as sealed ampoules or vials. Such containers may be frozen to preserve the stability of the formulation until. In certain embodiments, a unit dose comprises a modified cell as described herein at a dose of about 10⁷ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of modified cells or active compound calculated to produce the desired therapeutic effect in association with an appropriate pharmaceutical carrier.

As a solid composition for oral administration, the pharmaceutical composition may be formulated into a powder, granule, compressed tablet, pill, capsule, chewing gum, wafer or the like form. Exemplary solid compositions can contain one or more inert diluents or edible carriers. In addition, one or more additives may be present, including binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; or a coloring agent. When a pharmaceutical composition is in the form of a capsule, such as a gelatin capsule, it may contain, in addition to materials of the above type, a liquid carrier such as polyethylene glycol or oil or combinations thereof.

The pharmaceutical composition may be in the form of a liquid, such as an elixir, syrup, solution, emulsion, or suspension. In certain embodiments, a liquid composition may be formulated for oral administration or for delivery by injection, as two examples. When intended for oral administration, exemplary compositions may further contain, in addition to one or more compounds of this disclosure, a sweetening agent, preservative, dye/colorant, flavor enhancer, or any combination thereof. Exemplary compositions intended for administration by injection may further contain a surfactant, preservative, wetting agent, dispersing agent, suspending agent, buffer, stabilizer, isotonic agent, or any combination thereof.

Liquid pharmaceutical compositions of this disclosure, whether they are solutions, suspensions or other like forms, may further comprise adjuvants, including sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A pharmaceutical composition of this disclosure may be intended for topical administration, in which case the carrier may comprise a suitable solution, emulsion, ointment, gel base, or any combination thereof. The base, for example, may comprise petrolatum, lanolin, polyethylene glycols, bee wax, mineral oil, diluents such as water and alcohol, emulsifiers, stabilizers, or any combination thereof. Thickening agents may be present in a pharmaceutical composition of this disclosure for topical administration. If intended for transdermal administration, the composition may include a transdermal patch or iontophoresis device.

A pharmaceutical composition of this disclosure may be intended for rectal administration, in the form, for example, of a suppository, which will melt in the rectum and release the active compound(s). A composition for rectal administration may contain an oleaginous base as a suitable nonirritating excipient. Exemplary bases include lanolin, cocoa butter, polyethylene glycol, or any combination thereof.

A pharmaceutical composition of this disclosure may include various materials that modify the physical form of a solid or liquid dosage unit. For example, a composition may include materials that form a coating shell around the active ingredient(s). Exemplary materials for forming a coating shell may be inert, such as sugar, shellac, or other enteric coating agents. Alternatively, active ingredient(s) may be encased in a gelatin capsule.

In certain embodiments, compounds and compositions of this disclosure may be in the form of a solid or liquid. Exemplary solid or liquid formulations include semi-solid, semi-liquid, suspension, and gel forms. A pharmaceutical composition of this disclosure in solid or liquid form may further include an agent that binds to the compound of this disclosure and thereby assists in the delivery of the compound. Suitable agents that may act in this capacity include a monoclonal or polyclonal antibody, a protein, or a liposome.

A pharmaceutical composition of this disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of this disclosure may be delivered in single phase, bi-phasic, or tri-phasic systems in order to deliver the active ingredient(s). Delivery of the aerosol includes the necessary container, activators, valves, subcontainers, and the like, which together may form a kit.

Pharmaceutical compositions of this disclosure may be prepared by methodology well known in the pharmaceutical art. For example, a pharmaceutical composition intended to be administered by injection can be prepared by combining a compound of this disclosure with sterile, distilled water to form a solution. A surfactant may be added to facilitate the formation of a homogeneous solution or suspension. Surfactants are compounds that non-covalently interact with the compound of this disclosure to facilitate dissolution or homogeneous suspension of a compound in an aqueous delivery system.

Cells and compounds, or their pharmaceutically acceptable salts, of this disclosure are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Following administration of therapies according to the formulations and methods of this disclosure, test subjects will exhibit about a 10% up to about a 99% reduction in one or more symptoms associated with the disease or disorder being treated, as compared to placebo-treated or other suitable control subjects.

Cells and compounds, or pharmaceutically acceptable derivatives thereof, of this disclosure may also be administered simultaneously with, prior to, or after administration of one or more other therapeutic agents. Such combination therapy includes administration of a single pharmaceutical dosage formulation which contains a compound of this disclosure and one or more additional active agents, as well as administration of the compound of this disclosure and each active agent in its own separate pharmaceutical dosage formulation. For example, a cellular immunotherapy of this disclosure and another active agent can be administered to the patient together in a single dosage composition, or each agent administered in separate dosage formulations. Where separate dosage formulations are used, the cells and compounds of this disclosure and one or more additional active agents can be administered at essentially the same time, i.e., concurrently, or at separately staggered times, i.e., sequentially; combination therapy is understood to include all these regimens.

In some embodiments, compositions and host cells as described herein are administered with immune modulators (e.g., immunosuppressants, or inhibitors of immunosuppression components, such as immune checkpoint inhibitors). Immune checkpoint inhibitors include inhibitors of CTLA-4, A2AR, B7-H3, B7-H4, BTLA, HVEM, GAL9, IDO, KIR, LAG-3, PD-1, PD-L1, PD-L2, Tim-3, VISTA, TIGIT, LAIR1, CD160, 2B4, TGFR beta, CEACAM-1, CEACAM-3, CEACAM-5, CD244, or any combination thereof. An inhibitor of an immune checkpoint molecule can be an antibody or antigen binding fragment thereof, a fusion protein, a small molecule, an RNAi molecule, (e.g., siRNA, shRNA, or miRNA), a ribozyme, an aptamer, or an antisense oligonucleotide.

Cytokines are used to manipulate host immune response towards anticancer activity. See, e.g., Floros & Tarhini, Semin. Oncol. 42(4):539-548, 2015. Cytokines and growth factors useful for promoting immune anticancer or antitumor response include, for example, IFN-α, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-24, G-CSF, Meg-CSF, GM-CSF, TNF, thrombopoietin, stem cell factor, and erythropoietin, singly or in any combination with the binding proteins, cells expressing the same, or BTK inhibitors of this disclosure.

Exemplary chemotherapeutic agents include alkylating agents (e.g., cisplatin, oxaliplatin, carboplatin, busulfan, nitrosoureas, nitrogen mustards such as bendamustine, uramustine, temozolomide), antimetabolites (e.g., aminopterin, methotrexate, mercaptopurine, fluorouracil, cytarabine, gemcitabine), taxanes (e.g., paclitaxel, nab-paclitaxel, docetaxel), anthracyclines (e.g., doxorubicin, daunorubicin, epirubicin, idaruicin, mitoxantrone, valrubicin), bleomycin, mytomycin, actinomycin, hydroxyurea, topoisomerase inhibitors (e.g., camptothecin, topotecan, irinotecan, etoposide, teniposide), monoclonal antibodies (e.g., ipilimumab, pembrolizumab, nivolumab, avelumab, alemtuzumab, bevacizumab, cetuximab, gemtuzumab, panitumumab, rituximab, tositumomab, trastuzumab), vinca alkaloids (e.g., vincristine, vinblastine, vindesine, vinorelbine), cyclophosphamide, prednisone, leucovorin, oxaliplatin, hyalurodinases, or any combination thereof. In certain embodiments, a chemotherapeutic is vemurafenib, dabrafenib, trametinib, cobimetinib, sunitinib, erlotinib, paclitaxel, docetaxel, or any combination thereof. In some embodiments, a patient is first treated with a chemotherapeutic agent that inhibits or destroys other immune cells followed by a pharmaceutical composition described herein. In some cases, chemotherapy may be avoided entirely.

Certain embodiments of the presently disclosed methods, kits, and uses, comprise a Bruton's tyrosine kinase (BTK) inhibitor, discussed further herein. In some contexts, a BTK inhibitor is a molecule (e.g., small organic molecule, antibody, peptide), compound, or composition that inhibits (e.g., reduces, attenuates, slows, delays, or abrogates) one or more biological activity of BTK and/or inhibits BTK-mediated B cell signaling. BTK inhibitors include, for example, ibrutinib (PCI-32765), acalabrutinib, ONO-4059 (GS-4059), spebrutinib (AVL-202; CC-292), BHB-3111, and HM71224. Ibrutinib is an approved treatment for a number of hematological malignancies, including CLL. Acalabrutinib is approved for treating relapsed mantle cell lymphoma.

In any of the presently disclosed embodiments, the BTK inhibitor comprises ibrutinib, acalabrutinib (ACP-196), ONO-4059 (GS4059), spebrutinib, BGB-3111, HM71224, or any combination thereof. In certain embodiments, the BTK inhibitor comprises ibrutinib.

It will be understood that any of the compositions disclosed herein (e.g., modified immune cell or cell composition, chemotherapy (including lymphodepleting chemotherapies such as cyclophosphamide and fludarabine), checkpoint inhibitor, immune stimulatory compound, biologic agents (e.g., cytokine, fusion protein, peptide, antibody or antigen-binding fragment), small organic molecule (including, for example, a BTK inhibitor), including any combination thereof, may be provided for use in a diagnostic, disease management, and/or therapeutic method according to the present disclosure. That is, any one or more of the presently disclosed compositions is provided for use in a method of diagnosing, treating, and/or managing a disease (e.g., a hematological malignancy such as ALL (e.g., B-ALL), NHL, CLL, FL (including tFL)) according to the present disclosure. In certain embodiments, any one or more of the presently disclosed compositions is provided for use in the manufacture of a medicament for treating and/or managing a disease (e.g., a hematological malignancy such as ALL (e.g., B-ALL), NHL, CLL, FL (including tFL)) according to the present disclosure.

### Methods and Kits for Managing or Treating a Hematological Malignancy

In some aspects, the present disclosure provides methods for reducing the risk of relapse of a hematological malignancy in a human subject presenting with a Minimal Residual Disease-Negative Complete Response following administration to the subject of a first therapy comprising lymphodepleting chemotherapy and one or more infusion of modified immune cells containing a heterologous polynucleotide that encodes a binding protein that specifically binds to an antigen expressed by or associated with the hematological malignancy, the method comprising: (a) measuring a pre-first-therapy biomarker level in the subject, wherein the biomarker is selected from serum lactate dehydrogenase (LDH), platelets, or both; and (b) identifying the subject as being at-risk of relapse when the subject: (i) has a pre-first-therapy serum LDH level of about 210 U/L or more; (ii) does not receive a lymphodepleting chemotherapy comprised of cyclophosphamide and fludarabine; (iii) has a pre-first-therapy platelet count of less than about 100 U/L; (iv) has pre-first-therapy extramedullary disease;(v) has a pre-first-therapy International Prognostic Index (IPI) of 2, 3, or 4; (vi) has one or more diseased cells prior to and following the one or more infusions of modified immune cells, wherein the one or more diseased cells are optionally from the subject's bone marrow, wherein the one or more diseased cells are optionally identified by a mutant nucleotide sequence from at least a portion of an IgH gene (NCBI gene ID: 3492); an IgK gene (NCBI gene ID: 50802); a TRB gene (NCBI gene ID: 6957); a TRD gene (NCBI gene ID: 6964); a TRG gene (NCBI Gene ID: 6965), or any combination thereof; (vii) does not receive an allogeneic hematopoietic stem cell transplant (allo-HSCT); (viii) has a peak serum concentration of IL-7 by about 28 days following the one or more infusions of the modified immune cells at about the same or a lower concentration than the serum concentration of IL-7 immediately prior to a first of the one or more infusions; (ix) has a peak serum concentration of IL-7 by about 28 days following the one or more infusions of the modified immune cells of less than about 20 pg/mL; (x) has a serum concentration of MCP-1 immediately prior to a first of the one or more infusions of the modified immune cells of less than about 10³ pg/mL; less than about 10^{2.5} pg/mL, less than about 10² pg/mL, less than about 10^{1.5} pg/mL, or lower; (xi) has a serum concentration of MCP-1 immediately prior to a first of the one or more infusions of the modified immune cells lower than, or up to about 20% greater than, the serum concentration of MCP-1 at the time of a first administration of the lymphodepleting chemotherapy; (xii) has received the cyclophosphamide at one or more doses of less than about 40 mg/kg, less than about 35 mg/kg, or less than about 30 mg/kg; (xiii) has received a total dose of the cyclophosphamide of less than about 1500 mg/m²; (xiv) has a peak serum concentration of IL-18 by about 28 days following the one or more infusions of the modified immune cells of at least about 10³ pg/mL; or (xv) any combination of b(i)-b(xiv), wherein the at-risk subject is identified as a candidate for a second therapy to reduce the risk of relapse.

In certain embodiments, the second therapy comprises: (i) allogeneic hematopoietic stem cell transplant; (ii) radiation therapy; (iii) chemotherapy; (iv)

surgery; (v) one or more further infusion of the modified immune cell; (vi) immunosuppressive therapy; or (vii) any combination of (i)-(vi).

In some aspects, the present disclosure provides methods for treating a hematological malignancy in a human subject, wherein the subject had previously been administered lymphodepleting chemotherapy and one or more infusion of modified immune cells containing a heterologous polynucleotide that encodes a binding protein that specifically binds to an antigen expressed by or associated with the hematological malignancy, wherein the subject presents with a Minimal Residual Disease-Negative Complete Response following the one or more infusion, the method comprising: (a) administering an allogeneic hematopoietic stem cell transplant (allo HSCT) to the subject and/or monitoring the subject when the subject: (i) has a serum lactate dehydrogenase (LDH) level of less than about 210 U/L prior to receiving the lymphodepleting chemotherapy; (ii) has received a lymphodepleting chemotherapy comprised of cyclophosphamide and fludarabine, wherein the cyclophosphamide is administered at one or more doses of at least about 60 mg/kg, or wherein the lymphodepleting chemotherapy comprises a total of the cyclophosphamide of at least about 3,000 mg/m²; (iii) has a platelet count of about 100 U/L or more prior to receiving the lymphodepleting chemotherapy; (iv) has an increased level of serum MCP-1 prior to receiving the one or more infusions; (v) has an International Prognostic Index (IPI) of 0 or 1 prior to receiving the lymphodepleting chemotherapy and the one or more infusions;
(vi) has a reduced level of IL-18 when receiving the one or more infusions; (vii) has a peak serum concentration of IL-7 by about 28 days following the one or more infusions of the modified immune cells that is higher than the serum concentration of IL-7 immediately prior to a first of the one or more infusions; (viii) has a peak serum concentration of IL-7 by about 28 days following the one or more infusions of the modified immune cells of at least about 20, 25, or 30 pg/mL; (ix) has a serum concentration of MCP-1 immediately prior to a first of the one or more infusions of the modified immune cells of at least about 10³ pg/mL, or lower (x) has a serum concentration of MCP-1 immediately prior to a first of the one or more infusions of the modified immune cells of at least about 20% greater than the serum concentration of MCP-1 at the time of a first administration of the lymphodepleting chemotherapy; (xi)

has one or more diseased cells prior to, but not following, the one or more infusions of modified immune cells, wherein the one or more diseased cells are optionally from the subject's bone marrow, wherein the one or more diseased cells are optionally identified by a mutant nucleotide sequence from at least a portion of an IgH gene (NCBI gene ID: 3492); an IgK gene (NCBI gene ID: 50802); a TRB gene (NCBI gene ID: 6957); a TRD gene (NCBI gene ID: 6964); a TRG gene (NCBI Gene ID: 6965), or any combination thereof; (xii) has received allo-HSCT following the one or more infusions of the modified immune cells, or (xiii) has any combination of (a)(i)-(a)(xii); and (b) administering a therapy comprising allo-HSCT; radiation therapy; chemotherapy; surgery; one or more further infusion of the modified immune cells; immunosuppressive therapy; or any combination thereof, when the subject: (i) has a pre-first-therapy serum LDH level of about 210 U/L or more; (ii) did not receive a lymphodepleting chemotherapy comprised of cyclophosphamide and fludarabine; (iii) has a pre-first-therapy platelet count of less than about 100 U/L; (iv) has pre-first-therapy extramedullary disease; (v) has a pre-first-therapy International Prognostic Index (IPI) of 2, 3, or 4; (vi) has one or more diseased cells prior to and following the one or more infusions of modified immune cells, wherein the one or more diseased cells are optionally from the subject's bone marrow, wherein the one or more diseased cells are optionally identified by a nucleotide sequence from at least a portion of an IgH gene; an IgK gene; a TRB gene; a TRD gene; a TRG gene, or any combination thereof;
(vii) did not receive an allogeneic hematopoietic stem cell transplant (allo-HSCT);
(viii) has a peak serum concentration of IL-7 by about 28 days following the one or more infusions of the modified immune cells at about the same as or lower than the serum concentration of IL-7 immediately prior to a first of the one or more infusion;
(ix) has a peak serum concentration of IL-7 by about 28 days following the one or more infusions of the modified immune cells of less than about 20 pg/mL; (x) has a serum concentration of MCP-1 immediately prior to a first of the one or more infusions of the modified immune cells of less than about 10³ pg/mL; (xi) has a serum concentration of MCP-1 immediately prior to a first of the one or more infusions of the modified immune cells lower than, or up to about 20% greater than, the serum concentration of MCP-1 at the time of a first administration of the lymphodepleting chemotherapy; (xii) has received the cyclophosphamide at one or more doses of less than about 40 mg/kg (e.g., about 35 mg/kg, about 30 mg/kg, or less);
(xiii) has received a total dose of the cyclophosphamide of less than about 1500 mg/m²;
(xiv) has a peak serum concentration of IL-18 by about 28 days following the one or more infusions of the modified immune cells of at least about 10³ pg/mL; or (xv) any combination of b(i)-b(xiv), wherein the at-risk subject is identified as a candidate for a second therapy to reduce the risk of relapse.

In any of the herein disclosed embodiments, the hematological malignancy is selected from acute lymphoblastic leukemia (ALL), optionally B cell ALL, Hodgkin's lymphoma, non-Hodgkins lymphoma (NHL), primary central nervous system lymphomas, T cell lymphomas, small lymphocytic lymphoma (SLL), B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma (mucosa-associated lymphoid tissue (MALT) lymphoma), nodal marginal zone B-cell lymphoma, follicular lymphoma, mantle cell lymphoma, diffuse large B-cell lymphoma (DLBCL), mediastinal (thymic) large B-cell lymphoma, intravascular large B-cell lymphoma, primary effusion lymphoma, acute myeloid leukemia (AML), chronic lymphocytic leukemia (CLL), chronic myoblastic leukemia (CIVIL), Hairy cell leukemia (HCL), chronic myelomonocytic leukemia (CMML), juvenile myelomonocytic leukemia (JMML), large granular lymphocytic leukemia (LGL), blastic plasmacytoid dendritic cell neoplasm (BPDCN), Burkitt lymphoma/leukemia, T cell/histiocyte-rich large B cell lymphoma, pleomorphic mantle cell lymphoma, multiple myeloma, Bence-Jones myeloma, non-secretory myeloma, plasmacytoma, amyloidosis, monoclonal gammopathy of unknown significance (MGUS), or Waldenstrom's macroglobulinemia.

In certain embodiments, the hematological malignancy is B cell ALL.

In certain embodiments, the allo-HSCT is administered to the subject when the subject: (i) has a serum LDH level of less than about 210 U/L prior to receiving the lymphodepleting chemotherapy; (ii) has received cyclophosphamide and fludarabine as the lymphodepleting chemotherapy; and/or (iii) has a platelet count of about 100 U/L or more prior to receiving the lymphodepleting chemotherapy. In certain embodiments, the therapy is administered to the subject when the subject: (i) has a serum LDH level of about 210 U/L or more prior to receiving the lymphodepleting chemotherapy; (ii) did not receive lymphodepleting chemotherapy comprising cyclophosphamide and fludarabine; (iii) has a platelet count of less than about 100 U/L prior to receiving the lymphodepleting chemotherapy; and/or (iv) has one or more diseased cells prior to, but not following, the one or more infusions of modified immune cells, wherein the one or more diseased cells are optionally from the subject's bone marrow, wherein the one or more diseased cells are optionally identified by a mutant nucleotide sequence from at least a portion of an IgH gene; an IgK gene; a TRB gene; a TRD gene; a TRG gene, or any combination thereof.

In certain embodiments, the hematological malignancy is NHL. In certain embodiments, the allo-HSCT is administered to the subject and/or the subject is monitored when the subject: (i) has a serum LDH level of less than about 210 U/L prior to receiving the lymphodepleting chemotherapy; (ii) has an increased level of serum MCP-1 prior to receiving the one or more infusion; (iii) has an International Prognostic Index (IPI) of 0 or 1 prior to receiving the lymphodepleting chemotherapy and the one or more infusion; (vi) has a reduced level of IL-18 prior to receiving the lymphodepleting chemotherapy and prior to the subject receiving the one or more infusions; (v) has an increased peak level of IL-7 prior to receiving the lymphodepleting chemotherapy; (vi) has an increased level of serum IL-7 prior to receiving the lymphodepleting chemotherapy; (vii) has a peak serum concentration of IL-7 by about 28 days following the one or more infusions of the modified immune cells of less than about 20 pg/mL; (viii) has a serum concentration of MCP-1 immediately prior to a first of the one or more infusions of the modified immune cells of less than about 10³ pg/mL; (vi) has a serum concentration of MCP-1 immediately prior to a first of the one or more infusions of the modified immune cells that is lower than, or that is up to about 20% greater than, the serum concentration of MCP-1 at the time of a first administration of the lymphodepleting chemotherapy; (ix) has received the cyclophosphamide at one or more dose of less than about 40 mg/kg; and/or (x) has received a total dose of the cyclophosphamide of less than about 1500 mg/m². In certain embodiments, wherein the therapy is administered to the subject when the subject had a serum LDH level of 210 U/L or more prior to receiving the lymphodepleting chemotherapy.

In other aspects, methods are provided for treating a hematological malignancy in a human subject, wherein the subject had received a first therapy comprising a lymphodepleting chemotherapy and one or more infusion of a modified immune cell comprising a heterologous polynucleotide that encodes a binding protein that specifically binds to an antigen expressed by or associated with the hematological malignancy, the method comprising administering a second therapy comprising allogeneic hematopoietic stem cell transplant; radiation therapy; chemotherapy; surgery; one or more further infusion of the modified immune cell; immunosuppressive therapy; or any combination thereof, when: (a) by about 28 days following the one or more infusion, the subject had a peak serum concentration of the heterologous polynucleotide encoding the binding protein of about 10² or fewer copies per microgram of DNA; (b) by about 28 days following the one or more infusion, the subject had a peak serum concentration of the modified immune cell of about 10¹ or fewer cells per microliter, as determined by flow cytometry; (c) prior to receiving the lymphodepleting chemotherapy, the subject has a serum lactate dehydrogenase (LDH) level of 210 U/L or more; (d) the subject has a peak serum concentration of IL-7 by about 28 days following the one or more infusions of the modified immune cells of less than about 20 pg/mL; (e) the subject has a serum concentration of MCP-1 immediately prior to a first of the one or more infusions of the modified immune cells of less than about 10³ pg/mL, less than about 10²⁵ pg/mL, less than about 10² pg/mL, less than about 10^{1.5} pg/mL, or lower; (f) the subject has a serum concentration of MCP-1 immediately prior to a first of the one or more infusions of the modified immune cells lower than, or up to about 20% greater than, the serum concentration of MCP-1 at the time of a first administration of the lymphodepleting chemotherapy; (g) the subject has received the cyclophosphamide at one or more doses of less than about 40 mg/kg, less than about 35 mg/kg, or less than about 30 mg/kg; and/or (h) the subject has received a total dose of the cyclophosphamide of less than about 1500 mg/m².

In certain embodiments, the hematological malignancy is selected from acute lymphoblastic leukemia (ALL), optionally B cell ALL, Hodgkin's lymphoma, non-Hodgkins lymphoma (NHL), primary central nervous system lymphomas, T cell lymphomas, small lymphocytic lymphoma (SLL), B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma (mucosa-associated lymphoid tissue (MALT) lymphoma), nodal marginal zone B-cell lymphoma, follicular lymphoma, mantle cell lymphoma, diffuse large B-cell lymphoma (DLBCL), mediastinal (thymic) large B-cell lymphoma, intravascular large B-cell lymphoma, primary effusion lymphoma, acute myeloid leukemia (AML), chronic lymphocytic leukemia (CLL), chronic myoblastic leukemia (CIVIL), Hairy cell leukemia (HCL), chronic myelomonocytic leukemia (CMML), juvenile myelomonocytic leukemia (JMML), large granular lymphocytic leukemia (LGL), blastic plasmacytoid dendritic cell neoplasm (BPDCN), Burkitt lymphoma/leukemia, multiple myeloma, Bence-Jones myeloma, non-secretory myeloma, plasmacytoma, amyloidosis, monoclonal gammopathy of unknown significance (MGUS), T cell/histiocyte-rich large B cell lymphoma, pleomorphic mantle cell lymphoma, or Waldenstrom's macroglobulinemia.

In certain embodiments, the hematological malignancy is B-ALL. In certain embodiments, the hematological malignancy is NHL.

In any of the embodiments disclosed herein, the encoded binding protein can comprise a chimeric antigen receptor (CAR). In certain embodiments, the encoded binding protein comprises a CAR comprising an extracellular component comprising a binding domain specific for the antigen and a hinge region, an intracellular component, and a transmembrane component disposed between the extracellular component and the intracellular component, wherein the hinge region is disposed between the binding domain and the transmembrane component.

In any of the embodiments disclosed herein, the antigen is CD19 (see, e.g., Hammer O. MAbs 4:571 (2012), including the anti-CD19 antibodies disclosed therein, which antibodies are incorporated herein by reference in their entirety). In certain embodiments, the binding domain of the encoded binding protein is derived from FMC-63 antibody (see, e.g., Zola et al. Immunol. Cell Biol. 69(Pt 6):411-22 (1991) and WO 2015/095895, which FMC-63 antibody and scFv sequences are incorporated herein by reference), MOR208 (see, e.g., Horton et al., Cancer Res. 68(19): (2008); see also Meeker et al. Hybridoma 3:305 (1984)), blinatumomab (see, e.g., Mølhøj et al., Mol. Immunol. 44(8):1935 (2007)), MEDI-551 (see, e.g., Herbst et al., J. Pharmacol. Exp. Ther. 335:213 (2010)), Merck patent anti-CD19 antibody, Xmab5871 aka obexelimab, or MDX-1342; and/or the hinge region is derived from IgG4; and/or the transmembrane component is derived from CD28; and/or the intracellular component comprises a 4-1BB signaling domain and a CD3ζ domain.

In any of the embodiments disclosed herein, one or more of the infusions comprises modified CD4+ T cells and modified CD8+ T cells in about a 1:1 ratio. In certain embodiments, one or more of the infusions comprises modified CD4+ T cells and modified CD8+ T cells in a 1:1 ratio.

In any of the embodiments disclosed herein, the subject has previously received one or more infusion comprising 2×10⁵ to 2×10⁶ of the modified immune cells/kg.

In still further embodiments, a subject identified in a method of this disclosure as at risk for an adverse event receives a pre-emptive treatment (also referred to as a second therapy) for the potential relapse or an altered cellular immunotherapy regimen comprising administering the cellular immunotherapy at a reduced dose, a corticosteroid, an inflammatory cytokine antagonist, or any combination thereof. In particular embodiments, a pre-emptive treatment comprises a corticosteroid selected from dexamethasone, prednisone, or both. In related embodiments, a pre-emptive treatment comprises an inflammatory cytokine antagonist comprising an anti-IL-6 antibody, an anti-IL-6R antibody, or both. In further embodiments, a pre-emptive treatment comprises administering a corticosteroid and an inflammatory cytokine antagonist, such as dexamethasone with tocilizumab or siltuximab, or prednisone with tocilizumab or siltuximab.

In any of the aforementioned embodiments, provided herein are methods for reducing the risk of relapse or treating a hematological malignancy, wherein the subject has a hematologic malignancy is selected from Hodgkin's lymphoma, non-Hodgkins lymphoma (NHL), primary central nervous system lymphomas, T cell lymphomas, small lymphocytic lymphoma (SLL), B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma (mucosa-associated lymphoid tissue (MALT) lymphoma), nodal marginal zone B-cell lymphoma, follicular lymphoma, mantle cell lymphoma, diffuse large B-cell lymphoma (DLBCL), mediastinal (thymic) large B-cell lymphoma, intravascular large B-cell lymphoma, primary effusion lymphoma, acute lymphoblastic leukemia (ALL), acute myeloid leukemia (AML), chronic lymphocytic leukemia (CLL), chronic myoblastic leukemia (CIVIL), Hairy cell leukemia (HCL), chronic myelomonocytic leukemia (CMML), juvenile myelomonocytic leukemia (JMML), large granular lymphocytic leukemia (LGL), blastic plasmacytoid dendritic cell neoplasm (BPDCN), Burkitt lymphoma/leukemia, multiple myeloma, Bence-Jones myeloma, non-secretory myeloma, plasmacytoma, amyloidosis, monoclonal gammopathy of unknown dignificance (MGUS), or Waldenstrom's macroglobulinemia.

In certain embodiments, the present disclosure provides methods for reducing the risk of death, relapse, and/or disease progression wherein a biological sample is obtained before pre-conditioning, before cellular immunotherapy administration, or before both. In some embodiments, a subject having a hematologic malignancy and being treated with adoptive cellular immunotherapy (e.g., CAR-T cell therapy) will receive a “pre-conditioning” (or simply a “conditioning”) regimen to reduce the tumor burden and to suppress the recipient's immune system to allow engraftment of the adoptive cellular immunotherapy. The conditioning may be myeloablative in which total body irradiation (TBI) or alkylating agents are administered, at doses that do not allow autologous hematologic recovery and, therefore, include stem cell therapy. For example, myeloablative conditioning may comprise TBI at 10 Gy, with cyclophosphamide (CY) at 200 mg/kg and busulfan (BU) at 16 mg/kg. Other agents that can be used in a myeloablative conditioning regimen at high doses, and in different combinations with CY or TBI, include melphalan (MEL), thiotepa (THIO), etoposide (VP16), and dimethylbusulfan. Alternatively, the conditioning may be non-myeloablative in which less toxic treatments are used and stem cell therapy is not needed. For example, non-myeloablative conditioning regimens include fludarabine and cyclophosphamide (Flu/CY), TBI at 2 Gy or 1 Gy, total lymphoid radiation (TLI), and anti-thymocyte globulin (ATG). In certain embodiments, conditioning for subjects with a hematologic malignancy (e.g., lymphoma, leukemia, myeloma) comprises administration daily for two to five days of cyclophosphamide (CY) at 30-60 mg/kg alone or CY at 30-60 mg/kg and fludarabine (Flu) at 25-30 mg/m².

In certain embodiments, a biological sample of the aforementioned methods is obtained from the subject within 12 hours, 24 hours, 36 hours, or 48 hours after cellular immunotherapy.

Any of the aforementioned pre-emptive treatments or altered cellular immunotherapy regimens comprises administering the cellular immunotherapy at a reduced dose, a corticosteroid, an inflammatory cytokine antagonist, an endothelial cell stabilizing agent, or any combination thereof, apply to this method as well.

Also provided herein are kits for use in diagnosing or detecting the risk of a relapse of a hematological malignancy in a subject that presents with a MRD-negative CR following administration to the subject of lymphodepleting chemotherapy and one or more infusion of modified immune cells containing a heterologous polynucleotide that encodes a binding protein that specifically binds to an antigen expressed by or associated with the hematological malignancy, the kit comprising one or more reagent for: (i) measuring the amount of LDH in a serum sample from the subject; (ii) measuring the amount of platelets present in a serum sample from the subject; (iii) measuring a serum concentration of IL-7 in a sample from the subject; and/or (iv) measuring a serum concentration of MCP-1 in a sample from the subject, wherein the subject is identified as being at risk of relapse when the subject: (a) has a serum LDH level of about 210 or more U/L prior to receiving the lymphodepleting chemotherapy and/or (b) has a platelet count of less than about 100 U/L prior to receiving the lymphodepleting chemotherapy; (c) has a peak serum concentration of IL-7 by about 28 days following the one or more infusions of the modified immune cells of less than about 20 pg/mL; (d) has a serum concentration of MCP-1 immediately prior to a first of the one or more infusions of the modified immune cells of less than about 10³ pg/mL, less than about 10²⁵ pg/mL, less than about 10² pg/mL, less than about 10¹⁵ pg/mL, or lower; and/or(e) has a serum concentration of MCP-1 immediately prior to a first of the one or more infusions of the modified immune cells lower than, or up to about 20% greater than, the serum concentration of MCP-1 at the time of a first administration of the lymphodepleting chemotherapy.

In certain embodiments, a kit according to the present disclosure further comprises one or more reagent for measuring the amount of the heterologous polynucleotide in a serum sample from the subject.

In certain embodiments, a kit according to the present disclosure further comprises instructions for performing the measuring.

In certain embodiments, a kit according to the present disclosure further comprises instructions for providing a pre-emptive therapy when the subject is identified as being at-risk for relapse, wherein the pre-emptive therapy comprises allogeneic hematopoietic stem cell transplant; radiation therapy; chemotherapy; surgery; one or more further infusion of the modified immune cells; immunosuppressive therapy; or any combination thereof.

In certain embodiments, diagnostic procedures described herein may be performed by diagnostic laboratories, experimental laboratories, or practitioners. Materials and reagents for characterizing biological samples and diagnosing the risk of relapse in a hyperproliferative disease (or of failing to achieve a complete response) in a subject treated by immunotherapy according to the methods herein may be assembled together in a kit. In certain aspects, a kit comprises at least one reagent that specifically detects levels of one or more biomarkers disclosed herein, and instructions for using the kit according to a method of this disclosure.

In any of the presently disclosed embodiments, wherein the modified immune cells are autologous to the subject (e.g., were produced by a method comprising leukapheresis of the subject, wherein the leukapheresis occurred prior to administration of the lymphodepleting chemotherapy), and wherein following the leukapharesis and the prior to the lymphodepleting chemotherapy, the subject received a bridging therapy comprising one or more of: (i) chemotherapy; (ii) a corticosteroid, wherein the corticosteroid is optionally dexamethasone; (iii) a monoclonal antibody or antigen-binding fragment thereof; (iv) an immunomodulatory agent; (v) a targeted small molecule chemotherapeutic agent; or (vi) any combination of (i)-(v).

In certain embodiments, the present disclosure provides methods for treating a hematological malignancy, wherein the methods comprise administering to a subject in need thereof an effective amount of a Bruton's tyrosine kinase (BTK) inhibitor when the subject is receiving or has received a modified immune cell that specifically targets an antigen that is expressed by or associated with the hematological malignancy. In other embodiments, methods are provided for treating a hematological malignancy, wherein the methods comprise administering to a subject who is receiving or has received a BTK inhibitor an effective mount of a modified immune cell that specifically targets an antigen that is expressed by or associated with the hematological malignancy.

By way of background, Bruton's tyrosine kinase (also referred to as AGMX1, AT, ATK, BPK, IMD1, PSCTK1, XLA, and BtK) is in some contexts known as a kinase that is involved in B cell maturation and mast cell activation. BTK includes a pleckstrin homology (PH) domain that binds to phosphatidylinositol-3,4,5-trisphosphate (PIP3), which binding event is believed to induce BTK to phosphorylate phospholipase C, which can, in turn, hydrolyze the phosphatidylinositol phosphatidylinositol-4,5-bisphosphate (PIP2) into inositol triphosphate (IP3) and diacylglycerol (DAG). Without wishing to be bound by theory, IP3 and DAG can modulate the activity of downstream proteins during B cell signaling.

In some contexts, a BTK inhibitor is a molecule (e.g., small organic molecule, antibody, peptide), compound, or composition that inhibits (e.g., reduces, attenuates, slows, delays, or abrogates) one or more biological activity of BTK and/or inhibits BTK-mediated B cell signaling. BTK inhibitors include, for example, ibrutinib (PCI-32765), acalabrutinib, ONO-4059 (GS-4059), spebrutinib (AVL-202; CC-292), BHB-3111, and HM71224. Ibrutinib is an approved treatment for a number of hematological malignancies, including CLL. Acalabrutinib is approved for treating relapsed mantle cell lymphoma.

In any of the presently disclosed embodiments, the BTK inhibitor comprises ibrutinib, acalabrutinib (ACP-196), ONO-4059 (GS4059), spebrutinib, BGB-3111, HM71224, or any combination thereof. In certain embodiments, the BTK inhibitor comprises ibrutinib.

In certain embodiments, the subject receives or had received the BTK inhibitor for about one week to about six months, or longer, following a first administration of the modified immune cell to the subject. In certain embodiments, the subject receives or had received the BTK inhibitor for at least about one week to about five years prior to a first administration of the modified immune cell to the subject.

In certain embodiments, the hematological malignancy is selected from chronic lymphocytic leukemia (CLL), non-Hodgkin's lymphoma (NHL), mantle cell lymphoma, acute lymphoblastic leukemia (ALL), optionally B cell ALL, small lymphocytic lymphoma (SLL), Hodgkin's lymphoma, primary central nervous system lymphomas, T cell lymphomas, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma (mucosa-associated lymphoid tissue (MALT) lymphoma), nodal marginal zone B-cell lymphoma, follicular lymphoma, diffuse large B-cell lymphoma (DLBCL), mediastinal (thymic) large B-cell lymphoma, intravascular large B-cell lymphoma, primary effusion lymphoma, acute myeloid leukemia (AML), chronic myoblastic leukemia (CML), Hairy cell leukemia (HCL), chronic myelomonocytic leukemia (CMML), chronic myeloid leukemia, juvenile myelomonocytic leukemia (JMML), large granular lymphocytic leukemia (LGL), blastic plasmacytoid dendritic cell neoplasm (BPDCN), Burkitt lymphoma/leukemia, multiple myeloma, Bence-Jones myeloma, non-secretory myeloma, plasmacytoma, amyloidosis, myelodysplastic syndrome (MDS), monoclonal gammopathy of unknown significance (MGUS), or Waldenstrom's macroglobulinemia.

In particular embodiments, the hematological malignancy is CLL.

In certain embodiments, the subject had experienced a progression and/or a relapse of the hematological malignancy while receiving the BTK inhibitor and prior to administration of the modified immune cell.

In certain embodiments, the subject is intolerant of the BTK inhibitor. Intolerance of a BTK inhibitor includes allergic and other reactions, and can manifest as, for example, in adverse events such as arhtraliga, atrial fibrillation, bleeding, rash, brusing, CHF, diarrhea or colitis, fatigure, anorexia, dizziness, edema, hypertension, peridcadrial effusion, a change in mental or cognitive status, or the like.

In certain embodiments, the hematological malignancy is refractory to the BTK inhibitor. In some contexts, a disease is considered to be “refractory” if does not respond to (or is resistant to) one or more attempted forms of treatment. Refractory disease may be characterized, for example, by continued or increased growth, spread, or severity of disease, or failure to decrease in size or rate of growth or spread, or any combination thereof. In some instances, endogenous or acquired mutations in BTK can disrupt, prevent, or abrogate the binding by which ibrutinib is believed to inhibit BTK activity. For example, mutations in the Phospholipase C Gamma 2 (PCLG2) gene can allow a diseased B cell to bypass or override (e.g., in whole or in part) inhibition of BTK by ibrutinib. In these and certain other embodiments, a disease may be “refractory to” ibrutinib.

A state, status, diagnosis, or grade of a disease or condition (or risk thereof or risk relating thereto) may be with reference to one or more particular disease or condition. In certain embodiments, a certain term or terms used to describe, define, or diagnose a disease or condition will be known to those having ordinary (e.g., clinical or academic) familiarity with the disease or condition. For example, in some embodiments where the subject has CLL or the disease or condition is CLL, the subject is deemed to have bulky disease if the subject is assessed as having extensive nodal involvement and/or determined to have palpable lymph nodes and/or other organs having been infiltrated with disease, for example, as opposed to having disease confined to the bone marrow. See, e.g., Trageser, “Understanding Chronic Lymphocytic Leukemia,” IGLiving, available at www.igliving.com/magazine/articles/IGL_2012-10_AR_Understanding_Chronic_Lymphocytic_Leukemia.pdf (accessed on Oct. 26, 2018); Dohner et al., Blood 89:2516-2522 (1997); and Zent and Kay, Leuk. Lymphoma 52(8):1425-1434 (2011), the CLL-related risk factors, risk stratification systems, disease statuses and related terminology, and prognostic indicators and prognostic and diagnostic methodologies of which are incorporated by reference herein. In some aspects, the subject is determined to have bulky disease in CLL if the subject exhibits palpable nodes greater than a certain diameter, such as greater than 5 cm in diameter and/or exhibits a spleen palpable greater than 6 cm below costal margin. In some embodiments, a diameter of a subject organ, tumor, or tissue may be measured, approximated, or calculated using a CT scan, a PET scan, or both.

As a further example, in some contexts, CLL may be associated with a karyotype that is deemed a complex karyotype. In some embodiments, a complex karyotype may comprise three or more distinct chromosomal abnormalities present in more than one metaphase. See, e.g., Thompson et al., Cancer 121(20):3612-3621 (2015) and Turtle et al., J. Clin. Oncol. 35(26):3010 (2017). In some embodiments, a complex karyotype may comprise 5 or more distinct chromosomal abnormalities; e.g., present in more than one metaphase. See, e.g., Jaglowski et al., Br. J. Haematology 159(1):81-87 (2012). In some embodiments, a complex karyotype may comprise 4 or more distinct chromosomal abnormalities, e.g., present in more than one metaphase. See also Waheed et al., J. Oncol. (2018), Article ID 2019239.

In certain embodiments, the subject had received ibrutinib for about 6, 7, 8, 9, 10, 11, 12, 13, 14, 15, 16, 17, 18, 29, 20, 21, 22, 23, 24, 25, 26, 27, 28, 29, 30, 31, 32, 33, 34, 35, 36, or more months prior to experiencing a progression of the hematological malignancy.

In certain embodiments, wherein, prior to receiving the modified immune cell, the subject had a maximum Standard Uptake Value (SUV) of about 3, 4, 5, 6, 7, 8, 9, 10, 11, or more, wherein the SUV was optionally determined by positron emission tomography (PET) and/or x-ray computerized tomography (CT) comprising use of a labeled tracer molecule, wherein the labeled tracer molecule optionally comprised a radiolabeled tracer molecule, optionally 2-deoxy-2-[¹⁸F]fluoro-D-glucose (FDG).

In certain embodiments, prior to receiving the modified immune cell, the subject: (i) had a complex karyotype, optionally comprising a chromosome 17p deletion, a chromosome 11q deletion, a chromosome 13q deletion, trisomy 12, a TP53 stop or missense mutation, 3, 4, 5, or more distinct chromosomal abnormalities present in more than one metaphase, or any combination thereof; (ii) had a mutation in a BTK gene that affects the ability of ibrutinib to bind to BTK, wherein the mutation is optionally a substitution mutation at position C481, wherein the substitution mutation is optionally C481S; (iii) had a gain-of-function (GOF) mutation in a PLCG2 gene, wherein the GOF mutation optionally comprises R665W, L845F, S707Y, or any combination thereof; (iv) had a serum LDH concentration of about 130 U/L, 140 U/L, 150 U/L, 160 U/L, 170 U/L, 180 U/L, 190 U/L, 200 U/L, 210 U/L, 220 U/L, 230 U/L, 240 U/L, 250 U/L, 260 U/L, 270 U/L, 280 U/L, 290 U/L, 300 U/L, 310 U/L, or more; (v) had bulky disease; (vi) had extensive nodal involvement of the hematological malignancy; (vii) had palpable lymph nodes and/or infiltration of cells of the hematological malignancy in other organs or tissues; (viii) had malignant cells not confined to bone marrow; (ix) had palpable nodes greater than 5 cm in diameter and/or had a palpable spleen greater than 6 cm below the costal margin; (x) had a high-risk histology, optionally characterized by Richter's transformation, prolymphocytic leukemia, IPC, or any combination thereof (xi) had extramedullary disease; (xii) had about 10%, 15%, 20%, 25%, 30%, 35%, 40%, 45%, 50%, 55%, 60%, 65%, 70%, 75%, or 80% malignant cells in bone marrow, or more; (xiii) had about 5%, 10%, 15%, 20%, 25%, 30%, 35%, 40%, 45%, 50%, 55%, or 60% malignant cells in blood, or more; (xiv) had about 1×10⁹, 2×10⁹, 3×10⁹, 4×10⁹, 5×10⁹, 6×10⁹, 7×10⁹, 8×10⁹, 9×10⁹, or more malignant cells/L blood; (xv) had about 1×10⁹, 2×10⁹, 3×10⁹, 4×10⁹, 5×10⁹, 6×10⁹, 7×10⁹, 8×10⁹, 9×10⁹, or more lymphocytes/L blood; (xvi)had previously received allogeneic stem cell transplantation; or (xvii) had any combination of (i)-(xvi), wherein the hematological malignancy is optionally CLL.

In certain embodiments, the subject had previously been administered venetoclax, rituximab, idelalisib, or any combination thereof. In certain embodiments, the subject had experienced a progression and/or relapse of the hematological malignancy prior to administration of the modified immune cell and while receiving the venetoclax, rituximab, idelaisib, or combination thereof.

In certain embodiments, the antigen expressed by or associated with the hematological malignancy is a CD19 antigen.

In certain embodiments, the modified immune cell comprises an autologous immune cell obtained from the subject that is modified to contain the heterologous polynucleotide encoding the binding protein, wherein the subject is administered the BTK inhibitor at least once between the time the autologous immune cell is obtained from the subject and the time the modified immune cell is administered to the subject.

In certain embodiments, following the administering of the modified immune cell, the subject has: (i) a reduced tumor burden (e.g., reduced tumor size, surface area, density, number, and/or distribution) as compared to a reference subject who does not receive the modified immune cell; (ii) a reduced number and/or severity of immune cell-related toxicity events as compared to a reference subject who does not receive the modified immune cell; and/or (iii) has about the same as, or has an increased number of the modified immune cell (e.g., as determined using flow cytometry) as compared to a reference subject (i.e., a comparator subject of a same disease type and severity who receives a same therapy with the exception of the modified immune cell or the BTK inhibitor) but who does not receive the modified immune cell and/or who does not receive the BTK inhibitor.

Immune cell-related toxicity events include the production and release of excess amounts of cytokines following administration of an immune cell therapy; possible mechanisms, manifestations, symptoms, grading scales, and therapies for immune-cell related toxicity events (e.g., cytokine release syndrome, cytokine storm) are known in the art (see, e.g., Shimabukuro-Vornhagen et al., 6:56 (2018); Liu and Zhao, J. Hematol. Oncol. 11:121 (2018); Penn grading scale, CTCAE v4.0, 2014 Lee et al. scale, and MDACC grading, which detection methods, grading scales, and therapies are incorporated herein by reference). In certain embodiments, serum cytokine concentrations can be determined using a Luminex® Assay (see, e.g., Turtle et al., J. Clin. Invest. 126:2123-2138 (2016); Turtle et al., Sci. Transl. Med. 8:355ra116 (2016)).

In certain embodiments, the method further comprises performing nucleic acid sequencing on at least a portion of an IgH gene locus in a bone marrow sample from the subject before and/or after administering the modified immune cell. Nucleic acid sequencing techniques, processes, and reagents are known in the art.

In certain embodiments, the subject is disease-negative as determined by sequencing of the at least a portion of the IgH locus after administering the modified immune cell. As used herein, “disease-negative” means that the disease (e.g., a cell, cells characterized by the presence of a disease-associated marker, such as a genetic mutation or genotype associated with the disease) is not present at a detectable level in the subject or in a sample from the subject (e.g., bone marrow, blood), or is present at less than one malignant cell per million cells. Techniques and markers for sequencing a gene locus of interest (or a portion thereof) to identify the presence or absence of a hematologic malignancy are known and include those described in Scherer et al., Blood 130:440-452 (2017), which techniques and markers are incorporated herein by reference.

Also provided herein are methods for treating follicular lymphoma (FL) in a subject, wherein the methods comprise administering to the subject an effective amount of a modified immune cell containing a heterologous polynucleotide that encodes a binding protein that specifically binds to an antigen expressed by or associated with the FL, wherein the subject had previously received lymphodepleting chemotherapy prior to the modified immune cell, and wherein, following the administering of the modified immune cell, the subject: (i) is alive for at least 6, 12, 18, 24, 30, 36, or 38 months; (ii) presents with no progression (i.e., shows no statistically significant growth and/or spread) of the FL for at least 6, 12, 18, 24, 30, 36, or 38 months; and/or (iii) presents with a complete remission of the FL for at least 6, 12, 18, 24, 30, 36, or 38 months.

In certain embodiments, the FL comprises transformed follicular lymphoma (tFL). In some contexts, FL is typically an indolent disease, whereas tFL is a FL that has undergone a histologic transformation (referred to as “HT”) to a more aggressive form of cancer, such as an aggressive non-Hodgkin lymphoma, for example an aggressive B-cell lymphoma, or a diffuse large B cell lymphoma (DLBCL). Potential mechanisms and mutations (e.g., MYC, BCL2, and/or BCL6 gene rearrangements) that associate with HT from indolent FL to tFL are discussed in, for example, Casulo et al., 125:40-47 (2015), and Jack et al., Blood 122:78 (2013), and are incorporated herein.

In certain embodiments, the subject had received treatment prior to the lymphodepleting chemotherapy (e.g., during and/or after a leukapharesis or other procedure to obtain a modified immune cell that is autologous to the subject).

In certain embodiments, the subject had presented with a relapse and/or progression of disease following a prior therapy for the FL, wherein the prior therapy comprises a biological agent (e.g., a cytokine, a monoclonal antibody or an antigen-binding fragment thereof, such as a bispecific T cell engager molecule (BiTE), an antibody-drug conjugate, or the like), a chemotherapy, a hematopoietic stem cell transplantation (HCT), or any combination thereof.

In certain embodiments, the lymphodepleting chemotherapy comprises cyclophosphamide and/or fludarabine.

In certain embodiments, prior to receiving the modified immune cell, the subject has: stage III FL; stage IV FL; extranodal involvement of FL; an intermediate or high FL International Prognostic Index score; MYC and BLC2 and/or BCL6 rearrangement (DH/TH); or any combination thereof. Grading criteria and scales for identifying and diagnosing FL are known in the art and include those described in, for example, Swedlow et al., Blood 127(20):1385-2390 (2016); Freedman, Am. J. Hematol. 93(2):296-305 (2018); and the National Cancer Institute's PDQ Cancer Information Summaries (see Table 4), e.g., NBK65726.15 (Apr. 12, 2019 version), available online at ncbi.nlm.nih.gov/books NBK65726/table/CDR0000062933_557/; see also In: Edge et al., eds.: AJCC Cancer Staging Manual. 7th ed. New York, N.Y.: Springer, 2010, pp 607-11.

In any of the presently disclosed embodiments, the encoded binding protein comprises a chimeric antigen receptor (CAR).

In certain embodiments, the encoded binding protein comprises a CAR. In certain embodiments, the CAR comprises an extracellular component comprising a binding domain specific for the antigen and a hinge region, an intracellular component, and a transmembrane component disposed between the extracellular component and the intracellular component, wherein the hinge region is disposed between the binding domain and the transmembrane component.

In any of the presently disclosed embodiments, the antigen is a CD19 antigen and wherein the CAR comprises a binding domain comprising a scFv that specifically binds to the CD19 antigen.

In certain embodiments, the binding protein is a CAR and: the binding domain comprises CDRs from, or comprises a VH and/or a VL from, or comprises a VH and/or a VL having a least about 80%, 85%, 90%, 95%, 96%, 97%, 98%, or 99% identity to that of FMC-63 antibody, MOR208, blinatumomab, MEDI-551, Merck patent anti-CD19 antibody, Xmab5871, or MDX-1342. MOR208, blinatumomab, MEDI-551, Merck patent anti-CD19 antibody, Xmab5871, or MDX-1342; and/or the hinge region is derived from IgG4; and/or the transmembrane component is derived from CD28; and/or the intracellular component comprises a 4-1BB signaling domain and a CD3ζ domain.

In certain embodiments, the modified immune cell comprises a human immune cell.

In some embodiments, the modified immune cell comprises an autologous immune cell from the subject. In some embodiments, the human immune cell comprises a hematopoietic stem cell, a lymphoid progenitor cell, a T cell, a NK cell, a NK-T cell, a B cell, a myeloid progenitor cell, a monocyte, a macrophage, a dendritic cell, a megakaryocyte, a granulocyte, or any combination thereof.

In certain embodiments, the modified immune cell comprises modified CD4+ T cells and modified CD8+ T cells in about a 1:1 ratio. In some embodiments, the modified CD8+ T cells comprise central memory T cells.

In any of the presently disclosed embodiments, the subject is receiving or has received about 2×10⁵, about 2×10⁶, or about 2×10⁷ modified immune cells/kg.

In any of the presently disclosed embodiments, prior to receiving the modified immune cell, the subject received lymphodepleting chemotherapy. In certain embodiments, the lymphodepleting chemotherapy comprises cyclophosphamide and fludarabine.

Any of the therapeutic regimens disclosed herein can comprise a cellular immunotherapy and/or a BTK inhibitor in combination with one or more additional combination or adjunctive therapies, such as in combination with any one or more additional composition as provided herein. In certain embodiments, a therapeutic regimen comprises a modified immune cell and one or more of a chemotherapy as disclosed herein, a cytokine, a biologic therapy, a hormonal therapy, or any combination thereof.

Exemplary additional or adjunctive chemotherapeutic agents include, for example, alkylating agents (e.g., cisplatin, oxaliplatin, carboplatin, busulfan, nitrosoureas, nitrogen mustards such as bendamustine, uramustine, temozolomide), antimetabolites (e.g., aminopterin, methotrexate, mercaptopurine, fluorouracil, cytarabine, gemcitabine), taxanes (e.g., paclitaxel, nab-paclitaxel, docetaxel), anthracyclines (e.g., doxorubicin, daunorubicin, epirubicin, idaruicin, mitoxantrone, valrubicin), bleomycin, mytomycin, actinomycin, hydroxyurea, topoisomerase inhibitors (e.g., camptothecin, topotecan, irinotecan, etoposide, teniposide), monoclonal antibodies (e.g., ipilimumab, pembrolizumab, nivolumab, avelumab, alemtuzumab, bevacizumab, cetuximab, gemtuzumab, panitumumab, rituximab, tositumomab, trastuzumab), vinca alkaloids (e.g., vincristine, vinblastine, vindesine, vinorelbine), cyclophosphamide, fludarabine, oxaliplatin, prednisone, leucovorin, oxaliplatin, hyalurodinases, or any combination thereof.

In certain embodiments, a combination or adjunctive therapy further or alternatively comprises one or more of chemotherapy, a biologic therapy, a hormonal therapy, or any combination thereof.

In certain embodiments, a biologic therapy includes an antibody, an scFv, a nanobody, a fusion protein (e.g., chimeric antigen receptor (CAR), such as used in adoptive immunotherapy comprising a T cell expressing an antigen specific CAR on its cell surface), a tyrosine kinase inhibitor, an immunoreactive T cell, an immunoreactive Natural Killer cell (NKC), or any combination thereof. In certain further embodiments, an antibody comprises ipilimumab, pembrolizumab, nivolumab, avelumab, cetuximab, trastuzumab, bevacizumab, alemtuzumab, gemtuzumab, panitumumab, rituximab, tositumomab, or any combination thereof.

To practice coordinate administration of therapies of this disclosure, therapy regimens combine cellular immunotherapy (e.g., CAR-modified T cell) with an additional or adjunctive therapy simultaneously or sequentially in a coordinated treatment protocol. For example, a therapy regimen may combine a conditioning procedure with a cellular immunotherapy and an optional combination therapy comprising chemotherapy, radiation therapy or the like. In this example, an optional combination therapy may comprise one or more chemotherapeutic agents to be administered concurrently or sequentially, in a given order or otherwise with a conditioning regimen, a cellular immunotherapy, or both.

A coordinate administration of one or more therapies or agents may be done in any order, and there may be a time period while only one or both (or all) therapies, individually or collectively, exert their biological activities. A distinguishing aspect of all such coordinate treatment methods is that a treatment regimen elicits some favorable clinical response, which may or may not be in conjunction with a secondary clinical response provided by an additional therapeutic agent or process. For example, the coordinate administration of a cellular immunotherapy with a combination therapy as contemplated herein can yield an enhanced (e.g., synergistic) therapeutic response beyond the therapeutic response elicited by any of the therapies alone.

For the purposes of administration, the compounds of the present disclosure may be administered as a raw chemical or may be formulated as pharmaceutical compositions, as disclosed herein. Pharmaceutical compositions of the present disclosure may comprise a small molecule, chemical entity, nucleic acid molecule, peptide or polypeptide (e.g., antibody), cell, and a pharmaceutically acceptable carrier, diluent or excipient. The small molecule, chemical entity, nucleic acid molecule, peptide or polypeptide composition will be in an amount that is effective to treat a particular disease or condition of interest—that is, in an amount sufficient for reducing the risk of or treating a hyperproliferative disease or proliferative disease, such as hematologic malignancies or any of the other associated indications described herein, and preferably with acceptable toxicity to a patient. Compounds for use in the methods described herein can be determined by one skilled in the art, for example, as described in the Examples below. Appropriate concentrations and dosages can be readily determined by one skilled in the art.

Administration of the cells and compounds, or their pharmaceutically acceptable salts, in pure form or in an appropriate pharmaceutical composition, of this disclosure can be carried out using any mode of administration for agents serving similar utilities. The pharmaceutical compositions of this disclosure can be prepared by combining a cell or compound of this disclosure with an appropriate pharmaceutically acceptable carrier, diluent or excipient, and compounds may be formulated into preparations in solid, semi-solid, liquid or gaseous forms, such as tablets, capsules, powders, granules, ointments, solutions, suppositories, injections, inhalants, gels, microspheres, and aerosols. Exemplary routes of administering such pharmaceutical compositions include oral, topical, transdermal, inhalation, parenteral, sublingual, buccal, rectal, vaginal, and intranasal.

The term “parenteral” as used herein includes subcutaneous injections, intravenous, intramuscular, intrasternal injection or infusion techniques. Pharmaceutical compositions of this disclosure are formulated to allow the active ingredients contained therein to be bioavailable upon administration of the composition to a patient. Compositions that will be administered to a subject or patient take the form of one or more dosage units, where for example, a tablet may be a single dosage unit, and a container of a compound of this disclosure in aerosol form may hold a plurality of dosage units. Actual methods of preparing such dosage forms are known, or will be apparent, to those skilled in this art (see, e.g., Remington: The Science and Practice of Pharmacy, 22^{nd} Edition (Pharmaceutical Press, 2012). The composition to be administered will, in any event, contain a therapeutically effective amount of a compound of this disclosure, or a pharmaceutically acceptable salt thereof, for reducing the risk of or treating cancer, metastases arising from the cancer or other conditions of interest in accordance with the teachings of this disclosure.

### EXAMPLES

### Example 1

### Experimental Procedures

### Patient Characteristics, Lymphodepletion Chemotherapy and CAR-T Cell Infusion

A single-center study of factors impacting the strength and duration of responses to CAR-T cell therapy was conducted in 166 adult patients (age ≥18) with relapsed and/or refractory CD19⁺ B-ALL (n=57), NHL (n=79) and CLL (n=30) who received lymphodepletion chemotherapy followed by CD19-specific chimeric antigen receptor (CAR)-modified T cells (CAR-T cell) in a phase 1/2 CAR-T cell dose escalation/de-escalation clinical trial (Turtle et al. I, 2016; Turtle et al. II, 2016). The study was conducted with approval of the Fred Hutchinson Cancer Research Center (FHCRC) institutional review board, and is available at clinicaltrials.gov/ct2/show/NCT01865617. Informed consent was obtained from all patients.

CD19-specific CAR-modified T cells were manufactured as described in Turtle et al. I, 2016; and Turtle et al. II, 2016. In brief, patients underwent leukapheresis to obtain PBMC, from which CD4⁺ and CD8⁺ central memory T cell subsets were enriched. Enriched CD4⁺ and CD8⁺ central memory T cells were stimulated with anti-CD3/anti-CD28 coated paramagnetic beads and transduced with a lentivirus encoding a CAR comprising a FMC63-derived CD19-specific scFv, a modified IgG4-hinge spacer, a CD28 transmembrane domain, a 4-1BB costimulatory domain, and a CD3ζ signaling domain. A cell-surface human EGFRt was also encoded in the lentiviral vector separated from the CAR coding sequence cassette by a T2A ribosomal skip sequence to allow precise enumeration of transduced CD4⁺ and CD8⁺ CAR-T cells by flow cytometry. The modified T cells were formulated in a 1:1 ratio of CD3⁺/CD4⁺/EGFRt⁺ T cells to CD3⁺/CD8⁺/EGFRt⁺ T cells for infusion at one of three dose levels (DL) as follows: DL1=2×10⁵EGFRt⁺ cells/kg; DL2=2×10⁶EGFRt⁺ cells/kg; and DL3=2×10⁷EGFRt⁺ cells/kg. In patients with high circulating tumor burden or severe lymphopenia, selection of bulk CD8⁺ T cells rather than CD8⁺ central memory T cells could be performed. Patients received lymphodepletion chemotherapy with a cyclophosphamide-based regimen with or without fludarabine (Table 1), followed 2-4 days later by infusion of the CD19-specific CAR-modified T cells. Delay of CAR-T cell infusion was permitted for patients with clinical conditions (e.g., active and uncontrolled infection) that precluded CAR-T cell infusion at the scheduled time.

TABLE 1
Lymphodepletion Regimens prior to CAR-T Cell Infusion

| Lymphodepletion Regimen | Number of Patients |
|---|---|
| Cyclophosphamide 2 g/m² Day 1; | 2 |
| Etoposide 200 mg/m² Days 2-4 | |
| Cyclophosphamide 4 g/m² Day 1; | 3 |
| Etoposide 200 mg/m² Days 2-4 | |
| Cyclophosphamide 3 g/m² Day 1; | 2 |
| Etoposide 200 mg/m² Days 2-4 | |
| Cyclophosphamide 2 g/m² Day 1; | 2 |
| Etoposide 200 mg/m² Days 2-4 | |
| Cyclophosphamide 2 g/m² Day 1 | 14 |
| Cyclophosphamide 3 g/m² Day 1 | 2 |
| Cyclophosphamide 4 g/m² Day 1 | 1 |
| Bendamustine 90 mg/m² Day 1-2 | 1 |
| Fludarabine 25 mg/m² Day 1-3 | 2 |
| Cyclophosphamide 30 mg/kg Day 1; | 11 |
| Fludarabine 25 mg/m² Day 2-4 | |
| Cyclophosphamide 60 mg/kg Day 1; | 78 |
| Fludarabine 25 mg/m² Day 2-4 | |
| Cyclophosphamide 1 g/m² Day 1; | 1 |
| Fludarabine 25 mg/m² Day 2-4 | |
| Cyclophosphamide 60 mg/kg Day 1; | 11 |
| Fludarabine 25 mg/m² Day 2-6 | |
| Cyclophosphamide 300 mg/m² and | 1 |
| Fludarabine 30 mg/m² Day 1-3 | |
| Cyclophosphamide 500 mg/m² and | 2 |
| Fludarabine 30 mg/m² Day 1-3 | |
| Total | 133 |

### Evaluation of Clinical Laboratory Parameters, CAR-T Cell Counts, and Biomarker Concentrations

Blood was collected before lymphodepletion, on day 0 before CAR-T cell infusion, and at approximately 1, 3, 7, 10, 14, 21, and 28 days after CAR-T cell infusion. Complete blood counts and laboratory analyses of renal and hepatic function, and coagulation were performed using Clinical Laboratory Improvement Amendments (CLIA)-certified assays in clinical laboratories. CD4⁺ and CD8⁺ CAR-T cells were identified by flow cytometry as viable CD45⁺/CD3⁺/CD4⁺/CD8⁻/EGFRt⁺ and CD45⁺/CD3⁺/CD4⁻/CD8⁺/EGFRt⁺ events in a lymphocyte forward/side scatter (FS/SS) gate. The absolute CD4⁺ and CD8⁺ CAR-T cell counts in blood were determined by multiplying the percentage of CD4⁺ and CD8⁺ CAR-T cells, respectively, in a viable CD45⁺ lymphocyte FS/SS gate by the absolute lymphocyte count established by a complete blood count (CBC) performed on the same day (Turtle et al. I and II, 2016).

### Histology and Immunohistochemistry

Formalin-fixed paraffin-embedded (FFPE) tumor blocks were sectioned at 4 μm and mounted on positively charged slides. Aggressive vs. indolent histology was determined by a pathologist examining hematoxylin and eosin-stained slides.

Immunohistochemistry was performed on tumor sections using a standard automated immunodetection system (with anti-CD19 antibodies). Appropriate positive and negative controls were included with each antibody run.

### Statistical Analyses of Key Variables

Descriptive statistics are reported for key variables in the following Examples, Tables, and Figures.

### Example 2

### Factors Impacting Disease-Free Survival in Adult B-Cell All Patients Achieving MRD-Negative CR After CD19 CAR-T Therapy

### 2.1. Characteristics of B-ALL Patients Analyzed

Table 2 shows characteristics of patients with B-cell Acute Lymphoblastic Leukemia (B-ALL) who received anti-CD19 CAR-T cell therapy and who either did (“MRD-neg CR”) or did not (“Not MRD-neg CR”) achieve a Complete Response (“CR”) with Minimal Residual Disease (“MRD=Minimal Residual Disease”). ECOG=Eastern Cooperative Oncology Group Scale of Performance Status; allo-HSCT=allogeneic Hematopoietic Stem Cell Transplant; LDH=Lactate Dehydrogenase; PH+=Philadelphia chromosome rearrangement-positive; CNS=Central Nervous System; Cy/Flu=Cyclophosphamide/Fludarabine; Dose Level 2=2×106 EGFRt+ CAR-T cells/kg.

TABLE 2
Characteristics of B-ALL patients who received CD19 CAR- T cell
Therapy and did or did not achieve CR with MRP-Negative Disease

| | MRD-neg CR | | Not MRD-neg CR | |
|---|---|---|---|---|
| Characteristic | n = 45 | | n = 8 | |
| Age in years, median [range] | 39 | [20-76] | 42.5 | [20-66] |
| Female, n (%) | 23/45 | (51%) | 0/8 | (0%) |
| ECOG 0-1, n (%) | 44/45 | (98%) | 8/8 | (100%) |
| Prior regimens, median | 3 | [1-11] | 3.5 | [1-7] |
| [range] | | | | |
| Prior allo-HSCT¹, n (%) | 18/45 | (40%) | 5/8 | (62%) |
| Marrow blasts (%)², | 28 | [0-98] | 23 | [0.3-98] |
| median [range] | | | | |
| LDH (U/L), median [range] | 201 | [107-1027] | 195 | [153-3727] |
| Ph+ ALL³, n (%) | 10/45 | (22%) | 2/8 | (25%) |
| Extramedullary disease, n (%) | 14/45 | (31%) | 4/8 | (50%) |
| CNS disease, n (%) | 2/45 | (4%) | 3/8 | (38%) |
| Cy/Flu lymphodepletion, | 35/45 | (78%) | 7/8 | (88%) |
| n (%) | | | | |
| Dose level 2, n (%) | 19/45 | (42%) | 1/8 | (13%) |

¹Allogeneic hematopoietic stem cell transplant
²Measured by flow cytometry
³Philadelphia chromosome positive

Of the 57 B-ALL patients who received CAR-T cell therapy, one (1) was MRD-negative at baseline (as determined by flow cytometry), two (2) received CAR-T cells at DL3, and one (1) patient died prior to restaging. These four patients were excluded from the analysis. The remaining 53 patients (having bone marrow and/or extramedullary disease as determined by flow cytometry, having received DL1 or DL2 CAR-T cells, and being >1 yr. from receiving CAR-T cells) were included in the analysis. 45 patients (85%) achieved MRD-negative CR, while 8 (15%) did not.

Anti-tumor response after CAR-T cell infusion was assessed by bone marrow aspiration and biopsy, with PET-CT performed in patients with extramedullary disease. High resolution (1:10,000) flow cytometry was used to identify marrow MRD, and marrow from patients in MRD-neg CR was evaluated by high-throughput sequencing (HTS) of IGH, IGK, TRB, TRD, and TRG genes. Cox regression univariate and stepwise multivariable modeling were performed to identify factors associated with PFS, OS, and DFS.

### 2.2. Post-Treatment Survival

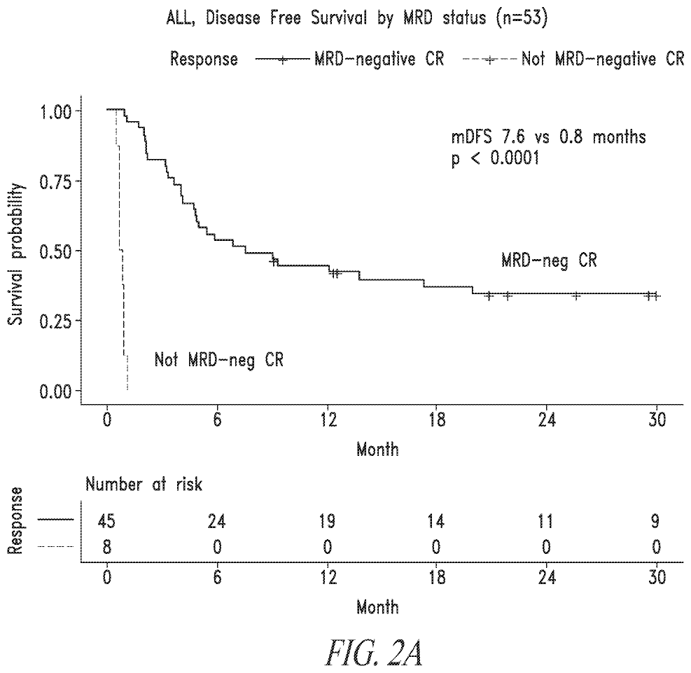

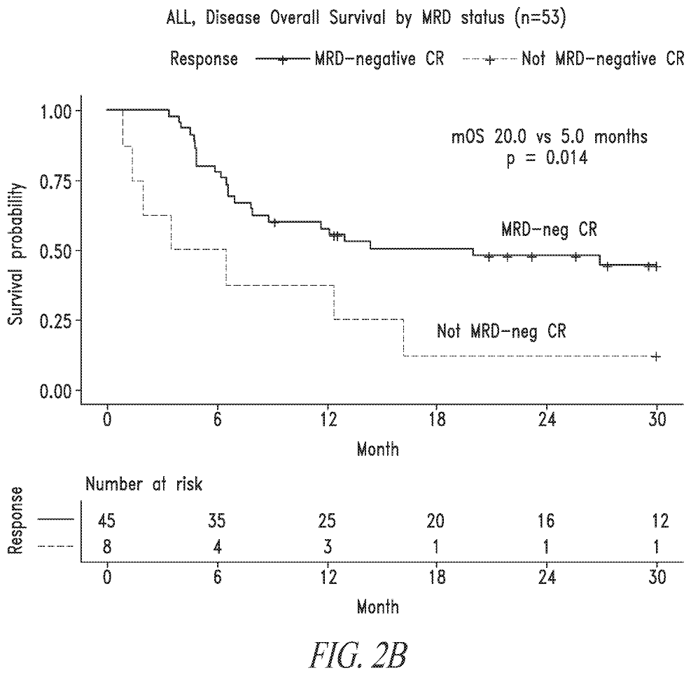

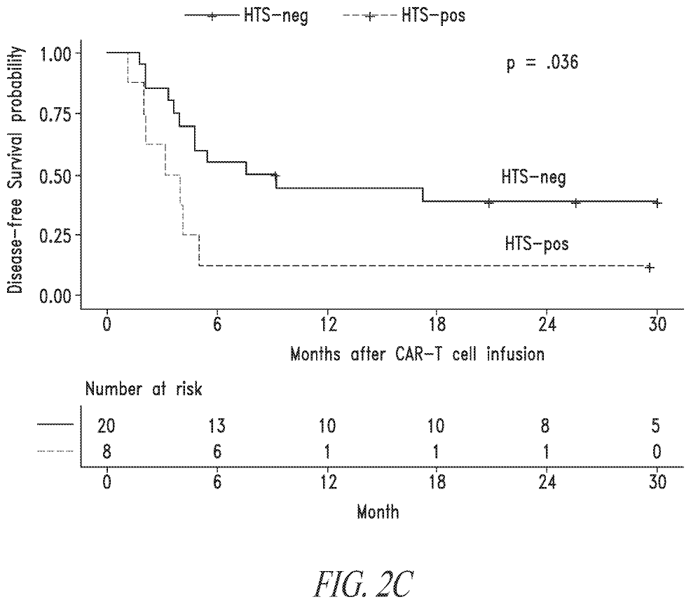

Post-treatment survival of the B-cell ALL patients administered CD19 CAR-T cell therapy was investigated. The median follow-up was 30.9 months after the first CAR-T infusion. Patients who achieved MRD-negative CR following CAR-T cell therapy had significantly better disease-free survival (DFS; FIG. 2A; median DFS, 7.6 vs 0.8 months, p<0.0001) and overall survival (OS; FIG. 2B; median OS, 20.0 vs 5.0 months, p=0.014) and were at lower risk for relapse as compared to patients who did not achieve MRD-negative CR. Twenty-eight of the 45 patients who achieved MRD-neg CR had a leukemic clone identified by HTS prior to CAR-T cell infusion, and in 20 of these pts (71%), the leukemic clone was not detected in marrow 3 weeks after CAR-T cell infusion. These patients had significantly better DFS (FIG. 2C; median DFS. 8.4 vs. 3.6 months, p=0.36, Logrank test) than those who had a persistent leukemic clone.

### 2.3. Univariate Analysis of Factors

To identify patient and treatment factors that may be correlated with achieving MRD-negative CR after CAR-T cell therapy, univariate statistical analysis was performed using pre- (and Day-28-post-CAR-T infusion variables (Table 3) Variables were assessed prior to and 28 days after receiving CAR-T cell infusion(s). Odds ratio (OR) is a measure of association between the identified variable and MRD-negative CR. FlapEF1α is a CAR transgene expression marker.

TABLE 3
Univariate Analysis of Factors associated with likelihood
of MRD-negative CR in B-ALL Patients receiving lymphodepleting
chemotherapy and CAR-T cells

| | | |
|---|---|---|
| | Odds Ratio | |
| Variable | (95% Cl) | P-value¹ |

Prior to CAR-T Cell Infusion

| | | |
|---|---|---|
| Age in years² | 1.00 (0.95-1.05) | 0.98 |
| ECOG (0 vs 1 vs 2) | 1.12 (0.27-5.00) | 0.87 |
| Prior regimens (n) | 0.94 (0.67-1.40) | 0.74 |
| Marrow blasts (%)³ | 1.00 (0.98-1.02) | 0.83 |
| Ph+ (Y) | 0.86 (0.17-6.46) | 0.86 |
| Extramedullary (Y) | 0.45 (0.09-2.16) | 0.31 |
| CNS (disease) (Y) | 0.08 (0.01-0.57) | 0.01 |
| Fludarabine⁴ (Y) | 0.50 (0.03-3.31) | 0.54 |
| Dose level (2 vs 1) | 5.12 (0.81-99.8) | 0.14 |

After CAR-T Cell Infusion to Day 28

| | | |
|---|---|---|
| CAR-T cells, peak⁵ | 2.39 (1.56-4.78) | 0.001 |
| CAR-T cells, AUC day 0-28⁵ | 3.94 (1.76-25.90) | 0.02 |
| Cytokine release syndrome (Y) | 7.71 (1.57-44.50) | 0.01 |
| IFN-γ, AUC day 0-28⁶ | 20.0 (2.69-418) | 0.02 |
| TNF-α, AUC day 0-28⁶ | 4.33 (1.63-19.7) | 0.01 |

¹Without multiplicity adjustment
²Per year increment
³Measured by flow, per % increment
⁴Added to cyclophosphamide-based lymphodepletion
⁵Measured by qPCR, FLAP-EF1α logₑ copies/μg DNA
⁶Serum cytokine increment in logₑ pg/mL

Raw data is provided in Table 4 below. The following abbreviation and symbols are used:
preLD=pre-lymphodepletion value;
day 0=date of CAR-T cell infusion
AUC28=area under the curve from day 0 to 28. AUC calculations are performed using unit denoted.
logₑ=natural log transformation
delta (triangle)=absolute change in value from pre-lymphodepletion to day 0
fold change=fold change in value from pre-lymphodepletion to day 0
CRS=cytokine release syndrome
>ULN=value greater than the upper limit of normal
95% CI=95% confidence interval for estimate
Cy/Flu=cyclophosphamide and fludarabine lymphodepleting chemotherapy

TABLE 4
Univariate Analysis of Associations with
the Development of MRP-Negative CR

| | Odds | Standard | |
|---|---|---|---|
| Variable | Ratio¹ | Error | P-value |
| CD8 CAR-T cells (logₑ | 2.11885023 | 0.23294806 | 0.00126700 |
| cells/μL, AUC28) | | | |
| FlapEF1α (logₑ copies/μg | 1.79695869 | 0.17802039 | 0.00127375 |
| DNA, peak) | | | |
| CD8 CAR-T cells (logₑ | 1.90139684 | 0.19972081 | 0.00129342 |
| cells/μL, peak) | | | |
| CD8 CAR-T cells (logₑ %, | 2.20822456 | 0.26561632 | 0.00285913 |
| peak) | | | |
| CD4 CAR-T cells (logₑ %, | 2.85834738 | 0.35930515 | 0.00346693 |
| peak) | | | |
| CD4 CAR-T cells (logₑ | 2.11386315 | 0.26364878 | 0.00452453 |
| cells/μL, AUC28) | | | |
| CD4 CAR-T cells (logₑ | 1.8714668 | 0.22258429 | 0.00486766 |
| cells/μL, peak) | | | |
| FlapEF1α (logₑ copies/μg | 3.94277899 | 0.58184129 | 0.01838186 |
| DNA, AUC28) | | | |
| CNS involvement (Y) | 0.07751938 | 1.02789373 | 0.01285243 |
| CRS (Y) | 7.7083330 | 0.8278646 | 0.01362687 |
| TNF-α (logₑ pg/mL, | 4.33187814 | 0.59811799 | 0.01424507 |
| AUC28) | | | |
| sTNFRp55 (logₑ | 39.1976773 | 1.50030572 | 0.01447531 |
| fold change) | | | |
| sTNFRp55 (pg/mL, Δ) | 1.00059876 | 0.00025458 | 0.01871317 |
| IFN-γ (logₑ | 19.9576304 | 1.27747592 | 0.01910991 |
| pg/mL, AUC28) | | | |
| CRS Grade (0-4, peak) | 2.8536506 | 0.4533035 | 0.02070953 |
| sTNFRp75 (logₑ, | 28.9734314 | 1.45559192 | 0.02073794 |
| fold change) | | | |
| sTNFRp75 (logₑ pg/mL, | 0.16957473 | 0.80279468 | 0.02708051 |
| preLD) | | | |
| IL-18 (logₑ pg/mL, preLD) | 0.39021773 | 0.44432024 | 0.03417885 |
| IL-18 (logₑ fold change) | 5.91481125 | 0.85217511 | 0.03699752 |
| TNF-α (pg/mL, day 0) | 2.56835775 | 0.46910651 | 0.04434944 |
| sIL-2Rα (fold change, | 5.05038982 | 0.83762752 | 0.05318747 |
| preLD to day 0) | | | |
| IL-15 (logₑ pg/mL, preLD) | 2.09914668 | 0.39620044 | 0.0612612 |
| TNF-α (fold change, | 235.259197 | 2.93104519 | 0.06245503 |
| preLD to day 0) | | | |
| IL-15 (logₑ pg/mL, day 0) | 2.88139446 | 0.58398889 | 0.06996331 |
| IL-22 (logₑ pg/mL, | 4.55136464 | 0.87392487 | 0.08290968 |
| AUC28) | | | |
| sIL2R-α (logₑ pg/mL, | 1.72294975 | 0.32012912 | 0.08923775 |
| peak) | | | |
| sTIM-3 (logₑ pg/mL, | 2.12728181 | 0.44763629 | 0.09173978 |
| AUC28) | | | |
| Normal B cells in marrow | 0.91095651 | 0.05584175 | 0.0949038 |
| (%, preLD) | | | |
| IL-22 (logₑ pg/mL, day 0) | 3.77435779 | 0.79816212 | 0.09609028 |
| MCP-1 (logₑ pg/mL, day 0) | 2.07521241 | 0.44421536 | 0.10028159 |

¹Higher odds ratio means more a higher likelihood of obtaining MRD-neg CR. For continuous variables, OR is per increment unit increase.

Univariate analysis was also conducted for associations with disease-free survival (Table 5) and overall survival (Table 6).

TABLE 5
Univariate Analysis of Associations with Disease-Free Survival in
Patients with MRP-Negative CR after CD19 CAR-T cells (DL2; n = 45)

| Variable | Estimate² | 95% CI low | 95% CI high | P-value |
|---|---|---|---|---|
| LDH (U/L, preLD) | 1.00397176 | 1.00201158 | 1.00593577 | 0.0000703 |
| Platelets (1000 cells/μL, preLD) | 0.98881471 | 0.98295027 | 0.99471413 | 0.00021036 |
| Extramedullary disease (Y, preLD) | 3.56826972 | 1.66291093 | 7.65678342 | 0.00109278 |
| TGF-β1 (logₑ pg/mL, preLD) | 0.54774783 | 0.37855492 | 0.79256052 | 0.00140651 |
| Cy/Flu (Y) | 0.29669636 | 0.13270453 | 0.66334385 | 0.00307792 |
| IL-6 (pg/mL, preLD) | 1.01781514 | 1.00578425 | 1.02998995 | 0.00360688 |
| TGF-β1 (logₑ pg/mL, Day 0) | 0.48809594 | 0.29730192 | 0.80133234 | 0.00457445 |
| LDH > ULN (Y, preLD) | 2.92051308 | 1.37605095 | 6.1984599 | 0.00524881 |
| TGF-β1 (logₑ pg/mL, AUC28) | 0.52225817 | 0.32850488 | 0.83028781 | 0.0060285 |
| Marrow blasts (%, preLD) | 1.01481755 | 1.00421663 | 1.02553039 | 0.00604525 |
| IL-2 (pg/mL, day 0) | 1.1707388 | 1.04050062 | 1.31727873 | 0.00879859 |
| TGF-β1 (logₑ pg/mL, peak) | 0.5470678 | 0.34475024 | 0.86811595 | 0.01045877 |
| IL-6 (pg/mL, day 0) | 1.03124043 | 1.00523768 | 1.0579158 | 0.01823137 |
| IL-18 (logₑ pg/mL, day 0) | 1.51824625 | 1.07218759 | 2.14987722 | 0.01863818 |
| MCP-1 (logₑ fold change) | 0.55548636 | 0.33813324 | 0.91255473 | 0.02027259 |
| sTIM-3 (logₑ pg/mL, AUC28) | 1.53308839 | 1.05195435 | 2.23427948 | 0.02617945 |
| Neutrophils (1000 | 0.7312747 | 0.55359204 | 0.96598694 | 0.02755105 |
| cells/μL, preLD) | | | | |
| IL-6 (pg/mL, Δ) | 0.98030116 | 0.96245867 | 0.99847441 | 0.03376469 |
| sTNFRp55 (logₑ pg/mL day 0) | 1.9827545 | 1.03144178 | 3.81147583 | 0.04009128 |
| IL-8 (logₑ pg/mL, preLD) | 1.28423839 | 1.00190817 | 1.64612717 | 0.04826653 |
| sTIM-3 (pg/mL, preLD) | 1.00005251 | 0.99999861 | 1.00010641 | 0.05621342 |
| sTIM-3 (pg/mL, day 0) | 1.00004122 | 0.99999755 | 1.00008489 | 0.06431401 |
| MCP-1 (logₑ pg/mL, preLD) | 1.3848474 | 0.97946677 | 1.95800654 | 0.06539411 |
| MLL rearrangement | 2.19132691 | 0.94895288 | 5.06022345 | 0.06617208 |
| (Y, preLD) | | | | |
| IL-18 (logₑ pg/mL, preLD) | 1.45767805 | 0.96695955 | 2.19742936 | 0.07193604 |
| IL-6 (logₑ pg/mL, AUC28) | 1.18840188 | 0.98420192 | 1.43496878 | 0.07274637 |
| sTNFRp75 (logₑ pg/mL, preLD) | 1.79365851 | 0.94403316 | 3.40794265 | 0.07440833 |
| IL-10 (logₑ pg/mL, AUC28) | 1.31591594 | 0.96152487 | 1.80092561 | 0.0863669 |
| Dose level 2 (Y) | 0.51270513 | 0.23722517 | 1.10808876 | 0.08932936 |
| Prior regimens (Number) | 1.13466301 | 0.97732157 | 1.31733524 | 0.09716198 |
| Time to apheresis from | 1.29161568 | 0.94932266 | 1.75732775 | 0.10333158 |
| last chemo (logₑ days) | | | | |

²Lower estimate is associated with longer DFS, higher estimate number is associated with a shorter DFS. For continuous variables, estimate is per increment unit increase.

TABLE 6
Univariate Analysis of Associations with Overall Survival in Patients
with MRD-negative CR after CD19 CAR-T cells (DL2; n = 45)

| Variable | Estimate² | 95% CI low | 95% CI high | P-value |
|---|---|---|---|---|
| platelets (1000 | 0.98613719 | 0.9791518 | 0.99317242 | 0.00011867 |
| cells/pL, preLD) | | | | |
| TGF-β1 (logₑ pg/mL, Day 0) | 0.35227778 | 0.2016737 | 0.61534863 | 0.00024617 |
| LDH (U/L, preLD) | 1.00321506 | 1.00148099 | 1.00495214 | 0.00027631 |
| TGF-β1 (logₑ pg/mL, peak) | 0.43883674 | 0.27276231 | 0.70602748 | 0.00068698 |
| TGF-β1 (logₑ pg/mL, preLD) | 0.49083177 | 0.3222057 | 0.74770814 | 0.00092037 |
| TGF-β1 (logₑ pg/mL, AUC28) | 0.45268214 | 0.27874593 | 0.73515375 | 0.00135714 |
| LDH > ULN (Y, preLD) | 3.32713159 | 1.48440792 | 7.45738721 | 0.00350899 |
| IL-6 (pg/mL, preLD) | 1.01709724 | 1.00520684 | 1.02912828 | 0.00471999 |
| Marrow blasts (%, preLD) | 1.01621588 | 1.00480428 | 1.02775708 | 0.00524199 |
| Neutrophils (1000 | 0.65054398 | 0.46731764 | 0.90560987 | 0.01085287 |
| cells/μL, preLD) | | | | |
| MCP-1 (logₑ fold change) | 0.4848236 | 0.27473719 | 0.85555915 | 0.01247932 |
| MLL rearrangement (Y) | 2.91538042 | 1.20768891 | 7.03777515 | 0.0173293 |
| IL-6 (pg/mL, day 0) | 1.02944117 | 1.00449386 | 1.05500807 | 0.02043911 |
| sTIM-3 (pg/mL, day 0) | 1.00005899 | 1.00000894 | 1.00010905 | 0.02089601 |
| Extramedullary disease (Y, preLD) | 2.59052554 | 1.14217114 | 5.87549654 | 0.02271998 |
| Female (Y) | 2.58666878 | 1.13601714 | 5.88974862 | 0.02359076 |
| IL-5 (fold change) | 1.05798773 | 1.0060773 | 1.11257659 | 0.02809151 |
| IL-6 (pg/mL, Δ) | 0.98208479 | 0.96552755 | 0.99892596 | 0.037175 |
| sTIM-3 (pg/mL, preLD) | 1.00006002 | 1.00000271 | 1.00011734 | 0.04011199 |
| Dose level 2 (Y) | 0.40489596 | 0.1702062 | 0.96318899 | 0.04087581 |
| Cy/Flu (Y) | 0.43489134 | 0.19501558 | 0.9698224 | 0.04186622 |
| TIM-3 (logₑ pg/mL, AUC28) | 1.53959705 | 0.99217953 | 2.38904251 | 0.05423673 |
| Vinca alkaloids 1 week | 3.76621929 | 0.85721732 | 16.5470383 | 0.07909593 |
| before apheresis (Y) | | | | |
| IL-18 (logₑ pg/mL, day 0) | 1.34611472 | 0.96077154 | 1.88601011 | 0.08409848 |
| IL-7 (logₑ pg/mL, AUC28) | 0.62132294 | 0.34981414 | 1.10356374 | 0.10443151 |

²Lower estimate is associated with longer OS; higher estimate number is associated with a shorter OS. For continuous variables, estimate is per increment unit increase.

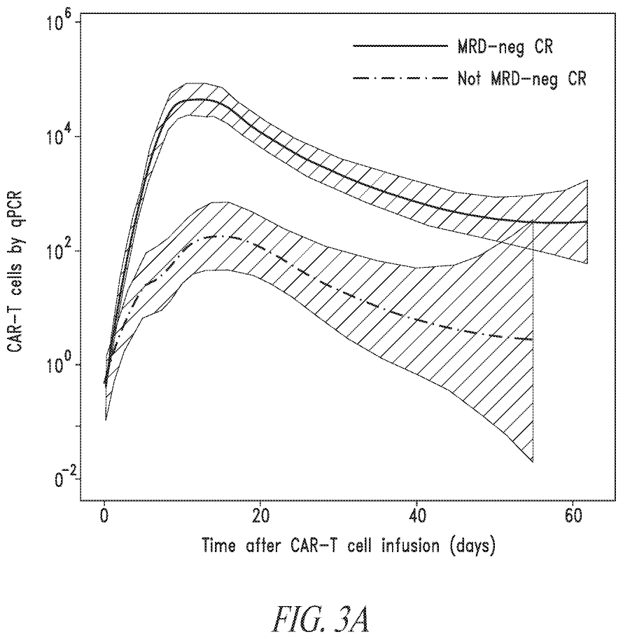

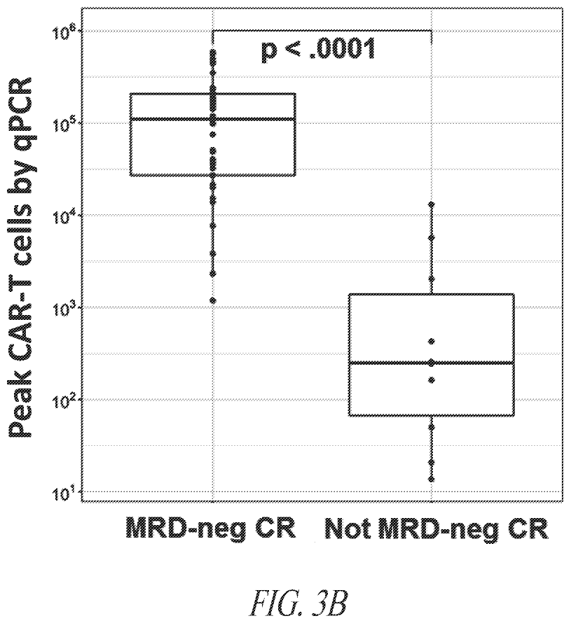

Of the pre-infusion variables, only patient CNS disease had a statistically significant (negative) association with MRD-negative CR (p=0.01). Five post-infusion variables had a statistically significant positive association with MRD-negative CR (Table 3), and in vivo CAR-T expansion was identified as the primary determinant of achieving MRD-negative CR (p=0.001; see also FIGS. 3A and 3B). Multivariable modelling for association with achieving CR could not be performed due to the low number of patients that did not achieve MRD-negative CR.

2.4. Multivariate Analysis of Association with Outcomes

A Day-28 multivariate analyses were performed using factors identified as significant in the univariate analysis and patient characteristics of interest, and predictive models were generated. Briefly, factors were examined for association with DFS and OS in MRD-negative CR using stepwise (Examples 2.4.1 and 2.4.3) elastic net multivariable analysis (Examples 2.4.2 and 2.4.4). For the models shown in Tables 7, 8, 9, 10, and 11, a higher hazard ratio (HR) is associated with worse outcome (i.e. shorter DFS or OS), while a lower hazard ratio is associated with an improved outcome (i.e. longer DFS or OS).

Table 7 shows summary data from a Day-28 landmark elastic net multivariate analysis (Cox regression model) of factors associated with improved DFS in B-ALL patients who achieved MRD-negative CR following lymophodepleting chemotherapy and CAR-T cell infusion.

TABLE 7
Multivariate Analysis of Factors Prior to Lymphodepletion

| Variable | HR (95% Cl) | P-value |
|---|---|---|
| LDH prior to lymphodepletion | 1.32 (1.01-1.73) | 0.04 |
| (per 100 U/L increment) | | |
| Cy/Flu Lymphodepletion | 0.24 (0.08-0.71) | 0.01 |

Table 8 shows summary data from a Day-28 landmark stepwise multivariate analysis (proportional hazard model) of factors associated with improved DFS in B-ALL patients who achieved MRD-negative CR following lymphodepleting chemotherapy and CAR-T cell infusion. HR=Hazard Ratio.

TABLE 8
Day-28 Stepwise Multivariable Landmark Analysis
in MRP-Negative CR Patients (n = 45)

| Variable | HR (95% Cl) | P-value¹ |
|---|---|---|
| LDH prior to lymphodepletion² | 1.39 (1.12-1.74) | 0.003 |
| Platelets prior to lymphodepletion³ | 0.65 (0.47-0.88) | 0.006 |
| Cy/Flu lymphodepletion | 0.34 (0.15-0.78) | 0.011 |

¹stepwise multivariable proportional hazard model, considering factors significant in univariate analysis and patient characteristics of interest
²per 100 U/L increment
³per 50,000 cells/μL increment

Table 9 shows summary data from a Day-28 landmark stepwise multivariate analysis (proportional hazard model) of factors associated with improved DFS in B-ALL patients who achieved MRD-negative CR following lymphodepleting chemotherapy and CAR-T cell infusion. HR=Hazard Ratio. * Patient with grade 3B follicular lymphoma per 2017 WHO Classification. † MYC rearrangement not available for 8 patients. ‡ Two patients with Burkitt lymphoma, one with T cell-/histiocyte-rich large B-cell lymphoma, one with primary cutaneous diffuse B-cell lymphoma, leg type, and one with high-grade B-cell lymphoma, NOS. § Scores on the IPI include low risk (0 or 1 point), low-intermediate risk (2 points), high-intermediate risk (3 points), and high risk (4 or 5 points). ¶ Maximum tumor diameter ≥10 cm.

TABLE 9
Day-28 Stepwise Multivariable Landmark Analysis in MRP-Negative CR Patients (n = 45)

| | | |
|---|---|---|
| Indolent | Aggressive | |
| Histology | Histology | All Pts |

Characteristics at baseline

| | | | |
|---|---|---|---|
| No. of Pts | 9 | 48 | 57 |

Disease histology - no. (%)

| Follicular lymphoma | 8 | (89) | 1 | (2)* | 9 | (16) |
|---|---|---|---|---|---|---|
| Marginal zone lymphoma | 1 | (11) | 0 | | 1 | (2) |
| Mantle cell lymphoma | 0 | | 6 | (12) | 6 | (10) |
| DLBCL, de novo | | | 18 | (37) | 18 | (32) |
| DLBCL, transformed | | | 10 | (21) | 10 | (17) |
| Other aggressive | | | 5 | (11) | 5 | (9) |

Age

| | | | | | | |
|---|---|---|---|---|---|---|
| Median (range) - yr | 56 | (33-69) | 56.5 | (27-71) | 56.5 | (27-71) |
| ≥65 yr - no. (%) | 1 | (11) | 8 | (17) | 9 | (16) |
| Male sex - no (%) | 5 | (56) | 35 | (73) | 40 | (70) |
| ECOG performance - | 4 | (44) | 20 | (42) | 24 | (42) |
| status score of 1 - no. (%) | | | | | | |

Disease stage - no. (%)

| | | | | | | |
|---|---|---|---|---|---|---|
| I or II | 2 | (22) | 1 | (2) | 3 | (5) |
| III or IV | 8 | (78) | 47 | (98) | 54 | (95) |

Extra nodal disease - no. (%)

| | | | | | | |
|---|---|---|---|---|---|---|
| Yes | 4 | (44) | 44 | (92) | 44 | (84) |
| No | 5 | (56) | 4 | (8) | 9 | (16) |

International Prognostic Index (IPI) score - no. (%) §

| | | | | | | |
|---|---|---|---|---|---|---|
| 0 or 1 | 4 | (44) | 7 | (15) | 11 | (19) |
| 2 | 4 | (44) | 16 | (33) | 20 | (35) |
| 3 or 4 | 1 | (12) | 25 | (52) | 26 | (46) |

Bulky disease (≥10 cm)

| Yes | 0 | | 8 | (17) | 8 | (14) |
|---|---|---|---|---|---|---|
| No | 9 | (100) | 40 | (83) | 49 | (86) |

Tumor cross-sectional area¶

| | | | | | | |
|---|---|---|---|---|---|---|
| Median (range) - mm² | 3511.1 | (406-8452) | 3248.9 | (124-16765) | 3342.5 | (124-16765) |
| ≥Median - no. (%) | 3 | (33) | 26 | (54) | 29 | (51) |

Prior therapies - no. (%)

| | | | | | | |
|---|---|---|---|---|---|---|
| Median (range) - no. | 4 | (2-7) | 4 | (1-11) | 4 | (1-11) |
| ≥Four prior lines of | 8 | (89) | 34 | (71) | 36 | (63) |
| therapy | | | | | | |

Prior autologous hematopoietic stem cell transplantation (HSCT) - no (%)

| | | | | | | |
|---|---|---|---|---|---|---|
| Yes | 3 | (33) | 19 | (40) | 22 | (39) |
| No | 6 | (67) | 29 | (60) | 35 | (61) |

Prior allogeneic hematopoietic stem cell transplantation (HSCT) - no (%)

| | | | | | | |
|---|---|---|---|---|---|---|
| Yes | 1 | (11) | 7 | (15) | 8 | (14) |
| No | 8 | (89) | 41 | (85) | 49 | (86) |

Bridging therapy between leukapheresis and lymphodepletion

| Intensive chemotherapy - | 0 | | 7 | (15) | 7 | (12) |
|---|---|---|---|---|---|---|
| no. (%)** | | | | | | |
| High dose steroid - no. | 1 | (11) | 9 | (19) | 10 | (18) |
| (%)†† | | | | | | |
| Other - no. (%)‡‡ | 0 | | 2 | (4) | 2 | (4) |
| Any therapy between | 1 | (11) | 12 | (25) | 13 | (23) |
| leukapheresis and | | | | | | |
| lymphodepletion - no. (%) | | | | | | |

2.4.1. Stepwise Multivariable Analysis of Association with Disease-Free Survival

A stepwise multivariable method was used to generate a proportional hazard model associated with DFS. Highly correlated variables (e.g., TGF-β1, which is highly correlated with platelet count) were not included in the analysis. Also, variables with data missing from 10% or more of the patients (e.g., data for TGF-β1, IL-18, and MCP-1 variables was missing from 7 or more patients was missing) were not included in order to preserve stability of the model. A p-value cutoff of 0.05 from the univariate analysis was used. The following factors were considered:
Age (years); ECOG (0-2); Dose level 2 (Y); Prior transplant (Y); Prior regimens (No.); Platelets (1000 cells/μL, preLD); LDH (U/L); Extramedullary disease (Y); Cy/Flu (Y); Marrow blasts (%, preLD); IL-2 (pg/mL, day 0); TIM-3 (logₑ pg/mL, AUC28); IL-6 (pg/mL, preLD); sTNFRp55 (logₑ pg/mL, day 0)

From these variables, LDH (U/L preLD), Cy/Flu (Y), and Platelets (1000 cells/μL preLD) were used to generate the final model shown in Table 8. Surprisingly, a high serum level of LDH prior to lymphodepletion posed a strong hazard risk for B-ALL patients.

2.4.2. Elastic Net Multivariable Analysis of Association with Disease Free Survival

An elastic net multivariate analysis was performed to identify factors associated with DFS. A p-value cutoff of 0.10 in the univariate analysis was used, and variables missing from more than 10% of patients were discarded. The following variables were considered:
Age (years); ECOG (0-2); Dose level 2 (Y); Prior transplant (Y); Prior regimens (No.); Platelets (1000 cells/uL preLD); LDH (U/L, preLD); Extramedullary disease (Y); Cy/Flu (Y); Marrow blasts (%, preLD); IL-6 (pg/mL, preLD); IL-8 (logₑ pg/mL, preLD); IL-2 (pg/mL day 0); IL-6 (pg/mL day 0); sTIM-3 (logₑ pg/mL, AUC28); Neutrophils (1000 cells/uL, preLD); sTNFRp55 (logₑ pg/mL, day 0); sTIM-3 (pg/mL, day 0); MLL rearrangement (Y); IL-6 (logₑ pg/mL AUC28); IL-10 (pg/mL AUC28); and time to apheresis from last chemotherapy treatment (logₑ days)

The variables chosen for inclusion in the association model were: Dose Level 2 (Y); Platelets (1000 cells/μL, preLD); LDH (U/L, preLD); Extramedullary Disease (Y); Marrow blasts (%); Cy/Flu lymphodepletion (Y); and IL-6 (pg/mL day0). The final model from the elastic net analysis is shown in Table 10.

TABLE 10
Elastic Net Model of Factors Associated with Disease-
Free Survival in MRP-Negative CR patients

| Variable | Hazard Ratio (95% CI)³ | P-value |
|---|---|---|
| Cy/Flu (Y) | 0.2777 (0.1095-0.7042) | 0.00696 |
| LDH (U/L, preLD, per 100 U/L | 1.3447 (1.0617-1.7030) | 0.01401 |
| increment) | | |
| Platelets (1000 cells/μL, preLD, per | 0.7541 (0.5423-1.0485) | 0.09329 |
| 50,000 cells/μL increment) | | |
| Extramedullary disease (Y) | 2.0981 (0.8331-5.2837) | 0.11582 |
| IL-6 (pg/mL, day 0, per 5 pg/mL | 1.1195 (0.9435-1.3284) | 0.19569 |
| increase) | | |
| Dose level 2 (Y) | 0.5956 (0.2253-1.5745) | 0.29611 |
| Marrow blasts (%) | 0.9991 (0.9852-1.0133) | 0.90113 |

An alternative model was constructed that excluded marrow blasts (%) due to high p-value; this model is shown in Table 11.

TABLE 11
Alternative Elastic Net Model of Factors Associated with
Disease-Free Survival in MRP-Negative CR patients

| Variable | Hazard Ratio (95% CI)³ | P-value |
|---|---|---|
| Cy/Flu (Y) | 0.2794 (0.1109-0.7042) | 0.00686 |
| LDH (U/L, preLD, per 100 | 1.3400 (1.0656-1.6849) | 0.01229 |
| U/L increment) | | |
| Platelets (1000 cells/μL, preLD, | 0.7588 (0.5535-1.0403) | 0.08643 |
| per 50 000 cells/μL increment) | | |
| Extramedullary Disease (Y) | 2.0807 (0.8334-5.1951) | 0.11654 |
| IL-6 (pg/mL, day 0, per 5 pg/mL | 1.1212 (0.9466-1.3279) | 0.18522 |
| increase) | | |
| Dose level 2 (Y) | 0.6129 (0.2586-1.4524) | 0.26608 |

Better DFS was seen in pts with a higher pre-lymphodepletion platelet count (hazard ratio, HR 0.65 [95% CI; 0.47-0.88] per 50,000/μL increment, p=0.006), lower pre-lymphodepletion LDH (HR 1.39 [1.12-1.74] per 100 U/L increment, p=0.003), and with incorporation of fludarabine into the cyclophosphamide-based lymphodepletion (Cy/Flu; HR 0.34 [0.15-0.78], P=0.011). Similar findings were noted in analysis of MRD-neg CR pts who had no malignant clone by HTS after CAR-T cells. Patients with platelets ≥100,000/μL and normal LDH before lymphodepletion who received Cy/Flu (good risk, n=15) had 2-year point estimates of DFS and OS of 78% and 86%, respectively.

2.4.3. Stepwise Multivariable Analysis of Factors Associated with Overall Survival

For this analysis, a p-value cutoff of 0.05 from the univariate study was used. Variables with data missing from more than 10% of patients were discarded, as were variables that were highly correlated with other variables. The following variables were considered:
Age (years); ECOG (0-2); Dose level 2 (Y); Prior transplant (Y); Prior regimens (No.); Platelets (1000 cells/μL, preLD); LDH (U/L, preLD); IL-6 (pg/mL, preLD);Extramedullary disease (Y); Cy/Flu (Y); Marrow blasts (%, preLD); IL-2 (pg/mL, day 0); sTIM-3 (pg/mL, day 0); Neutrophils (1000cells/μL, preLD); and MLL rearrangement (Y)

The variables chosen for the final model were Platelets (1000 cells/μL, preLD) and MLL rearrangement (Y), as shown in Table 12. LDH (U/L) was not included in the final model because the p-value, after adjusting for platelets and MLL rearrangement, was 0.10.

TABLE 12
Stepwise Multivariable Model of Factors Associated with
Overall Survival in MRD-Negative CR Patients (n = 45)

| Variable | Hazard Ratio (95% CI)³ | P-value |
|---|---|---|
| Platelets (1000 cells/μL, preLD, | 0.5186 (0.3691-0.7286) | 0.000154 |
| per 50 000 cells/μL increment) | | |
| MLL rearrangement (Y) | 3.1369 (1.2306-7.9959) | 0.016635 |

2.4.4. Elastic Net Multivariable Analysis of Factors Associated with Overall Survival

For this analysis, a p-value cutoff of 0.10 from the univariate study was used. Variables with data missing from more than 10% of patients were discarded. The following variables were considered:
Age (years); ECOG (0-2); Dose level 2 (Y); Prior transplant (Y); Prior regimens (No.); Platelets (1000 cells/μL, preLD); LDH (U/L, preLD), IL-6 (pg/mL, preLD); Extramedullary disease (Y); Cy/Flu (Y); Marrow blasts (%, preLD); IL-6 (pg/mL, day 0); sTIM-3 (pg/mL, day 0); Neutrophils (1000cells/μL, preLD); MLL rearrangement (Y); IL-6 (pg/mL, 4); sTIM-3 (pg/mL, preLD); sTIM-3 (loge pg/mL AUC28); and Vinca alkaloids 1-week before apheresis (Y)

The variables chosen for the final model were: Dose level 2 (Y), Platelets (1000 cells/μL, preLD); LDH (U/L, preLD); IL-6 (pg/mL, preLD), Neutrophils (1000 cells/μL, preLD); and MLL rearrangement (Y), as shown in Table 13.

TABLE 13
Elastic Net Model of Factors Associated with Overall
Survival in MRD-neg CR patients (n = 45)

| Variable | Hazard Ratio (95% CI)³ | P-value |
|---|---|---|
| Platelets (1000 cells/μL, preLD, per | 0.6122 (0.4238-0.8843) | 0.00891 |
| 50 000 cells/μL increment) | | |
| LDH (U/L, preLD, per 100 U/L | 1.1950 (0.9758-1.4634) | 0.08497 |
| increment) | | |
| MLL rearrangement (Y) | 2.6743 (1.0119-7.0673) | 0.04727 |
| Dose level 2 (Y) | 0.5489 (0.1853-1.6262) | 0.27906 |
| Neutrophils (1000 cells/μL, preLD, | 0.8457 (0.6313-1.1329) | 0.26126 |
| per 1000 cells/μL increment)) | | |
| IL-6 (pg/mL, preLD, per 5 pg/mL | 1.0023 (0.9154-1.0974) | 0.96039 |
| increment) | | |

An alternative model was generated that excluded IL-6 (preLD), which had a high p-value in the elastic net model; this alternative model is shown in Table 14.

TABLE 14
Alternative Elastic Net Model of Factors Associated
with Overall Survival in MRD-neg CR patients (n = 45)

| Variable | Hazard Ratio (95% CI)³ | P-value |
|---|---|---|
| Platelets (1000 cells/μL, preLD, per | 0.6127 (0.4292-0.8746) | 0.00698 |
| 50,000 cells/μL increment) | | |
| LDH (U/L, preLD, per 100 U/L | 1.2070 (1.0099-1.4425) | 0.03862 |
| increment) | | |
| MLL rearrangement (Y) | 2.5755 (0.9918-6.6879) | 0.05201 |
| Dose level 2 (Y) | 0.5862 (0.2300-1.4937) | 0.26305 |
| Neutrophils (1000 cells/μL, preLD, | 0.8083 (0.6092-1.0725) | 0.14030 |
| per 1000 cells/μL increment) | | |

2.5. Significant Hazard-Ratio Variables vs. Survival

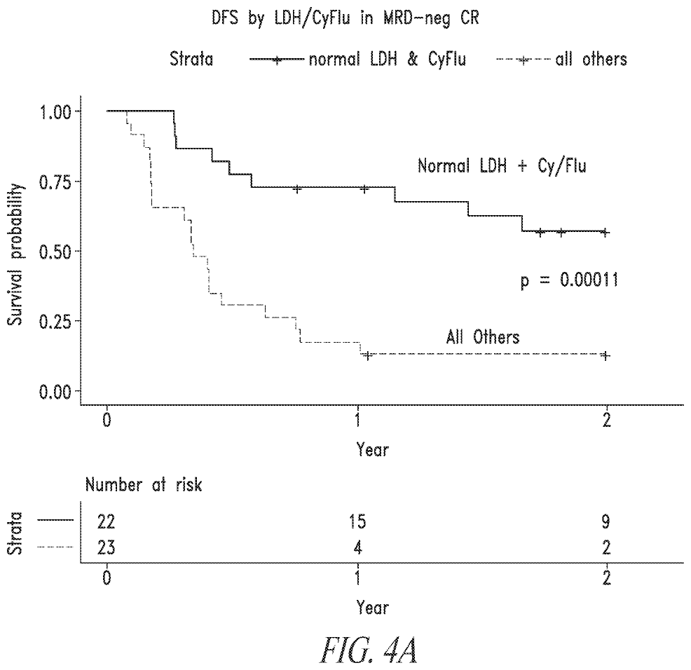

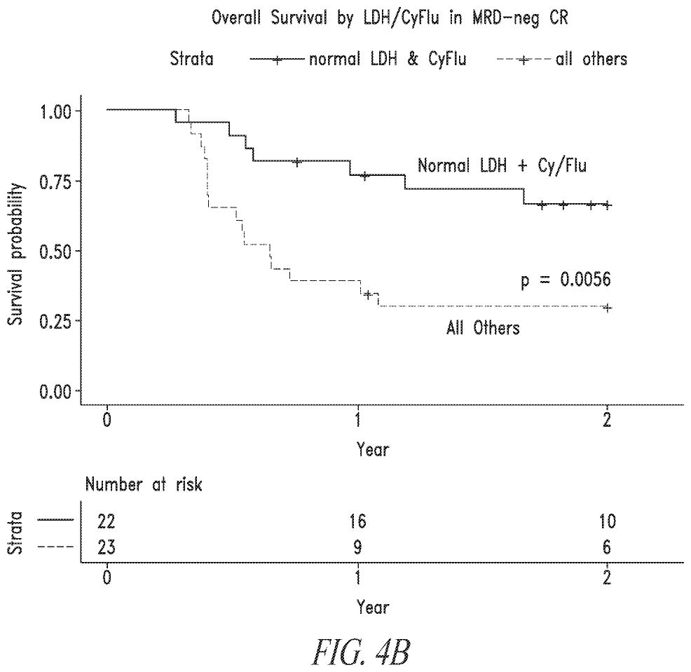

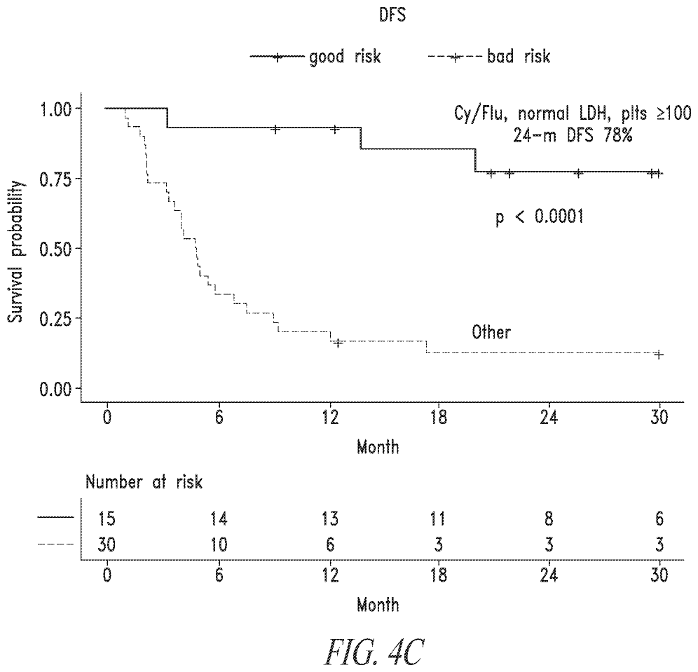

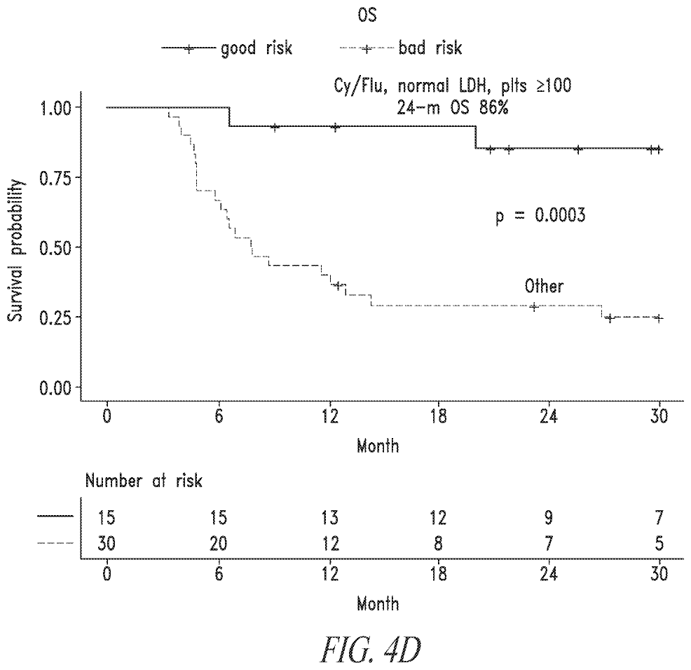

Post-CAR-T survival curves considering the results of the multivariate analysis were generated. Patients with normal LDH (<210 units/liter) and who received Cy/Flu lymphodepleting chemotherapy had significantly better DFS (FIG. 4A) and OS (FIG. 4B) as compared to other patients who achieved MRD-negative CR. Adding pre-lymphodepletion platelet counts of ≥100 U/L as a factor in the survival curves provided similar results as the other multivariate factors (FIGS. 4C and 4D).

### 2.6. Effect of Post-CAR-T Cell Infusion Allogeneic Hematopoietic Stem Cell Transplantation on Survival and Relapse

Following CAR-T infusion, 18 MRD-negative CR patients received allo-HSCT. Characteristics of patients who did or did not receive allo-HSCT after achieving MRD-negative CR following CAR-T cell infusion are shown in Table 15.

TABLE 15
Characteristics of Patients Undergoing Allo-HSCT After
Achieving MRP-Negative CR After CD19 CAR-T Cells

| | Transplant | | No Transplant¹ | |
|---|---|---|---|---|
| Characteristic | n = 18 | | (n = 24) | |
| Age in years, median [range] | 35 | [22-73] | 45.5 | [20-76] |
| Female, n (%) | 7/18 | (39%) | 15/24 | (63%) |
| ECOG 0-1, n (%) | 17/18 | (94%) | 24/24 | (100%) |
| Prior regimens, median [range] | 2.5 | [1-6] | 3.5 | [2-11] |
| Prior allo-HSCT, n (%) | 2/18 | (11%) | 15/24 | (63%) |
| Marrow blasts (%)², | 11 | [0-80] | 43 | [0-98] |
| median [range] | | | | |
| LDH (U/L), median [range] | 177 | [116-334] | 209 | [107-741] |
| Ph+ ALL, n (%) | 4/18 | (22%) | 5/24 | (21%) |
| Cy/Flu lymphodepletion, n (%) | 14/18 | (78%) | 19/24 | (79%) |
| Dose level 2, n (%) | 10/18 | (56%) | 7/24 | (29%) |
| CRS grade, median [range] | 1 | [0-3] | 2 | [0-4] |

¹three patients underwent transplant after relapse and were not included in analysis
²by flow cytometry
CRS = Cytokine Release Syndrome.

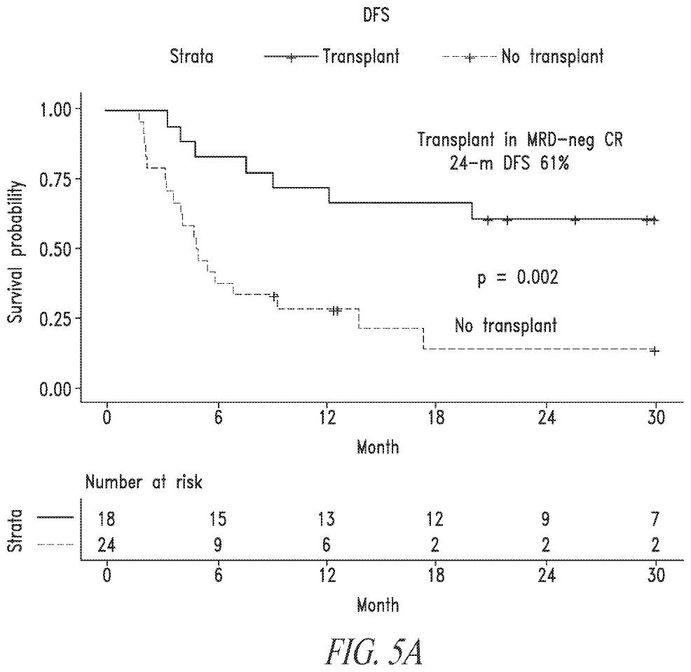

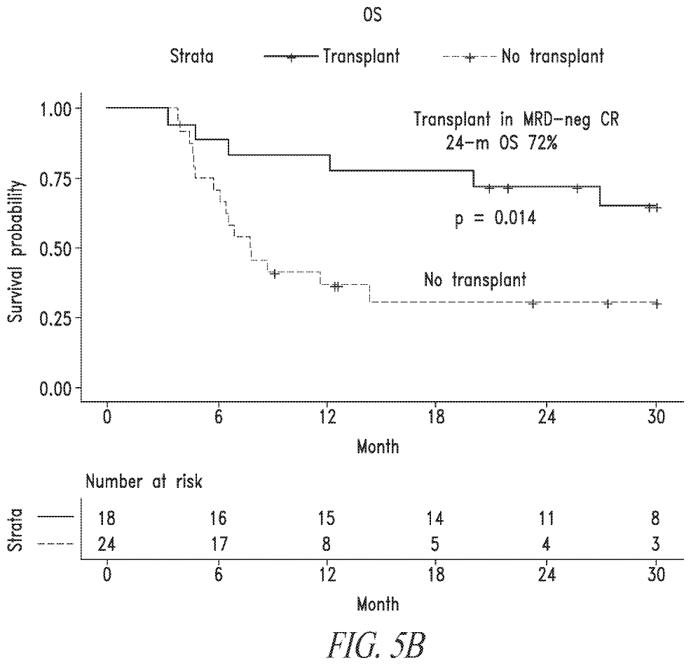

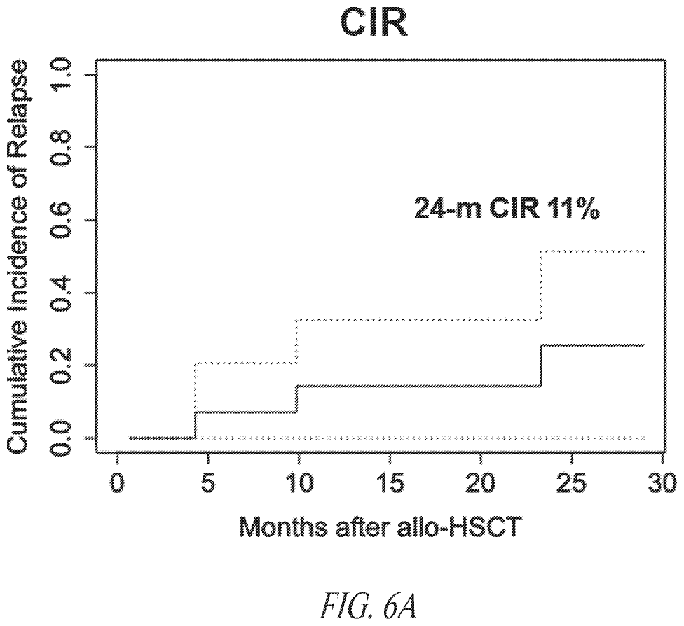

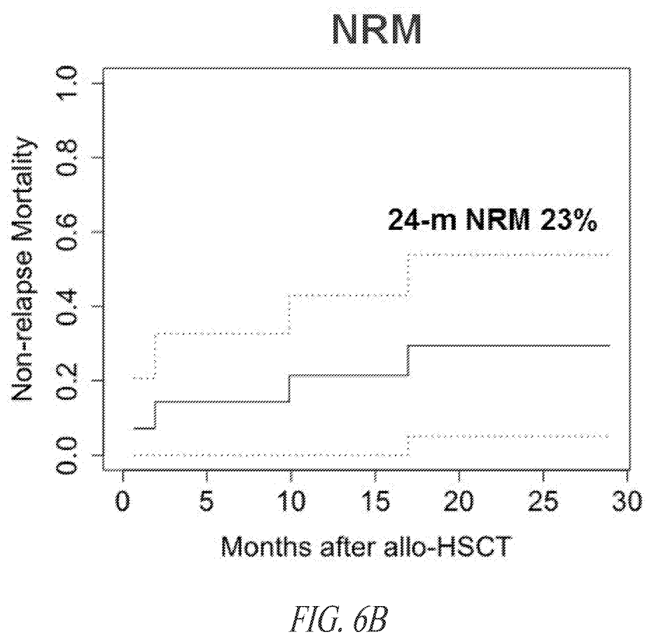

The effect of post-CAR-T allo-HSCT on survival and relapse was investigated. As shown in FIGS. 5A and 5B, patients who received allo-HSCT when in MRD-negative CR had significantly better DFS (FIG. 5A) and OS (FIG. 5B) as compared to patients who did not receive allo-HSCT. As prior allo-HSCT was more common in the patient arm that did not receive post-CAR-T allo-HSCT, the analysis was restricted to patients with no prior HSCT history and similar results were observed (data not shown). The patients who received allo-HSCT also had a low cumulative incidence of relapse (CIR; FIG. 6A) and low non-relapse mortality (NRM; FIG. 6B) at 24 months after allo-HSCT.

### 2.7. Combined Effect of Allo-HSCT and Significant Hazard-Ratio Variables on Survival

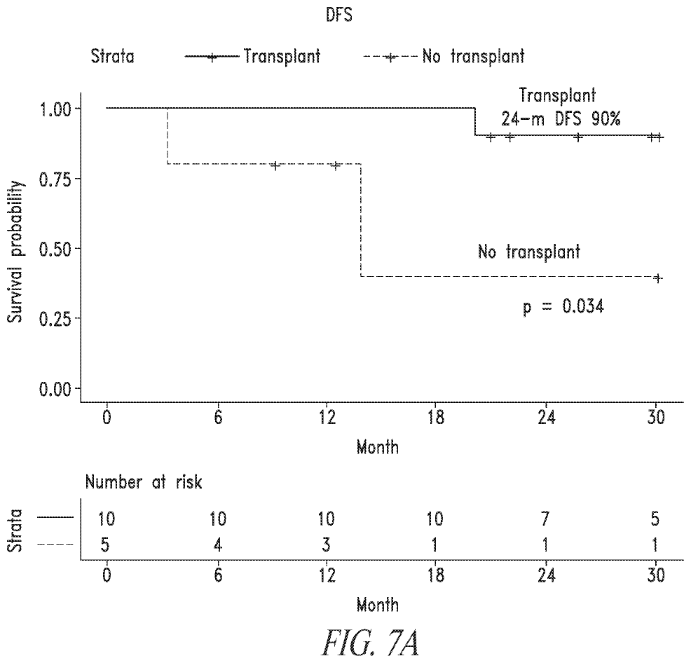

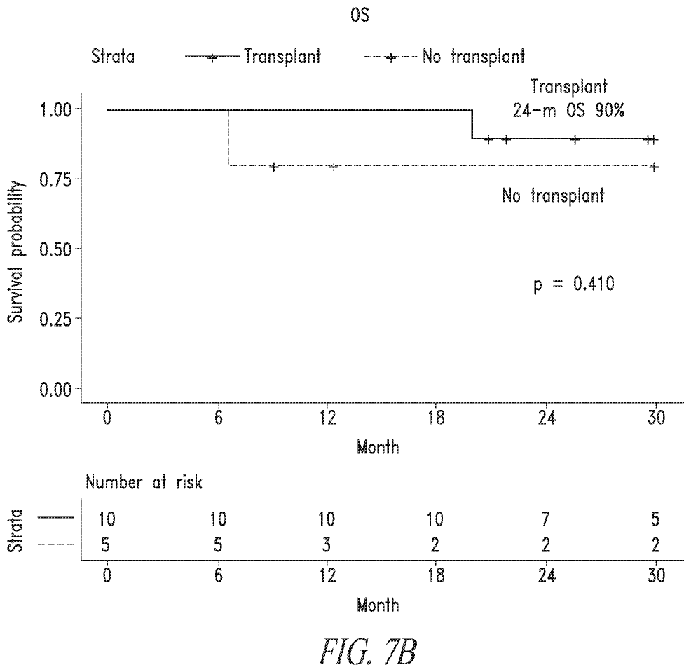

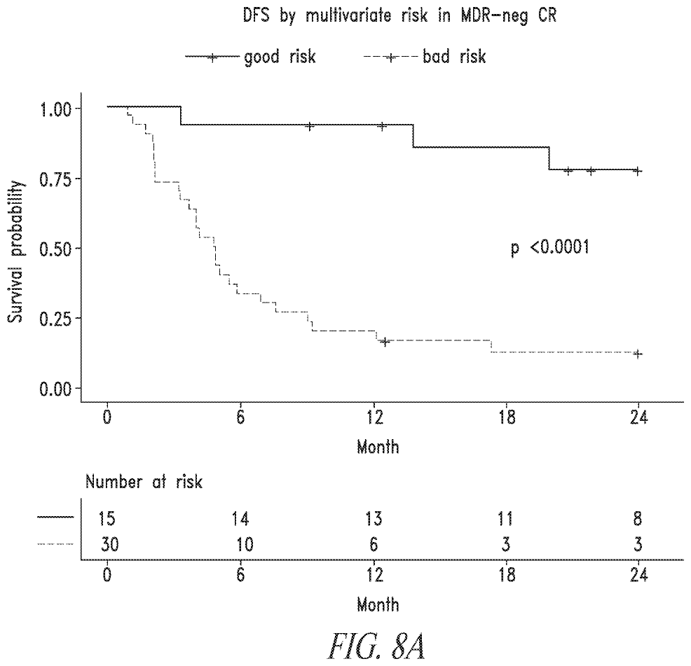

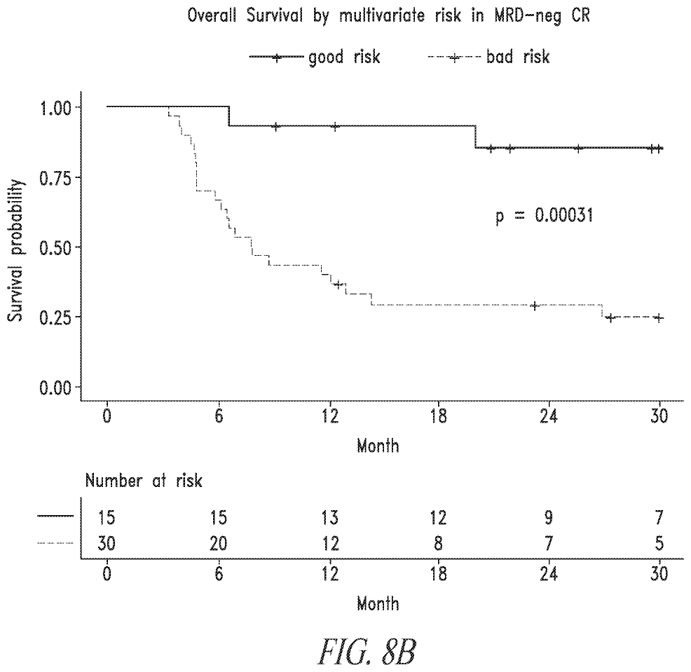

Patient survival was then investigated by evaluating the combined effects of the variables described in Examples 2.4 and 2.5. As shown in FIGS. 7A and 7B, patients with (i) normal LDH and platelets ≥100 prior to lymphodepletion, who (ii) received Cy/Flu as lymphodepleting chemotherapy prior to CAR-T cell infusion, and who (iii) received allo-HSCT following CAR-T cell infusion and achievement of MRD-negative CR had a statistically significant improvement in 24-mo. DFS, and showed a non-statistically significant improvement in OS, versus other MRD-negative CR patients. Overall DFS of patients with “good risk” (pre-lymphodepletion serum LDH of less than 210 U/L; pre-lymphodepletion platelet counts at or above 100 U/L; received Cy/Flu lymphodepletion chemotherapy; pink lines) or “bad risk” (pre-lymphodepletion serum LDH of 210 or more U/L; pre-lymphodepletion platelet counts below 100 U/L; did not receive Cy/Flu) profiles is shown in FIG. 8A. OS is shown in FIG. 8B.

2.8. Association of Allo-HSCT with Outcome

Allogeneic hematopoietic cell transplantation (HCT) is standard of care in suitable R/R adult B-ALL pts after achieving MRD-neg CR. Eighteen patients in MRD-neg CR after treatment CAR-T cells underwent HCT a median of 2.3 months after CAR-T cell infusion. Post-CAR-T cell allo-HSCT correlated with improved survival and low incidences of relapse in B-ALL patients (see Example 2.7). To further investigate the association of allo-HSCT with outcomes, univariate and multivariate analyses were performed with transplant as a time-dependent covariate. Data from the univariate analyses are shown in Tables 16 (association with DFS) and 17 (association with OS). In Tables 16-21, “exp(coef)” is the hazard ratio (HR).

TABLE 16
Univariate Association with DFS with Transplant
as Time-Dependent Co-variate

| | Exp | Exp | Lower | Upper | |
|---|---|---|---|---|---|
| Variable | (Coef) | (−Coef) | (.95) | (.95) | P-value |
| Transplant (Y) | 0.315 | 3.174 | 0.1256 | 0.7902 | 0.0138 |

TABLE 17
Univariate Association with OS with Transplant
as Time-Dependent Co-variate

| | Exp | Exp | Lower | Upper | |
|---|---|---|---|---|---|
| Variable | (Coef) | (−Coef) | (.95) | (.95) | P-value |
| Transplant (Y/N) | 0.3502 | 2.856 | 0.1446 | 0.8478 | 0.02 |

Data from the multivariate analyses are shown in Tables 18 (association with DFS) and 19 (association with OS).

TABLE 18
Multivariate Associations with DFS with
Transplant as Time-Dependent Co-variate

| | Exp | Exp | Lower | Upper | |
|---|---|---|---|---|---|
| Variable | (Coef) | (−Coef) | (.95) | (.95) | P-value |
| Pre-LD LDH | 1.3868 | 0.7211 | 1.1092 | 1.7339 | 0.00412 |
| Cy/Flu (Y) | 0.2490 | 4.0154 | 0.0982 | 0.6316 | 0.00341 |
| Pre-LD Platelets | 0.7357 | 1.3593 | 0.5280 | 1.0250 | 0.06965 |
| Transplant (Y) | 0.3905 | 2.5609 | 0.1326 | 1.1496 | 0.08784 |

TABLE 19
Multivariate Associations with OS with
Transplant as Time-Dependent Co-variate

| | Exp | Exp | Lower | Upper | |
|---|---|---|---|---|---|
| Variable | (Coef) | (−Coef) | (.95) | (.95) | P-value |
| MLL_ALL | 2.2222 | 0.450 | 0.9044 | 5.4600 | 0.08170 |
| Pre-LD Platelets | 0.6130 | 1.631 | 0.4541 | 0.8274 | 0.00138 |
| Transplant (Y) | 0.6432 | 1.555 | 0.2294 | 1.8036 | 0.40155 |

The potential interaction between allo-HSCT with the other significant hazard ratio variables described in Example 2.7 (pre-lymphodepletion LDH, pre-lymphodepletion platelets, Cy/Flu chemotherapy) was investigated. After adjusting for LDH, platelets, and Cy/Flu lymphodepletion, patients undergoing HCT after CAR-T cell therapy had lower risk of failure for DFS compared to those who did not undergo HCT. Table 20 shows the association of “good” versus “bad” risk variables with outcomes, adjusting for post-CAR-T cell allo-HSCT as a time-dependent covariate.

TABLE 20
Association of Risk Factors with Allo-
HSCT as Time-Dependent Co-variate

| | Exp | Exp | Lower | Upper | |
|---|---|---|---|---|---|
| Variable | (Coef) | (−Coef) | (.95) | (.95) | P-value |
| Risk | 8.2505 | 0.1212 | 2.4055 | 28.298 | .000791 |
| Transplant (Y) | 0.4959 | 2.0164 | 0.1988 | 1.237 | 0.132641 |

Table 21 summarizes the interaction effects between allo-HSCT and other risk factors.

TABLE 21
Interaction Effect of Allo-HSCT and Risk Factors

| | Exp | Exp | Lower | Upper | |
|---|---|---|---|---|---|
| Variable | (Coef) | (−Coef) | (.95) | (.95) | P-value |
| Risk | 5.952 | 0.168 | 1.38665 | 25.548 | 0.0164 |
| Transplant (Y) | 0.239 | 4.185 | 0.02095 | 2.725 | 0.2491 |
| Risk-Bad: | 2.326 | 0.430 | 0.17654 | 30.637 | 0.5211 |
| Transplant (Y) | | | | | |

No significant interaction effect was seen between HCT and risk group. With a median follow-up of 28.4 months after HCT, 2-year point estimates of DFS and OS were 61% and 72% respectively. The 2-year cumulative incidence of relapse was 17% and non-relapse mortality was 23%. These data show that post-CAR-T allo-HSCT had a significant association with outcome in the univariate analyses and an important association in the multivariate analyses. The data also indicate that there is no interaction effect between the “good” risk factors identified (Cy/Flu, normal LDH, and platelets >100) and allo-HSCT after CAR-T cell infusion, suggesting that allo-HSCT has an independent effect on outcome regardless of these risk factors.

### 2.9. Conclusions

These data show that in adult patients with relapsed or refractory CD19+ B cell ALL who received CD19 CAR-T cells, the development of MRD-negative CR is strongly associated with in vivo CAR-T cell expansion, and also show that of those patients who achieved MRD-negative CR, patients with lower (pre-lymphodepletion) LDH and higher (pre-lymphodepletion) platelet counts who received Cy/Flu as lymphodepleting chemotherapy prior to CAR-T infusion have longer disease-free survival. Further, allo-HSCT, independent of other factors, may improve disease-free survival and overall survival in patients with MRD-negative CR after CD19 CAR-T cell therapy.

### Example 3

### Factors Associated with Improved Progression-Free Survival and Overall Survival in NHL Patients Who Achieve CR After CD19 CAR-T Therapy

### 3.1. Characteristics of NHL Patients Treated

Patients with indolent or aggressive NHL were eligible for treatment if they were beyond first remission and had received previous treatment with chemoimmunotherapy. Patients with aggressive histology should have relapsed following, or not be eligible for, high-dose therapy and autologous HSCT. Baseline patient characteristics are shown in Table 9. Patients received low or high intensity lymphodepletion chemotherapy with a Cy/Flu-based regimen (low intensity included Cy 30 mg/kg or ≤1500 mg/m2 total dose; high intensity included Cy 60 mg/kg). Two to 4 days after completion of lymphodepletion, CD19 CAR-T cells comprising a 1:1 ratio of CD4+:CD8+ CAR-T cells were infused at a dose of 2×10⁶ EGFRt+ cells/kg.

Specific lympodepletion regimens and patient histologies are shown in Table 22.

TABLE 22
Lymphodepletion Regimens and Histology of NHL patients

| | Indolent | Aggressive | All |
|---|---|---|---|
| Lymphodepletion | histology | histology | patients |
| No. of patients | 9 | 48 | 57 |
| High intensity - no. (%) | 5 (56) | 31 (65) | 36 (63) |
| Cy 60 mg/kg × 1 + Flu | 5 (56) | 30 (63) | 30 (53) |
| 25 mg/m² × 3 | | | |
| Cy 60 mg/kg × 1 + Flu | 0 | 1 (2) | 1 (2) |
| 25 mg/m² × 5 | | | |
| Low intensity - no. (%) | 4 (44) | 17 (35) | 21 (37) |
| Cy 30 mg/kg × 1 + Flu | 0 | 6 (13) | 6 (10) |
| 25 mg/m² × 3 | | | |
| Cy 300 mg/m² × 3 + Flu | 4 (44) | 10 (21) | 14 (25) |
| 30 mg/m² × 3 | | | |
| Cy 500 mg/m² × 3 + Flu | 0 | 1 (2) | 1 (2) |
| 30 mg/m² × 3 | | | |

Cy = cyclophosphamide;
Flu = fludarabine.

### 3.2. Analysis and Results (Patient Cohort Receiving DL2 CAR-T Cells)

3.2.1 Univariate and Multivariate Analysis of Factors Associated with CR

Of 79 NHL patients treated in this study, 57 (median age, 56.5 years; range, 27 to 71 years) received Cy/Flu followed by CAR-T cells at DL2. These patients had received a median of 4 prior treatment regimens (range, 1 to 11), and 47% (n=27) had failed prior autologous (n=19), allogeneic (n=5), or both autologous and allogeneic (n=3) HSCT. Between leukapheresis and lymphodepletion, 13 of 57 patients (23%) required bridging therapy consisting of intensive chemotherapy (n=7), dexamethasone ≥20 mg or equivalent corticosteroid dose (n=10), or combinations of monoclonal antibody, immunomodulatory, and targeted small molecule drugs (n=2). Forty-eight patients (84%) had aggressive histology, including diffuse large B-cell lymphoma (DLBCL; not otherwise specified [NOS] and transformed from indolent), high grade B-cell lymphoma with MYC and BCL2 and/or BCL6 rearrangements (HGBL-DH/TH), grade 3B FL, and other aggressive subtypes. Nine patients (16%) had indolent histology, including 8 with grade 1-3A follicular lymphoma (FL), and 1 with marginal zone lymphoma. Most patients with aggressive NHL (98%) had stage III or IV disease; 90% had extranodal involvement, and 85% had an intermediate or high International Prognostic Index (IPI) score assessed before lymphodepletion).

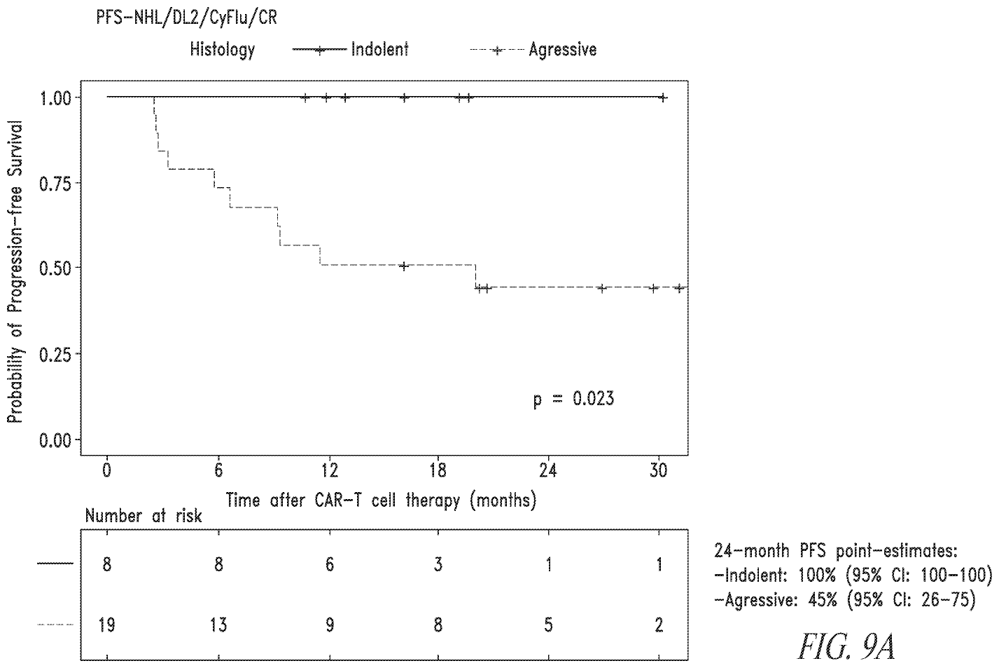

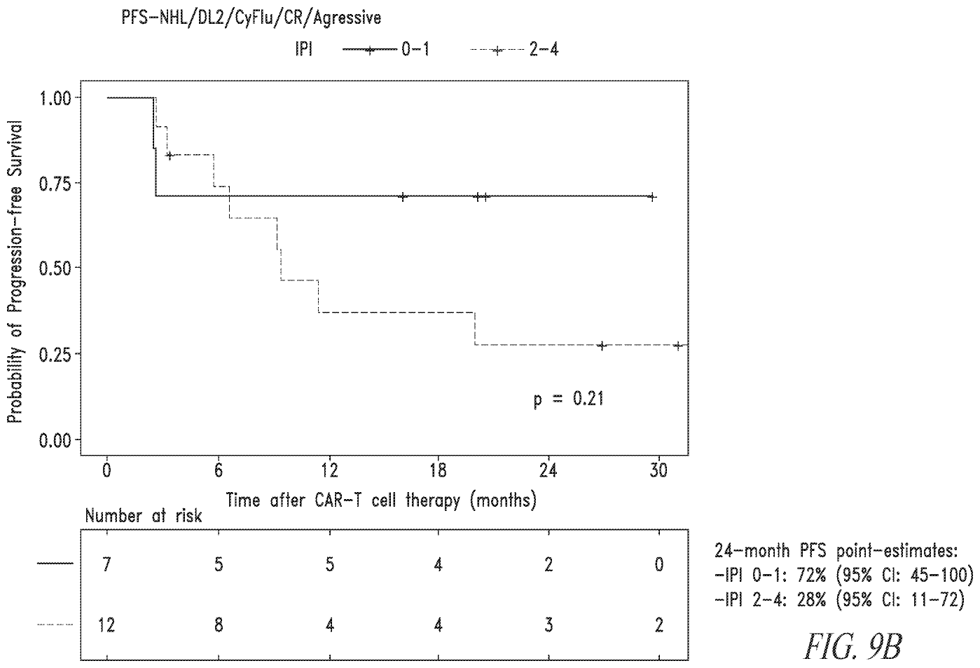

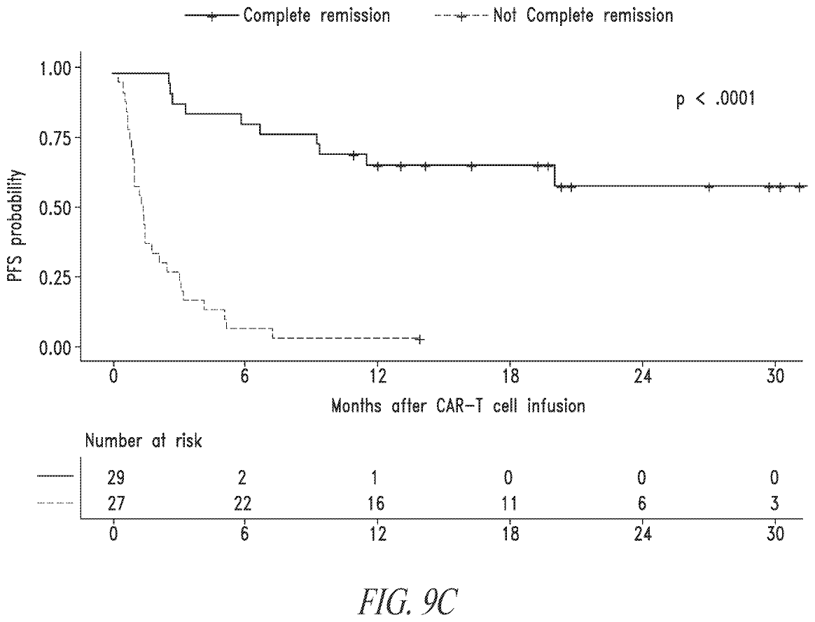

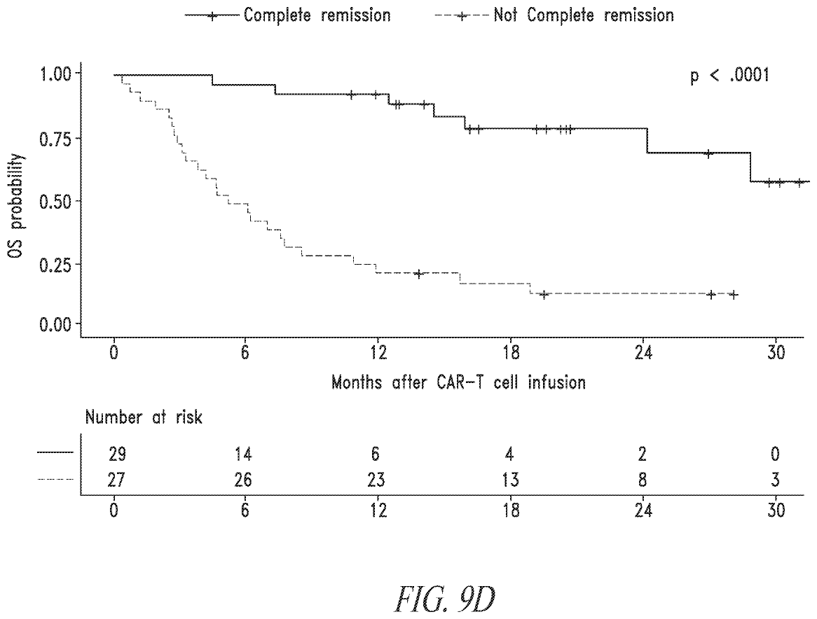

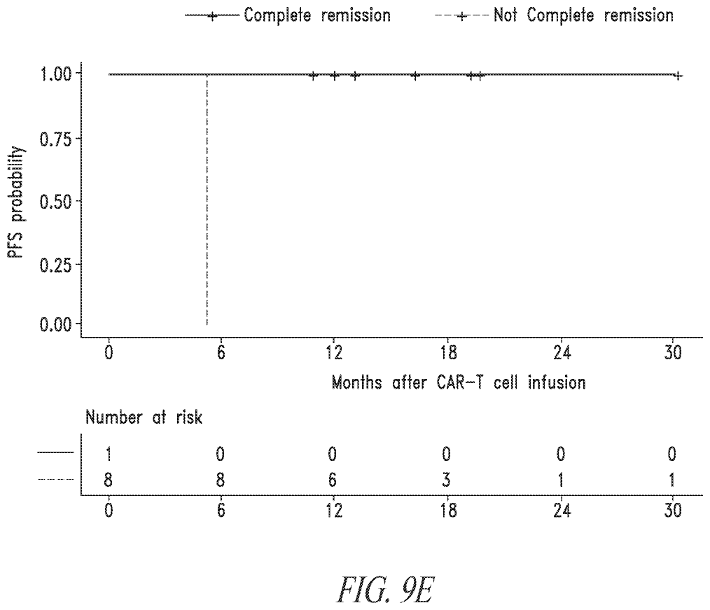

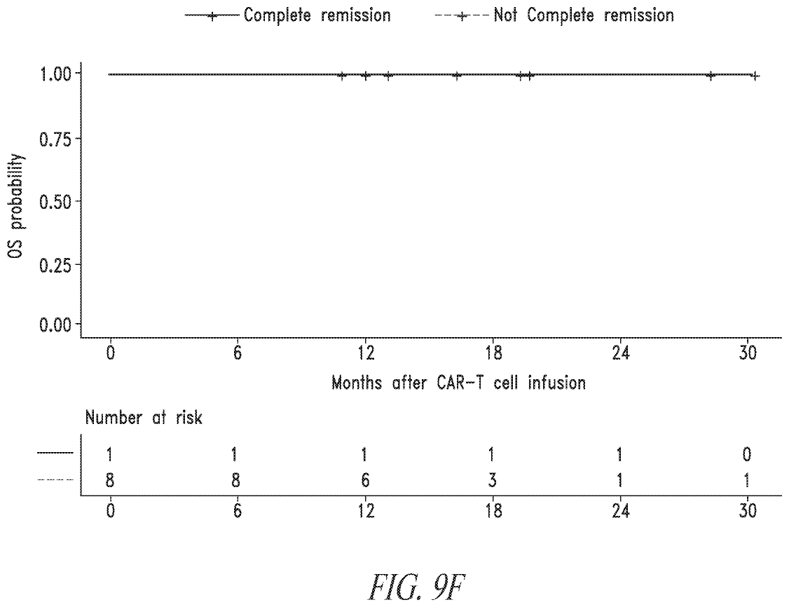

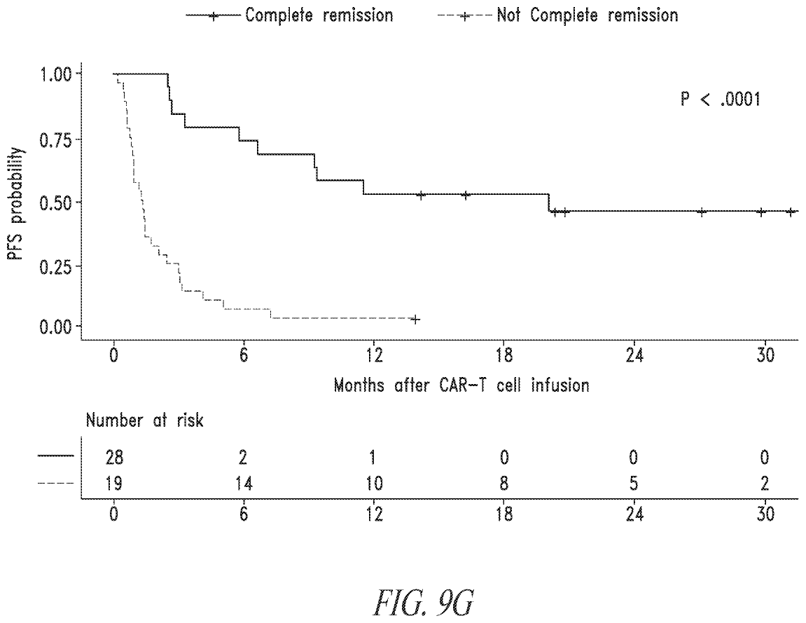

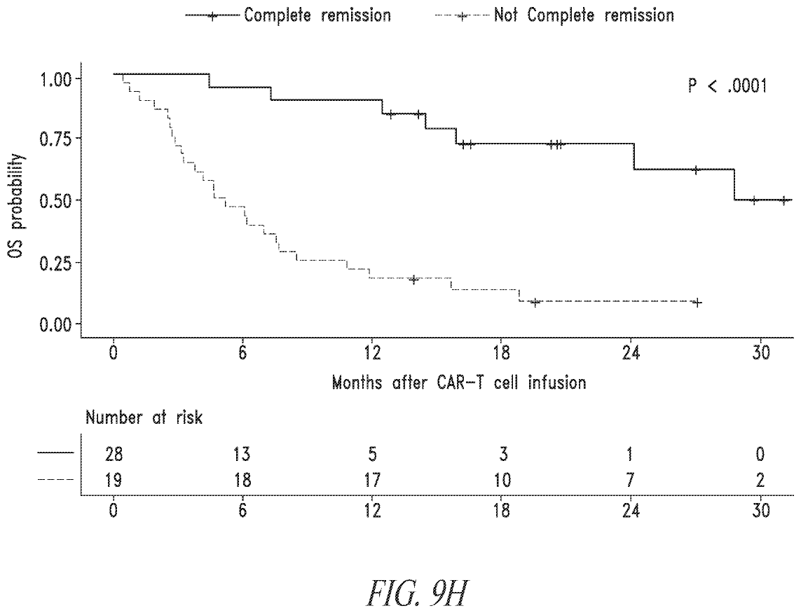

From this group, the best OR rate without additional therapy after CAR-T cells was 57% (95% CI, 43 to 70%), including 48% CR (95% CI, 35 to 62%). The median PFS of those achieving CR was not reached at a median follow-up of 20.2 months (range, 2.5 to 32.4 months). The 24-month probabilities of PFS and OS were 59% (95% CI, 41 to 84%) and 79% (95% CI, 64 to 97%), respectively (FIG. 9C-9E). The median time to best response was 1.0 month (range, 0.9 to 6.3 months). All patients with partial response (PR) or stable disease (SD) on initial restaging 4 weeks after CAR-T cell infusion who did not receive additional therapy (n=4; 2 PR and 2 SD) subsequently converted to CR a median of 1.9 months (range, 1.1 to 5.0 months) after the first response assessment. None of the patients who proceeded to new therapies (11 of 15, 73%) subsequently achieved CR.

Eight of 9 patients with indolent histology achieved CR (89%; 95% CI, 51 to 99%) after CAR-T cell immunotherapy, and all remain in remission at a median follow-up of 14.5 months (range, 10.7 to 30.1 months) (FIG. 9E-9F). One patient with follicular lymphoma who had stable disease at first restaging received radiation therapy starting 2.3 months after CAR-T cell infusion and remains alive with no further treatment 28.0 months after CAR-T cell infusion. Patients with indolent NHL had received a median of 4 prior treatment regimens (range, 2 to 7); 78% (n=7) were refractory to the last therapy, and 44% (n=4) had relapsed after autologous (n=3) or allogeneic (n=1) HSCT.

For the 47 patients with any aggressive NHL histology, the OR rate was 51% (95% CI, 36 to 66%), including 40% CR (95% CI, 27 to 56%). Among aggressive histology subtypes, patients with DLBCL (NOS and transformed from indolent) had OR and CR rates of 50% (95% CI, 33 to 67%) and 43% (95% CI, 25 to 63%), respectively, while patients with confirmed HGBL-DH/TH had OR and CR rates of 38% (95% CI, 10 to 74%) and 25% (95% CI, 4 to 64%), respectively. In patients with any aggressive histology who achieved CR, the median PFS was 20.0 months (95% CI, 9.2 to could not be estimated) at a median follow-up of 26.9 months (range, 2.5 to 32.4 months), and 24-month probabilities of PFS and OS were 46% (95% CI, 28 to 76%) and 72% (95% CI, 54 to 96%), respectively (FIG. 9G-9H). Most relapses occurred in the first year after CAR-T cell infusion; beyond 12 months, only one patient relapsed (CD19-positive relapse at 20.0 months after CAR-T cell infusion).

A univariate analysis of CR patient and treatment factors was performed. Results are shown in Table 23.

TABLE 23
Univariate Analysis of Factors Impacting the Likelihood of Achieving
CR in NHL Patients Receiving CAR-T Cell Therapy (n = 57)

| | Odds | |
|---|---|---|
| Variable | ratio* | P-value |
| LDH above normal limit (Y) | 0.125 | 0.001 |
| International Prognostic Index (IPI) at enrollment | 0.266 | 0.001 |
| Serum LDH preLD† (U/L, per U/L increase increment) | 0.991 | 0.002 |
| CD3+ CAR-T cells (logₑ %, AUC⋄ day 0-28) | 1.593 | 0.006 |
| CD8+ CAR-T cells (logₑ %, peak) | 1.563 | 0.011 |
| B-cell reconstitution (Y) | 7.941 | 0.013 |
| CD8+ CAR-T cells (logₑ cells/μL, peak) | 1.438 | 0.014 |
| Aggressive histology (Y) | 0.069 | 0.015 |
| Tumor cross-sectional area at enrollment (mm², per | 1.000 | 0.017 |
| mm² increase increment) | | |
| Time from leukapheresis to last intensive chemotherapy | 1.734 | 0.022 |
| (logₑ days, per logₑ increase increment) | | |
| CD8+ CAR-T cells (logₑ cells/μL, AUC peak-day 30) | 1.398 | 0.030 |
| IL-7 (pg/mL, fold change preLD to day 0) | 0.620 | 0.045 |
| IFN-γ (logₑ pg/mL, AUC day 0-28) | 3.139 | 0.051 |
| IL-2Rα (logₑ pg/mL, peak, per logₑ increase increment) | 1.710 | 0.054 |
| Normal B-cells in marrow (%, preLD) | 1.235 | 0.056 |
| TGF-β1 (logₑ pg/mL, peak, per logₑ increase increment) | 2.123 | 0.062 |
| Extranodal disease (Y) | 0.204 | 0.063 |
| IL-2 (logₑ pg/mL, AUC day 0-28) | 0.412 | 0.063 |
| CD8+ CAR-T cells (logₑ cells/μL, AUC day 0-28) | 1.314 | 0.066 |
| Corticosteroid after leukapheresis (Y) | 0.220 | 0.072 |
| s-FAS (logₑ pg/mL, AUC day 0-28) | 4.634 | 0.075 |
| FlapEF1α (logₑ copies/μg DNA, peak) | 1.278 | 0.082 |
| MCP-1 (logₑ pg/mL, delta from preLD to day 0) | 1.002 | 0.087 |
| TGF-β1 (logₑ pg/mL, AUC day 0-28) | 2.103 | 0.090 |
| CD8+ CAR-T cells (logₑ cells/μL, slope peak to day 30) | 0.864 | 0.092 |
| CD4+ CAR-T cells (logₑ cells/μL, peak) | 1.277 | 0.097 |

†PreLD = pre-lymphodepletion
⋄AUC = area under the curve
*Higher odds ratio means higher likelihood of achieving CR

In aggressive and indolent NHL patients (n=56), univariate analysis suggested that the peak of CAR-T cell expansion in blood was associated with the development of CR (odds ratio [OR] 2.23 for each log 10 CD8+ CAR-T cells/μL increment, P=0.02). Because only one patient with indolent lymphoma failed to achieve CR after CAR-T cell immunotherapy, the multivariate logistic regression analysis focused on patients with aggressive NHL histology (n=47) and considered the following factors:
Age; aggressive lymphoma (Y); serum LDH pre-lymphodepletion (U/L); time from leukapheresis to last intensive chemotherapy (logₑ days); corticosteroid after leukapheresis (Y); MCP-1 (logₑ pg/mL, day 0); IL-15 (logₑ pg/mL, day 0); CD3+CD4+ post-bead removal-pre-LCL projected fold expansion; CD4+ CAR-T cells fold expansion day 6-15; CD3+CD8+ post-bead removal-pre-LCL projected fold expansion; CD8+ CAR-T cells fold expansion day 6-15; CD4+ CAR-T cells (logₑ cells/μL, peak); CD8+ CAR-T cells (logₑ cells/μL, peak); CRS grade (0-4, peak); IL-7 (pg/mL, peak); IFN-γ (pg/mL, peak); TGF-I31 (1 logₑ pg/mL, peak)

A p-value cutoff of 0.10 from univariate analysis was applied, and variables with data missing from more than 10% of the patients were excluded. Variables that were highly correlated with other variables were also removed. For CAR-T cell expansion, CD8+ CAR-T variables were used instead of FlapEF1α.

Aggressive histology, pre-lymphodepletion LDH, time from leukapharesis to last intensive chemotherapy, and expansion of post-infusion CD8+ CAR-T cells were the factors included in the final multivariable model, as shown in Table 24.

TABLE 24
Multivariable Logistic Regression Model of Factors
Impacting the Likelihood of Achieving CR in
NHL Patients Receiving CAR-T Cell Therapy

| Variable | OR* | 95% CI | P-value |
|---|---|---|---|
| Aggressive histology (Y) | 0.24 | 0.01-2.12 | 0.25 |
| Serum LDH (U/L, per 100 U/L increase | 0.51 | 0.26-0.90 | 0.03 |
| increment) | | | |
| Time from leukapheresis to last | 1.79 | 0.95-3.75 | 0.09 |
| intensive chemotherapy (logₑ days, per | | | |
| logₑ increase increment) | | | |
| CD8+ CAR-T cells (logₑ cells/μL, peak) | 1.42 | 1.03-2.16 | 0.06 |

*Higher Odds Ratio (OR) means higher likelihood of achieving CR

These data show that lower pre-lymphodepletion serum LDH concentration and higher in vivo peak CD8+ CAR-T cell count are associated with higher likelihood of CR. In particular, the multivariable analysis demonstrated that higher pre-lymphodepletion serum LDH was negatively associated with the likelihood of achieving CR, with odds ratio (OR, [95% Confidence Interval, CI]) of 0.51 [0.26-0.90] for each U/L increase (P=0.03), while higher in vivo peak CD8+ CAR-T cell by flow cytometry analysis of peripheral blood (OR 1.42[1.03-2.16], P=0.05) was correlated with the development of CR.

3.2.2 Analysis of Factors Associated with PFS

To identify baseline and therapy related factors that were associated with a more durable PFS, three chronological landmark analyses (pre-lymphodepletion, Day-0 pre-CAR-T cell infusion were performed in all patients (n=57), and day-28 landmark in CR patients including (n=27) and excluding (n=19) patients with indolent histology (since there was no PFS/OS event in that subgroup) using penalized Cox regression model via elastic net approach (Hui and Trevor, Journal of the Royal Statistical Society: Series B (Statistical Methodology) 67(2):301 (2005)) including clinical/biological relevant variables, factors significant in preliminary univariable analysis, and any significant factor from the previous step in the pool of selection variables for the subsequent model.

Univariable analysis of the entire treatment cohort is summarized in Table 25. Univariable analysis of the CR sub-cohort is summarized in Table 26.

TABLE 25
Univariable Analysis of Factors Correlated with PFS -
whole NHL cohort, n = 57 (Cox regression model)

| Variable | HR* | 95% CI | P-value |
|---|---|---|---|
| CD3+ CAR-T cells (logₑ %, AUC | 0.559 | 0.444-0.703 | 7.32E−07 |
| day 0-28) | | | |
| CD8+ CAR-T cells (logₑ %, peak) | 0.570 | 0.452-0.718 | 1.83E−06 |
| IPI at enrollment | 2.361 | 1.547-3.602 | 6.77E−05 |
| LDH above normal limit (Y) | 4.869 | 2.137-11.096 | 1.65E−04 |
| CD8+ CAR-T cells (logₑ cells/μL, | 0.762 | 0.654-0.888 | 0.001 |
| peak) | | | |
| FlapEF1α (logₑ copies/μg DNA, | 0.731 | 0.609-0.878 | 0.001 |
| peak) | | | |
| Tumor cross-sectional area at | 1.000 | 1.000-1.000 | 0.002 |
| enrollment (mm², per increase | | | |
| increment) | | | |
| IL-7 (logₑ pg/mL, AUC day 0-28) | 0.387 | 0.208-0.717 | 0.003 |
| Serum LDH preLD (logₑ U/L, per | 1.003 | 1.001-1.004 | 0.005 |
| U/L increase increment) | | | |
| TGF-β1 (logₑ pg/mL, AUC day 0- | 0.449 | 0.255-0.790 | 0.006 |
| 28) | | | |
| CD8+ CAR-T cells (logₑ cells/μL, | 0.790 | 0.666-0.938 | 0.007 |
| AUC peak to day 30) | | | |
| MCP-1 (logₑ pg/mL, day 0) | 0.589 | 0.398-0.871 | 0.008 |
| BAFF (logₑ pg/mL, AUC | 0.258 | 0.094-0.706 | 0.008 |
| day 0-28) | | | |
| IL-7 (pg/mL, peak) | 0.963 | 0.937-0.991 | 0.009 |
| FlapEF1α (logₑ copies/μg DNA, | 0.721 | 0.561-0.926 | 0.010 |
| AUC peak to day 30) | | | |
| CD8+ CAR-T cells (logₑ cells/μL, | 0.794 | 0.665-0.948 | 0.011 |
| AUC day 0-28) | | | |
| TGF-β1 (logₑ pg/mL, preLD) | 0.528 | 0.322-0.866 | 0.011 |
| Aggressive histology (Y) | 12.189 | 1.657-89.671 | 0.014 |
| IFN-γ (pg/mL, Δ preLD to day 0) | 2.117 | 1.158-3.868 | 0.015 |
| TGF-β1 (logₑ pg/mL, peak) | 0.520 | 0.304-0.889 | 0.017 |
| Cy(50%)Flu3 lymphodepletion (Y) | 4.266 | 1.284-14.172 | 0.018 |
| IL-7 (pg/mL, preLD) | 0.937 | 0.887-0.990 | 0.020 |
| MCP-1 (logₑ pg/mL, peak) | 0.644 | 0.442-0.938 | 0.022 |
| IL-7 (pg/mL, fold change preLD to | 1.288 | 1.035-1.602 | 0.023 |
| day 0) | | | |
| CD4+ CAR-T cells (logₑ %, peak) | 0.789 | 0.635-0.982 | 0.034 |
| Bulky disease (yes) | 2.836 | 1.058-7.606 | 0.038 |
| MCP-1 (logₑ pg/mL, preLD) | 0.596 | 0.364-0.974 | 0.039 |
| CD4+ CAR-T cells (logₑ cells/μL, | 0.811 | 0.662-0.993 | 0.043 |
| AUC day 0-28) | | | |
| MCP-1 (logₑ pg/mL, AUC | 0.625 | 0.392-0.994 | 0.047 |
| day 0-28) | | | |
| CD4+ CAR-T cells (logₑ cells/μL, | 0.831 | 0.691-0.999 | 0.049 |
| peak) | | | |
| FlapEF1α (logₑ copies/μg DNA, | 0.802 | 0.644-0.999 | 0.049 |
| AUC day 0-28) | | | |
| TNFRp75 (logₑ pg/mL, peak) | 1.759 | 0.993-3.115 | 0.053 |
| IL-18 (logₑ pg/mL, AUC day 0-28) | 1.638 | 0.993-2.701 | 0.053 |
| IL-7 (pg/mL, day 0) | 0.971 | 0.942-1.001 | 0.055 |
| Time from leukapheresis to last | 0.761 | 0.573-1.011 | 0.059 |
| intensive chemotherapy (log10 | | | |
| days, per 1 day increase increment) | | | |
| IFN-γ (pg/mL, preLD†) | 0.405 | 0.158-1.042 | 0.061 |
| TNFRp75 (logₑ pg/mL, AUC⋄ | 2.022 | 0.962-4.249 | 0.063 |
| day 0-28) | | | |
| IL-8 (logₑ pg/mL, peak) | 0.805 | 0.640-1.013 | 0.064 |
| IL-15 (logₑ pg/mL, AUC day | 0.577 | 0.321-1.036 | 0.066 |
| 0-28) | | | |
| IL-18 (logₑ pg/mL, preLD) | 1.569 | 0.970-2.539 | 0.066 |
| B-cell reconstitution (Y) | 0.408 | 0.156-1.067 | 0.068 |
| IL-18 (logₑ pg/mL, peak) | 1.418 | 0.974-2.066 | 0.069 |
| BAFF (pg/mL, peak) | 0.985 | 0.970-1.001 | 0.071 |
| IL-8 (logₑ pg/mL, AUC day 0-28) | 0.720 | 0.504-1.028 | 0.071 |
| IFN-γ (logₑ pg/mL, AUC | 0.516 | 0.249-1.071 | 0.076 |
| day 0-28) | | | |
| MCP-1 (logₑ pg/mL, Δ preLD to | 0.998 | 0.997-1.000 | 0.076 |
| day 0) | | | |
| TNFRp75 (logₑ pg/mL, preLD) | 1.636 | 0.943-2.837 | 0.080 |
| TNF-α (logₑ pg/mL, AUC | 0.719 | 0.494-1.047 | 0.086 |
| day 0-28) | | | |
| FlapEF1α (logₑcopies/μg DNA, | 0.827 | 0.664-1.029 | 0.089 |
| AUC day 28-60) | | | |
| IL-22 (logₑ pg/mL, peak) | 0.595 | 0.326-1.083 | 0.089 |
| IL-15 (logₑ pg/mL, day 0) | 0.648 | 0.391-1.074 | 0.092 |

†preLD = pre-lymphodepletion
⋄AUC = area under the curve
*Lower HR is associated with longer PFS

TABLE 26
Univariable Analysis of Factors Correlated
with PFS (Cox regression model) (n = 27)

| Variable | HR* | 95% CI | P-value |
|---|---|---|---|
| IL-7 (logₑ pg/mL, AUC⋄ day 0-28) | 0.202 | 0.067-0.611 | 0.005 |
| TGF-β1 (logₑpg/mL, AUC day 0-28) | 0.158 | 0.034-0.725 | 0.018 |
| BAFF (logₑ pg/mL, AUC day 0-28) | 0.011 | 0.000-0.551 | 0.024 |
| CD8+ CAR-T cells (logₑ %, peak) | 0.505 | 0.277-0.920 | 0.026 |
| TNFRp75 (logₑ pg/mL, AUC day 0- | 4.138 | 1.183-14.481 | 0.026 |
| 28) | | | |
| TNFRp75 (logₑ pg/mL, preLD†) | 3.450 | 1.152-10.329 | 0.027 |
| IL-7 (pg/mL, peak) | 0.940 | 0.889-0.993 | 0.028 |
| CD3+ CAR-T cells (logₑ %, AUC day | 0.535 | 0.302-0.947 | 0.032 |
| 0-28) | | | |
| IFN-γ (pg/mL, peak) | 1.096 | 1.008-1.192 | 0.032 |
| IL-18 (logₑ pg/mL, day 0) | 3.402 | 1.102-10.504 | 0.033 |
| IL-18 (logₑ pg/mL, peak) | 1.886 | 1.040-3.420 | 0.037 |
| IL-18 (logₑ pg/mL, AUC day 0-28) | 2.220 | 1.027-4.798 | 0.043 |
| IL-18 (logₑ pg/mL, preLD) | 2.413 | 1.006-5.789 | 0.049 |
| IL-7 (pg/mL, preLD) | 0.877 | 0.767-1.003 | 0.056 |
| TNFRp75 (logₑ pg/mL, day 0) | 2.513 | 0.946-6.676 | 0.065 |
| IL-8 (logₑ pg/mL, AUC day 0-28) | 0.578 | 0.319-1.047 | 0.070 |
| FlapEF1α (logₑ copies/μg DNA, AUC | 0.762 | 0.567-1.024 | 0.072 |
| day 28-60) | | | |
| IL-7 (pg/mL, day 0) | 0.950 | 0.899-1.005 | 0.072 |
| BAFF (pg/mL, peak) | 0.954 | 0.906-1.005 | 0.078 |
| CD8+ central memory selection (Y) | 3.846 | 0.807-18.326 | 0.091 |
| TGF-β1 (logₑ pg/mL, preLD) | 0.321 | 0.085-1.221 | 0.096 |
| Tumor cross-sectional area at | 1.000 | 1.000-1.001 | 0.097 |
| enrollment (logₑ mm², per mm² | | | |
| increase increment) | | | |

⋄AUC = area under the curve
†preLD = pre-lymphodepletion
*Lower HR is associated with longer PFS

From the univariate data, elastic net multivariable penalized Cox regression models were generated for pre-lymphodepletion, Day-0, and Day-28 landmarks. N=48 (excluding patients with indolent histology (n=9) due to no PFS/OS event). A p-value cutoff of 0.10 from univariate analysis was applied, and variables with data missing from more than 10% of the patients were excluded. Variables that were highly correlated with other variables were also removed. The pre-lymphodepletion landmark analysis considered the following variables:
Age; Serum LDH pre-lymphodepletion U/L); Normal B-cells in marrow (%); Time from leukapheresis to last intensive chemotherapy (logₑ days); Treatment after leukapheresis (Y); IL-7 (pg/mL, preLD); MCP-1 (logₑ pg/mL, preLD); TGF-I31 (logₑ pg/mL, preLD); IFN-γ (pg/mL, preLD); IL-18 (logₑ pg/mL, preLD); and TNFRp75 (logₑ pg/mL, preLD)
   The final pre-lymphodepletion landmark model is shown in Table 27.

TABLE 27
Penalized Cox Regression Models for NHL PFS Using Elastic Net
Approach - pre-lymphodepletion landmark; n = 48 (Excluding
Patients with Indolent Histology [n = 9] due to no PFS/OS event)

| Factors | HR* | 95% CI | P-value |
|---|---|---|---|
| Serum LDH (U/L, per 100 U/L increase | 1.39 | 1.12-1.71 | 0.002 |
| increment) | | | |
| IL-7 (pg/mL, preLD†, per 1 pg/mL | 0.92 | 0.87-0.97 | 0.002 |
| increase increment) | | | |
| IL-18 (logₑ pg/mL, preLD, per logₑ | 1.90 | 1.10-3.29 | 0.02 |
| increase increment) | | | |

†preLD = pre-lympodepletion
*Lower HR is associated with longer PFS

A p-value cutoff of 0.10 from univariate analysis was applied, and variables with data missing from more than 10% of the patients were excluded. Variables that were highly correlated with other variables were also removed. The Day-0 landmark elastic net multivariable analysis considered the following variables:
Serum LDH preLD (U/L); IL-7 (pg/mL, preLD); IL-18 (logₑ pg/mL, preLD); CD3+CD4+ post-bead removal-pre-LCL projected fold expansion; CD4+ CAR-T cells fold expansion day 6-15; CD3+CD8+ post-bead removal-pre-LCL projected fold expansion; CD8+ CAR-T cells fold expansion day 6-15; IL-7 (pg/mL, day 0); MCP-1 (logₑ pg/mL, day 0); IL-15 (logₑ pg/mL, day 0); IL-2Rα (logₑ pg/mL, day 0); IL-18 (logₑ pg/mL, day 0); and TNFRp75 (logₑ pg/mL, day 0).
   The final Day-0 landmark model is shown in Table 28.

TABLE 28
Penalized Cox Regression Models for NHL PFS using Elastic
Net Approach - Day-0 Landmark; n = 48 (Excluding Patients
with Indolent Histology [n = 9] due to no PFS/OS event)

| Factors | HR* | 95% CI | P-value |
|---|---|---|---|
| Serum LDH (log10 U/L, per 1 U/L | 1.59 | 1.24-2.04 | 0.001 |
| increase increment) | | | |
| IL-7 (pg/mL, preLD†, per 1 pg/mL | 0.95 | 0.90-1.01 | 0.10 |
| increase increment) | | | |
| MCP-1 (logₑ pg/mL, day 0, per logₑ | 0.51 | 0.31-0.85 | 0.009 |
| increase increment) | | | |
| IL-18 (logₑ pg/mL, day 0, per logₑ | 2.32 | 1.30-4.10 | 0.004 |
| increase increment) | | | |

†preLD = pre-lympodepletion
*Lower HR is associated with longer PFS

A p-value cutoff of 0.10 from univariate analysis was applied, and variables with data missing from more than 10% of the patients were excluded. Variables that were highly correlated with other variables were also removed. The Day-28 landmark elastic net multivariable analysis considered the following variables:
Serum LDH preLD (U/L); MCP-1 (logₑ pg/mL, day 0); IL-18 (logₑ pg/mL, day 0); CD4+ CAR-T cells (logₑ cells/μL, peak); CD8+ CAR-T cells (logₑ cells/μL, peak); IL-7 (pg/mL, peak); MCP-1 (logₑ pg/mL, peak); TGF-I31 (logₑ pg/mL, peak); IFN-γ (pg/mL, peak); IL-15 (logₑ pg/mL, peak); IL-8 (logₑ pg/mL, peak); IL-22 (logₑ pg/mL, peak) .
   The final Day-28 landmark model is shown in Table 29.

TABLE 29
Penalized Cox regression models for NHL PFS using elastic
net approach - Day-28 landmark; n = 27 (CR patients)

| Factors | HR* | 95% CI | P-value |
|---|---|---|---|
| Serum LDH (log10 U/L, per 100 U/L | 1.27 | 1.04-1.54 | 0.02 |
| increase increment) | | | |
| CD8+ CAR-T cells (logₑ cells/μL, peak) | 0.81 | 0.68-0.95 | 0.01 |
| IL-7 (pg/mL, peak, per 1 pg/mL increase | 0.97 | 0.93-1.00 | 0.06 |
| increment) | | | |
| MCP-1 (logₑ pg/mL, peak, per logₑ | 0.79 | 0.48-1.31 | 0.36 |
| increase increment) | | | |

*Lower HR is associated with longer PFS

A Day-28 landmark multivariate analysis was also performed using data from CR patients only and excluding patients with indolent histology. Data is shown in Table 30:

TABLE 30
Penalized Cox Regression Model for PFS using Elastic Net Approach -
Day-28 landmark; n = 19 (CR patients excluding patients
with indolent histology [n = 9] due to no PFS/OS event)

| Variables | HR* | 95% CI | P-value |
|---|---|---|---|
| IL-18 (logₑ pg/mL, day 0, per logₑ increase | 7.24 | 1.34-39.24 | 0.02 |
| increment) | | | |
| IL-7 (pg/mL, peak, per 1 pg/mL increase | 0.93 | 0.86-1.00 | 0.05 |
| increment) | | | |

These data show that all three landmark models showed shorter PFS in adult NHL patients who receive CD19 CAR-T cell therapy and have higher pre-lymphodepletion serum LDH concentration, with HR of 1.39 [1.12-1.71], 1.59 [1.24-2.03], and 1.27 [1.04-1.54] for each 100 U/L increase, and P=0.002, <0.001, and 0.02, respectively.

Additionally, the three landmark multivariable models indicate that lower serum IL-18 pre-lymphodepletion (HR 1.90 [1.10-3.29] for each logₑ pg/mL increase, P=0.02) and immediately before CAR-T cell infusion (HR 2.32 [1.30-4.10] and 7.24 [1.34-39.24] for each logₑ pg/mL increase, P=0.004 and 0.02 for day-0 and day-28 landmarks, respectively), and higher serum IL-7 pre-lymphodepletion (HR 0.92 [0.87-0.97] for each pg/mL increase, P=0.002) and peak after CAR-T cell infusion (HR 0.93 [0.86-1.00] for each pg/mL increase, P=0.05) are associated with longer PFS. In patients achieving CR with low serum pre-lymphodepletion IL-18 and high serum IL-7 concentration after CAR-T cell infusion, the probability of 2-year PFS was 100%. Higher serum MCP-1 immediately before CAR-T cell infusion also correlates with better PFS in the day-0 landmark model (HR 0.51 [0.31-0.85] for each logₑ pg/mL increase, P=0.009).

Survival curves were constructed that estimated the probability of PFS or OS based on tumor histology (FIG. 9A), International Prognostic Index score upon enrollment (IPI; FIG. 9B), achievement of CR following CAR-T infusion (FIGS. 9C and 9D), or CR to indolent (FIGS. 9E and 9F) or aggressive (FIGS. 9G and 9H; Table 31) disease following CAR-T infusion.

TABLE 31
Day 28 Landmark Analysis of Factors Associated With
Improved PFS in All NHL Patients Who Achieved CR Using
an Elastic Net Multivariate Cox Regression Model

| Variable | HR (95% Cl) | P-value |
|---|---|---|
| Aggressive histology | 136.32 (5.11-3633.64) | 0.003 |

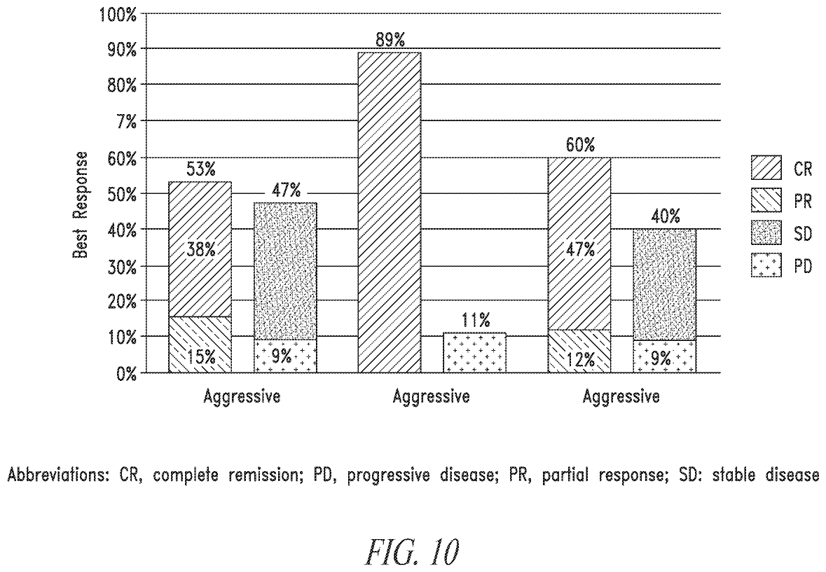

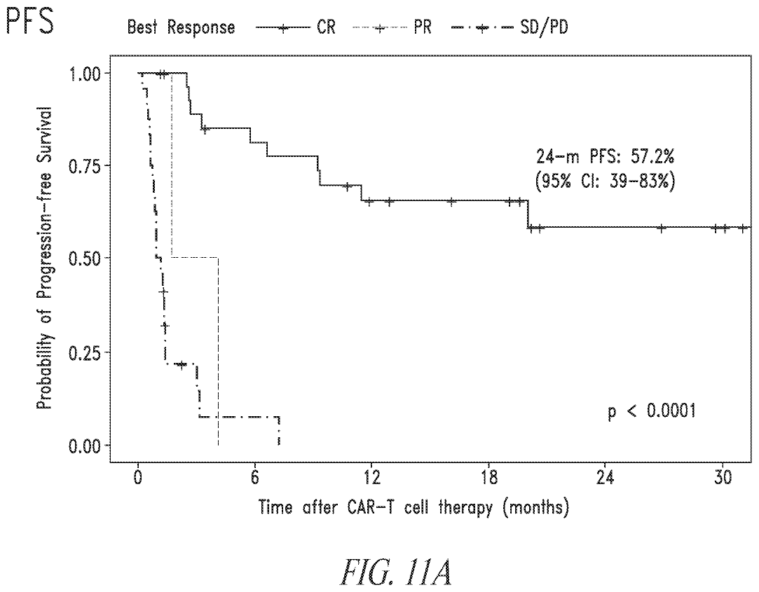

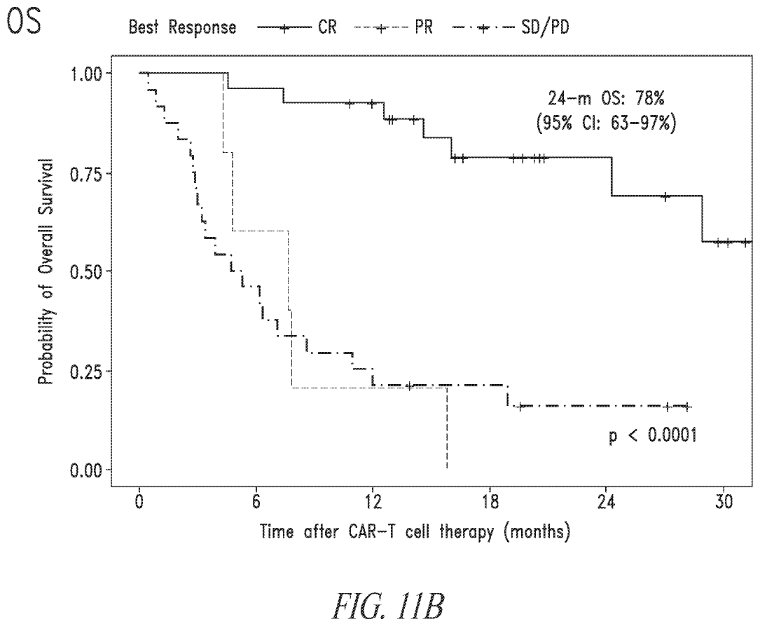

Patient outcome by tumor histology is summarized in FIG. 10. It was also determined that complete response is associated with better PFS (FIG. 11A) and OS (overall survival) (FIG. 11B).

3.3. Analysis of Factors Associated with Achieving CR in NHL

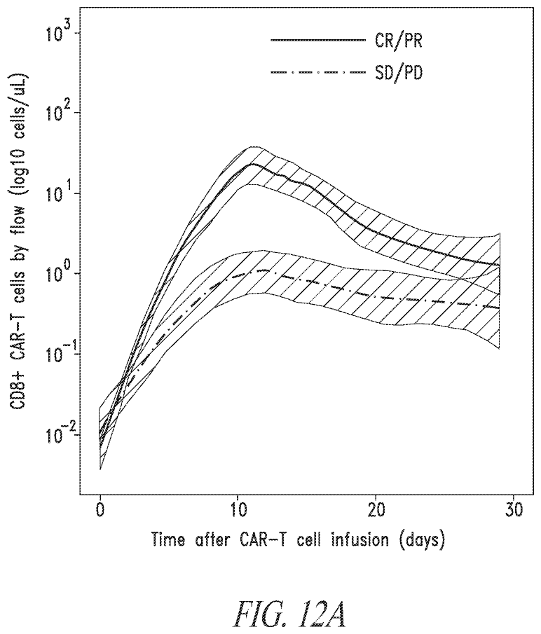

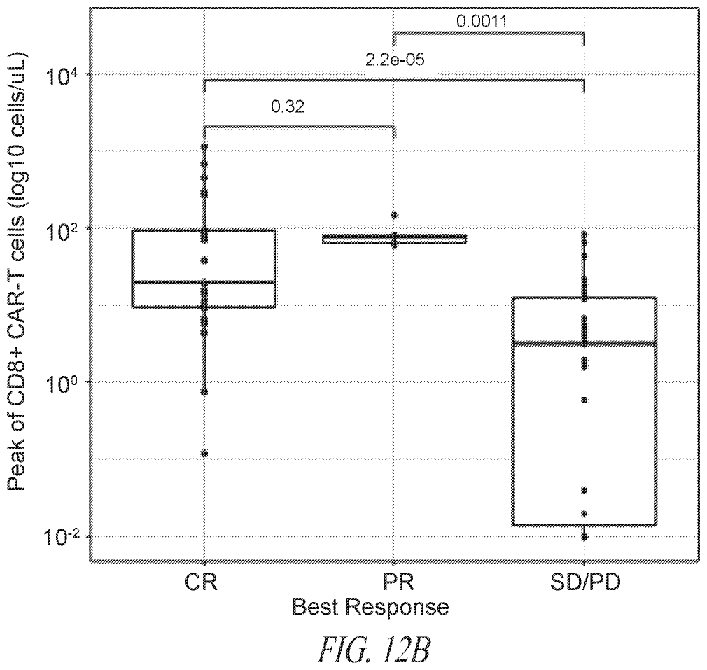

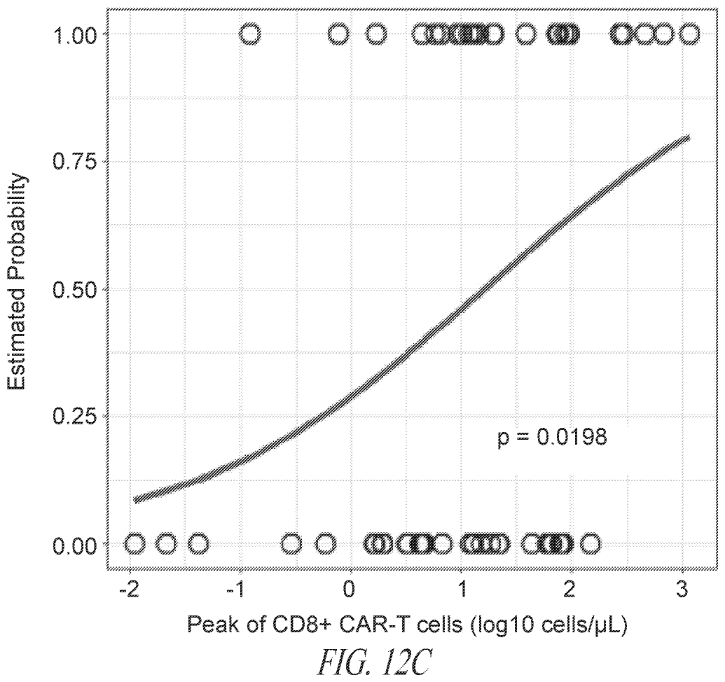

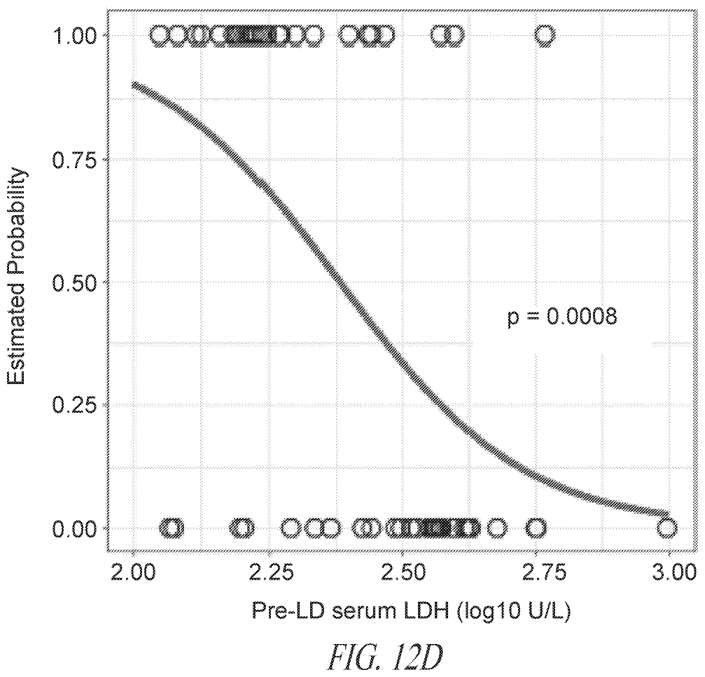

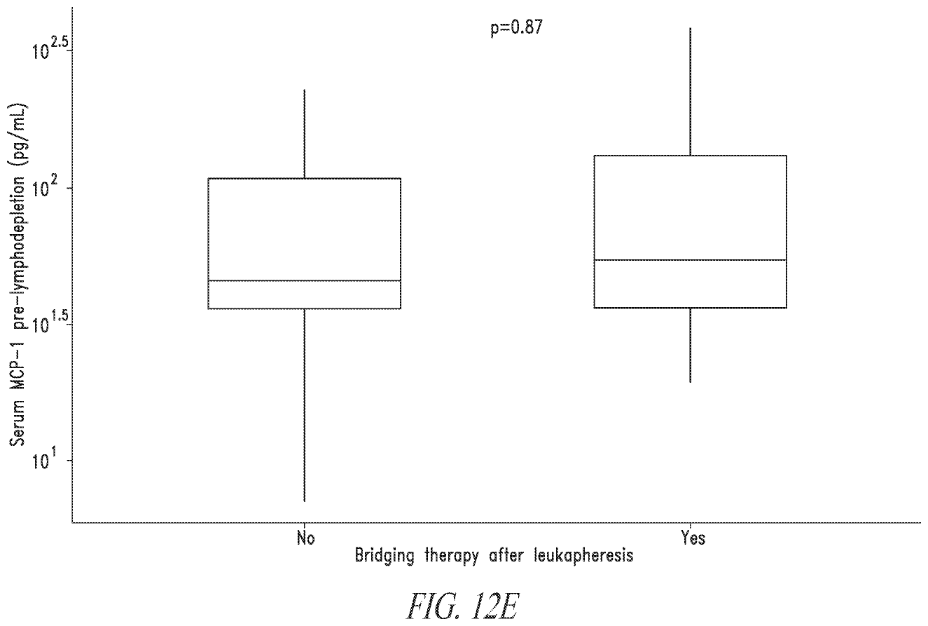

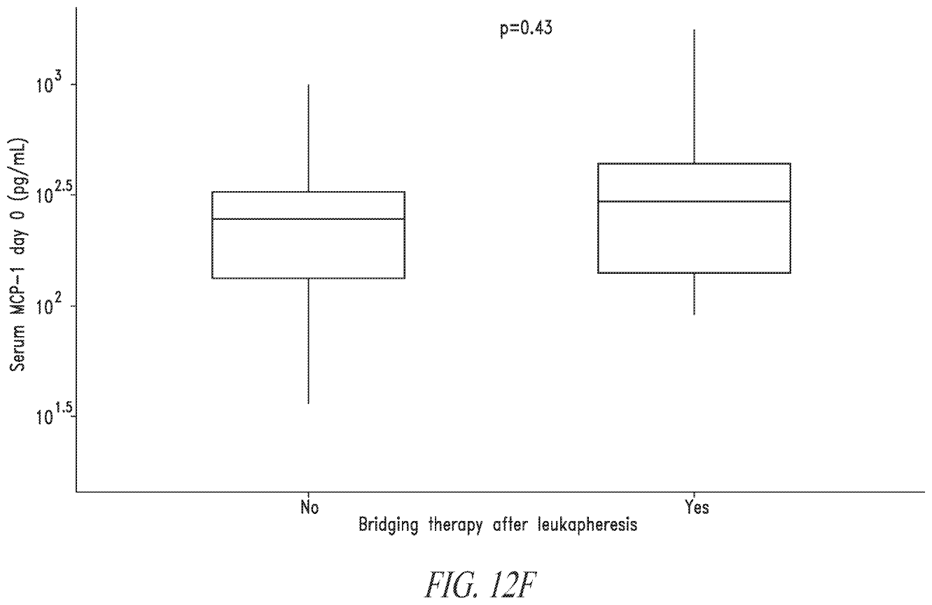

In vivo expansion of CAR-T cells and pre-lymphodepletion serum LDH were shown to be associated with CR in B-ALL patients (see FIGS. 3A and 3B, and Table 8). Analysis of NHL patients showed a similar association, as shown in FIGS. 12A-12D. From these data, a multivariable logistic regression model for factors impacting the probability of NHL patients achieving CR following CAR-T cell therapy was generated (Table 32).

TABLE 32
Multivariable Logistic Regression Model for
Factors Impacting the Probabilitv of CR

| Variables | OR | 95% Cl | P-value |
|---|---|---|---|
| Aggressive histology | 0.24 | 0.01-2.12 | 0.25 |
| Serum LDH (pre-LD)* | 0.51 | 0.26-0.90 | 0.03 |
| Time from last intensive chemotherapy | 1.79 | 0.95-3.75 | 0.09 |
| to leukapheresis† | | | |
| CD8+ CAR-T cells (peak)§ | 1.42 | 1.03-2.16 | 0.06 |

*OR calculated for 100 U/L increments, a higher OR being associated with a lower probability of achieving CR
†for 1 logₑ days increments
§calculated from absolute lymphocyte counts multiplied by the % of viable CD45+/CD3+/CD8+/CD4−/EGFRt+ events

Further analysis again showed that lower pre-lymphodepletion serum lactate dehydrogenase (LDH; hazard ratio [HR] 0.24 [95% CI, 0.08-0.53] per 100 U/L increment, P=0.003) and also showed that greater increase in serum monocyte chemoattractant protein-1 (MCP-1) concentration from lymphodepletion to the day of CAR-T cell infusion (MCP-14; HR 1.36 [95% CI, 1.12-1.79] per 50 pg/mL increment, P=0.007) were independently associated with development of CR in aggressive NHL. Correlations between pre-lymphodepletion serum LDH, the IPI score and tumor burden (the sum of the product of the perpendicular diameters of up to 6 index lesions [SPD]) suggested that high LDH before lymphodepletion reflected more aggressive disease (data not shown). The data suggest that the probability of CR after CAR-T cell immunotherapy may be better in patients who have less aggressive disease before treatment and higher MCP-1 increase in response to lymphodepletion.

It was considered that bridging chemotherapy to control aggressive disease during CAR-T cell manufacturing might reduce serum MCP-14; however, there was no difference in pre-lymphodepletion or day 0 MCP-1 or MCP-14 in patients who did or did not receive bridging therapy between leukapheresis and lymphodepletion (FIGS. 12F-12G). These data suggest that the probability of CR after CAR-T cell immunotherapy may be better in patients who have less aggressive disease before treatment and higher MCP-14 after lymphodepletion.

3.4. Analysis of Factors Associated with PFS in Aggressive NHL

To further understand the factors associated with PFS in aggressive Non-Hodgkin's Lymphoma, uni- and multivariate analyses were performed on the subcohort of patients with aggressive NHL histology (n=48). Results are summarized in Table 33, below.

TABLE 33
Factors associated with PFS in aggressive NHL

| | Univariate* | | Multivariate† | |
|---|---|---|---|---|
| Variable | HR (95% CI) | P-value | HR (95% CI) | P-value |
| International Prognostic Index | 1.91 (1.30-2.79) | 0.001 | | |
| score prior to lymphodepletion | | | | |
| Serum LDH pre- | 3.70 (1.70-8.06) | 0.001 | | |
| lymphodepletion > ULN‡ | | | | |
| Abnormal B cells in blood | 1.07 (1.02-1.11) | 0.003 | | |
| (cells/μL)§ | | | | |
| Corticosteroid dose 6 weeks | 1.88 (1.20-2.93) | 0.01 | | |
| before apheresis¶ | | | | |
| Corticosteroid requirement after | 3.42 (1.43-8.22) | 0.01 | | |
| apheresis∥ | | | | |
| Tumor cross-sectional area** | 1.01 (1.00-1.02) | 0.01 | | |
| Abnormal B cells in marrow (%)†† | 1.03 (1.01-1.05) | 0.01 | | |
| Serum LDH pre- | 1.24 (1.04-1.47) | 0.02 | 1.37 (1.14-1.63) | 0.0006 |
| lymphodepletion‡‡ | | | | |
| Low intensity Cy/Flu§§ | 1.92 (0.99-3.72) | 0.05 | | |
| MCP-1, day 0 (pre-CAR-T cell | 0.25 (0.10-0.60) | 0.002 | 0.29 (0.09-0.90) | 0.03 |
| infusion)¶¶ | | | | |
| IL-7, peak∥∥ | 0.84 (0.74-0.95) | 0.01 | 0.89 (0.77-1.04) | 0.14 |

HR, Hazard Ratio;
95% CI, 95% Confidence Interval;
*Univariate Cox regression model; pre-treatment clinical characteristics with P-value ≤ 0.05 and variables chosen for the final multivariate model are presented.
†Cox regression model using elastic net was performed to select variables associated with PFS, where log10 values were used to transform data as appropriate, with 0.001 substituting for values of 0.
‡Above upper limit of normal (210 U/L), yes versus no.
§Per 10³/μL increment.
¶Cumulative corticosteroid dose up to 6 weeks before leukapheresis (per log10 mg/m2 prednisone equivalent dose increment).
∥Yes versus no.
**SPD per cm2 increment.
††Per percent increment.
‡‡Per 100 U/L increment.
§§Low intensity Cy/Flu versus high intensity Cy/Flu regimen.
¶¶Per log 10 pg/mL serum concentration increment.
∥∥Per 5 pg/mL serum concentration increment.

In univariate analysis of baseline and treatment characteristics (data not shown), the probability of durable PFS was higher in patients with a lower serum LDH, IPI, SPD, and abnormal B cell burden in the blood and marrow before lymphodepletion, a lower cumulative corticosteroid dose within 6 weeks before leukapheresis, no requirement for corticosteroid therapy after leukapheresis, and use of high-intensity Cy/Flu lymphodepletion. Univariate analysis of biomarkers that might be associated with durable PFS also showed that the concentrations of distinct cytokines and higher in vivo CAR-T cell expansion in the blood measured either by the peak of CD8+ CAR-T cells/μL (HR 0.70 [95% CI, 0.50-0.97] for each log 10 cells/μL increment, P=0.03) or CAR transgene copies/μg of DNA (HR 0.69 [95% CI, 0.47-1.01] for each log 10 copies/μg of DNA increment, P=0.05) were associated with better PFS (data not shown). A summary of significant variables identified by the univariate analysis is shown in (Table 34).

TABLE 34
Variables associated with PFS identified by univariate analysis

| Factors | Variable | Pre-LD | Day 0‡ | Day 28 |
|---|---|---|---|---|
| Disease- | Serum LDH† | HR > 1 | | |
| related | IPI at enrollment† | HR > 1 | | |
| | SPD† | HR > 1 | | |
| | Bulky disease† | HR > 1 | | |
| | Abnormal marrow B cells | HR > 1 | | |
| | Steroids after leukapheresis | HR > 1 | | |
| Biomarkers | IL-15 | | HR < 1 | HR < 1 |
| | MCP-1 | | HR < 1 | HR < 1 |
| | IL-7 | HR < 1 | | HR < 1 |
| | TGFβ-1 | HR < 1 | | HR < 1 |
| | TNF-α | | | HR < 1 |
| | IFN-γ | HR < 1 | | HR < 1 |
| | IL-8 | | | HR < 1 |
| | IL-2 | HR > 1 | HR > 1 | |
| | TNFRp75 | HR > 1 | | HR > 1 |
| | IL-18 | HR > 1 | HR > 1 | |
| CAR-T cell | CD8⁺ CAR-T cells | | | |
| kinetics | Peak (cells/μL) | | | HR < 1 |
| | AUC day 0-28 | | | HR < 1 |
| | CAR-T cells by qPCR | | | |
| | Peak | | | HR < 1 |
| | AUC day 0-28 | | | HR < 1 |

For HR > 1 and HR < 1, P < 0.05
*174 variables were initially evaluated in the univariate analysis; all biomarkers are described in serum concentrations
‡= Pre-CAR-T cell infusion
†= variables with strong inter-correlation that were not considered for variable selection in the elastic net multivariable analysis.

The variables considered for the multivariate models are shown in Table 35.

TABLE 35
Variables associated with PFS identified by multivariate analysis

| Pre-LD Landmark | Day 28 Landmark |
|---|---|
| Age‡ | Serum LDH (U/L, pre-LD) |
| Serum LDH (U/L) | CD4+ CAR-T cell (logₑ |
| | cells/μL, peak)† |
| Abnormal B-cells in marrow (%) | CD8+ CAR-T cell (logₑ |
| | cells/μL, peak) |
| Steroids after leukapheresis (yes/no) | CD8+ CAR-T cell AUC day 0-28 |
| | (logₑ) |
| Bridging treatment after | IL-15 (logₑ pg/mL, peak) |
| leukapheresis (yes/no)‡ | |
| MCP-1 (logₑ, pg/mL)† | MCP-1 (logₑ pg/mL, peak) |
| IL-7 (pg/mL) | IL-7 (pg/mL, peak) |
| TGF-β1 (logₑ pg/mL) | TGF-β1 (logₑ pg/mL, peak) |
| IFN-γ (pg/mL) | TNF-α (pg/mL, peak) |
| IL2 (pg/mL | IFN-γ (pg/mL, peak) |
| TNFRp75 (logₑ pg/mL) | IL-8 (logₑ pg/mL, peak) |
| IL-18 (logₑ pg/mL) | IL-18 (logₑ pg/mL, day 0) |
| | IL-22 (logₑ pg/mL, peak)† |

†= variable with 0.05 < P-value < 0.15 in the univariate analysis
‡= variables considered due to clinical and/or biological relevance

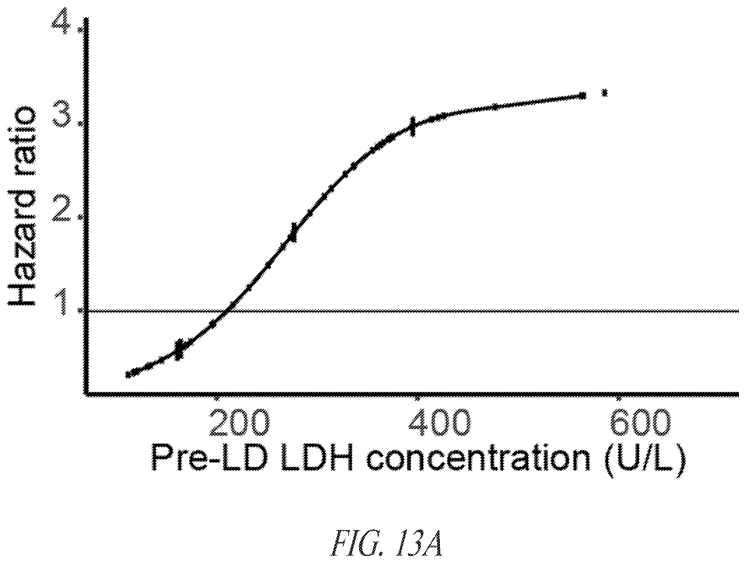

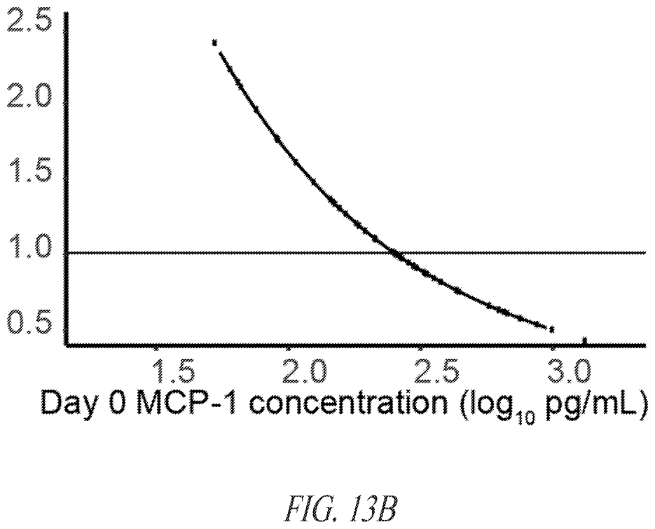

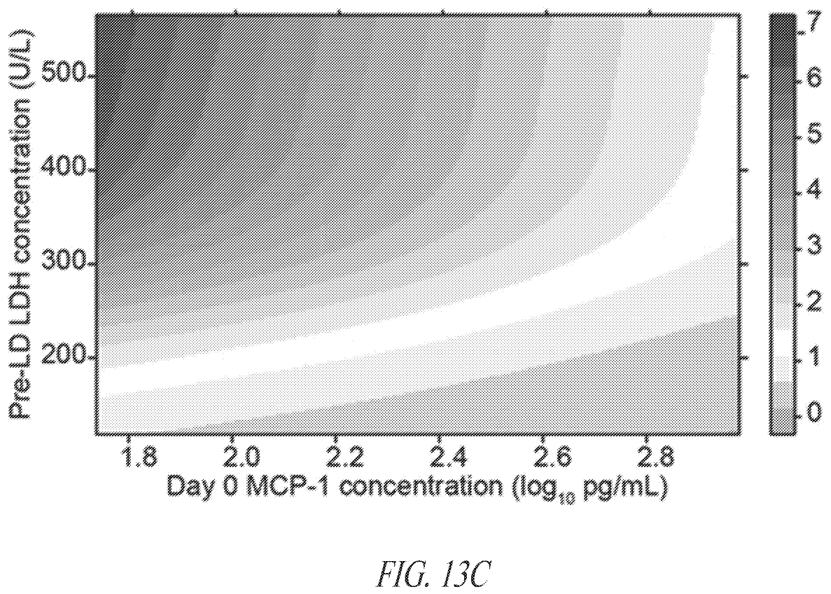

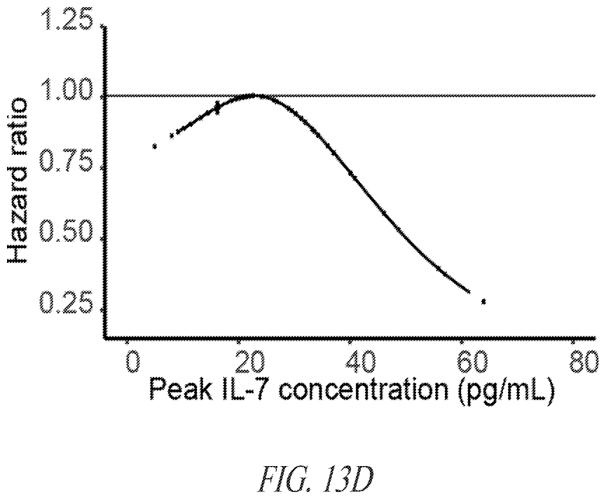

Multivariate Cox regression analysis of clinical factors and biomarkers after elastic net variable selection showed that lower pre-lymphodepletion serum LDH (FIG. 13A), along with higher serum MCP-1 on the day of CAR-T cell infusion (day 0; FIG. 13B-13C), and higher serum IL-7 peak after CAR-T cell infusion (FIG. 13E-13F) were associated with longer PFS (Table 35). The combined effect of high LDH with high MCP-1 (FIG. 13C) or IL-7 (FIG. 13D) resulted in reduced hazard of a PFS event. Similar findings were obtained when the model was adjusted to account for new treatment after CAR-T cell infusion as a time-dependent covariate (Table 36).

TABLE 36
Multivariate model for factors impacting PFS in aggressive
NHL adjusting for new treatment after CAR-T cell
infusion as a time-dependent covariate*

| Variable | Hazard Ratio | 95% CI | P-value |
|---|---|---|---|
| Serum LDH pre-lymphodepletion† | 1.37 | 1.14-1.63 | 0.0007 |
| MCP-1, day 0 (pre-CAR-T cell | 0.29 | 0.09-0.90 | 0.03 |
| infusion)‡ | | | |
| IL-7, peak§ | 0.89 | 0.77-1.04 | 0.14 |
| New treatment¶ | 1.12 | 0.45-2.78 | 0.80 |

*Cox regression was performed to assess the association between PFS and variables of interest where log₁₀ values were used to transform data as appropriate.
†Per 100 U/L increment.
‡Per 50 pg/mL serum concentration increment.
§Per 5 pg/mL serum concentration increment.
¶Second CAR-T cell infusion, new anti-tumor therapy, or hematopoietic stem cell transplantation.

Multivariable models for PFS at the pre-LD landmark, at the Day-28 landmark, and at the Day-28 landmark in CR patients only are shown in Tables 37-39 . For Tables 38 and 39: HR was calculated for 100 U/L increments; † for 1pg/mL increments; § for 1 logₑ cell/uL increments; ‡ for 1 logₑ cell/pg increments; a higher HR being associated with lower probability of PFS.

TABLE 37
Multivariable Model for PFS (pre-LD Landmark, N = 48)

| Variable | HR | 95% Cl | P-value |
|---|---|---|---|
| Serum LDH (pre-LD)* | 1.29 | 1.01-1.65 | 0.05 |
| IL-7 (pre-LD)† | 093 | 0.88-0.99 | 0.01 |
| IL-18 (pre-LD)§ | 2.08 | 1.15-3.76 | 0.01 |

*HR calculated for 100 U/L increments;
†for 1 pg/mL increments
§for 1 logₑ pg/mL increments

TABLE 38
Multivariable Model for PFS (Day 28 Landmark, N = 48)

| Variables | HR | 95% Cl | P |
|---|---|---|---|
| Serum LDH (pre-LD)* | 1.26 | 1.04-1.54 | 0.02 |
| IL-7 (peak)† | 0.96 | 0.93-1.00 | 0.06 |
| CD8+ CAR-T cells (peak)§ | 0.81 | 0.68-0.95 | 0.01 |

TABLE 39
Multivariable Model for PFS in
CR pts (Day 28 Landmark, N = 19)

| Variables | HR | 95% Cl | P |
|---|---|---|---|
| IL-7 (peak)† | 0.93 | 0.86-1.00 | 0.05 |
| IL-18 (day 0)‡ | 7.24 | 1.33-39.24 | 0.02 |

These models did not reflect the multivariate analyses of biomarkers described in FIGS. 13C-13E or Tables 35 and 36.

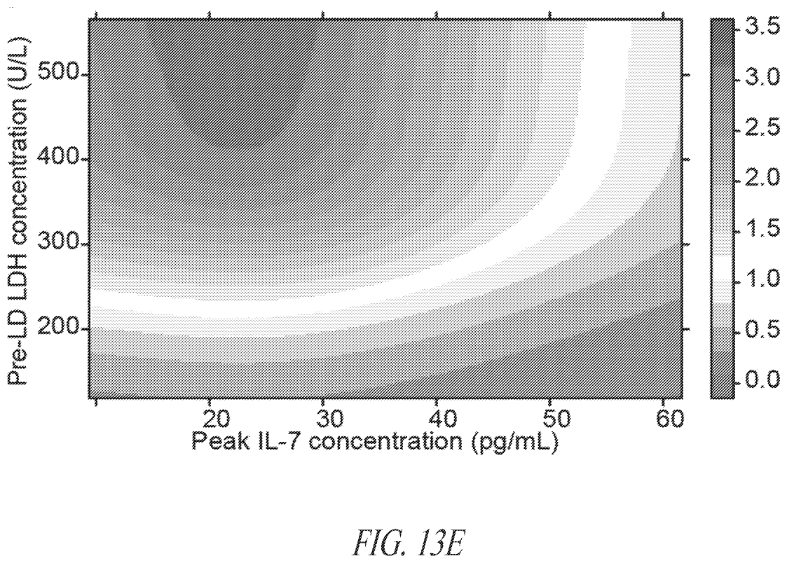

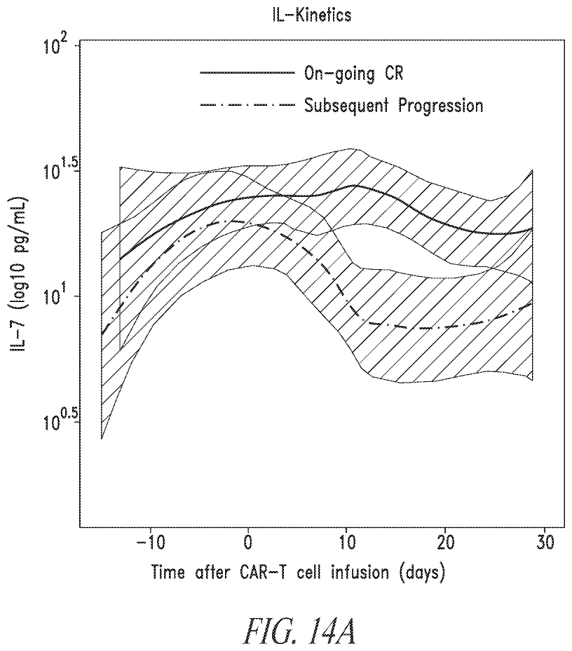

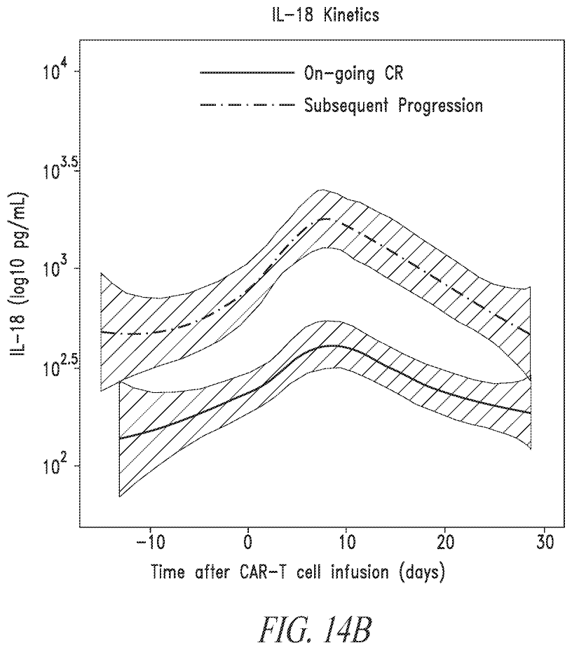

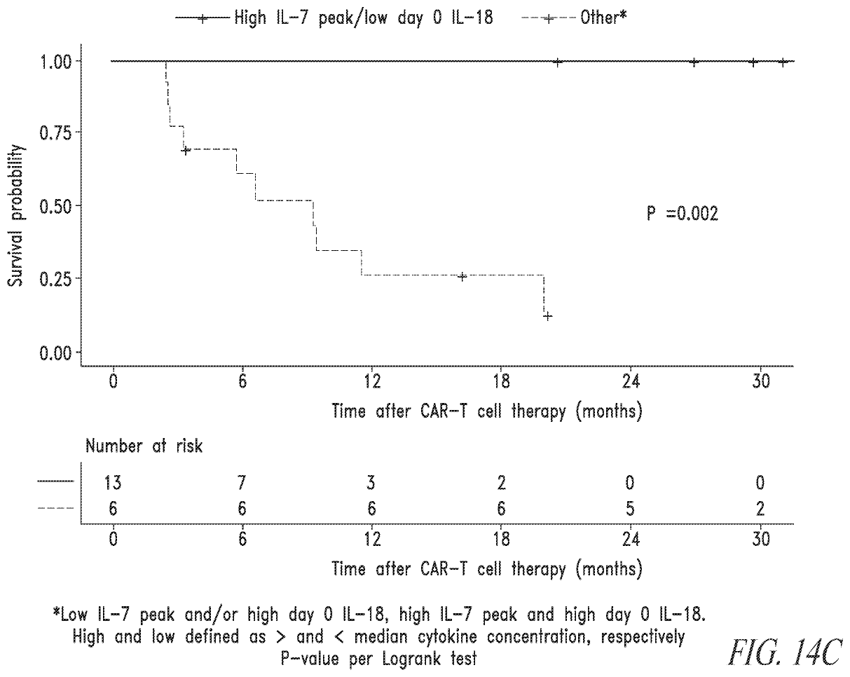

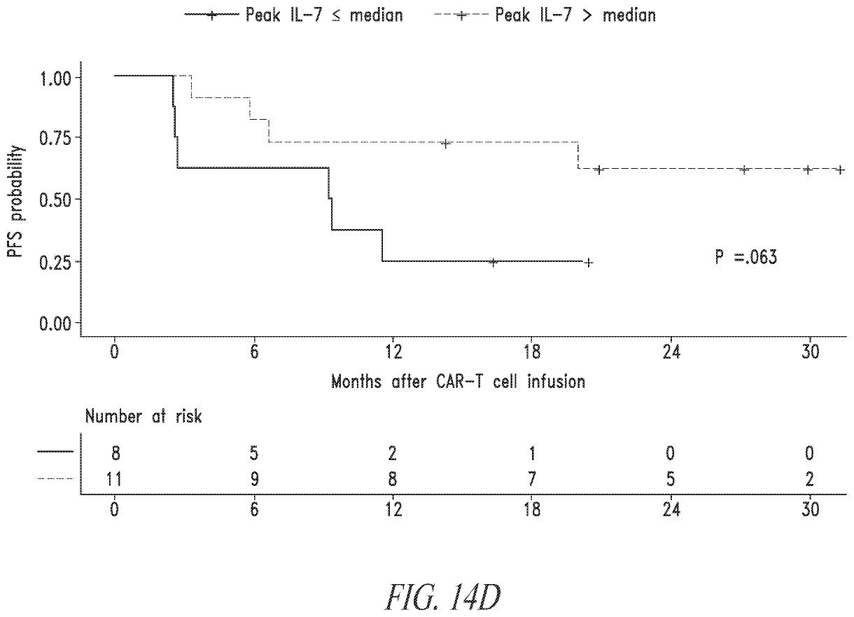

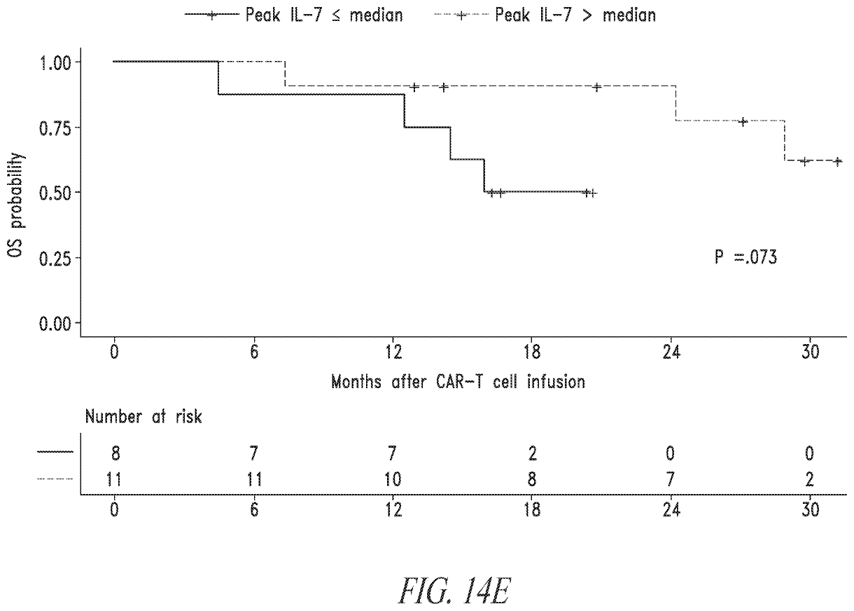

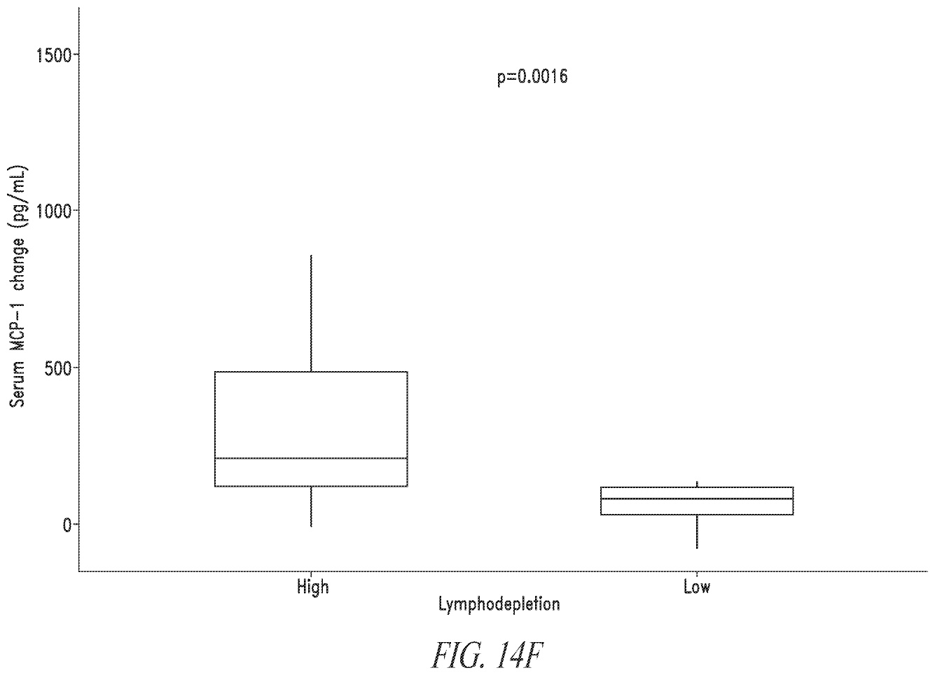

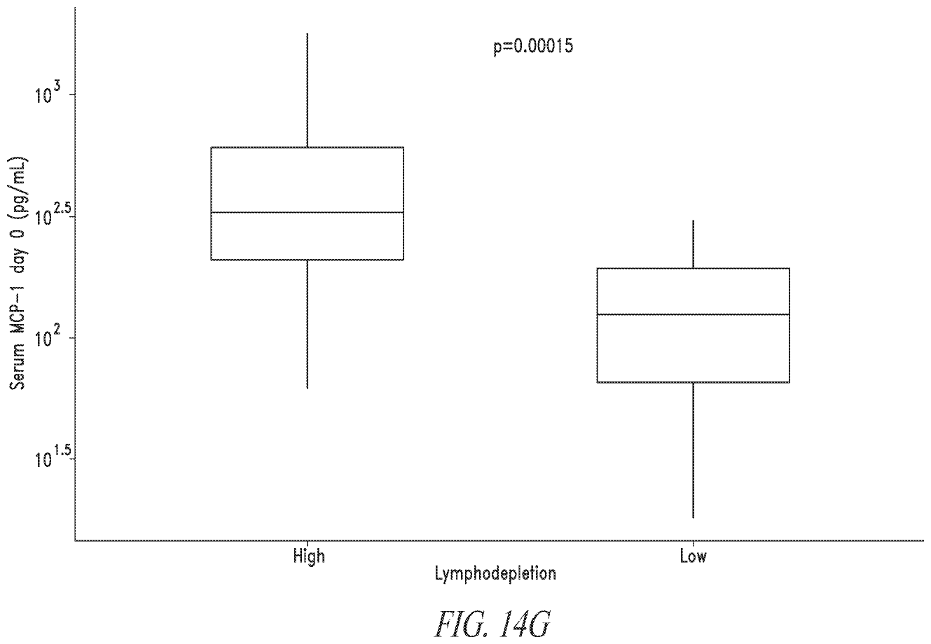

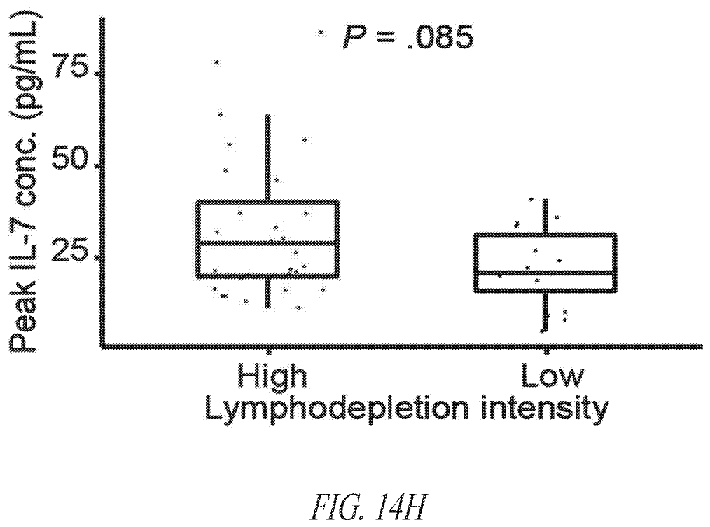

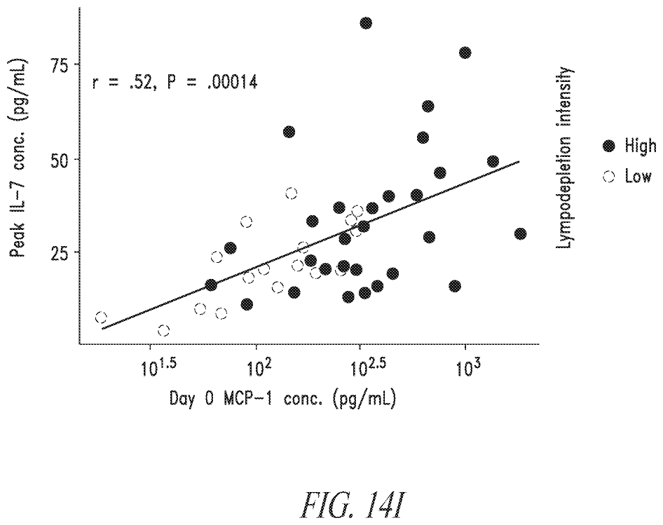

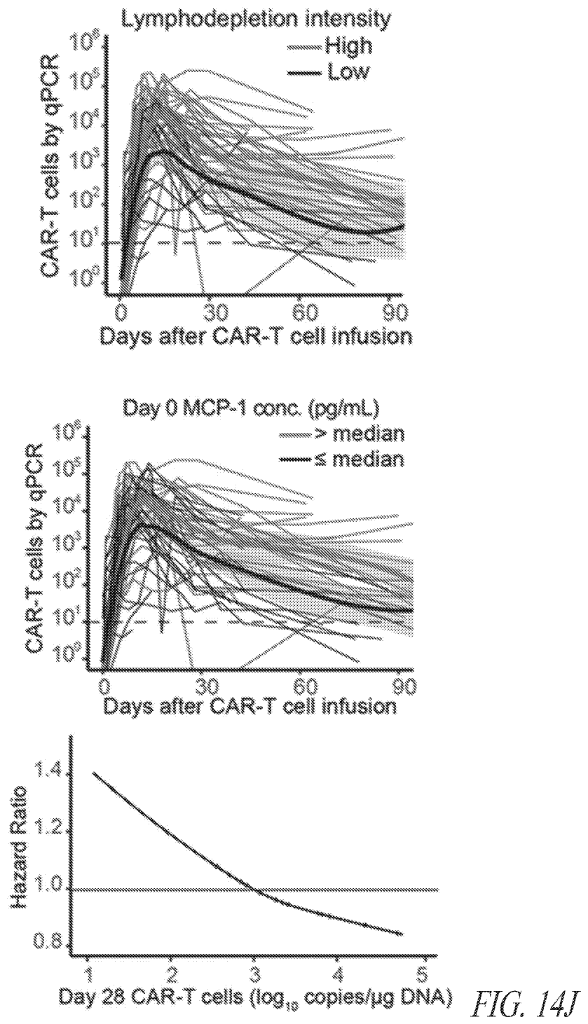

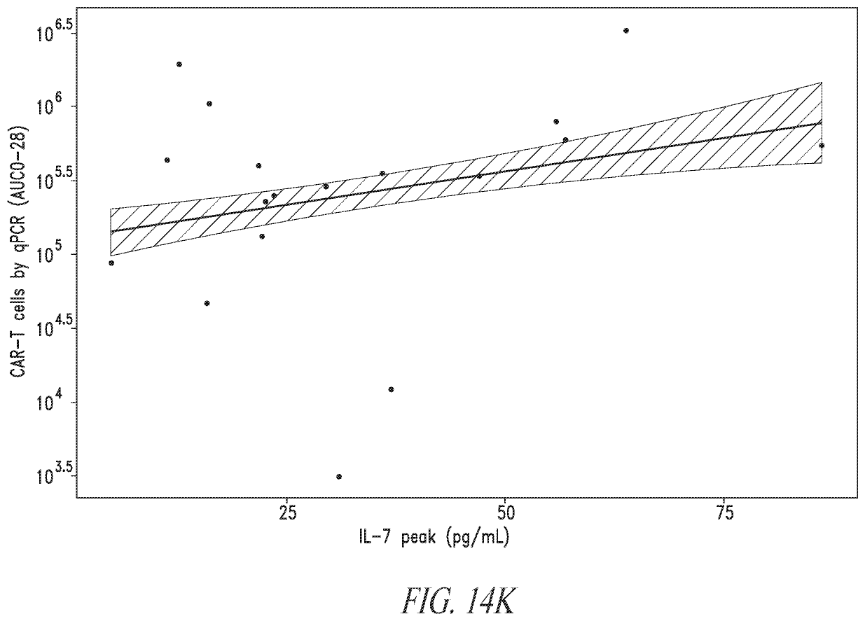

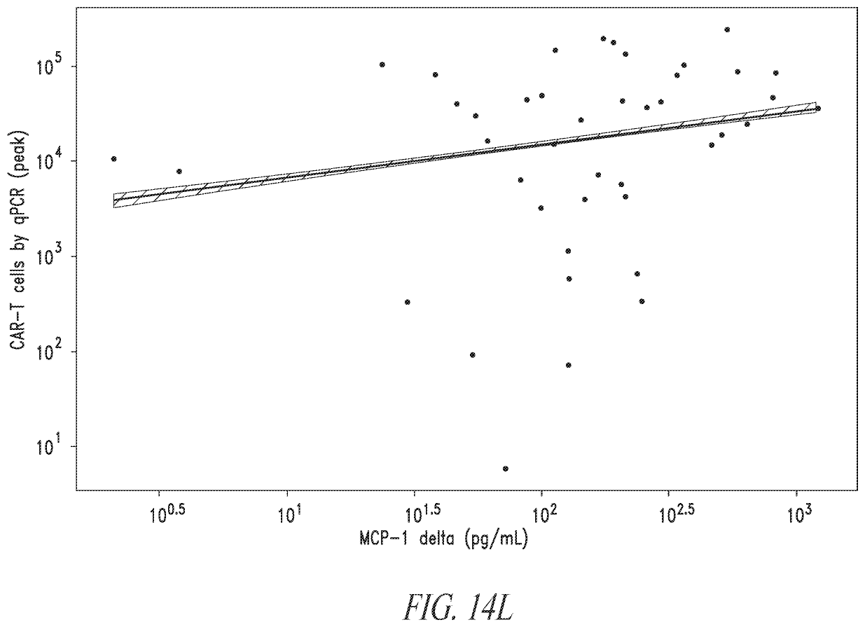

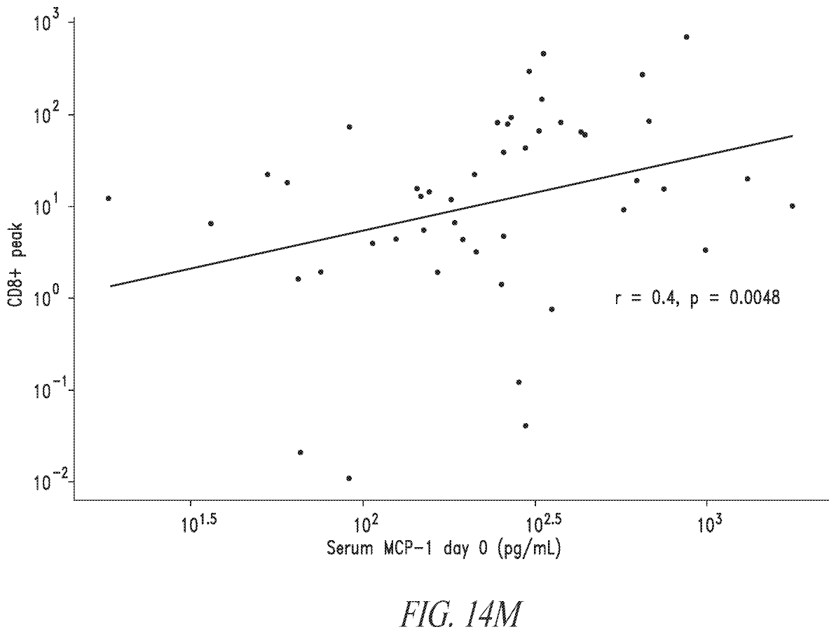

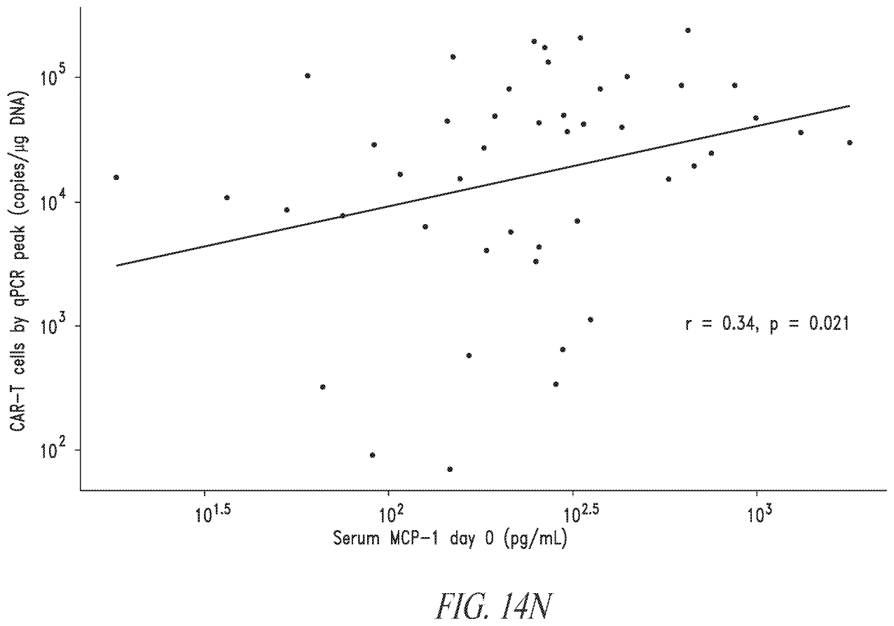

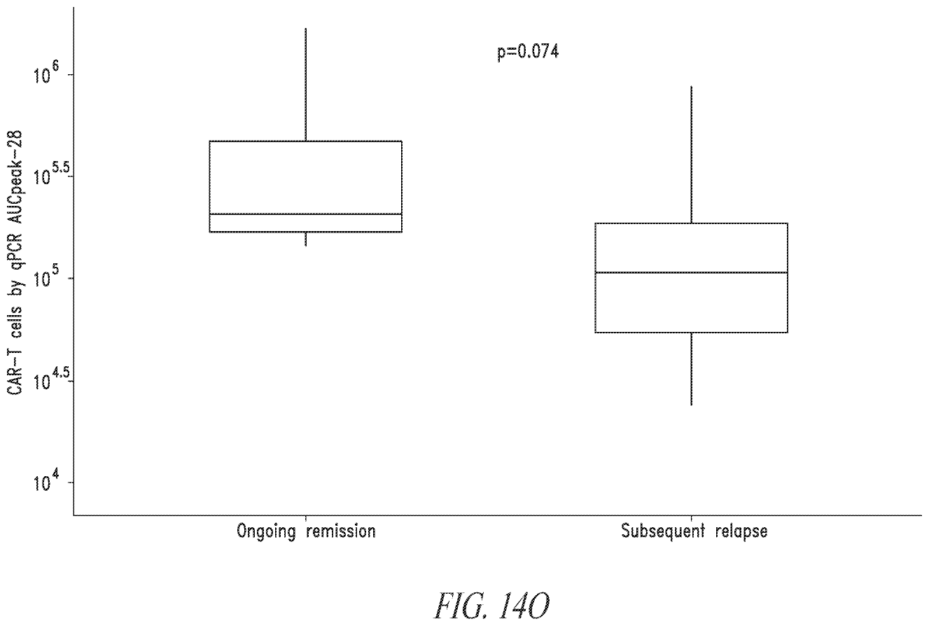

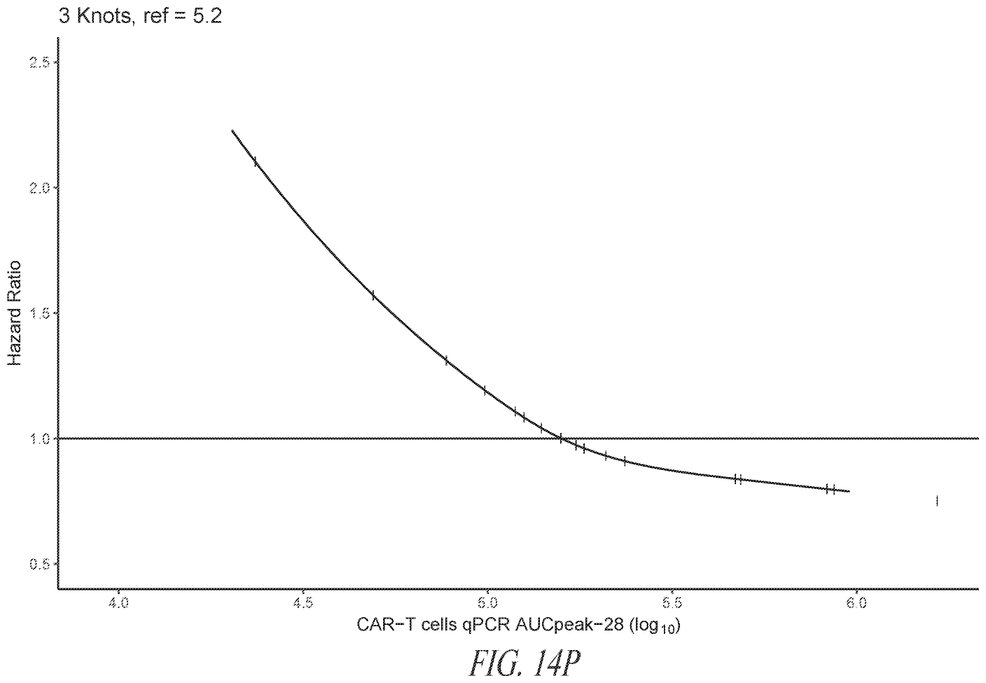

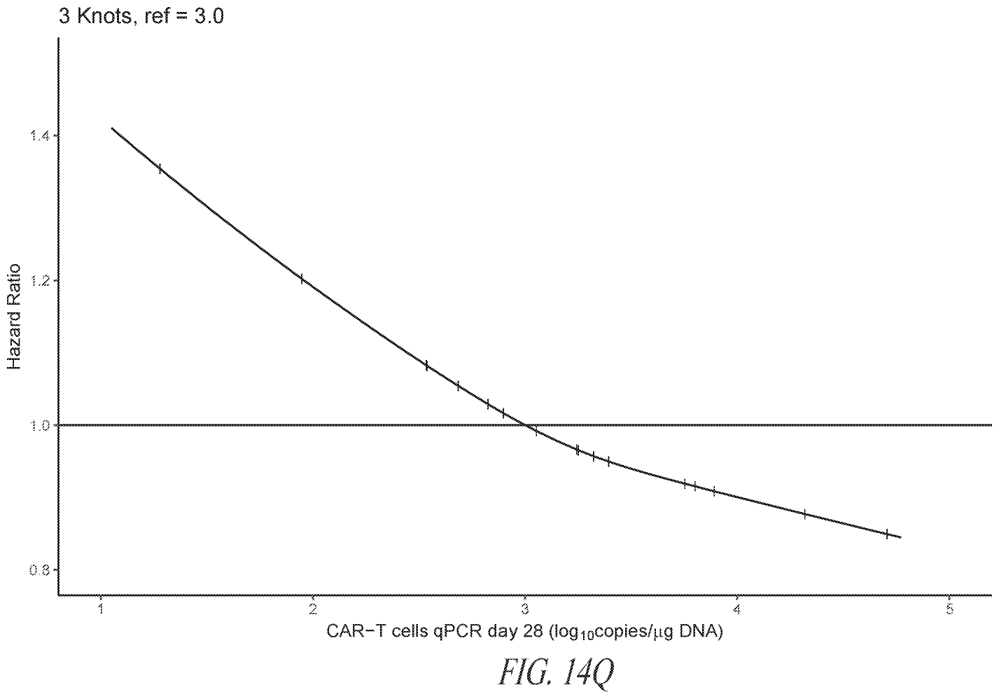

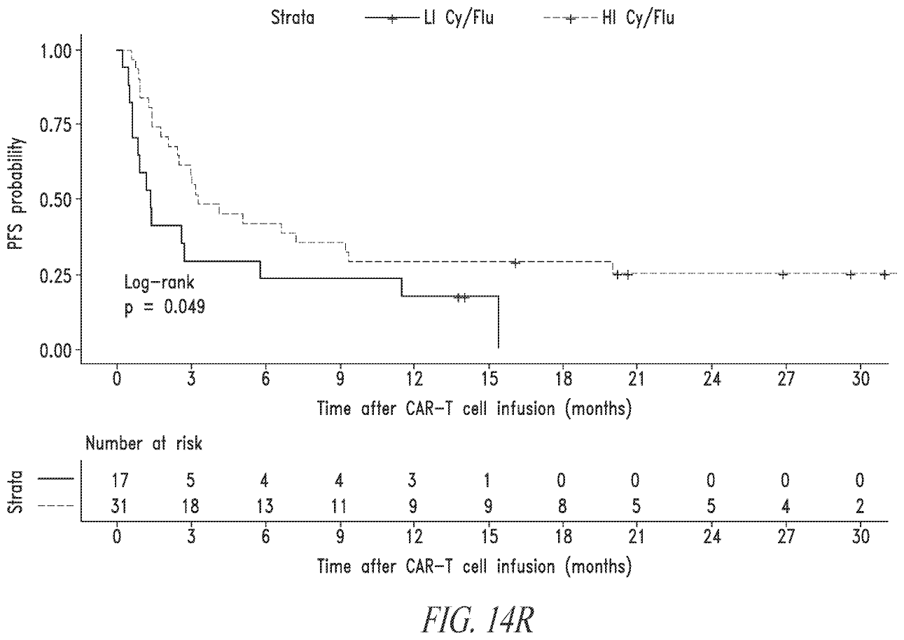

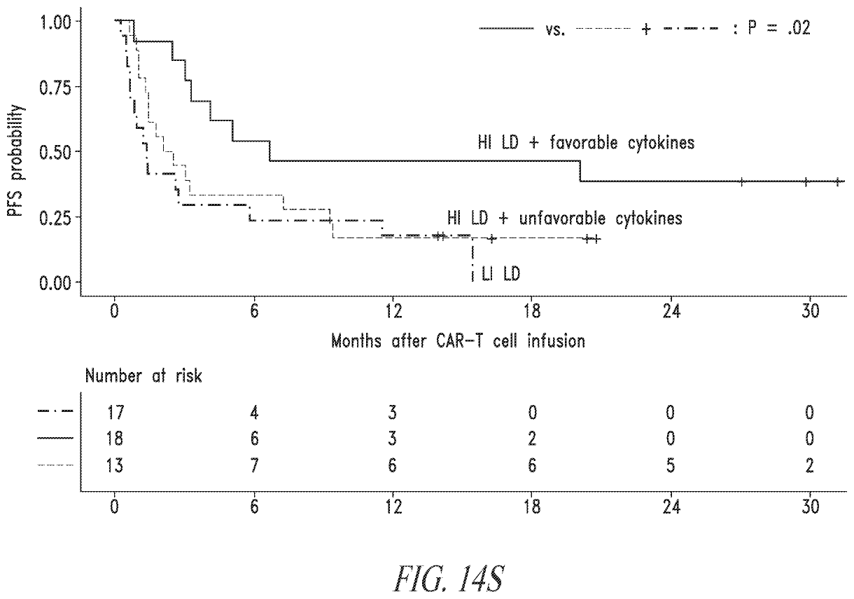

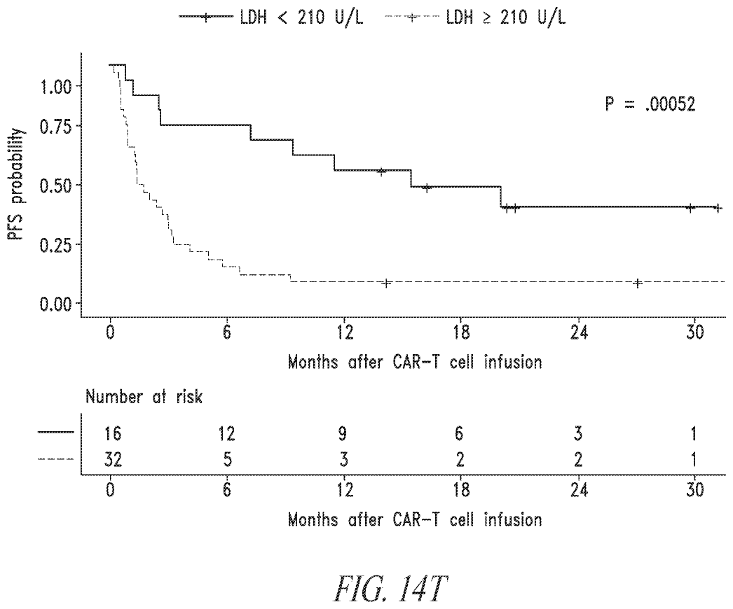

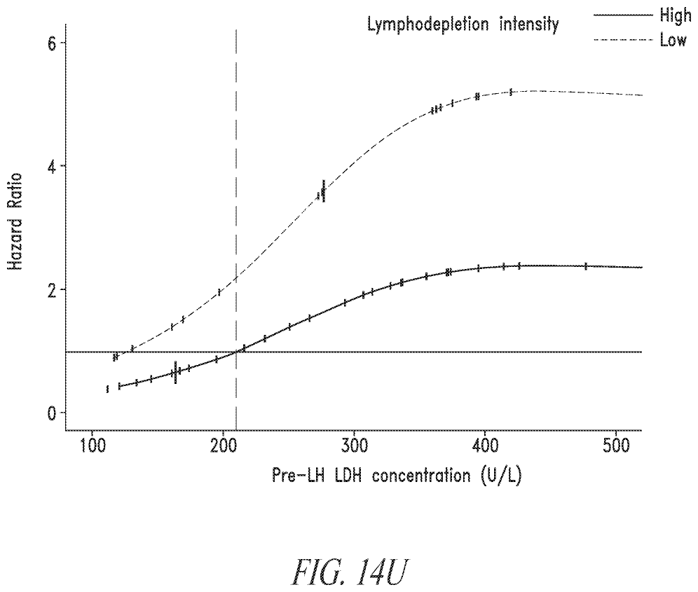

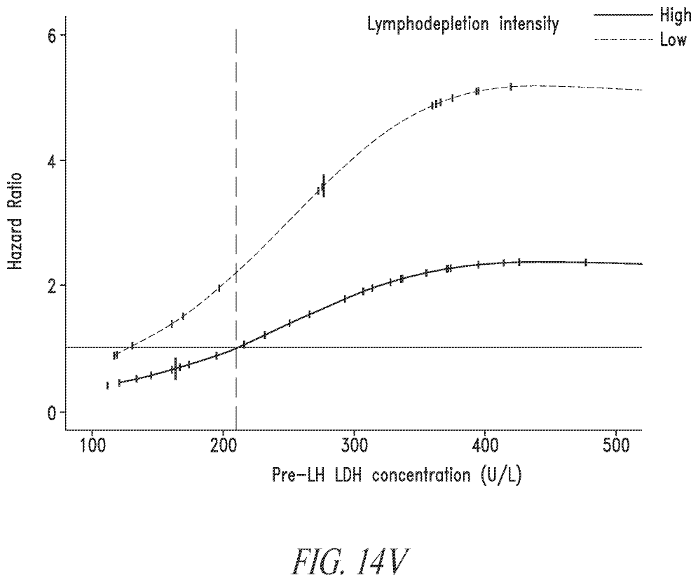

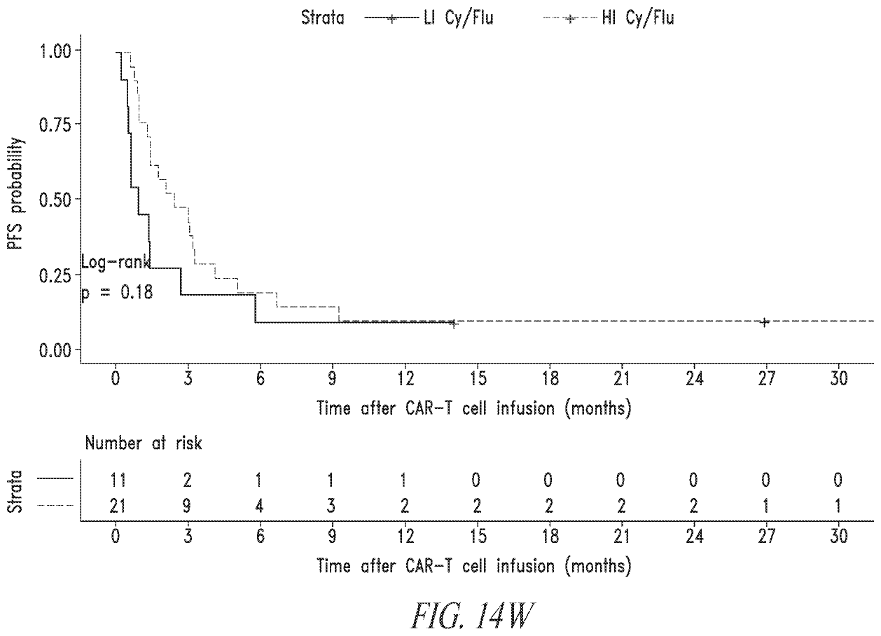

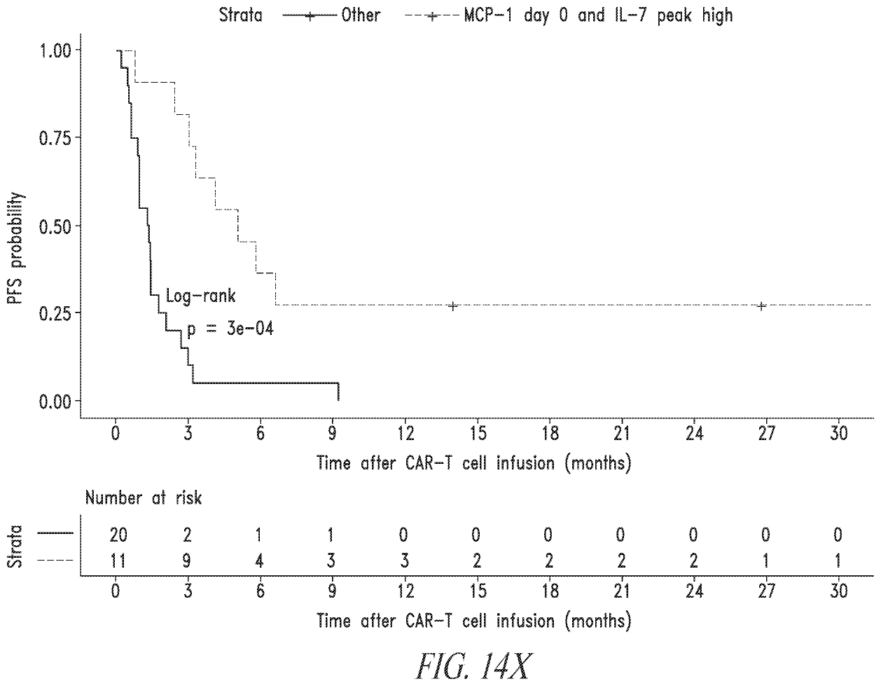

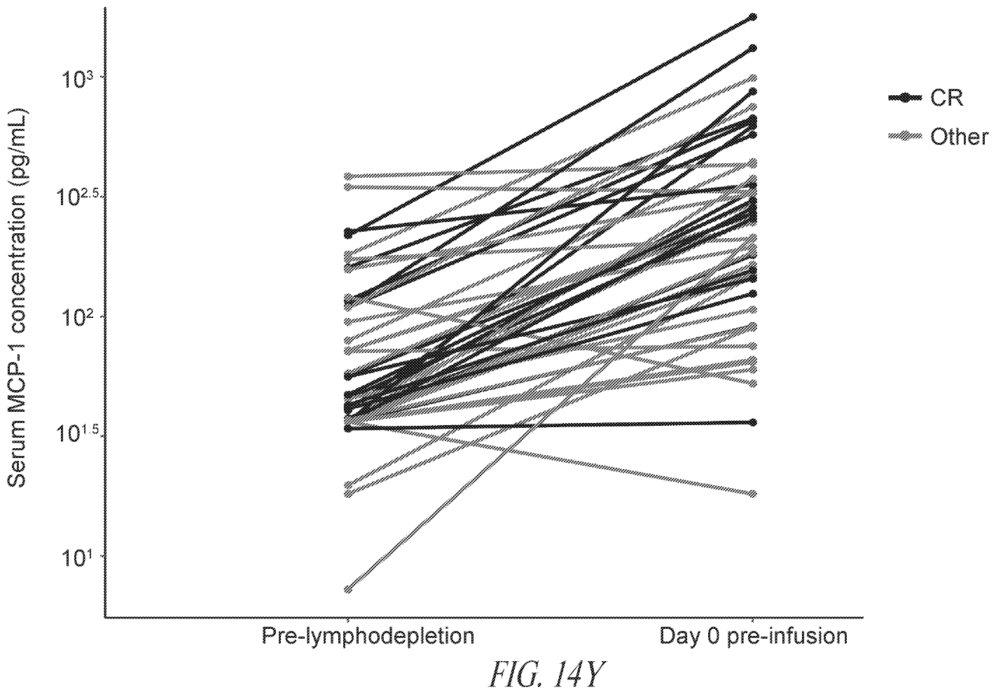

In addition to serum LDH, MCP-1, and peak CD8+ CAR-T expansion in vivo, IL-7 and IL-18 were significantly associated with PFS. The dynamics of these two cytokines following CAR-T cell infusion were studied. Results are shown in FIGS. 14A and 14B. As shown in FIG. 14C, in CR patients with aggressive pre-treatment NHL, a higher peak concentration of IL-7 and a lower day 0 IL-18 concentration increased the probability of PFS (100% at 24-mo.). The factors associated with durable PFS in patients who achieve CR may differ from those identified before the anti-tumor response is known. To examine this, a subgroup analysis of aggressive NHL patients who achieved CR after CAR-T cell immunotherapy (n=19) was performed, testing whether pre-lymphodepletion LDH, day 0 MCP-1, and peak IL-7 impacted PFS. A higher serum IL-7 peak after CAR-T cell infusion was significantly associated with longer PFS in this subgroup (Table 40).

TABLE 40
Multivariate model for factors impacting PFS
in aggressive NHL patients who achieved CR*

| Variable | Hazard Ratio | 95% CI | P-value |
|---|---|---|---|
| Serum LDH pre-lymphodepletion† | 1.26 | 0.67-2.38 | 0.47 |
| MCP-1, day 0 (pre-CAR-T cell | 1.13 | 0.21-6.26 | 0.89 |
| infusion)‡ | | | |
| IL-7, peak§ | 0.70 | 0.49-1.00 | 0.05 |

*Cox regression model was performed to assess the association between PFS and variables of interest as above, where log10 values were used to transform data as appropriate.
†Per 100 U/L increment.
‡Per 50 pg/mL serum concentration increment.
§Per 5 pg/mL serum concentration increment.

Patients who achieved CR and had a serum IL-7 peak after CAR-T cell infusion above the median (n=11) had better PFS than those with serum IL-7 peak below the median (median follow-up of 26.9 months [range, 2.5 to 32.4 months]), with a 24-month probability of PFS of 62% (95% CI, 39 to 100%); 24-mo (FIGS. 14D and 14E; Probability of OS not shown).

3.5. Cytokines Associated with Better PFS in Aggressive NHL are Increased by Lymphodepletion

The cytokines that were identified to be associated with CR and/or better PFS in aggressive NHL might be influenced by bridging or lymphodepletion chemotherapy. Prior to lymphodepletion, serum MCP-1 and IL-7 concentrations were similar between those who received or did not receive systemic bridging therapy to control disease progression between leukapheresis and lymphodepletion (Table 40), suggesting that bridging chemotherapy did not affect pre-lymphodepletion cytokine concentrations. Consistent with the effects of lymphodepletion, serum MCP-1 and IL-7 concentrations were higher on the day of CAR-T cell infusion compared to their respective levels before lymphodepletion To examine this, the MCP-14, day 0 MCP-1, and peak IL-7 concentrations in patients who received high-intensity or low-intensity Cy/Flu lymphodepletion were compared, and patients who received high-intensity Cy/Flu lymphodepleting chemotherapy had greater MCP-14 (FIG. 14F), higher day 0 serum MCP-1 (FIG. 14G), and higher peak serum IL-7 concentrations (FIG. 14H) were observed. The serum IL-7 peak occurred a median of 4.4 days after CAR-T cell infusion and correlated with day 0 IL-7, consistent with an effect of lymphodepletion. Serum MCP-14 and day 0 MCP-1 correlated with peak IL-7 concentrations, and were higher in patients who received high-intensity compared to low-intensity lymphodepletion (FIG. 14I). These data show that more intensive lymphodepletion was associated with changes in serum cytokines that may enhance the in vivo homeostatic environment for transferred CAR-T cells.

3.6. Patients who Received High Intensity Lymphodepletion have Higher CAR-T Cell Counts

Patients who received high-intensity lymphodepletion had better PFS compared to those who were treated with low-intensity chemotherapy (FIG. 14R), as did those with serum day 0 MCP-1 concentration above compared to below the median (day 0 MCP-1, median 253.6 pg/mL; FIG. 14J). Cox regression analyses showed that patients in CR with >1000 copies of the CAR transgene per μg DNA in blood on day 28 after infusion had a higher probability of remaining progression-free (FIGS. 14K-14N).

A correlation between the serum day 0 MCP-1 and peak IL-7 concentrations was identified, but it was established that there was variability in the levels of these cytokines within the subsets of patients who received either high- or low-intensity lymphodepletion (FIG. 14I). While several patients who received high-intensity lymphodepletion had serum day 0 MCP-1 and/or peak IL-7 concentrations above the levels achieved by those who received low-intensity lymphodepletion, a subset of recipients of high-intensity lymphodepletion had day 0 MCP-1 and peak IL-7 concentrations that were no higher than those who received low-intensity lymphodepletion (FIG. 14I). These data show that cytokines identified in multivariate analyses to be associated with CR and/or durable PFS are higher in patients who received high-intensity lymphodepletion, but that not all patients who receive high-intensity lymphodepletion develop a favorable MCP-1 and IL-7 cytokine profile.

No correlation between peak CAR-T cell counts and peak IL-7 concentration was found. It was considered that IL-7 might enhance CAR-T cell survival and contribute to more prolonged CAR-T cell-mediated antitumor activity. Compared with those who subsequently relapsed, patients who remained in CR had a higher CAR-T cell AUCpeak-28 (FIG. 14O). Cox regression analyses showed that patients in CR with higher AUCpeak-28 or with >1000 copies of the CAR transgene per μg DNA in blood on day 28 after infusion had a higher probability of remaining progression-free (FIGS. 14P, 14Q).

3.7. Cytokine Changes After Lymphodepletion are More Closely Associated with CR and PFS than the Intensity of Cy/Flu Lymphodepletion

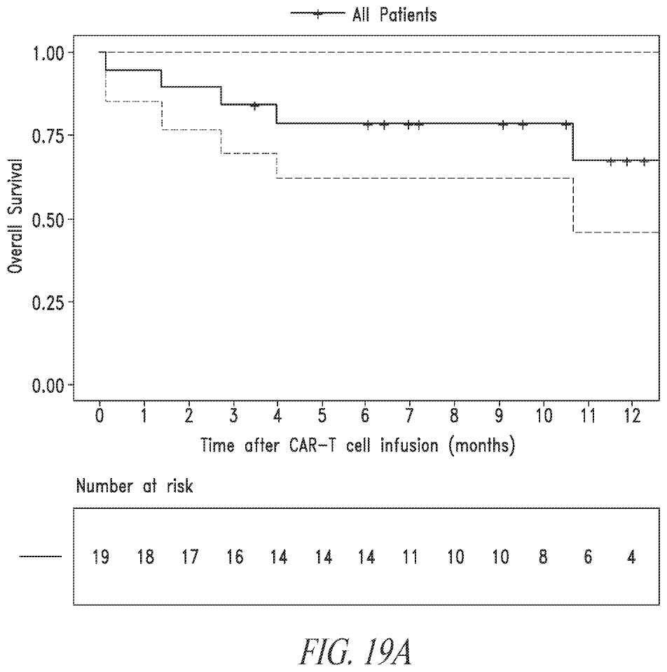

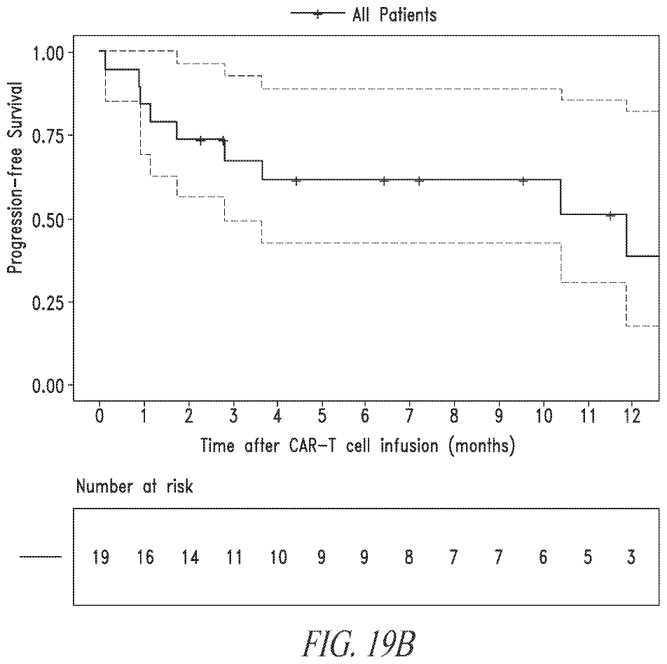

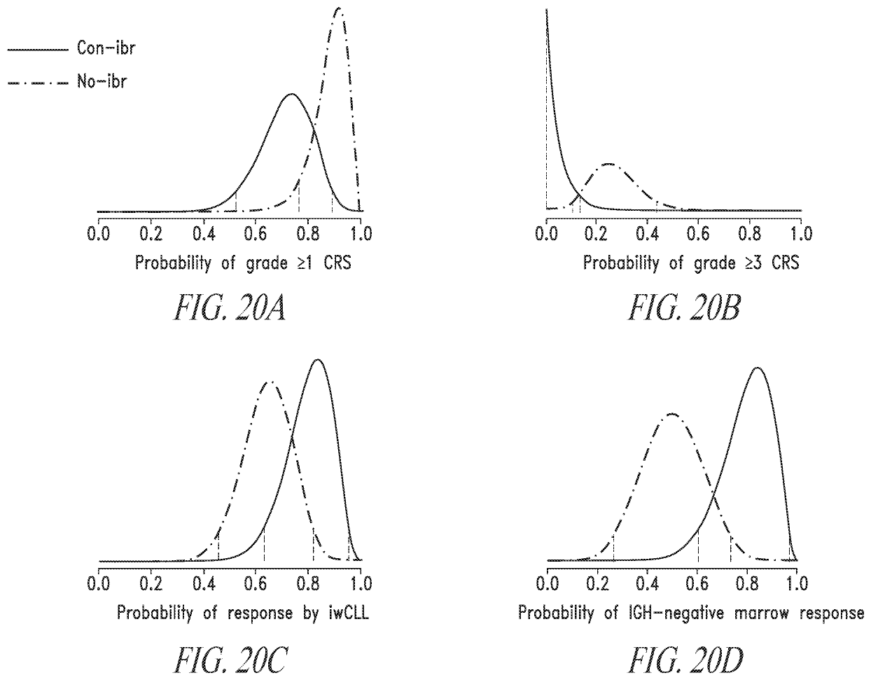

Patients with a favorable cytokine profile, defined as day 0 MCP-1 and peak IL-7 above their respective medians (n=16), had better PFS than those with one or both cytokines below the median. The multivariate model showed that, unlike a favorable cytokine profile, lymphodepletion intensity was not independently associated with PFS. It was observed that a subset of patients who received high-intensity lymphodepletion failed to achieve a favorable cytokine profile, suggesting that the biological outcome of lymphodepletion, evaluated by the concentrations of MCP-1 and IL-7, could be more closely associated with disease response and PFS than the delivery of a high- or low-intensity lymphodepletion regimen. Indeed, patients who received high-intensity lymphodepletion and achieved serum day 0 MCP-1 and peak IL-7 concentrations above the median had a higher probability of CR compared to those who received low-intensity lymphodepletion or high-intensity lymphodepletion without a favorable cytokine profile (odds ratio 3.35 [95% CI, 0.91-13.5], P=0.07). Patients who had a favorable cytokine profile after receiving high-intensity lymphodepletion also had more durable PFS than those without a favorable cytokine profile or after treatment with low-intensity lymphodepletion (FIG. 20S), suggesting that the benefit of high-intensity lymphodepletion may be lost in patients who do not achieve a favorable cytokine profile.

Because multivariate studies showed that, in addition to low serum MCP-1 and IL-7 concentrations, high pre-lymphodepletion serum LDH was associated with failure to achieve CR and/or short PFS, the impact on PFS of a favorable or unfavorable cytokine profile in patients with different pre-lymphodepletion LDH concentrations was investigated. High pre-lymphodepletion LDH was associated with short PFS (FIG. 14T). However, a favorable cytokine profile decreased the risk of a PFS event, particularly in high-risk patients with pre-lymphodepletion serum LDH concentrations above the normal range (LDH >210 U/L) [FIG. 14U]. In contrast, the effect of utilization of a high-compared to low-intensity lymphodepletion regimen was less clear (FIG. 14V). These findings suggest that higher serum day 0 MCP-1 and peak IL-7, reflecting a more favorable cytokine profile achieved by some patients after high-intensity lymphodepletion, is associated with better disease response and PFS after treatment of aggressive NHL patients with CD19 CAR-T cells.

### Example 4

### Clinical Study of Therapies for CLL

### 4.1 Background: Anti-CD19 CAR-T Cell Therapy for High-Risk CLL

Prior studies have described CAR-T cells obtained from patients with prior ibrutinib therapy for CLL (Fraietta et al., Blood 127(9):1117-1127 (2016)), as well as co-administration of ibrutinib with high doses of CAR-T cells (e.g., 5-10 million cells; above the expected therapeutic threshold) in mouse xenograft tumor models (Fraietta et al.; Ruella et al., Leukemia 234 (2017)). However, the effect of ibrutinib on various aspects of CD19 CAR-T cell therapy for CLL in humans remain unknown, and animal models have exhibited limitations in the context of CAR-T therapy (see Siegler and Wang, Hum. Gene Ther. 29(5):534-546 (2018)).

Nineteen (19) additional CLL patients were enrolled in the single-center clinical study described in Example 1 and a total of 47 CLL patients received treatment. Twenty-four (24) of the patients had relapsed or refractory disease following treatment with the Bruton's Tyrosine Kinase (BTK) inhibitor ibrutinib and were categorized as “high-risk.” Briefly, patients with disease that progresses on ibrutinib have poor clinical outcomes (see, e.g., Jain et al., (2016), reporting 25 deaths out of 33 total patients studied and median overall survival of 3.1 months after progression).

Characteristics of the high-risk (HR)-CLL patients were assessed prior to CAR-T cell therapy and included: whether the patient had received prior ibrutinib; whether disease was ibrutinib-refractory, was characterized by mutations in BTK or the BTK-interacting enzyme PLCG2 that are believed to confer resistance to ibrutinib resistance; whether the patient was ibrutinib-intolerant; whether disease was venetoclax-refractory; whether the patient had a complex metaphase karyotype; and whether the patient had a deletion on the short arm of chromosome 17 (del17p13.1). Data are shown in Table 41.

TABLE 41
Patient Characteristics: High-Risk CLL Population

| Characteristic | N = 24 | |
|---|---|---|
| Age at infusion, median [range], years | 61 | [40-73] |
| Prior lines of therapy, median [range] | 5 | (3-9) |
| Prior allogeneic HCT | 4 | (17%) |
| Prior Ibrutinib | 24 | (100%) |
| Ibrutinib-refractory | 19 | (79%) |
| BTK or PLCG2 mutation | 9/19 | (47%) |
| Ibrutinib-intolerant | 3 | (13%) |
| Venetoclax-refractory | 6 | (25%) |
| High-risk cytogenetics, N (%) | 23 | (96%) |
| Complex karyotype | 16 | (67%) |
| 17p del | 14 | (58%) |
| High-risk histology (Richter's/IPC/PLL), N (%) | 8 | (33%) |
| Extramedullary disease, N (%) | 23 | (96%) |
| Cross-sectional area, median [range], mm2 | 3093 | [546-20406] |
| FDG-avid disease on PET, N (%) | 14/15 | (93%) |
| SUVMAX, median [range] | 7.1 | [3.4-27.5] |
| Marrow abnormal B cells, median [range], % | 64.5 | [0-96] |

Pre-therapy absolute abnormal B cell count in blood:
Median 1.1 (×10³/mL)
Range 0-76.68 (×10³/mL)
CD19 CAR-T cell product was manufactured in 100% of patients
22/24 (92%) products were formulated in the defined CD4+:CD8+ composition

Patients treated previously on the same phase I/II trial without concurrent administration of ibrutinib received a 1:1 ratio of CD4+:CD8+ CD19 CAR-T cells at 2×10⁵ (dose level [DL] 1), 2×10⁶ (DL2), or 2×10⁷ (DL3) cells/kg. Three (3) patients who received a lymphodepletion regimen other than CyFlu, and one patient who received DL3 (DL3 was considered above the maximum tolerated dose), were excluded. A total of 24 patients, all of whom had discontinued ibrutinib prior to leukapheresis or lymphodepletion (No-ibr cohort), were considered for comparison with the Con-ibr cohort. All patients in the No-ibr received CyFlu lymphodepletion. Five patients (21%) received DL1, while 19 (79%) received DL2.

Comparisons of continuous variables between two categories were made using the exact Wilcoxon test and of categorical variables between two categories using Fisher's exact test.

A Bayesian beta-binomial model was used to compare posterior probability distributions for response and toxicity in each cohort (Porter et al., Sci Transl. Med. 7:303ra139 (2015); Lampson et al., Blood 129:2581 (2017)). A uniform non-informative prior distribution was used. The posterior probability distributions were computed using three simultaneous Markov chains with 100,000 iterations each. Multivariable logistic regression was performed to assess predictors for the occurrence of grade ≥1 CRS, grade ≥2 CRS, or IGH-negative marrow response by adjusting for the Ibrutinib cohort variable and another baseline clinical factor, as described. For time-to-event analyses, the Kaplan-Meier method was used to estimate survival distributions, and the reverse Kaplan-Meier method was used to estimate median follow-up time; log-rank tests were used to compare between-group differences in survival curves. All statistical analyses were performed using RStudio software (version 1.1.456, RStudio, Boston, Mass.) and the following packages: ggplot2, dplyr, tidyr, rjags, BayesianFirstAid, rms. ADD in references package list.

Data are summarized in Turtle et al., Abstract #56, American Society for Hematology (ASH) Annual Meeting & Exposition (2016) and in Turtle et al., J. Clin. Oncol. 35:26 (2017), the treatment schemes and related data of which are incorporated herein by reference in their entireties. At four weeks after infusion, patients who had received Cy/Flu lymphodepletion exhibited high overall (73% in all patients and in inbrutinib-refractory patients) and complete (64% in all patients and in inbrutinib-refractory patients) response rates, with one additional patient achieving PR and CR eight weeks later without additional therapy. HR-CLL patients who had a lymph node response (CT scan at 4 weeks; IWCLL nodal response criteria) to Cy/Flu and CAR-T cell therapy had longer PFS and OS compared to non-responders. In total, 44% (7/16) ibrutinib-refractory patients achieved PR by IWCLL criteria. Moreover, IgH sequencing of bone marrow was determined to be potentially useful as an identifier of patients with decreased risk of disease progression after CAR-T cell therapy. Higher CAR-T cell counts in blood following infusion were associated with better BM response and reduced hazard of progression or death in HR-CLL patients. CAR-T cell expansion also correlated with tumor burden of bone marrow and lymph nodes. In general, HR-CLL patients experienced few severe cytokine release syndrome (CRS) (grade 0, n=4; grade 1, n=8; grade 2, n=10, grade 3, n=0; grade 4, n=1; grade 5, n=1 (grading described in Lee et al., Blood (2014)) or neurotoxicity-related (grade 0, n=16; grade 1, n=0; grade 2, n=2, grade 3, n=5; grade 4, n=0; grade 5, n=1 (grading per CTCAE v4.03)) events following CAR-T cell therapy. Thus, CD19-specific CAR-T cells can be used to effectively reduce tumor burden in HR-CLL patients, can promote durable responses (PFS and OS), and have acceptable early toxicity profiles.

### 4.2 Combined Effects of Ibrutinib and CAR-T Cell Therapy

### 4.2.1 Patient Cohorts

HR-CLL patients who had received ibrutinib prior to CAR-T cell infusion were divided into two cohorts, one of which continued to receive ibrutinib up to approximately 3 months after CAR-T cell infusion; and the other of which did not receive ibrutinib after CAR-T cell infusion.

Nineteen patients (median age: 65 years) received ibrutinib concurrently with lymphodepletion and at least one CD19 CAR-T cell infusion (Table 42).

TABLE 42
Characteristics of patients receiving CD19 CAR-T
cell therapy with concurrent ibrutinib

| Number of patients - n | 19 | |
|---|---|---|
| Age (years) | | |
| Median [IQR] | 65 | [56-69] |
| Range | 40-71 | |
| Sex - n (%) | | |
| Female | 7 | (37) |
| Male | 12 | (63) |
| ECOG - n (%) | | |
| 0 | 10 | (53) |
| 1 | 9 | (47) |
| Prior history of Richter's | 4 | (21) |
| transformation - n (%) | | |
| High risk cytogenetics* - n (%) | 17 | (89) |
| 17p deletion - n (%) | 14 | (74) |
| Complex karyotype - n (%) | 14 | (74) |
| Cross-sectional tumor area† (mm²) | | |
| Median [IQR]) | 2,624 | [1,458-4,149] |
| Range | 100-9,097 | |
| Maximum SUV | | |
| Median [IQR]) | 4.4 | [3.4-7.0] |
| Range | 0.0-24.0 | |
| Serum LDH concentration (UI/L) | | |
| Median [IQR]) | 155 | [135-206] |
| Range | 90-387 | |
| Absolute lymphocyte count (10⁹ cells/L) | | |
| Median [IQR]) | 1.12 | [0.84-3.95] |
| Range | 0.20-59.00 | |
| Blood CLL cell count (10⁹ cells/L) | | |
| Median [IQR]) | 0.45 | [0.13-3.13] |
| Range | 0.01-54.00 | |
| Marrow CLL burden (%) | | |
| Median [IQR]) | 26 | [12-60] |
| Range | 2-90 | |
| Prior therapies (number) | | |
| Median [IQR]) | 5 | [4-7] |
| Range | 1-10 | |
| Prior allogeneic stem cell | 3 | (16) |
| transplantation - n (%) | | |
| Prior intolerance to ibrutinib - n (%) | 2 | (10) |
| Duration of last treatment with | | |
| ibrutinib§ (days) | | |
| Median [IQR]) | 248 | [26-764] |
| Range | 14-2185 | |
| Prior idelalisib treatment - n (%) | 5 | (26) |
| Prior venetoclax treatment - n (%) | 11 | (58) |

*Defined as 17p deletion and/or complex karyotype.
†In patients with evaluable nodal disease (n = 17); sum of the product of the diameters of up to six of the largest lymph nodes or nodal masses evaluated on the pre-lymphodepletion CT scan
§Duration of the last continuous ibrutinib therapy before leukapheresis
All variables were assessed prior to lymphodepletion chemotherapy, unless specified.
Abbreviations:
IQR, interquartile range,
ECOG, eastern cooperative oncology group;
SUV, standardized uptake value,
LDH, lactate dehydrogenase;
CLL, chronic lymphocytic leukemia,
CAR-T cell, chimeric antigen receptor-engineered T cells

TABLE 43
Comparison of baseline characteristics between the Con-ibr and the No-ibr cohort

| | Con-ibr cohort | | No-ibr cohort | | |
|---|---|---|---|---|---|
| | n = 19 | | n = 24 | | p |
| Age (years - median [IQR]) | 65 | [56-69] | 61 | [53-64] | 0.24 |
| Female gender (n - (%)) | 7 | (37) | 9 | (37) | 1 |
| ECOG performance status 1 (n - (%)) | 9 | (47) | 11 | (46) | 1 |
| Richter's Transformation (n - (%)) | 4 | (21) | 4 | (17) | 1 |
| 17p deletion (n - (%)) | 14 | (74) | 17 | (71) | 1 |
| 11q abnormality (n - (%)) | 5 | (26) | 10 | (43) | 0.34 |
| Complex karyotype (n - (%)) | 14 | (74) | 18 | (78) | 1 |
| Cross-sectional tumor area* | 2,624 | [1,458-4,149] | 3,225 | [1,959-4,887] | 0.36 |
| (mm² - median [IQR]) | | | | | |
| Maximum SUV (median [IQR]) | 4.4 | [3.4-7.0] | 5.1 | [4.8-9.6] | 0.23 |
| Serum LDH concentration | 155 | [135-206] | 234 | [189-322] | 0.01 |
| (UI/L - median [IQR]) | | | | | |
| Blood absolute lymphocyte | 1.12 | [0.84-3.95] | 2.98 | [1.00-11.65] | 0.19 |
| count (10⁹ cells/L - median [IQR]) | | | | | |
| Blood CLL cell count (10⁹ | 0.45 | [0.13-3.13] | 2.13 | [0.18-7.29] | 0.41 |
| cells/L - median [IQR]) | | | | | |
| Marrow CLL burden (% - | 26 | [12-60] | 59 | [32-78] | 0.09 |
| median [IQR]) | | | | | |
| Prior therapies (number - | 5 | [4-7] | 5 | [4-6] | 0.39 |
| median [IQR]) | | | | | |
| Prior stem cell transplantation | 3 | (16) | 3 | (12) | 1 |
| (n - (%)) | | | | | |
| Prior treatment with venetoclax | 11 | (58) | 6 | (25) | 0.06 |
| (n - (%)) | | | | | |
| Duration of last treatment with | 248 | [26-764] | 384 | [120-642] | 0.50 |
| ibrutinib prior to leukapheresis | | | | | |
| (days - median [IQR]) | | | | | |
| CAR-T cell dose (n - (%)) | | | | | |
| 2 × 10⁵ CAR-T cells/kg | 0 | | 5 | (21) | 0.06 |
| 2 × 10⁶ CAR-T cells/kg | 19 | (100) | 19 | (79) | |

*In patients with evaluable nodal disease

All variables were assessed prior to lymphodepletion chemotherapy, unless specified. P values per Wilcoxon Sum Rank test (one-sided) or Fisher's test, as appropriate. Abbreviations: Con-ibr, concurrent ibrutinib; No-ibr, ibrutinib discontinued prior to lymphodepletion; IQR, interquartile range, ECOG, eastern cooperative oncology group; SUV, standardized uptake value, LDH, lactate dehydrogenase; CLL, chronic lymphocytic leukemia, CAR-T cell, chimeric antigen receptor-engineered T cells

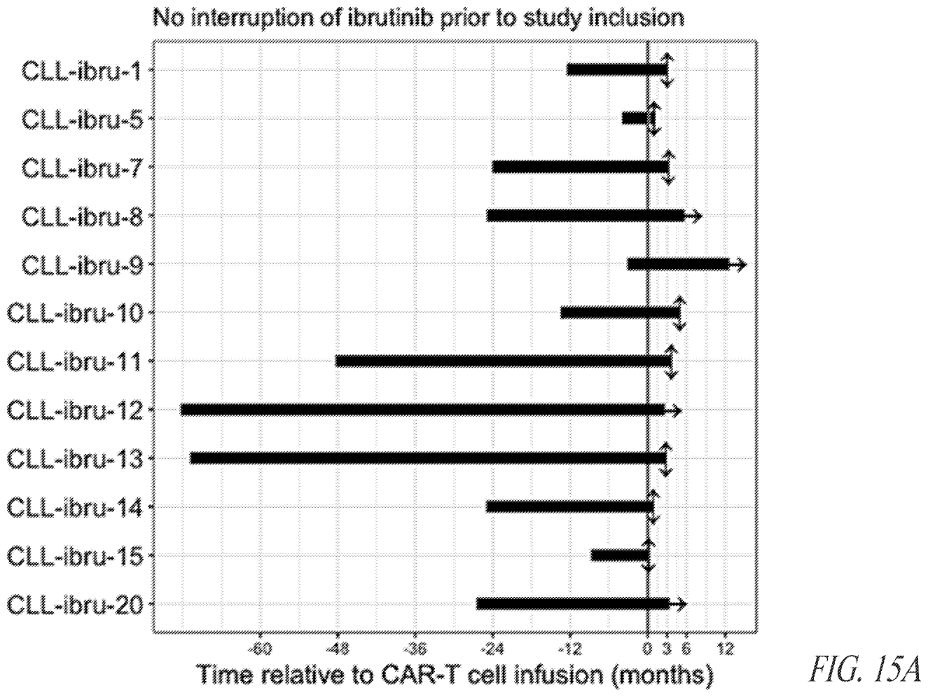

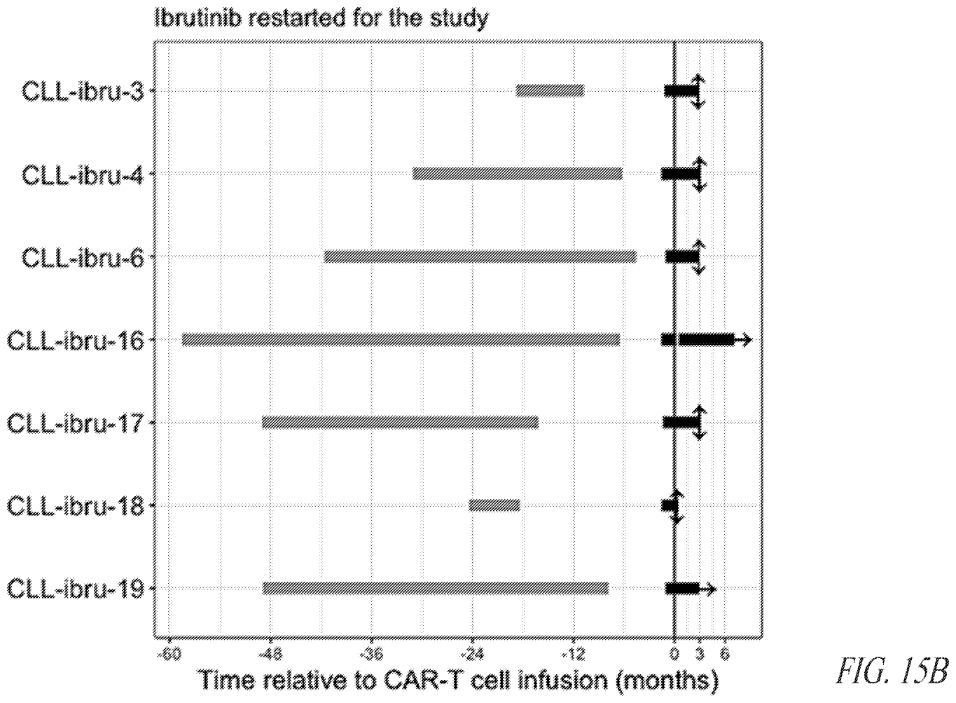

Prior to lymphodepletion, all patients had high-risk disease, including 17 (89%) with high-risk cytogenetics. All patients had bone marrow disease (median abnormal B cells by flow cytometry, 26%), and 17 (89%) had measurable lymph node disease (median cross-sectional tumor area, 2,624 mm²). The median number of prior therapies was 5 (range, 1-10). Five patients (26%) had previously been treated with idelalisib and 11 (58%) had received venetoclax. Two patients (10%) with 17p deletion had received ibrutinib or an ibrutinib-based combination as first-line therapy. Twelve patients (63%), all of whom had failed ibrutinib (progressive disease, PD, n=11; stable disease, SD, n=1), were still receiving ibrutinib at study enrollment (median time on ibrutinib before leukapheresis, 726 days; range, 78-2185; FIG. 15A). Seven patients (37%) who had previously ceased ibrutinib due to PD recommenced it shortly before leukapheresis (median time on ibrutinib before leukapheresis, 24 days; range, 15-30; FIG. 15B).

TABLE 44
Patient characteristics (n = 36; Cy/Flu, 2 × 10⁶ CAR-T cells/kg)

| | Ibrutinib cohort | | No-Ibrutinib cohort | | |
|---|---|---|---|---|---|
| | (n = 17) | | (n = 19) | | p |
| Age (median [IQR]) | 65 | [55, 69] | 61 | [54, 64] | 0.34 |
| Sex (n, (%)) | | | | | |
| Female | 7 | (41) | 7 | (37) | 1.00 |
| Male | 10 | (59) | 12 | (63) | |
| ECOG (n, (%)) | | | | | |
| 0 | 8 | (47) | 9 | (47) | 1.00 |
| 1 | 9 | (53) | 10 | (53) | |
| Richter's transformation (n, (%)) | 3 | (18) | 4 | (21) | 1.00 |
| Complex karyotype (n, (%)) | 13 | (76) | 16 | (89) | 0.40 |
| Chromosome 17p deletion (n, (%)) | 13 | (76) | 12 | (63) | 0.48 |
| Tumor cross-sectional area (mm²; | 2624 | [1454, 4016] | 3226 | [2414, 5156] | 0.20 |
| median [IQR]) | | | | | |
| Bulky adenopathy ≥5 cm (n, (%)) | 3 | (18) | 5 | (26) | 0.70 |
| Maximum SUV (median [IQR]) | 4 | [3, 7] | 5 | [5, 11] | 0.12 |
| Blood LDH concentration (UI/L; | 153 | [131, 229] | 225 | [196, 306] | 0.02 |
| median [IQR]) | | | | | |
| Number of prior therapies | 5 | [4, 7] | 5 | [4, 6] | 0.55 |
| (median [IQR]) | | | | | |
| Prior hematopoietic cell transplant | 2 | (12) | 3 | (16) | 0.69 |
| (n, (%)) | | | | | |
| Prior progression on ibrutinib (n, (%)) | 16 | (94) | 18 | (95) | 1.00 |
| Time to progression or intolerance | 24 | [18, 36] | 13 | [9, 20] | 0.02 |
| to ibrutinib prior to treatment | | | | | |
| with anti-CD19 CAR-T cells | | | | | |
| (months, median [IQR]) | | | | | |
| Prior progression on venetoclax | 6 | (35) | 5 | (26) | 0.72 |
| Percentage of CLL cells in BM | 26 | [13, 64] | 59 | [28, 78] | 0.30 |
| (median [IQR]) | | | | | |
| Percentage of CLL cells in blood | 14 | [6, 42] | 22 | [0, 47] | 0.95 |
| (median [IQR]) | | | | | |
| Absolute number of CLL cells in | 1 | [0, 4] | 1 | [0, 7] | 0.84 |
| blood (109 cells/L; median [IQR]) | | | | | |
| Absolute lymphocyte count (109 | 1 | [1, 5] | 2 | [1, 8] | 0.37 |
| cells/L; median [IQR]) | | | | | |

All variables assessed prior to lymphodepletion unless specified. P-values per Fisher's or Kuskal-Wallis tests as appropriate

The determined causes for discontinuance/reduction of ibrutinib and the number of days after CAR-T cell infusion that the discontinuance/reduction was made are shown in (Table 45).

TABLE 45
Causes and Timing of ibrutinib Dose Reduction or Discontinuation
after CD19 CAR-T Cell Therapy with Concurrent Ibrutinib

| | Cause of first | Day of first | Total duration |
|---|---|---|---|
| | ibrutinib dose | ibrutinib dose | of concurrent |
| | reduction or | reduction or | ibrutinib |
| Patient # | discontinuation | discontinuation* | therapy* |
| CLL-ibru-3 | Disease progression | 84 | 84 |
| CLL-ibru-5 | Grade 3 neurologic | 4 | 25 |
| | toxicity | | |
| CLL-ibru-6 | Grade 3 | 7 | 89 |
| | thrombocytopenia§ | | |
| CLL-ibru-14 | Grade 3 neurologic | 21 | 24 |
| | toxicity | | |
| CLL-ibru-16 | Disseminated | 6 | 6 |
| | intravascular | | |
| | coagulation during | | |
| | grade 2 CRS | | |
| CLL-ibru-18 | Microembolic strokes | 8 | 8 |
| | in the context of DIC | | |
| | during grade 3 | | |
| | neurologic toxicity | | |
| CLL-ibru-15 | Sudden death from | N/A | 4 |
| | presumed cardiac | | |
| | arrhythmia during | | |
| | grade 2 CRS not | | |
| | requiring vasopressors | | |

*After CAR-T cell infusion
§CLL-ibru-6 continued on ibrutinib at a reduced dose.
Abbreviations:
CAR-T cell, chimeric antigen receptor-engineered T cell;
CRS, cytokine release syndrome;
DIC, disseminated intravascular coagulation

### 4.2.2 Response Rate

Responses to therapy were analyzed for both cohorts at four weeks following CAR-T cell infusion. Responses were graded according to criteria including the 2018 IWCLL criteria. Data are summarized in Tables 46 and 47.

TABLE 46
Response at 4 weeks after CD19 CAR-T Cell
Therapy with Concurrent Ibrutinib

| | N (%) | |
|---|---|---|
| Response by 2018 iwCLL criteria | | |
| ORR (CRi + PR) | 15/18 | (83) |
| CRi* | 4/18 | (22) |
| PR | 11/18 | (61) |
| SD | 0/18 | (0) |
| PD | 3/18 | (17) |
| Marrow response | | |
| No detectable disease by flow cytometry (sensitivity: 10⁻⁴) | 13/18 | (72) |
| No detectable disease by IGH sequencing (sensitivity: 10⁻⁶) | 11/18 | (61) |
| Nodal response by 2018 iwCLL CT criteria§ | | |
| ORR (CR + PR) | 10/14 | (71) |
| CR | 1 | (7) |
| PR | 9 | (64) |
| SD | 1 | (7) |
| PD | 3 | (22) |

*Three (3) patients without evaluable nodal disease by CT achieved CRi by 2018 iwCLL criteria
§Fourteen (14) patients had evaluable nodal disease by CT.
Abbreviations:
CAR-T cell, chimeric antigen receptor-engineered T cell;
iwCLL, international workshop chronic lymphocytic leukemia;
ORR, overall response rates;
CR, complete response,
PR, partial response,
SD, stable disease,
PD, progressive disease,
CT, computed tomography

TABLE 47
Summary - Response

| | Four weeks | |
|---|---|---|
| Response | Ibrutinib | No-Ibrutinib |
| Marrow response by flow cytometry* | 12/16 (75%) | 11/17 (65%) |
| Marrow response by IGH seq^{#} | 10/12 (83%) | 6/10 (60%) |
| Nodal response\hat{ } | 9/12 (75%) | 10/17 (59%) |
| PET response@ | 7/8 (88%) | 8/12 (67%) |
| 2018 IWCLL response | 14/16 (88%) | 12/18 (67%) |

*Patients with marrow disease before CAR-T cells
^{#}Among flow-negative patients with a trackable clone
\hat{ }Among those with nodal disease before CAR-T cells
@Among those with available PET scans and nodal disease

### 4.2.3 Statistical Analysis of Variables

Univariate analysis was performed to identify factors correlating with 4-week responses to treatment. The analysis included the factors shown in the left-hand panel of (Table 49). Those factors having a p-value of less than 0.10 based on the univariate analysis were considered for a stepwise multivariable analysis (backward/forward direction based on BIC criterion), and are shown in the right-hand panel of (Table 48).

TABLE 48
Univariate Analysis - Factors Correlating With
Response at 4 Weeks by 2008 IWCLL Criteria

| | Variables With P < 0.10 in |
|---|---|
| | Univariate Analysis Considered |
| | for Stepwise Multivariable |
| Variables Evaluated in Univariate Analysis | Analysis |
| Ibr cohort, age, gender, ECOG, Richter's | Ibr cohort |
| transformation, 11q abnormality, complex karyotype, | 11q abnormality |
| tumor cross-sectional area, maximum SUV, bulky | Maximum SUV |
| disease, LDH blood concentration, number of prior | LDH blood concentration |
| therapies, prior autologous stem cell transplantation, | Prior allogeneic stem cell |
| prior allogeneic stem cell transplantation, time to | transplantation |
| progression or intolerance to ibrutinib prior to | Peak CAR-T cell in blood by |
| treatment with anti-CD19 CAR-T cells, prior treatment | qPCR |
| with idelalisib, prior treatment with venetoclax, | |
| percentage of BM abnormal B cells, abnormal B cells | |
| in blood (percentages and counts), time from | |
| leukapheresis to lymphodepletion, time from | |
| leukapheresis to anti-CD19 CAR-T cell infusion, | |
| ALC, CD8+ and CD4+ anti-CD19 CAR-T cell counts | |
| and percentages in blood (peak and AUC), anti-CD19 | |
| CAR-T cells by qPCR in blood (peak and AUC) | |

Two factors (patient cohort and pre-treatment max SUV per unit) were identified as strongly correlated with responses based on the stepwise analysis.

Binomial logistic regression was performed using these two factors. Data are shown in Table 49.

TABLE 49
Binomial Logistic Regression for Response
at 4 weeks by 2008 IWCLL Criteria

| | Adjusted OR (95% CI) | | P (LR test) |
|---|---|---|---|
| | 1 | | 0.11 |
| | 10.83 | (0.31-381.2) | |
| Pre-treatment max SUV (per unit) | 0.73 | (0.57, 0.94)* | <0.001 |

A stepwise approach was used for variable selection with backward/forward direction based on the BIC criterion. Time to progression or intolerance to ibrutinib prior to treatment with anti-CD19 CAR-T cells was not included in the multivariable analysis (OR 1.02, P=0.38).

Pre-treatment max SUV correlated strongly with adjusted overall response (p<0.001, likelihood-ratio test). Cohort did not significantly correlate with adjusted overall response (p=0.11).

### 4.2.4 Progression Free Survival (PFS) and Overall Survival (OFS)

Of the 23 patients (64%) with no detectable bone marrow disease by flow cytometry at 4 weeks, 22 (96% of the 23) were evaluated by IgH sequencing, and PFS probability curves were generated for each cohort.

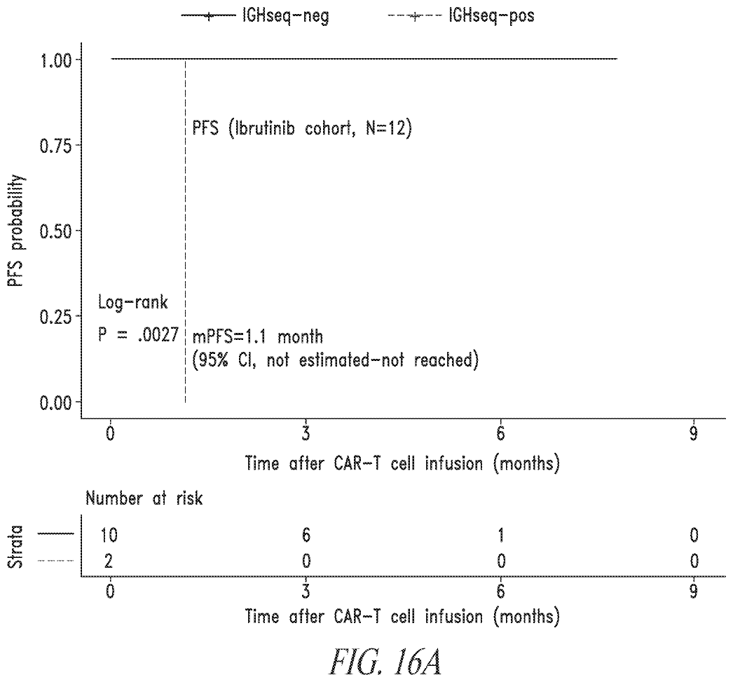

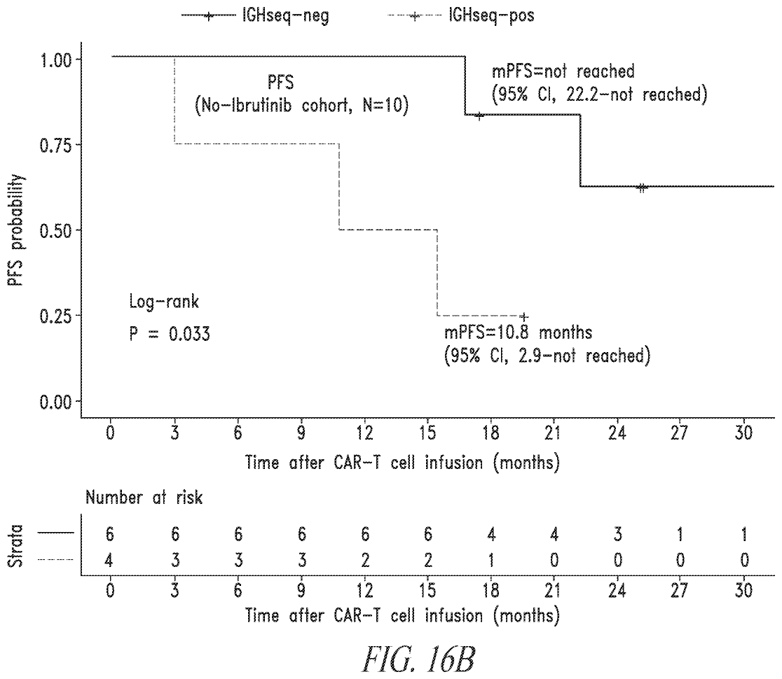

Data for the ibrutinib cohort are shown in FIG. 16A. Ibrutinib-cohort patients with disease-negative IgH sequencing results had 100% PFS probability at approximately 7.5 months after CAR-T cell infusion. Ibrutinib-cohort patients with disease-positive IgH sequencing results had a sharp drop to 0% probability of PFS by 1.1 months, though the 95% confidence interval (CI) was not reached.

Data for the no-ibrutinib cohort are shown in FIG. 16B. IgH sequencing again appeared to identify patients at lower risk for disease progression, though neither IgH-sequence-result group reached 95% CI.

These data show that HR-CLL patients who continue to receive ibrutinib and are disease-negative by IgH sequencing may have an increased probability of achieving PFS versus those who are disease-positive by sequencing. The data also show that patients who do not receive ibrutinib following CAR-T cell infusion and are disease-negative by IgH sequencing may have improved probability of achieving PFS compared to those who are disease-positive by sequencing.

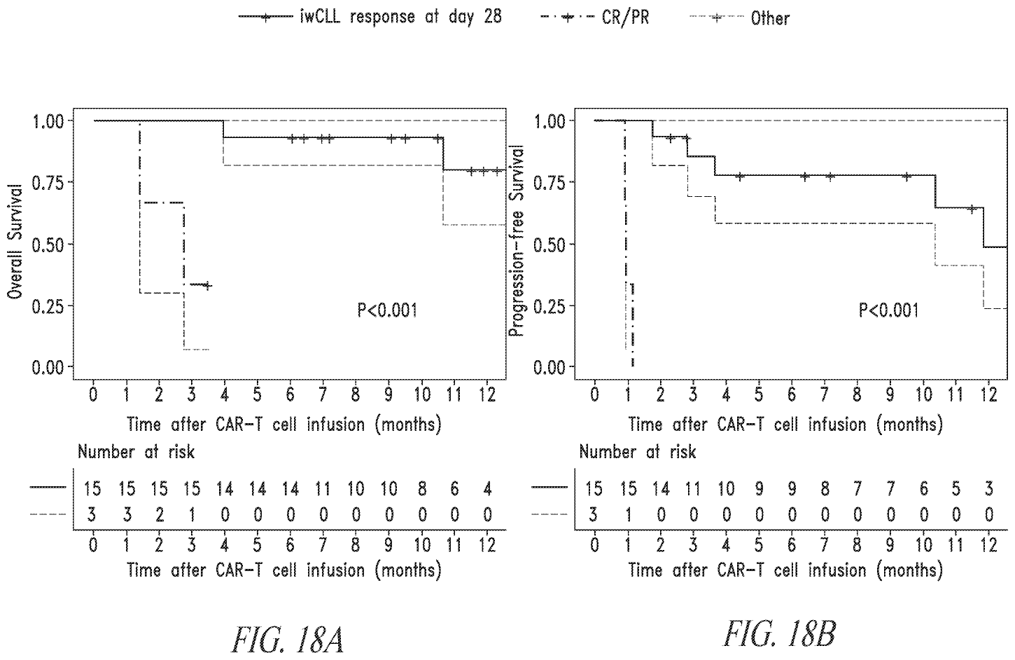

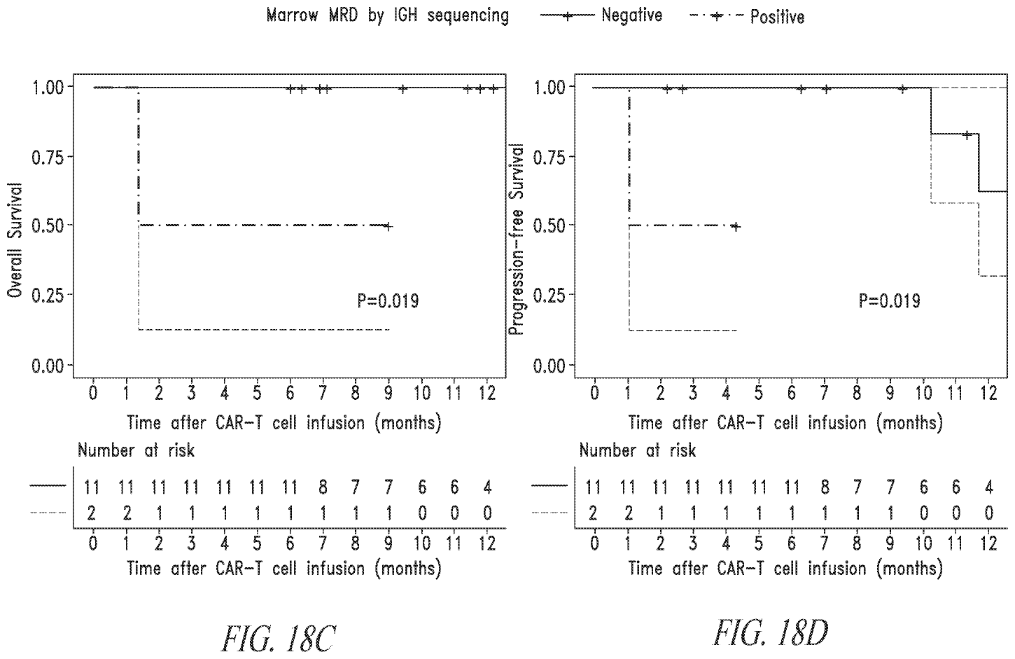

Responses by iwCLL criteria to CD19 CAR-T cell immunotherapy with concurrent ibrutinib were durable and responding patients had superior OS and PFS compared to non-responding patients. In responding patients by iwCLL criteria the 1-year probability of OS and PFS were 80% (95% CI: 57-100) and 49% (95% CI: 23-100), respectively (FIGS. 18A-18B). In patients who achieved an MRD-negative marrow response by flow cytometry (n=13) the 1-year probabilities of OS and PFS were 92% (95% CI: 79-100) and 58% (95% CI: 29-100), respectively. The depth of marrow response was associated with PFS. Among patients who cleared disease from marrow by flow cytometry four weeks after CAR-T infusion, achieving MRD-negativity. In patients who achieved MRD-negative marrow response by IGH sequencing (n=11), the 1-year OS and PFS probabilities were 100% (95% CI: 12-100) and 62% (95% CI: 32-100), respectively (FIGS. 18C-18D). In this group, two late relapses were observed at 10.2 and 12 months after infusion. OS and PFS of the entire cohort are shown in FIGS. 19A and 19B.

### 4.2.5 Toxicity: Cytokine Release Syndrome (CRS) and Neurotoxicity (NT)

CRS and NT events were also analyzed when grouping by cohort. In general, the ibrutinib cohort experienced fewer and less severe CRS and NT events, and notably experienced zero grade 3-5 CRS events (compare to 5 grade 3-5 events in the no-ibrutinib cohort). The differences in CRS outcomes between the two cohorts was statistically significant (p=0.05, Fisher's test), but the difference in NT events was not (p=0.48).

TABLE 50
Cytokine Release Syndrome (CRS) and Neurotoxicity
Events observed in the two Patient Cohorts

| | | Ibrutinib | | No Ibrutinib | |
|---|---|---|---|---|---|
| | | (n = 19) | | (n = 19) | |
| CRS | Absent | 4 | (24) | 2 | (11) |
| | Grade 0-2 | 17 | (100) | 14 | (74) |
| | Grade 3-5 | 0 | (0) | 5 | (26) |
| | Any grade | 13 | (76) | 17 | (89) |
| Neurotoxicity | Absent | 12 | (71) | 11 | (58) |
| | Grade 0-2 | 13 | (76) | 12 | (63) |
| | Grade 3-5 | 4 | (24) | 7 | (37) |

TABLE 51
Grade 3-4 adverse events according to CTCAE 4.03 after
CD19 CAR-T cell therapy with concurrent ibrutinib

| Adverse event | N | % |
|---|---|---|
| Neutropenia | 19 | 100 |
| Lymphopenia | 19 | 100 |
| Anemia | 15 | 79 |
| Thrombocytopenia | 13 | 68 |
| Hypertension | 7 | 37 |
| Hypophosphatemia | 5 | 26 |
| Hyponatremia | 4 | 21 |
| Hyperglycemia | 4 | 21 |
| Elevated ALT | 2 | 11 |
| Thromboembolic event | 2 | 11 |
| Muscle weakness | 2 | 11 |
| Elevated AST | 2 | 11 |

*occurring in >10% of patients.
Abbreviations:
CTCAE, Common Terminology Criteria for Adverse Events;
CAR-T cell, chimeric antigen receptor-engineered T cell;
ALT, alanine transaminase;
AST, aspartate transaminase

These data suggest that HR-CLL patients who continue to receive ibrutinib following infusion CAR-T cells experience fewer and less severe CRS events, and may also have better NT outcomes. To determine whether the CRS difference between the two cohorts reflects earlier intervention (tocilizumab/dexamethasone) in the ibrutinib group, the time and grade of CRS/NT event at the first instance of intervention are analyzed.

To determine if the addition of ibrutinib to CAR-T cell therapy might contribute to the low risk of severe CRS, patients treated with concurrent CD19 CAR-T cells and ibrutinib (Con-ibr cohort, n=19) were retrospectively compared with patients who received the same CD19 CAR-T cell product without ibrutinib (No-ibr cohort, n=24; Table 44).

Bayesian analyses were used to determine if there was a difference in the risk of CRS between the cohorts. Lower posterior probabilities of CRS were calculated in the Con-ibr cohort (grade ≥1 CRS, 72%, 95% credible interval 53-90; grade ≥3 CRS, 3%, 95% credible interval 0-14, FIGS. 20A-20B) compared to the No-ibr cohort (grade ≥1 CRS, 89%, 95% credible interval 76-98; grade ≥3 CRS, 26%, 95% credible interval 11-43). Despite 5 of 24 patients in the No-ibr cohort receiving <2×10⁶/kg CAR-T cells, the probabilities of lower rates of grade ≥1 CRS and grade ≥3 CRS in the Con-ibr cohort were 94% and 99%, respectively. The estimated differences in rates of grade ≥1 and ≥3 CRS between cohorts were −17% (95% credible interval 4-40) and −22% (95% credible interval 3-41), respectively. These data indicate a high probability that the incidence and severity of CRS was lower in the cohort of patients who received ibrutinib with CAR-T cells.

The occurrence of CRS may be associated with tumor burden in CLL patients (Turtle et al., J. Clin. Oncol., online (2017)). Continuation of ibrutinib therapy through leukapheresis and CAR-T cell manufacturing could minimize the risks of rapid tumor progression after ibrutinib withdrawal and reduce tumor burden before CAR-T cell immunotherapy. Consistent with this approach, patients in the Con-ibr cohort had lower LDH concentrations (median 155 versus 234 UI/L, p=0.01) and a lower percentage of CLL in the marrow (median 26% versus 59%, p=0.09) compared to those in the No-ibr cohort (Table 45).

The data indicated that lower marrow tumor burden in the Con-ibr cohort could contribute to the reduced risk of severe CRS. To determine if ibrutinib might contribute to changes in factors other than the tumor burden that decreased the risk of CRS, analysis was performed to investigate whether treatment in the Con-ibr cohort was independently associated with the risk of CRS after adjusting for the percentage of abnormal B cells in bone marrow before lymphodepletion. In a logistic regression model after adjusting for marrow tumor burden, treatment in the Con-ibr cohort was independently associated with lower probabilities of grade ≥1 (OR=0.32, p=0.23) and grade ≥2 CRS (OR=0.71, p=0.61), indicating that ibrutinib may decrease the severity of CRS by mechanisms other than reduction in tumor burden.

### 4.2.6 CAR-T Cell Kinetics

In vivo expansion of CAR-T cells in the two patient cohorts was examined over 30 days post-infusion by flow cytometry.

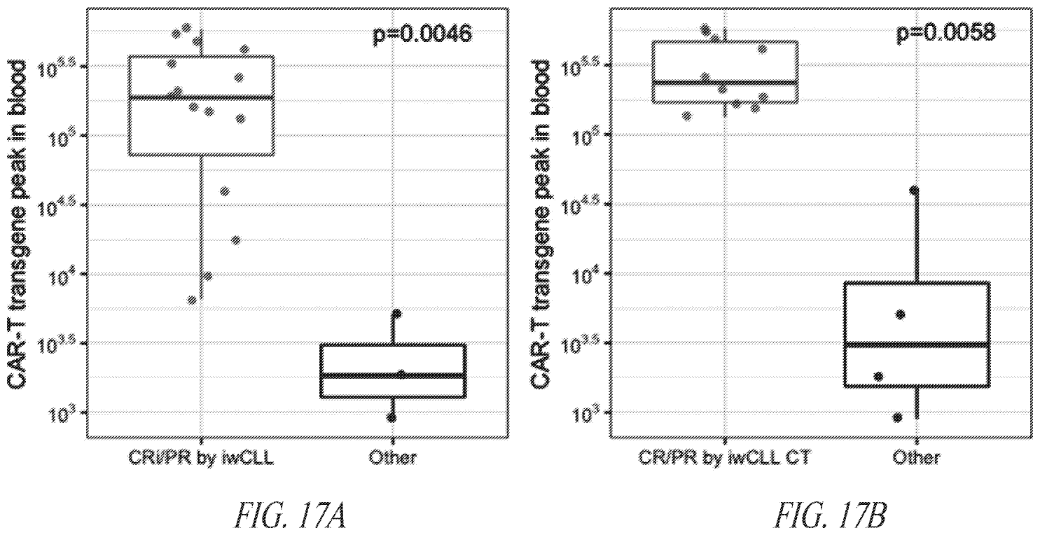

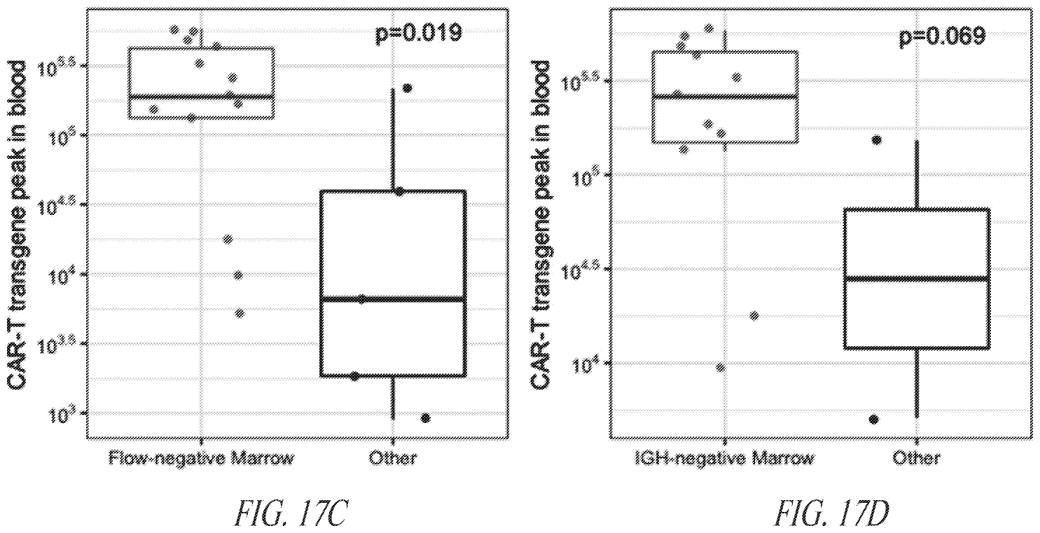

Data are summarized in FIGS. 17A-17D. In vivo CAR-T cell expansion was associated with antitumor efficacy, with higher CAR-T transgene copies in responders by iwCLL criteria and by iwCLL CT criteria (A, B). Higher CAR-T cell transgene copies were also observed in patients without detectable marrow disease by flow cytometry or by IGH sequencing (C, D).

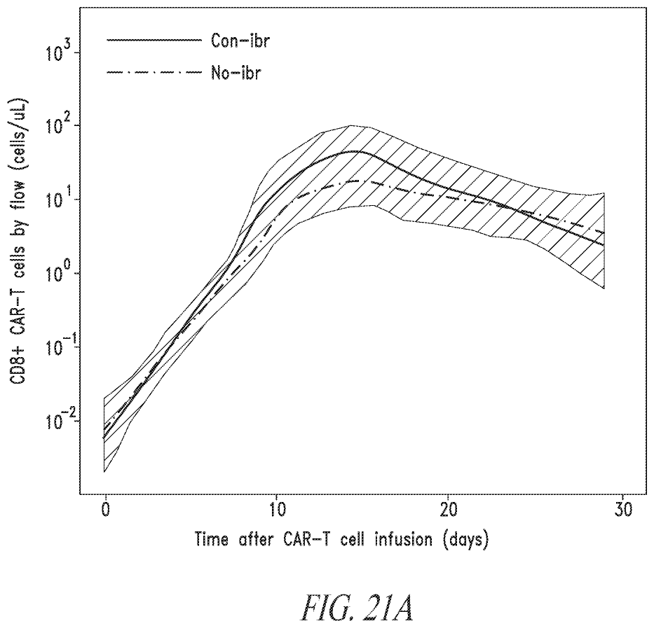

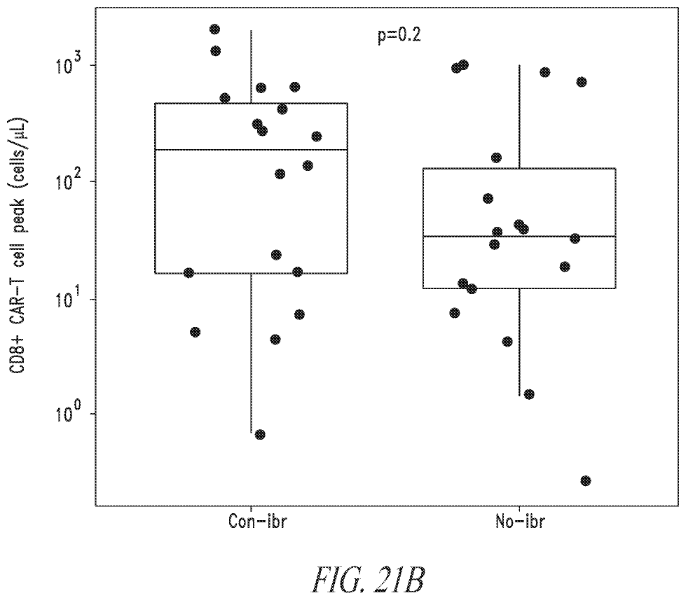

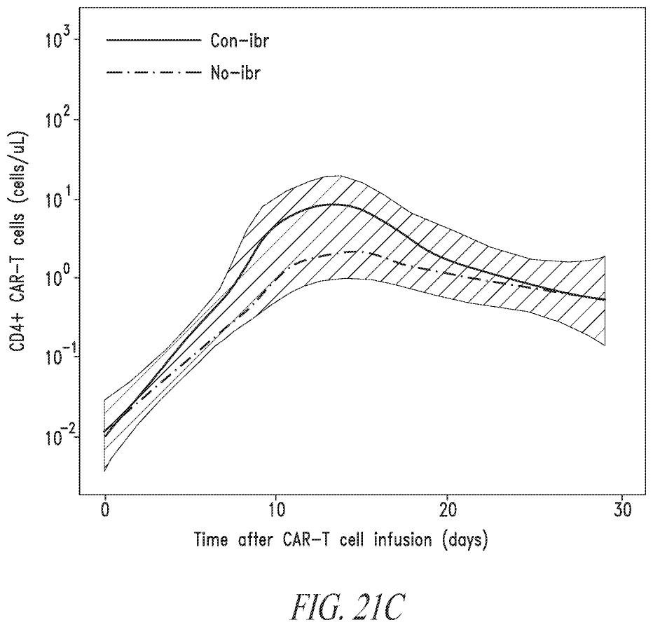

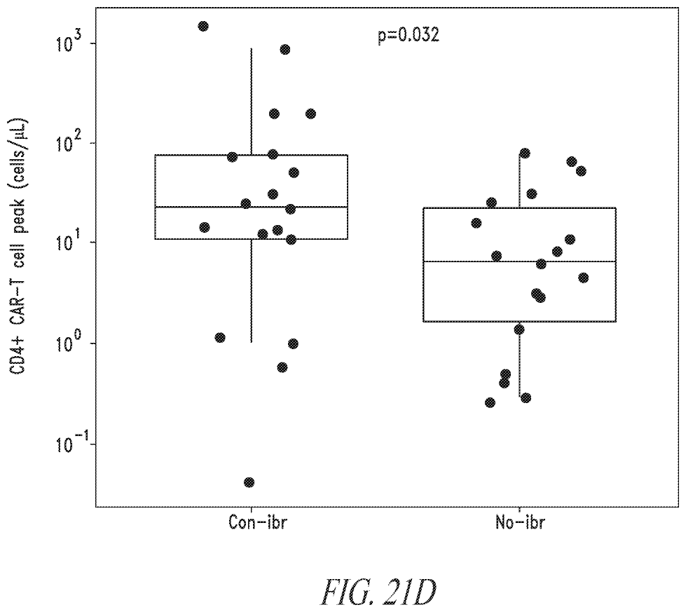

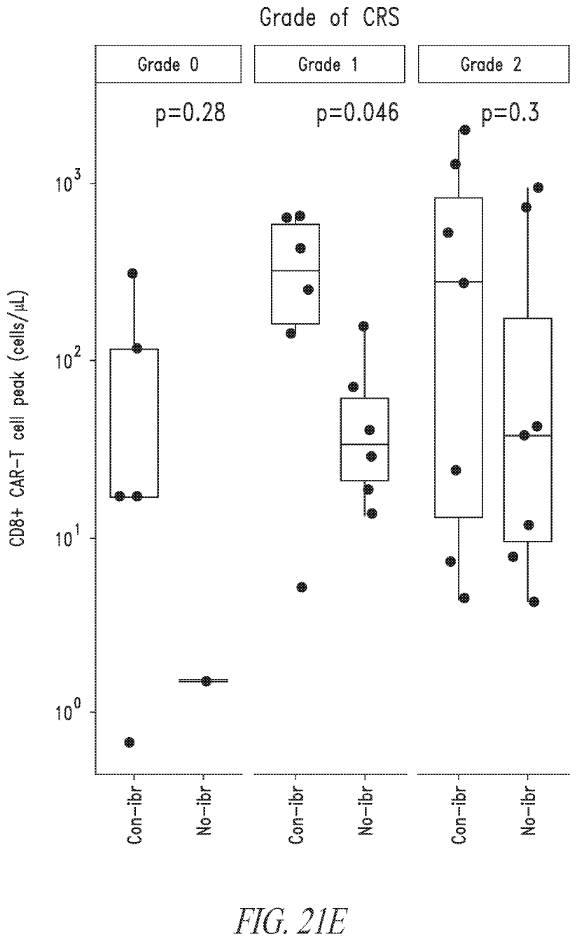

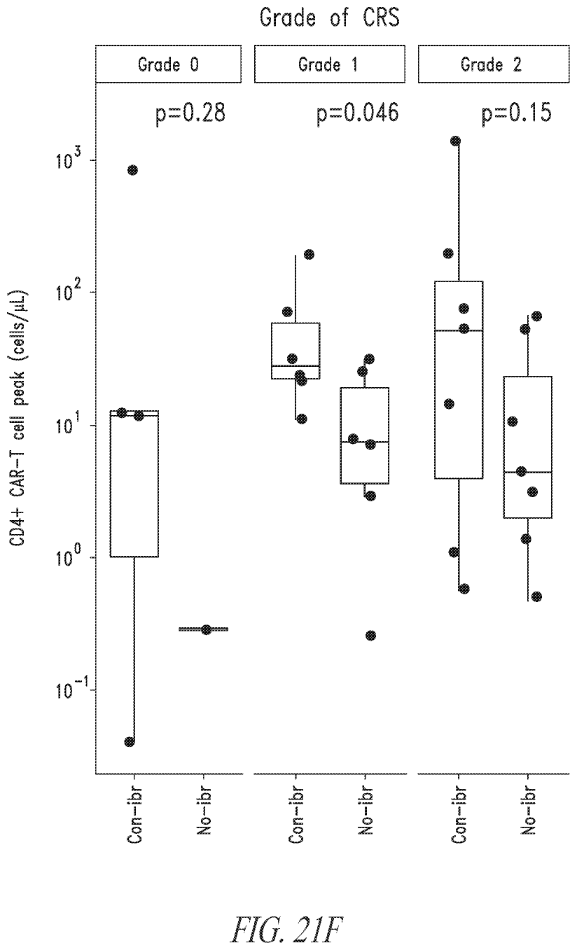

Because ibrutinib inhibits ITK and might affect antigen-dependent signaling in T cells, studies were performed to determine whether there were differences in in vivo CAR-T cell expansion that could contribute to the lower incidence of CRS in the Con-ibr cohort. Despite the lower incidence of grade ≥3 CRS, comparison of CAR-T cell kinetics in patients who received the same CAR-T cell dose revealed that patients in the Con-ibr cohort had higher CD4+ and comparable CD8+ peak CAR-T cell counts in blood compared to those in the No-ibr cohort (CD4+, median 23.2 cells/μL versus 6.5 cells/μL, P=0.03, respectively; FIGS. 21A-21D). Patients in the Con-ibr cohort also achieved higher peak CAR-T cell counts compared to those in the No-ibr cohort when analyses were performed within sub-cohorts stratified by the CRS grade (grade 1; CD8+, p=0.046, CD4+, p=0.046, FIG. 21E-21F). These data indicate that ibrutinib-associated impairment of CAR-T cell expansion is unlikely to be the cause of the lower incidence of CRS in the Con-ibr cohort.

### 4.2.7 Kinetics of Toxicity Related Cytokines

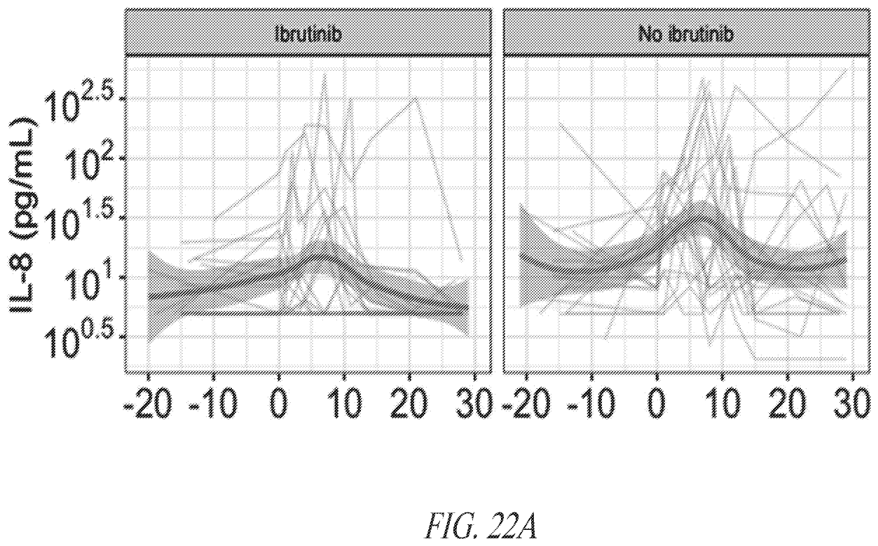

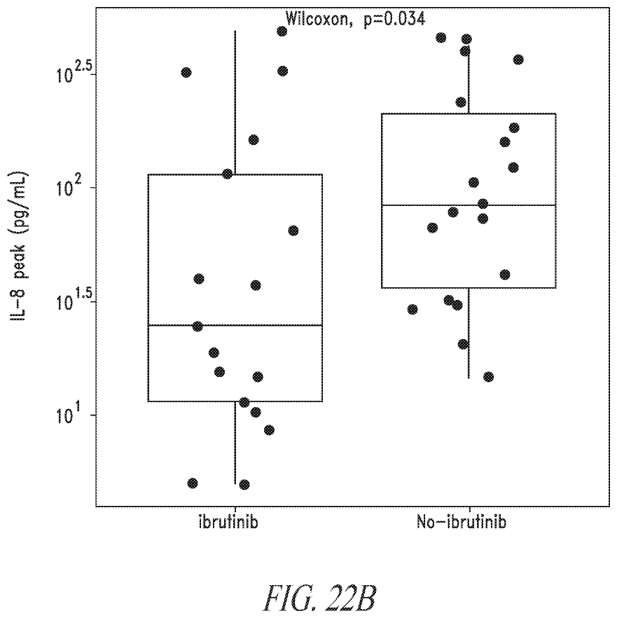

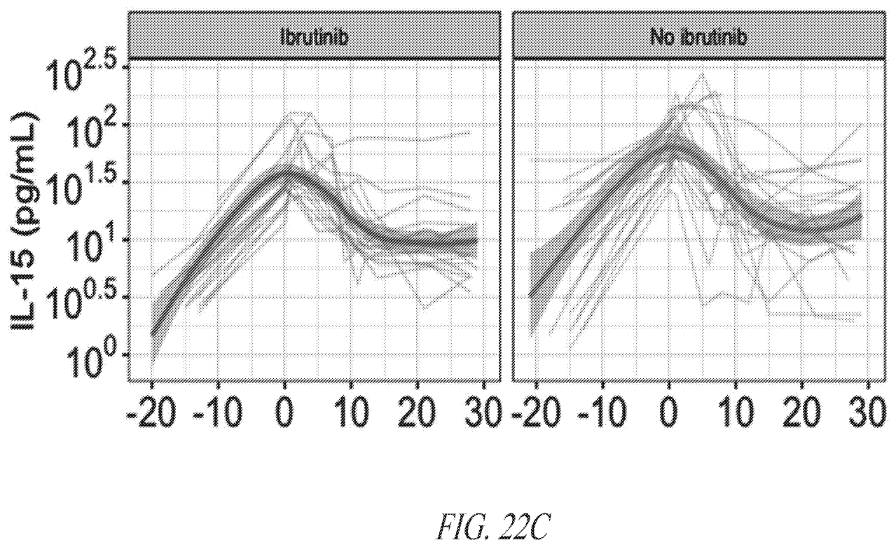

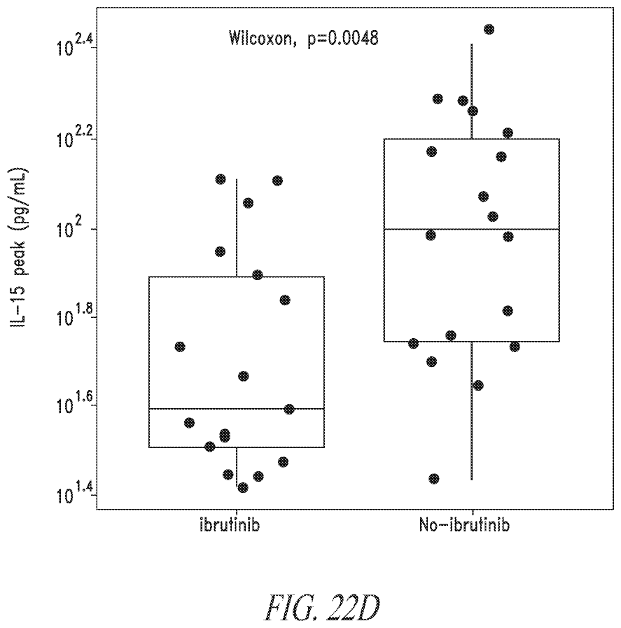

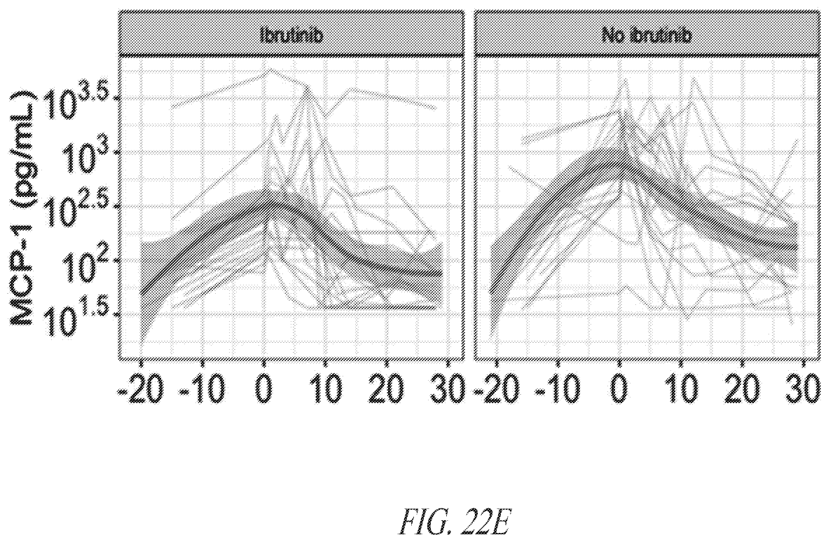

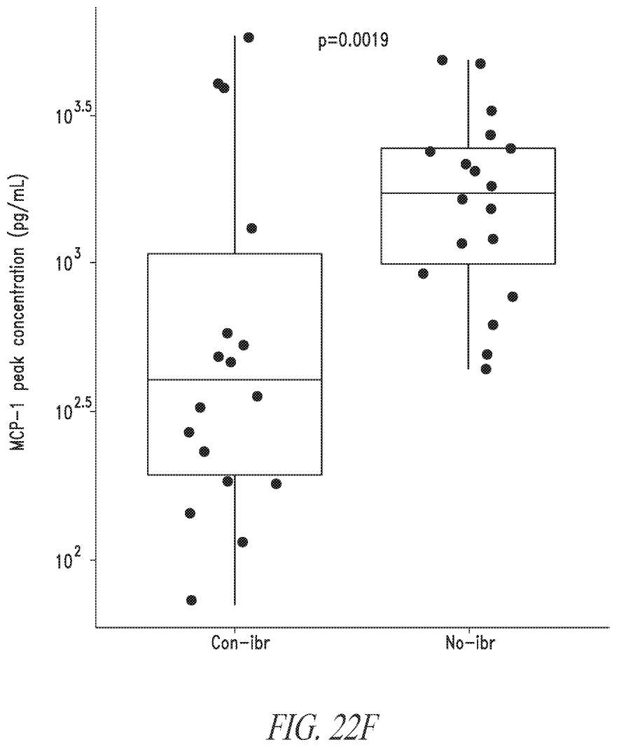

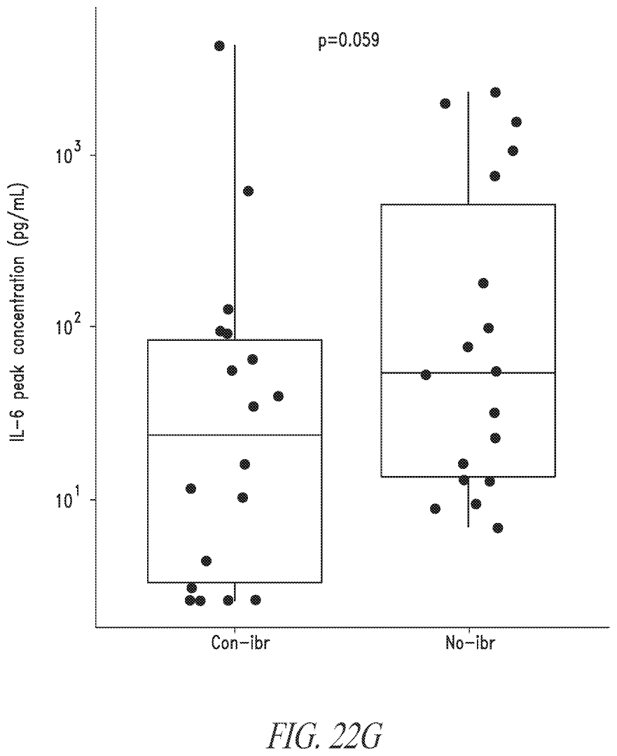

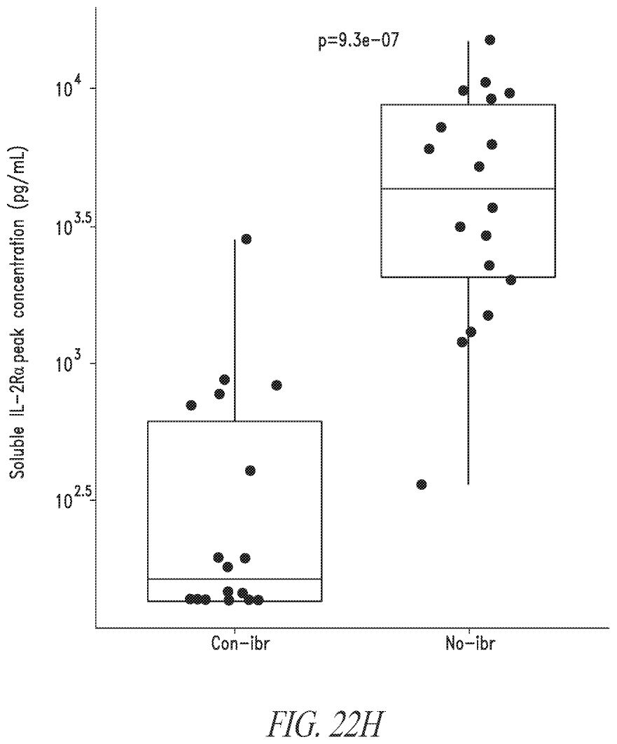

In vivo kinetics of interleukin-8 (IL-8), interleukin-15 (IL-15), Monocyte Chemoattractant Protein-1 (MCP-1), interleukin-6 (IL-6), and soluble interleukin-2 receptor alpha (sIL-2Rα) were evaluated with respect to the two treatment cohorts. Data are shown in FIGS. 22A-22H. The ibrutinib group experienced lower levels (both peak and over time) of all markers. The peak concentration differences between the cohorts were significant for all but one of the measured molecules (Wilcoxon test; IL-8: p=0.034; IL-15: p=0.0048; MCP-1: p=0.0019; sIL-2Rα: p=9.3e⁻⁷). The difference in IL-6 expression between the two cohorts approached statistical significance (p=0.059).

These data show that continued treatment with ibrutinib following CAR-T cell infusion correlates with lower levels of toxicity associated cytokines in HR-CLL patients, in spite of robust CAR-T cell expansion.

### 4.2.8 CAR-T Cell Kinetics by CRS Grade

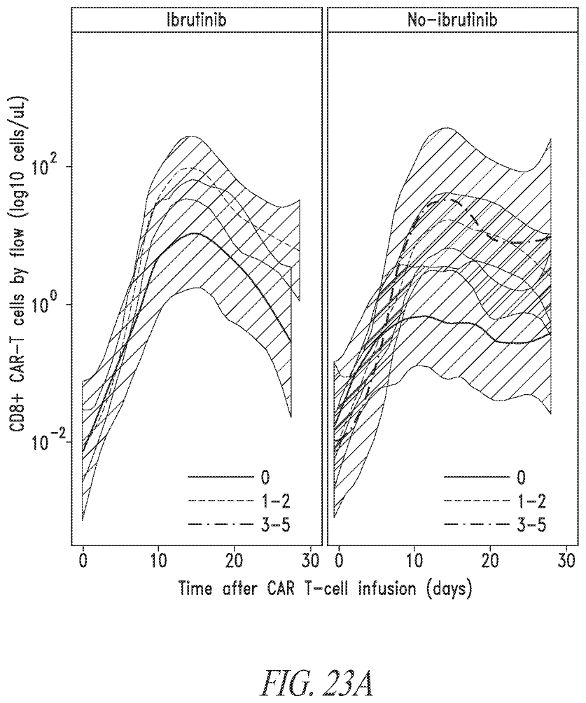

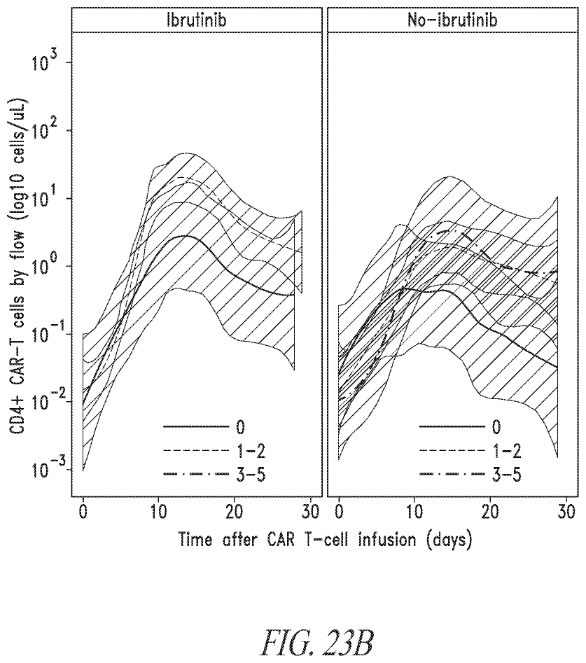

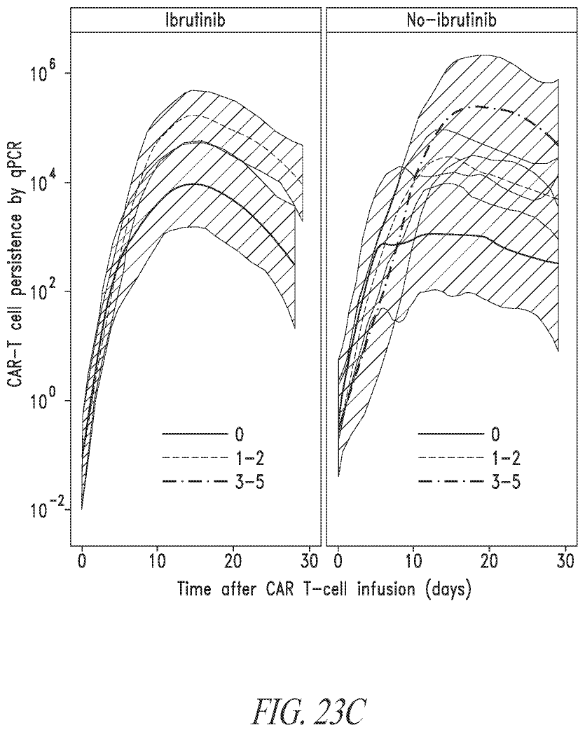

Expansion and persistence of CAR-T cells in each cohort was also analyzed in regard to the severity of CRS events experienced. Data is shown in FIGS. 23A-23C.

4.2.9 Deeper Marrow Response in CLL Patients Receiving CAR-T Cells with Concurrent Ibrutinib

The higher CD4+ and equivalent CD8+ CAR-T cell counts in the Con-ibr cohort suggested that ibrutinib does not impair the efficacy of CAR-T cells. The posterior probabilities of response by iwCLL criteria in the Con-ibr cohort and in the No-ibr cohort were 81% (95% credible interval, 63-95) and 64% (95% credible interval, 45-81), respectively (data not shown); and the probability of higher response by iwCLL in the Con-ibr cohort was 89%. The estimated difference in response rates by iwCLL criteria between the two cohorts was 16% (95% credible interval, -9-41).

Among patients with an identified malignant IGH sequence who achieved MRD-negative marrow by flow cytometry, the posterior probabilities of eliminating malignant IGH sequences in the marrow in the Con-ibr cohort and No-ibr cohort were 82% (95% credible interval, 60-97) and 50% (95% credible interval, 27-74), respectively (data not shown), with a 97% probability of higher IGH-negative marrow response rates in the Con-ibr cohort. The estimated difference in IGH-negative marrow response rates between the two cohorts was 31% (95% credible interval, 0-60). After adjusting for the percentage of abnormal marrow B cells (OR: 27.27, 95% CI: 1.35-551.69, P=0.03), the pre-lymphodepletion LDH concentration (OR: 6.35, 95% CI 0.95-42.66, P=0.06), and the pre-lymphodepletion SPD (OR: 6.41, 95% CI: 0.88-46.39, P=0.06), logistic regression analysis showed the addition of ibrutinib was independently associated with higher probabilities of IGH-negative marrow response.

The data indicate that the lower risk of CRS in the Con-ibr cohort was associated with equivalent or better anti-tumor efficacy compared to the No-ibr cohort.

### 4.3 Conclusion

This study investigated whether concurrent administration of ibrutinib through leukapheresis, lymphodepletion, and CD19 CAR-T cell infusion could improve outcomes of CAR-T cell immunotherapy in heavily pre-treated high-risk CLL patients who had previously failed ibrutinib. Combination therapy with ibrutinib and CD19 CAR-T cells resulted in high response rates by iwCLL (ORR 83%) and high rates of MRD-negative marrow response by flow cytometry (72%) and IGH sequencing (61%) with a low incidence of severe toxicity. Concurrent administration of ibrutinib with CAR-T cells was well-tolerated in most patients; however, one sudden death from probable cardiac arrhythmia was observed in the context of mild grade 2 CRS not requiring vasopressors. Ibrutinib is known to be arrhythmogenic, and ventricular arrhythmia and sudden death have been reported (Lampson et al., Blood 129:2581-2584 (2017); Tang et al., Leukemia & Lymphoma:1-11 (2017); Cheng et al., Leukemia & Lymphoma:1-2 (2018)). A fatal cardiac arrhythmia occurred in another study after treatment with CTL119 and concurrent ibrutinib (Gill et al., ASH Abstract (2018)), indicating caution is warranted in patients with CRS while receiving ibrutinib after CAR-T cell therapy.

A retrospective comparison between patients in this study (Con-ibr cohort) and those who received CD19 CAR-T cells without ibrutinib (No-ibr cohort) demonstrated a lower risk of severe CRS after combined ibrutinib and CAR-T cell therapy, in part due to the lower tumor burden from ibrutinib treatment two weeks before leukapheresis through pre-treatment staging. In the context of increased in vivo T cell expansion and a reduced risk of CRS, the decrease in tumor burden was surprising, and suggested a possible correlative effect of ibrutinib on the CAR-T cells. Moreover, even after adjustment for the tumor burden, treatment with concurrent ibrutinib remained associated with a lower risk of CRS, indicating that ibrutinib likely contributed to changes in factors other than the tumor Frburden that decreased the risk of CRS.

Ibrutinib-induced inhibition of in vivo CAR-T cell expansion did not appear responsible for the lower risk of CRS; despite the lower tumor burden, there were equivalent or greater numbers of CAR-T cells in blood in patients who received concurrent ibrutinib compared to those treated with CAR-T cells alone.

In contrast to the higher CAR-T cell counts observed in patients treated with concurrent ibrutinib, lower concentrations of CRS-associated cytokines, MCP-1 and IL-6 were observed. The data indicate that ibrutinib is associated with a decrease in the capacity of CAR-T cell therapy to induce CRS, but does not impair CAR-T cell proliferation. The probabilities of response by iwCLL criteria and clearance of IGH sequence from marrow were higher in patients receiving concurrent ibrutinib and CAR-T cells compared to those receiving CAR-T cells without ibrutinib.

CD19 CAR-T cell therapy with concurrent ibrutinib was feasible in most patients and led to high response rates without severe CRS. The patients in this study had high-risk disease and had failed ibrutinib. Larger studies are warranted to address the role of combination therapy with ibrutinib and CAR-T cells in CLL patients with lower risk disease who have not yet progressed on ibrutinib.

### Example 5

### Durable Complete Remissions in Follicular Lymphoma Following CD19 CAR-T Cell Therapy

Follicular lymphoma is the second most frequent subtype of Non-Hodgkin's Lymphoma (NHL). Patients who relapse within 2 years after initial chemoimmunotherapy have limited survival (5-year overall survival [OS], 50%; Casulo et al., J. Clin. Oncol. 33(23):2516 (2015)), as do those who fail multiple regimens (5-year progression-free survival [PFS], 23%; Rivas-Delgado et al., Br. J. Haematol, 184(5):753 (2019)) or develop histologic transformation to large cell NHL (median PFS 1 year); Wagner-Johnston et al., Blood 126(7):851 (2015). Some studies of administering CD19 CAR-T cell immunotherapy to follicular lymphoma patients have been reported (see Schuster et al., NEMJ 377(26):2545 (2017); Turtle et al. Sci. Transl. Med. 8(355):355ra116 (2017)), in some instances in the context of multiple aggressive lymphomas; however, the incidence and durability of responses in patients with follicular lymphoma without transformation (FL) or with histologic transformation (tFL) are unknown.

### 5.1. Methods

A phase 1/2 clinical trial (NCT01865617) of CD19 CAR-T cell immunotherapy in adults with R/R CD19+ B-cell malignancies was conducted (Turtle et al. Sci. Transl. Med. 8(355):355ra116 (2017); Turtle et al., J. Clin. Invest. 126(6):2123 (2016); Turtle et al., J. Clin. Oncol. 35(26):3010 (2017)). The study was conducted according to the Declaration of Helsinki with informed consent and approval by the Institutional Review Board.

In one arm of the study, patients with FL and tFL received lymphodepletion with a cyclophosphamide and fludarabine (Cy/Flu)-containing regimen followed by 2×10⁶ CD19 CAR-T cells/kg. Manufacturing of CD19 CAR-T cells with 4-1BB costimulation was as previously described (Turtle et al. Sci. Transl. Med. 8(355):355ra116 (2017); Turtle et al., J. Clin. Invest. 126(6):2123 (2016)). Bridging antitumor therapy was permitted after leukapheresis. Patients were monitored for response and toxicities, as previously described (Turtle et al., 2017). Best responses were reported according to the Lugano criteria (Cheson et al., J. Clin. Oncol. 32(27):3059 (2014)), with rates calculated by the Clopper-Pearson method. Kaplan-Meier (KM) analyses were used to estimate PFS, DOR, and OS. Follow-up time was estimated by a reverse KM estimator (Schemper and Smith, Controlled Clinical Trials 17(4)L343 (1996)). Fisher's exact and Wilcoxon rank-sum tests were used to compare categorical and non-categorical variables, respectively. Data were analyzed using R version 3.4.1 (R Foundation for Statistical Computing, Vienna, Austria) and RStudio version 1.0.153 (RStudio, Boston, Mass.).

### 5.2. Patient Characteristics

Twenty-one patients (median age, 56 years; interquartile range [IQR], 51-62) who received a Cy/Flu lymphodepletion regimen and 2×10⁶ CD19 CAR-T cells/kg were included in the study (Table 52). Eight patients (38%) had FL and 13 (62%) had tFL.

TABLE 52
Patient and Treatment Characteristics

| | Follicular | Transformed | All |
|---|---|---|---|
| Characteristic | lymphoma | FL* | patients |
| Number (no.) of patients | 8 | 13 | 21 |

Age

| | | | | | |
|---|---|---|---|---|---|
| Median (interquartile | 53 (49-57) | 60 | (52-64) | 56 | (51-62) |
| range) - years | | | | | |
| ≥65 years - n (%) | 1 (13) | 2 | (15) | 3 | (14) |
| Male sex - n (%) | 5 (63) | 9 | (69) | 14 | (67) |
| ECOG performance- | 3 (38) | 5 | (38) | 8 | (38) |
| status score ≥1 - n (%) | | | | | |
| LDH, pre- | 1 (13) | 9 | (69) | 10 | (48) |
| lymphodepletion > | | | | | |
| ULN - n (%) | | | | | |

Disease stage - n (%)†

| I or II | 2 (25) | 0 | | 2 | (10) |
|---|---|---|---|---|---|
| III or IV | 6 (75) | 13 | (100) | 19 | (90) |

Extranodal disease - n (%)

| | | | | | |
|---|---|---|---|---|---|
| Yes | 5 (62) | 12 | (92) | 17 | (81) |
| No | 3 (38) | 1 | (8) | 4 | (19) |

FLIPI score - n (%)‡

| | | | | | |
|---|---|---|---|---|---|
| 0-1 | 2 (25) | 1 | (8) | 3 | (14) |
| 2 | 2 (25) | 2 | (15) | 4 | (19) |
| ≥3 | 4 (50) | 10 | (77) | 14 | (67) |

Bulky disease§

| | | | | | |
|---|---|---|---|---|---|
| Yes | 2 (25) | 6 | (46) | 8 | (38) |
| No | 6 (75) | 7 | (54) | 13 | (62) |

Bone marrow involvement

| | | | | | |
|---|---|---|---|---|---|
| Yes | 4 (50) | 2 | (15) | 6 | (29) |
| No | 4 (50) | 11 | (85) | 15 | (71) |

Tumor cross-sectional area∥

| | | | |
|---|---|---|---|
| Median - mm² | 2995 | 4695 | 3343 |
| Interquartile range - mm² | 2079-4208 | 2849-7265 | 2555-5794 |

Prior therapies

| | | | | | |
|---|---|---|---|---|---|
| Median (range) | 4 (2-7) | 5 | (2-8) | 5 | (2-8) |
| ≥4 prior lines of therapy - | 7 (88) | 11 | (85) | 18 | (86) |
| n (%) | | | | | |

Prior autologous hematopoietic cell transplantation - n (%)

| | | | | | |
|---|---|---|---|---|---|
| Yes | 3 (38) | 7 | (54) | 10 | (48) |
| No | 5 (62) | 6 | (46) | 11 | (52) |

Prior allogeneic hematopoietic cell transplantation - n (%)

| | | | | | |
|---|---|---|---|---|---|
| Yes | 1 (12) | 2 | (15) | 3 | (14) |
| No | 7 (88) | 11 | (85) | 18 | (86) |

Bridging therapy between leukapheresis and lymphodepletion¶

| | | | | | |
|---|---|---|---|---|---|
| Yes | 1 (12) | 4 | (31) | 5 | (24) |
| No | 7 (88) | 9 | (69) | 16 | (76) |

Lymphodepletion regimen - n (%)

| | | | | | |
|---|---|---|---|---|---|
| Cy 60 mg/kg × 1 + Flu 25 | 5 (62) | 6 | (46) | 11 | (52) |
| mg/m² × 3 | | | | | |
| Cy 30 mg/kg × 1 + Flu 25 | 0 | 3 | (23) | 3 | (14) |
| mg/m² × 3 | | | | | |
| Cy 300 mg/m² × 3 + Flu | 3 (38) | 4 | (31) | 7 | (34) |
| 30 mg/m² × 3 | | | | | |

Cy, cyclophosphamide;
ECOG, Eastern Cooperative Oncology Group;
FLIPI, Follicular Lymphoma International Prognostic Index;
Flu, fludarabine;
LDH, lactate dehydrogenase;
Transformed FL, diffuse large B-cell lymphoma transformed from follicular lymphoma;
ULN, upper limit of normal
*Four patients with high-grade B-cell lymphoma with MYC and BCL2 and/or BCL6 rearrangements; MYC rearrangement not available for 4 patients.
†Ann Arbor stage.
‡FLIPI scores include low risk (0 or 1 factor), intermediate risk (2 factors), and high risk (≥3 factors).
§A tumor lesion with a greatest diameter ≥7 cm or ≥3 nodes with a greatest diameter at each site ≥3 cm (Morschhauser et al., Cancer 116(18): 4299 (2010).
∥Sum of the product of the perpendicular diameters of up to 6 target measurable nodes and extranodal sites (Cheson et al., J. Clin. Oncol. 32(27): 3059 (2014)).
¶Chemotherapy (e.g. bendamustine + rituximab) or more than one dose of dexamethasone 20 mg or equivalent not as part of chemotherapy regimen.

The FL patients had received a median of 4 prior treatment regimens (range, 2-7); all had failed chemoimmunotherapy including an anti-CD20 antibody and alkylating agents; 7 of 8 patients had failed prior anthracycline exposure; 75% (n=6) had progressive disease (PD) after the last therapy; and 50% (n=4) had failed prior autologous (n=3) or allogeneic (n=1) hematopoietic cell transplantation (HCT). The 4 FL patients who had not failed HCT were not considered suitable candidates for HCT due to refractory disease. Before lymphodepletion, 75% of FL patients had stage III or IV disease; 62% had extranodal involvement; 75% had intermediate or high FL International Prognostic Index (FLIPI) score;13 and the median tumor burden, estimated by the sum of the product of the perpendicular diameters of up to 6 index lesions (SPD), was 2995 mm² (IQR, 2079-4208).

Twelve (12) of the 13 patients with tFL had documented diffuse large cell transformation at the last biopsy and one (1) had grade 3A FL with focal grade 3B disease. Four patients had confirmed high-grade B-cell lymphoma with MYC and BCL2 and/or BCL6 rearrangements (DH/TH). The median time from first histologic documentation of transformation to pre-lymphodepletion evaluation was 17.9 months (range, 7-83). The tFL patients had received a median of 5 prior treatment regimens (range, 2-8), and 62% (n=8) had failed prior autologous (n=6), allogeneic (n=1), or both autologous and allogeneic (n=1) HCT. Between leukapheresis and lymphodepletion, 4 of 13 tFL patients (31%) required systemic bridging therapy to control disease progression. Before lymphodepletion, all patients had stage III or IV disease; 92% had extranodal involvement; 92% had intermediate or high FLIPI score; and the median tumor SPD was 4695 mm² (IQR, 2849-7265). Patients with FL and tFL had comparable baseline and treatment characteristics (Table 53); however, more tFL patients had elevated lactate dehydrogenase (LDH; FL vs tFL, 13% vs 69%, P=0.02) and fewer had bone marrow involvement (50% vs 15%, P=0.15).

### 5.3. Results and Discussion

Twenty-one patients (median age, 56 years; interquartile range [IQR], 51-62) who received a Cy/Flu lymphodepletion regimen and 2×10⁶ CD19 CAR-T cells/kg were included in this study. Eight patients (38%) had FL and 13 (62%) had tFL.

The FL patients had received a median of 4 prior treatment regimens (range, 2-7); all had failed chemoimmunotherapy including an anti-CD20 antibody and alkylating agents; 7 of 8 patients had failed prior anthracycline exposure; 75% (n=6) had progressive disease (PD) after the last therapy; and 50% (n=4) had failed prior autologous (n=3) or allogeneic (n=1) hematopoietic cell transplantation (HCT). The 4 FL patients who had not failed HCT were not considered suitable candidates for HCT due to refractory disease. Before lymphodepletion, 75% of FL patients had stage III or IV disease; 62% had extranodal involvement; 75% had intermediate or high FL International Prognostic Index (FLIPI) score (Solal-Celigny et al., Blood 104(5):1258-1265 (2004)); and the median tumor burden, estimated by the sum of the product of the perpendicular diameters of up to 6 index lesions (SPD), was 2995 mm² (IQR, 2079-4208).

Twelve of the 13 patients with tFL had documented diffuse large cell transformation at the last biopsy and one had grade 3A FL with focal grade 3B disease. Four patients had confirmed high-grade B-cell lymphoma with MYC and BCL2 and/or BCL6 rearrangements (DH/TH). The median time from first histologic documentation of transformation to pre-lymphodepletion evaluation was 17.9 months (range, 7-83). The tFL patients had received a median of 5 prior treatment regimens (range, 2-8), and 62% (n=8) had failed prior autologous (n=6), allogeneic (n=1), or both autologous and allogeneic (n=1) HCT. Between leukapheresis and lymphodepletion, 4 of 13 tFL patients (31%) required systemic bridging therapy to control disease progression. Before lymphodepletion, all patients had stage III or IV disease; 92% had extranodal involvement; 92% had intermediate or high FLIPI score; and the median tumor SPD was 4695 mm² (IQR, 2849-7265). Patients with FL and tFL had comparable baseline and treatment characteristics (Table 52); however, more tFL patients had elevated lactate dehydrogenase (LDH; FL vs tFL, 13% vs 69%, P=0.02) and fewer had bone marrow involvement (50% vs. 15%, P=0.15).

Seven (7) of 8 patients with FL achieved CR (88%; 95% confidence interval [CI], 47-99) after CAR-T cells. The median time to CR was 29 days (range, 27-42), and all who achieved CR remained in remission without additional therapy (median follow-up, 24 months, range, 5-37). One patient with stable disease at first restaging received radiation 2.3 months after CAR-T cells and has not progressed 36 months after CAR-T cell infusion. These data demonstrate a remarkably high rate of durable CR in high-risk FL patients treated with CD19 CAR-T cells.

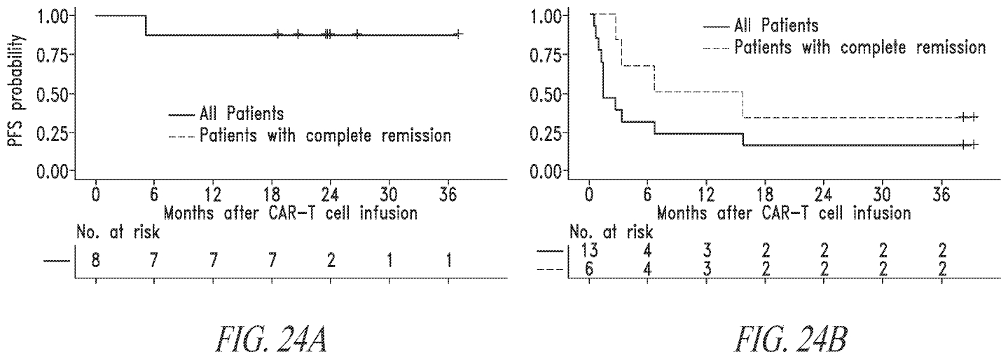

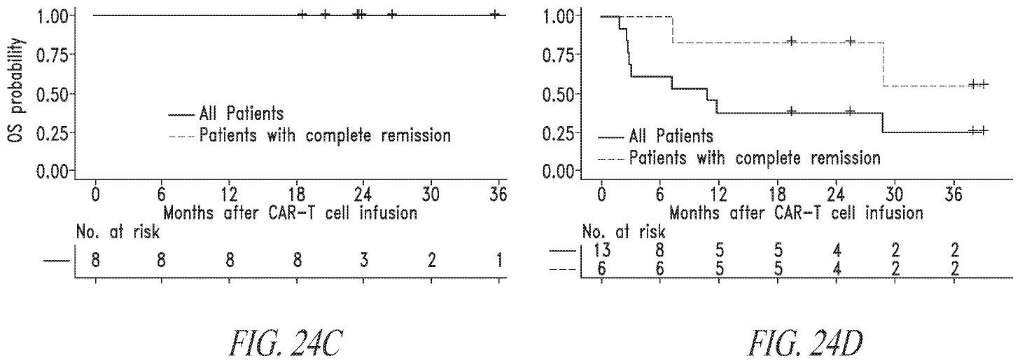

For the 13 patients with tFL, the best ORR without additional therapy after CAR-T cells was 46% (95% CI, 20-74), with all responding patients achieving CR. Three of 4 patients with tFL with DH/TH had PD. For those who achieved CR, at a median follow-up of 38 months (range, 3-39) the median PFS was 11.2 months (95% CI, 3.3-NR). For all patients with tFL, the median DOR and PFS were 10.2 months (95% CI, 2.3-NR) and 1.4 months (95% CI, 1.2-NR), respectively (FIGS. 24A-24D). No relapses had occurred after 15.6 months, with durable remissions observed for up to 39 months after CAR-T cell infusion.

CD19 CAR-T cell immunotherapy was well-tolerated in most patients. No differences were observed in the incidence or severity of cytokine release syndrome (CRS; grade ≥1; FL vs tFL, 50% vs 39%, P=0.35), or neurotoxicity (NT; grade ≥1; FL vs tFL, 50% vs 23%, P=0.67) between FL and tFL patients. Severe (grade ≥3) CRS and NT were not observed. Although CR and PFS rates differed between FL and tFL patients, the peak CAR-T cell counts and the area under the curve until day 28 (AUC0-28; transgene copies/μg DNA) in these groups were similar. The duration of CAR-T cell detection by quantitative polymerase chain reaction was also similar (FL, median 4.9 months, range, 0.9-24.0; tFL, median 3.0 months, range 0.5-24.5; P=0.33), as were pre-lymphodepletion, day 0, peak, and AUC0-28 serum IFN-γ, IL-2Rα, IL-5, IL-6, IL-7, IL-10, IL-15, IL-18, IL-22, MCP-1, MIP-1β, soluble FAS, soluble IL-6R, TGFβ-1, TIM3, TNF-α, TNFRp55, and TNFRp75 concentrations. The serum IL-8 concentration on day 0 was higher in tFL compared to FL patients (9.6 vs 2.5 pg/mL, P=0.01). Patients with tFL had higher pre-treatment LDH, which could reflect more aggressive disease and a more immunosuppressive tumor microenvironment (Hallek et al., Blood 131:2745 (2018); Cheson et al., J. Clin. Onol. 32:3059 (2014); Lee et al., Blood 124:188 (2014); Porter et al., Sci. Transl. Med. 7:303ra139 (2015)). These data suggest that differences in the tumor microenvironment may contribute to differences in outcomes in FL and tFL patients; however, a contribution from the antitumor effect of Cy/Flu lymphodepletion cannot be excluded.

CD19 CAR-T cell immunotherapy was well-tolerated and resulted in a remarkably high CR rate (88%) in patients with clinically aggressive R/R FL, and all patients remained in CR after a single CAR-T cell infusion.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 62/679,468, filed Jun. 1, 2018, U.S. Provisional Patent Application No. 62/751,466, filed Oct. 26, 2018, and U.S. Provisional Patent Application No. 62/754,524, filed Nov. 1, 2018, are incorporated herein by reference, in their entirety. Aspects of the embodiments can be modified, if necessary to employ concepts of the various patents, applications and publications to provide yet further embodiments.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for reducing the risk of relapse of a hematological malignancy in a human subject presenting with a Minimal Residual Disease-Negative Complete Response following administration to the subject of a first therapy comprising lymphodepleting chemotherapy and one or more infusion of modified immune cells containing a heterologous polynucleotide that encodes a binding protein that specifically binds to an antigen expressed by or associated with the hematological malignancy, the method comprising:
(a) measuring a pre-first-therapy biomarker level in the subject, wherein the biomarker is selected from serum lactate dehydrogenase (LDH), platelets, or both; and
(b) identifying the subject as being at-risk of relapse when the subject:
(i) has a pre-first-therapy serum LDH level of about 210 U/L or more;
(ii) does not receive a lymphodepleting chemotherapy comprised of cyclophosphamide and fludarabine;
(iii) has a pre-first-therapy platelet count of less than about 100 U/L;
(iv) has pre-first-therapy extramedullary disease;
(v) has a pre-first-therapy International Prognostic Index (IPI) of 2, 3, or 4;
(vi) has one or more diseased cells prior to and following the one or more infusions of modified immune cells, wherein the one or more diseased cells are optionally from the subject's bone marrow, wherein the one or more diseased cells are optionally identified by a mutant nucleotide sequence from at least a portion of an IgH gene; an IgK gene; a TRB gene; a TRD gene; a TRG gene, or any combination thereof;
(vii) does not receive an allogeneic hematopoietic stem cell transplant (allo-HSCT);
(viii) has a peak serum concentration of IL-7 by about 28 days following the one or more infusions of the modified immune cells at about the same or a lower concentration than the serum concentration of IL-7 immediately prior to a first of the one or more infusions;
(ix) has a peak serum concentration of IL-7 by about 28 days following the one or more infusions of the modified immune cells of less than about 20 pg/mL;
(x) has a serum concentration of MCP-1 immediately prior to a first of the one or more infusions of the modified immune cells of less than about 10³ pg/mL;
(xi) has a serum concentration of MCP-1 immediately prior to a first of the one or more infusions of the modified immune cells that is lower than, or that is up to about 20% greater than, the serum concentration of MCP-1 at the time of a first administration of the lymphodepleting chemotherapy;
(xii) has received the cyclophosphamide at one or more doses of less than about 40 mg/kg;
(xiii) has received a total dose of the cyclophosphamide of less than about 1500 mg/m²;
(xiv) has a peak serum concentration of IL-18 by about 28 days following the one or more infusions of the modified immune cells of at least about 10³ pg/mL; or
(xv) any combination of b(i)-b(xiv),
wherein the at-risk subject is identified as a candidate for a second therapy to reduce the risk of relapse.

2. The method of claim 1, wherein the second therapy comprises:
(i) allogeneic hematopoietic stem cell transplant;
(ii) radiation therapy;
(iii) chemotherapy;
(iv) surgery;
(v) one or more further infusion of the modified immune cell;
(vi) immunosuppressive therapy; or
(vii) any combination of (i)-(vi).

3. A method for treating a hematological malignancy in a human subject, wherein the subject had previously been administered lymphodepleting chemotherapy and one or more infusion of modified immune cells containing a heterologous polynucleotide that encodes a binding protein that specifically binds to an antigen expressed by or associated with the hematological malignancy, wherein the subject presents with a Minimal Residual Disease-Negative Complete Response following the one or more infusion, the method comprising:
(a) administering an allogeneic hematopoietic stem cell transplant (allo-HSCT) to the subject and/or monitoring the subject when the subject:
(i) has a serum lactate dehydrogenase (LDH) level of less than about 210 U/L prior to receiving the lymphodepleting chemotherapy;
(ii) has received a lymphodepleting chemotherapy comprised of cyclophosphamide and fludarabine, wherein the cyclophosphamide is administered at one or more doses of at least about 60 mg/kg, or wherein the lymphodepleting chemotherapy comprises a total of the cyclophosphamide of at least about 3,000 mg/m²;
(iii) has a platelet count of about 100 U/L or more prior to receiving the lymphodepleting chemotherapy;
(iv) has an increased level of serum MCP-1 prior to receiving the one or more infusions;
(v) has an International Prognostic Index (IPI) of 0 or 1 prior to receiving the lymphodepleting chemotherapy and the one or more infusions;
(vi) has a reduced level of IL-18 when receiving the one or more infusions;
(vii) has a peak serum concentration of IL-7 by about 28 days following the one or more infusions of the modified immune cells that is higher than the serum concentration of IL-7 immediately prior to a first of the one or more infusions;
(viii) has a peak serum concentration of IL-7 by about 28 days following the one or more infusions of the modified immune cells of at least about 20 pg/mL;
(ix) has a serum concentration of MCP-1 immediately prior to a first of the one or more infusions of the modified immune cells of at least about 10³ pg/mL, or lower
(x) has a serum concentration of MCP-1 immediately prior to a first of the one or more infusions of the modified immune cells that is at least about 20% greater than the serum concentration of MCP-1 at the time of a first administration of the lymphodepleting chemotherapy;
(xi) has one or more diseased cells prior to, but not following, the one or more infusions of modified immune cells, wherein the one or more diseased cells are optionally from the subject's bone marrow, wherein the one or more diseased cells are optionally identified by a mutant nucleotide sequence from at least a portion of an IgH gene; an IgK gene; a TRB gene; a TRD gene; a TRG gene, or any combination thereof;
(xii) has received allo-HSCT following the one or more infusions of the modified immune cells, or
(xiii) has any combination of (a)(i)-(a)(xii); and
(b) administering a therapy comprising allo-HSCT; radiation therapy; chemotherapy; surgery; one or more further infusion of the modified immune cells; immunosuppressive therapy; or any combination thereof, when the subject:
(i) has a pre-first-therapy serum LDH level of about 210 U/L or more;
(ii) did not receive a lymphodepleting chemotherapy comprised of cyclophosphamide and fludarabine;
(iii) has a pre-first-therapy platelet count of less than about 100 U/L;
(iv) has pre-first-therapy extramedullary disease;
(v) has a pre-first-therapy International Prognostic Index (IPI) of 2, 3, or 4;
(vi) has one or more diseased cells prior to and following the one or more infusions of modified immune cells, wherein the one or more diseased cells are optionally from the subject's bone marrow, wherein the one or more diseased cells are optionally identified by a nucleotide sequence from at least a portion of an IgH gene; an IgK gene; a TRB gene; a TRD gene; a TRG gene, or any combination thereof;
(vii) did not receive an allogeneic hematopoietic stem cell transplant (allo-HSCT);
(viii) has a peak serum concentration of IL-7 by about 28 days following the one or more infusions of the modified immune cells at about the same as or lower than the serum concentration of IL-7 immediately prior to a first of the one or more infusion;
(ix) has a peak serum concentration of IL-7 by about 28 days following the one or more infusions of the modified immune cells of less than about 20 pg/mL;
(x) has a serum concentration of MCP-1 immediately prior to a first of the one or more infusions of the modified immune cells of less than about 10³ pg/mL;
(xi) has a serum concentration of MCP-1 immediately prior to a first of the one or more infusions of the modified immune cells that is lower than, or that is up to about 20% greater than, the serum concentration of MCP-1 at the time of a first administration of the lymphodepleting chemotherapy;
(xii) has received the cyclophosphamide at one or more doses of less than about 40 mg/kg;
(xiii) has received a total dose of the cyclophosphamide of less than about 1500 mg/m²;
(xiv) has a peak serum concentration of IL-18 by about 28 days following the one or more infusions of the modified immune cells of at least about 10³ pg/mL; or
(xv) any combination of b(i)-b(xiv),
wherein the at-risk subject is identified as a candidate for a second therapy to reduce the risk of relapse.

4. The method of any one of claims 1-3, wherein the hematological malignancy is selected from acute lymphoblastic leukemia (ALL), optionally B cell ALL, Hodgkin's lymphoma, non-Hodgkins lymphoma (NHL), primary central nervous system lymphomas, T cell lymphomas, small lymphocytic lymphoma (SLL), B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma (mucosa-associated lymphoid tissue (MALT) lymphoma), nodal marginal zone B-cell lymphoma, follicular lymphoma, mantle cell lymphoma, diffuse large B-cell lymphoma (DLBCL), mediastinal (thymic) large B-cell lymphoma, intravascular large B-cell lymphoma, primary effusion lymphoma, acute myeloid leukemia (AML), chronic lymphocytic leukemia (CLL), chronic myoblastic leukemia (CIVIL), Hairy cell leukemia (HCL), chronic myelomonocytic leukemia (CMML), juvenile myelomonocytic leukemia (JMML), large granular lymphocytic leukemia (LGL), blastic plasmacytoid dendritic cell neoplasm (BPDCN), Burkitt lymphoma/leukemia, T cell/histiocyte-rich large B cell lymphoma, pleomorphic mantle cell lymphoma, multiple myeloma, Bence-Jones myeloma, non-secretory myeloma, plasmacytoma, amyloidosis, monoclonal gammopathy of unknown significance (MGUS), or Waldenstrom's macroglobulinemia.

5. The method of claim 4, wherein the hematological malignancy is B cell ALL.

6. The method of claim 5, wherein the allo-HSCT is administered to the subject when the subject:
(i) has a serum LDH level of less than about 210 U/L prior to receiving the lymphodepleting chemotherapy;
(ii) has received cyclophosphamide and fludarabine as the lymphodepleting chemotherapy; and/or
(iii) has a platelet count of about 100 U/L or more prior to receiving the lymphodepleting chemotherapy.

7. The method of claim 5 or 6, wherein the therapy is administered to the subject when the subject:
(i) has a serum LDH level of about 210 U/L or more prior to receiving the lymphodepleting chemotherapy;
(ii) did not receive lymphodepleting chemotherapy comprising cyclophosphamide and fludarabine;
(iii) has a platelet count of less than about 100 U/L prior to receiving the lymphodepleting chemotherapy; and/or
(iv) has one or more diseased cells prior to, but not following, the one or more infusions of modified immune cells, wherein the one or more diseased cells are optionally from the subject's bone marrow, wherein the one or more diseased cells are optionally identified by a mutant nucleotide sequence from at least a portion of an IgH gene; an IgK gene; a TRB gene; a TRD gene; a TRG gene, or any combination thereof.

8. The method of claim 4, wherein the hematological malignancy is NHL.

9. The method of claim 8, wherein the allo-HSCT is administered to the subject and/or the subject is monitored when the subject:
(i) has a serum LDH level of less than about 210 U/L prior to receiving the lymphodepleting chemotherapy;
(ii) has an increased level of serum MCP-1 prior to receiving the one or more infusion;
(iii) has an International Prognostic Index (IPI) of 0 or 1 prior to receiving the lymphodepleting chemotherapy and the one or more infusion;
(vi) has a reduced level of IL-18 prior to receiving the lymphodepleting chemotherapy and prior to the subject receiving the one or more infusions;
(v) has an increased peak level of IL-7 prior to receiving the lymphodepleting chemotherapy;
(vi) has an increased level of serum IL-7 prior to receiving the lymphodepleting chemotherapy;
(vii) has a peak serum concentration of IL-7 by about 28 days following the one or more infusions of the modified immune cells of less than about 20 pg/mL;
(viii) has a serum concentration of MCP-1 immediately prior to a first of the one or more infusions of the modified immune cells of less than about 10³ pg/mL;
(vi) has a serum concentration of MCP-1 immediately prior to a first of the one or more infusions of the modified immune cells that is lower than, or that is up to about 20% greater than, the serum concentration of MCP-1 at the time of a first administration of the lymphodepleting chemotherapy;
(ix) has received the cyclophosphamide at one or more dose of less than about 40 mg/kg; and/or
(x) has received a total dose of the cyclophosphamide of less than about 1500 mg/m².

10. The method of claim 8 or 9, wherein the therapy is administered to the subject when the subject had a serum LDH level of 210 U/L or more prior to receiving the lymphodepleting chemotherapy.

11. A method for treating a hematological malignancy in a human subject, wherein the subject had received a first therapy comprising a lymphodepleting chemotherapy and one or more infusion of a modified immune cell comprising a heterologous polynucleotide that encodes a binding protein that specifically binds to an antigen expressed by or associated with the hematological malignancy, the method comprising administering a second therapy comprising allogeneic hematopoietic stem cell transplant; radiation therapy; chemotherapy; surgery; one or more further infusion of the modified immune cell; immunosuppressive therapy; or any combination thereof, when:
(a) by about 28 days following the one or more infusion, the subject had a peak serum concentration of the heterologous polynucleotide encoding the binding protein of about 10² or fewer copies per microgram of DNA;
(b) by about 28 days following the one or more infusion, the subject had a peak serum concentration of the modified immune cell of about 10¹ or fewer cells per microliter, as determined by flow cytometry;
(c) prior to receiving the lymphodepleting chemotherapy, the subject has a serum lactate dehydrogenase (LDH) level of 210 U/L or more;
(d) the subject has a peak serum concentration of IL-7 by about 28 days following the one or more infusions of the modified immune cells of less than about 20 pg/mL;
(e) the subject has a serum concentration of MCP-1 immediately prior to a first of the one or more infusions of the modified immune cells of less than about 10³ pg/mL;
(f) the subject has a serum concentration of MCP-1 immediately prior to a first of the one or more infusions of the modified immune cells that is lower than, or that is up to about 20% greater than, the serum concentration of MCP-1 at the time of a first administration of the lymphodepleting chemotherapy;
(g) the subject has received the cyclophosphamide at one or more doses of less than about 40 mg/kg; and/or
(h) the subject has received a total dose of the cyclophosphamide of less than about 1500 mg/m².

12. The method of claim 11, wherein the hematological malignancy is selected from acute lymphoblastic leukemia (ALL), optionally B cell ALL, Hodgkin's lymphoma, non-Hodgkins lymphoma (NHL), primary central nervous system lymphomas, T cell lymphomas, small lymphocytic lymphoma (SLL), B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma (mucosa-associated lymphoid tissue (MALT) lymphoma), nodal marginal zone B-cell lymphoma, follicular lymphoma, mantle cell lymphoma, diffuse large B-cell lymphoma (DLBCL), mediastinal (thymic) large B-cell lymphoma, intravascular large B-cell lymphoma, primary effusion lymphoma, acute myeloid leukemia (AML), chronic lymphocytic leukemia (CLL), chronic myoblastic leukemia (CML), Hairy cell leukemia (HCL), chronic myelomonocytic leukemia (CMML), juvenile myelomonocytic leukemia (JMML), large granular lymphocytic leukemia (LGL), blastic plasmacytoid dendritic cell neoplasm (BPDCN), Burkitt lymphoma/leukemia, multiple myeloma, Bence-Jones myeloma, non-secretory myeloma, plasmacytoma, amyloidosis, monoclonal gammopathy of unknown significance (MGUS), T cell/histiocyte-rich large B cell lymphoma, pleomorphic mantle cell lymphoma, or Waldenstrom's macroglobulinemia.

13. The method of claim 12, wherein the hematological malignancy is B-ALL.

14. The method of claim 12, wherein the hematological malignancy is NHL.

15. The method of any one of claims 1-14, wherein the encoded binding protein comprises a chimeric antigen receptor (CAR).

16. The method of claim 15, wherein the encoded binding protein comprises a CAR comprising an extracellular component comprising a binding domain specific for the antigen and a hinge region, an intracellular component, and a transmembrane component disposed between the extracellular component and the intracellular component, wherein the hinge region is disposed between the binding domain and the transmembrane component.

17. The method of any one of claims 1-16, wherein the antigen is CD19.

18. The method of claim 17, wherein:
the binding domain of the encoded binding protein is derived from FMC-63 antibody, MOR208, blinatumomab, MEDI-551, Merck patent anti-CD19 antibody, Xmab5871, or MDX-1342; and/or
the hinge region is derived from IgG4; and/or
the transmembrane component is derived from CD28; and/or
the intracellular component comprises a 4-1BB signaling domain and a CD3ζ domain.

19. The method of any one of claims 1-18, wherein one or more of the infusions comprises modified CD4+ T cells and modified CD8+ T cells in about a 1:1 ratio.

20. The method of claim 19, wherein one or more of the infusions comprises modified CD4+ T cells and modified CD8+ T cells in a 1:1 ratio.

21. The method any one of claims 1-20, wherein the subject has previously received one or more infusion comprising 2×10⁵ to 2×10⁶ of the modified immune cells/kg.

22. A kit for use in diagnosing or detecting the risk of a relapse of a hematological malignancy in a subject that presents with a MRD-negative CR following administration to the subject of lymphodepleting chemotherapy and one or more infusion of modified immune cells containing a heterologous polynucleotide that encodes a binding protein that specifically binds to an antigen expressed by or associated with the hematological malignancy, the kit comprising one or more reagent for:
(i) measuring the amount of LDH in a serum sample from the subject;
(ii) measuring the amount of platelets present in a serum sample from the subject;
(iii) measuring a serum concentration of IL-7 in a sample from the subject; and/or
(iv) measuring a serum concentration of MCP-1 in a sample from the subject, wherein the subject is identified as being at risk of relapse when the subject:
(a) has a serum LDH level of about 210 or more U/L prior to receiving the lymphodepleting chemotherapy and/or
(b) has a platelet count of less than about 100 U/L prior to receiving the lymphodepleting chemotherapy;
(c) has a peak serum concentration of IL-7 by about 28 days following the one or more infusions of the modified immune cells of less than about 20 pg/mL;
(d) has a serum concentration of MCP-1 immediately prior to a first of the one or more infusions of the modified immune cells of less than about 10³ pg/mL; and/or
(e) has a serum concentration of MCP-1 immediately prior to a first of the one or more infusions of the modified immune cells lower than, or up to about 20% greater than, the serum concentration of MCP-1 at the time of a first administration of the lymphodepleting chemotherapy.

23. The kit of claim 22, further comprising one or more reagent for measuring the amount of the heterologous polynucleotide in a serum sample from the subject.

24. The kit of claim 22 or 23, further comprising instructions for performing the measuring.

25. The kit of any one of claims 22-24, further comprising instructions for providing a pre-emptive therapy when the subject is identified as being at-risk for relapse, wherein the pre-emptive therapy comprises allogeneic hematopoietic stem cell transplant; radiation therapy; chemotherapy; surgery; one or more further infusion of the modified immune cells; immunosuppressive therapy; or any combination thereof.

26. A method for treating a hematological malignancy in a human subject, the method comprising administering to the subject an effective amount of a Bruton's Tyrosine Kinase (BTK) inhibitor, wherein the subject is receiving or has received a modified immune cell containing a heterologous polynucleotide that encodes a binding protein that specifically binds to an antigen expressed by or associated with the hematological malignancy.

27. A method for treating a hematological malignancy in a human subject who is receiving or has received a Bruton's Tyrosine Kinase (BTK) inhibitor, the method comprising administering to the subject an effective amount of a modified immune cell containing a heterologous polynucleotide that encodes a binding protein that specifically binds to an antigen expressed by or associated with the hematological malignancy.

28. The method of claim 26 or 27, wherein the subject receives the BTK inhibitor for about one week to about six months, or longer, following a first administration of the modified immune cell to the subject.

29. The method of any one of claims 26-28, wherein the subject had received the BTK inhibitor for at least about one week to about five years prior to a first administration of the modified immune cell to the subject.

30. The method of any one of claims 26-28, wherein the hematological malignancy is selected from chronic lymphocytic leukemia (CLL), non-Hodgkin's lymphoma (NHL), mantle cell lymphoma, acute lymphoblastic leukemia (ALL), optionally B cell ALL, small lymphocytic lymphoma (SLL), Hodgkin's lymphoma, primary central nervous system lymphomas, T cell lymphomas, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma (mucosa-associated lymphoid tissue (MALT) lymphoma), nodal marginal zone B-cell lymphoma, follicular lymphoma, diffuse large B-cell lymphoma (DLBCL), mediastinal (thymic) large B-cell lymphoma, intravascular large B-cell lymphoma, primary effusion lymphoma, acute myeloid leukemia (AML), chronic myoblastic leukemia (CIVIL), Hairy cell leukemia (HCL), chronic myelomonocytic leukemia (CMML), chronic myeloid leukemia, juvenile myelomonocytic leukemia (JMML), large granular lymphocytic leukemia (LGL), blastic plasmacytoid dendritic cell neoplasm (BPDCN), Burkitt lymphoma/leukemia, multiple myeloma, Bence-Jones myeloma, non-secretory myeloma, plasmacytoma, amyloidosis, myelodysplastic syndrome (MDS), monoclonal gammopathy of unknown significance (MGUS), or Waldenstrom's macroglobulinemia.

31. The method of claim 30, wherein the hematological malignancy is CLL.

32. The method of any one of claims 27-31, wherein the subject had experienced a progression and/or a relapse of the hematological malignancy while receiving the BTK inhibitor and prior to administration of the modified immune cell.

33. The method of any one of claims 27-32, wherein the subject is intolerant of the BTK inhibitor.

34. The method of any one of claims 27-33, wherein the hematological malignancy is refractory to the BTK inhibitor.

35. The method of any one of claims 27-34, wherein the BTK inhibitor comprises ibrutinib, acalabrutinib (ACP-196), ONO-4059 (GS4059), spebrutinib, BGB-3111, HM71224, or any combination thereof.

36. The method of claim 35, wherein the BTK inhibitor comprises ibrutinib.

37. The method of claim 35 or 36, wherein the subject had received ibrutinib for about 6, 7, 8, 9, 10, 11, 12, 13, 14, 15, 16, 17, 18, 29, 20, 21, 22, 23, 24, 25, 26, 27, 28, 29, 30, 31, 32, 33, 34, 35, 36, or more months prior to experiencing a progression of the hematological malignancy.

38. The method of any one of claims 26-37, wherein, prior to receiving the modified immune cell, the subject had a maximum Standard Uptake Value (SUV) of about 3, 4, 5, 6, 7, 8, 9, 10, 11, or more, wherein the SUV was optionally determined by positron emission tomography (PET) and/or x-ray computerized tomography (CT) comprising use of a labeled tracer molecule, wherein the labeled tracer molecule optionally comprised a radiolabeled tracer molecule, optionally 2-deoxy-2-[¹⁸F]fluoro-D-glucose (FDG).

39. The method of any one of claims 26-38, wherein prior to receiving the modified immune cell, the subject:
(i) had a complex karyotype, optionally comprising a chromosome 17p deletion, a chromosome 11q deletion, a chromosome 13q deletion, trisomy 12, a TP53 stop or missense mutation, 3, 4, 5, or more distinct chromosomal abnormalities present in more than one metaphase, or any combination thereof;
(ii) had a mutation in a BTK gene that affects the ability of ibrutinib to bind to BTK, wherein the mutation is optionally a substitution mutation at position C481, wherein the substitution mutation is optionally C481S;
(iii) had a gain-of-function (GOF) mutation in a PLCG2 gene, wherein the GOF mutation optionally comprises R665W, L845F, S707Y, or any combination thereof
(iv) had a serum LDH concentration of about 130 U/L, 140 U/L, 150 U/L, 160 U/L, 170 U/L, 180 U/L, 190 U/L, 200 U/L, 210 U/L, 220 U/L, 230 U/L, 240 U/L, 250 U/L, 260 U/L, 270 U/L, 280 U/L, 290 U/L, 300 U/L, 310 U/L, or more;
(v) had bulky disease;
(vi) had extensive nodal involvement of the hematological malignancy;
(vii) had palpable lymph nodes and/or infiltration of cells of the hematological malignancy in other organs or tissues;
(viii) had malignant cells not confined to bone marrow;
(ix) had palpable nodes greater than 5 cm in diameter and/or had a palpable spleen greater than 6 cm below the costal margin;
(x) had a high-risk histology, optionally characterized by Richter's transformation, prolymphocytic leukemia, IPC, or any combination thereof;
(xi) had extramedullary disease;
(xii) had about 10%, 15%, 20%, 25%, 30%, 35%, 40%, 45%, 50%, 55%, 60%, 65%, 70%, 75%, or 80% malignant cells in bone marrow, or more;
(xiii) had about 5%, 10%, 15%, 20%, 25%, 30%, 35%, 40%, 45%, 50%, 55%, or 60% malignant cells in blood, or more;
(xiv) had about 1×10⁹, 2×10⁹, 3×10⁹, 4×10⁹, 5×10⁹, 6×10⁹, 7×10⁹, 8×10⁹, 9×10⁹, or more malignant cells/L blood;
(xv) had about 1×10⁹, 2×10⁹, 3×10⁹, 4×10⁹, 5×10⁹, 6×10⁹, 7×10⁹, 8×10⁹, 9×10⁹, or more lymphocytes/L blood;
(xvi) had previously received allogeneic stem cell transplantation; or
(xvii) had any combination of (i)-(xvi),
wherein the hematological malignancy is optionally CLL.

40. The method of any one of claims 26-39, wherein the subject had previously been administered venetoclax, rituximab, idelalisib, or any combination thereof.

41. The method of claim 40, wherein the subject had experienced a progression and/or relapse of the hematological malignancy prior to administration of the modified immune cell and while receiving the venetoclax, rituximab, idelaisib, or combination thereof.

42. The method of any one of claims 26-41, wherein the antigen expressed by or associated with the hematological malignancy is a CD19 antigen.

43. The method of any one of claims 26-42, wherein the modified immune cell comprises an autologous immune cell obtained from the subject that is modified to contain the heterologous polynucleotide encoding the binding protein, wherein the subject is administered the BTK inhibitor at least once between the time the autologous immune cell is obtained from the subject and the time the modified immune cell is administered to the subject.

44. The method of any one of claims 26-43, wherein, following the administering of the modified immune cell, the subject has:
(i) a reduced tumor burden as compared to a reference subject who does not receive the modified immune cell;
(ii) a reduced number and/or severity of immune cell-related toxicity events as compared to a reference subject who does not receive the modified immune cell; and/or
(iii) has about the same as, or has an increased number of the modified immune cell as compared to a reference subject who does not receive the modified immune cell.

45. The method of any one of claims 26-44, further comprising performing nucleic acid sequencing on at least a portion of an IgH gene locus in a bone marrow sample from the subject before and/or after administering the modified immune cell.

46. The method of any one of claims 26-45, wherein the subject is disease-negative as determined by sequencing of the at least a portion of the IgH locus after administering the modified immune cell.

47. A method for treating follicular lymphoma (FL) in a subject, the method comprising administering to the subject an effective amount of a modified immune cell containing a heterologous polynucleotide that encodes a binding protein that specifically binds to an antigen expressed by or associated with the FL, wherein the subject had previously received lymphodepleting chemotherapy prior to the modified immune cell, and wherein, following the administering of the modified immune cell, the subject:
(i) is alive for at least 6, 12, 18, 24, 30, 36, or 38 months;
(ii) presents with no progression of the FL for at least 6, 12, 18, 24, 30, 36, or 38 months; and/or
(iii) presents with a complete remission of the FL for at least 6, 12, 18, 24, 30, 36, or 38 months.

48. The method of claim 47, wherein the FL comprises transformed follicular lymphoma (tFL).

49. The method of any claim 47 or 48, wherein the subject had received treatment prior to the lymphodepleting chemotherapy.

50. The method of any one of claims 47-49, wherein the subject had presented with a relapse and/or progression of disease following a prior therapy for the FL, wherein the prior therapy comprises a biological agent, a chemotherapy, a hematopoietic stem cell transplantation (HCT), or any combination thereof.

51. The method of any one of claims 47-50, wherein the lymphodepleting chemotherapy comprises cyclophosphamide and/or fludarabine.

52. The method of any one of claims 47-51, wherein, prior to receiving the modified immune cell, the subject has: stage III FL; stage IV FL; extranodal involvement of FL; an intermediate or high FL International Prognostic Index score; MYC and BLC2 and/or BCL6 rearrangement (DH/TH); or any combination thereof.

53. The method of any one of claims 26-52, wherein the encoded binding protein comprises a chimeric antigen receptor (CAR).

54. The method of claim 53, wherein CAR comprises an extracellular component comprising a binding domain specific for the antigen and a hinge region, an intracellular component, and a transmembrane component disposed between the extracellular component and the intracellular component, wherein the hinge region is disposed between the binding domain and the transmembrane component.

55. The method of any one of claim 53 or 54, wherein the antigen is a CD19 antigen and wherein the CAR comprises a binding domain comprising a scFv that specifically binds to the CD19 antigen.

56. The method of any one of claims 53-55, wherein the binding protein is a CAR and:
the binding domain comprises CDRs from, or comprises a VH and/or a VL from, or comprises a VH and/or a VL having a least about 80%, 85%, 90%, 95%, 96%, 97%, 98%, or 99% identity to that of FMC-63 antibody, MOR208, blinatumomab, MEDI-551, Merck patent anti-CD19 antibody, Xmab5871, or MDX-1342. MOR208, blinatumomab, MEDI-551, Merck patent anti-CD19 antibody, Xmab5871, or MDX-1342; and/or
the hinge region is derived from IgG4; and/or
the transmembrane component is derived from CD28; and/or
the intracellular component comprises a 4-1BB signaling domain and a CD3ζ domain.

57. The method of any one of claims 26-56, wherein the modified immune cell comprises a human immune cell.

58. The method of claim 57, wherein the modified immune cell comprises an autologous immune cell from the subject.

59. The method of claim 58, wherein the human immune cell comprises a hematopoietic stem cell, a lymphoid progenitor cell, a T cell, a NK cell, a NK-T cell, a B cell, a myeloid progenitor cell, a monocyte, a macrophage, a dendritic cell, a megakaryocyte, a granulocyte, or any combination thereof.

60. The method of any one of claims 26-59, wherein the modified immune cell comprises modified CD4+ T cells and modified CD8+ T cells in about a 1:1 ratio.

61. The method of claim 60, wherein the modified CD8+ T cells comprise central memory T cells.

62. The method of any one of claims 26-61, wherein the subject is receiving or has received about 2×10⁵, about 2×10⁶, or about 2×10⁷ modified immune cells/kg.

63. The method of any one of claims 26-62, wherein, prior to receiving the modified immune cell, the subject received lymphodepleting chemotherapy.

64. The method of claim 63, wherein the lymphodepleting chemotherapy comprises cyclophosphamide and fludarabine.

65. The method of any one of claims 1-21, wherein the modified immune cells were produced by a method comprising leukapheresis of the subject, wherein the leukapheresis occurred prior to administration of the lymphodepleting chemotherapy, and wherein following the leukapharesis and the prior to the lymphodepleting chemotherapy, the subject received a bridging therapy comprising one or more of:
(i) chemotherapy;
(ii) a corticosteroid, wherein the corticosteroid is optionally dexamethasone;
(iii) a monoclonal antibody or antigen-binding fragment thereof;
(iv) an immunomodulatory agent;
(v) a targeted small molecule chemotherapeutic agent; or
(vi) any combination of (i)-(v).
